(19) United States
(12) Patent Application Publication
(10) Pub. No.: US 2009/0326003 A1
(43) Pub. Date: Dec. 31, 2009

(54) PYRAZOLO (3, 4-B) PYRIDINE DERIVATIVES AS PDE4 INHIBITORS

(76) Inventors: David George Allen, Hertfordshire (GB); Michael Dennis Dowle, Hertfordshire (GB); Christopher David Edlin, Hertfordshire (GB); Leanda Jane Kindon, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/374,331

(22) PCT Filed: Jul. 19, 2007
(86) PCT No.: PCT/EP2007/057493

§ 371 Date: Jan. 19, 2009

(30) Foreign Application Priority Data
Jul. 21, 2006 (GB) 0614570.0

(51) Int. Cl. A61K 31/437; C07D 471/04; A61P 11/06; A61P 17/06

(52) U.S. Cl. 514/303; 546/119

(57) ABSTRACT

The present invention provides a compound of formula (I) or a salt thereof (in particular, a pharmaceutically acceptable salt thereof):

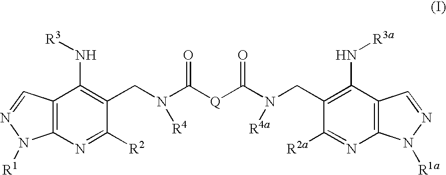

The invention also provides the use of the compounds or salts as inhibitors of phosphodiesterase type IV (PDE4) for the treatment or prophylaxis of inflammatory or allergic diseases such as chronic obstructive pulmonary disease (COPD), asthma, rhinitis, atopic dermatitis or psoriasis, e.g. in a mammal such as a human.

The present invention relates to pyrazolo[3,4-b]pyridine compounds or salts thereof, processes for their preparation, intermediates usable in these processes, and pharmaceutical compositions containing the compounds or salts. The invention also relates to the use of the pyrazolo[3,4-b]pyridine compounds or salts thereof in therapy, for example as inhibitors of phosphodiesterase type IV (PDE4) and/or for the treatment and/or prophylaxis of inflammatory and/or allergic diseases such as chronic obstructive pulmonary disease (COPD), asthma, rhinitis (e.g. allergic rhinitis), atopic dermatitis or psoriasis.

### BACKGROUND TO THE INVENTION

U.S. Pat. No. 3,979,399, U.S. Pat. No. 3,840,546, and U.S. Pat. No. 3,966,746 (E.R. Squibb & Sons) disclose 4-amino derivatives of pyrazolo[3,4-b]pyridine-5-carboxamides wherein the 4-amino group NR₃R₄ can be an acyclic amino group wherein R₃ and R₄ may each be hydrogen, lower alkyl (e.g. butyl), phenyl, etc.; NR₃R₄ can alternatively be a 3-6-membered heterocyclic group such as pyrrolidino, piperidino and piperazino. The compounds are disclosed as central nervous system depressants useful as ataractic, analgesic and hypotensive agents.

U.S. Pat. No. 3,925,388, U.S. Pat. No. 3,856,799, U.S. Pat. No. 3,833,594 and U.S. Pat. No. 3,755,340 (E.R. Squibb & Sons) disclose 4-amino derivatives of pyrazolo[3,4-b]pyridine-5-carboxylic acids and esters. The 4-amino group NR₃R₄ can be an acyclic amino group wherein R₃ and R₄ may each be hydrogen, lower alkyl (e.g. butyl), phenyl, etc.; NR₃R₄ can alternatively be a 5-6-membered heterocyclic group in which an additional nitrogen is present such as pyrrolidino, piperidino, pyrazolyl, pyrimidinyl, pyridazinyl or piperazinyl. The compounds are mentioned as being central nervous system depressants useful as ataractic agents or tranquilizers, as having antiinflammatory and analgesic properties. The compounds are mentioned as increasing the intracellular concentration of adenosine-3′,5′-cyclic monophosphate and for alleviating the symptoms of asthma.

H. Hoehn et al., J. Heterocycl. Chem., 1972, 9(2), 235-253 discloses a series of 1H-pyrazolo[3,4-b]pyridine-5-carboxylic acid derivatives with 4-hydroxy, 4-chloro, 4-alkoxy, 4-hydrazino, and 4-amino substituents. Ethyl 4-(n-butylamino)-1-ethyl-1H-pyrazolo[3,4-b]-pyridine-5-carboxylate is disclosed therein; this compound is cartazolate.

The compound tracazolate, ethyl 4-(n-butylamino)-1-ethyl-6-methyl-1H-pyrazolo[3,4-b]-pyridine-5-carboxylate, is known as an anxiolytic agent (e.g. see J. B. Patel et al., Eur. J. Pharmacol., 1982, 78, 323). Other 1-substituted 4-(NH₂ or NH-alkyl)-1H-pyrazolo[3,4-b]-pyridine-5-carboxylic acid esters and amides are disclosed as potential anxiolytic agents in T. M. Bare et al., J. Med. Chem., 1989, 32, 2561-2573.

CA 1003419, CH 553 799 and T. Denzel, Archiv derPharmazie, 1974, 307(3), 177-186 disclose 4,5-disubstituted 1H-pyrazolo[3,4-b]pyridines unsubstituted at the 1-position.

Japanese laid-open patent application IP-2002-20386-A (Ono Yakuhin Kogyo K K) published on 23 Jan. 2002 discloses pyrazolopyridine compounds of the following formula:

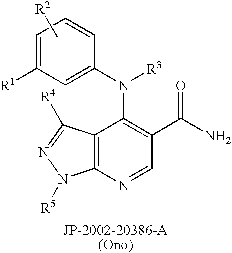

wherein R¹ denotes 1) a group —OR⁶, 2) a group —SR⁷, 3) a C2-8 alkynyl group, 4) a nitro group, 5) a cyano group, 6) a C1-8 alkyl group substituted by a hydroxy group or a C1-8 alkoxy group, 7) a phenyl group, 8) a group —C(O)R⁸, 9) a group —SO₂NR⁹R¹⁰, 10) a group —NR¹¹SO₂R¹², 11) a group —NR¹³C(O)R¹⁴ or 12) a group —CH═NR¹⁵. R⁶ and R⁷ denote i) a hydrogen atom, ii) a C1-8 alkyl group, iii) a C1-8 alkyl group substituted by a C1-8 alkoxy group, iv) a trihalomethyl group, v) a C3-7 cycloalkyl group, vi) a C1-8 alkyl group substituted by a phenyl group or vii) a 3-15 membered mono-, di- or tricyclic hetero ring containing 1-4 nitrogen atoms, 1-3 oxygen atoms and/or 1-3 sulphur atoms. R² denotes 1) a hydrogen atom or 2) a C1-8 alkoxy group. R³ denotes 1) a hydrogen atom or 2) a C1-8 alkyl group. R⁴ denotes 1) a hydrogen atom, 2) a C1-8 alkyl group, 3) a C3-7 cycloalkyl group, 4) a C1-8 alkyl group substituted by a C3-7 cycloalkyl group, 5) a phenyl group which may be substituted by 1-3 halogen atoms or 6) a 3-15 membered mono-, di- or tricyclic hetero ring containing 1-4 nitrogen atoms, 1-3 oxygen atoms and/or 1-3 sulphur atoms. R⁵ denotes 1) a hydrogen atom, 2) a C1-8 alkyl group, 3) a C3-7 cycloalkyl group, 4) a C1-8 alkyl group substituted by a C3-7 cycloalkyl group or 5) a phenyl group which may be substituted by 1-3 substituents. In group R³, a hydrogen atom is preferred. In group R⁴, methyl, ethyl, cyclopropyl, cyclobutyl or cyclopentyl are preferred. The compounds of IP-2002-20386-A are stated as having PDE4 inhibitory activity and as being useful in the prevention and/or treatment of inflammatory diseases and many other diseases.

1,3-Dimethyl-4-(arylamino)-pyrazolo[3,4-b]pyridines with a 5-C(O)NH₂ substituent similar or identical to those in IP-2002-20386-A were disclosed as orally active PDE4 inhibitors by authors from Ono Pharmaceutical Co. in: H. Ochiai et al., Bioorg. Med. Chem. Lett., 2004, vol. 14(1), pp. 29-32. Full papers on these and similar compounds as orally active PDE4 inhibitors are: H. Ochiai et al., Bioorg. Med. Chem., 2004, 12(15), 4089-4100, and H. Ochiai et al., Chem. Pharm. Bull., 2004, 52(9), 1098-1104.

EP 0 076 035 A1 (ICI Americas) discloses pyrazolo[3,4-b]pyridine derivatives as central nervous system depressants useful as tranquilizers or ataractic agents for the relief of anxiety and tension states.

J. W. Daly et al., Med. Chem. Res., 1994, 4, 293-306 and D. Shi et al., Drug Development Research, 1997, 42, 41-56 disclose a series of 4-(amino)substituted 1H-pyrazolo[3,4-b]pyridine-5-carboxylic acid derivatives, including ethyl 4-cyclopentylamino-1-methyl-1H-pyrazolo[3,4-b]pyridine-5-carboxylate, and their affinities and antagonist activities at A₁- and A_{2A}-adenosine receptors, and the latter paper discloses their affinities at various binding sites of the GABA_{A}-receptor channel. S. Schenone et al., Bioorg. Med. Chem. Lett., 2001, 11, 2529-2531, and F. Bondavalli et al., J. Med. Chem., 2002, 45(22), pp. 4875-4887 disclose a series of 4-amino-1-(2-chloro-2-phenylethyl)-1H-pyrazolo[3,4-b]pyridine-5-carboxylic acid ethyl esters as A₁-adenosine receptor ligands.

WO 02/060900 A2 appears to disclose, as MCP-1 antagonists for treatment of allergic, inflammatory or autoimmune disorders or diseases, a series of bicyclic heterocyclic compounds with a —C(O)—NR⁴—C(O)—NR⁵R⁶ substituent, including isoxazolo[5,4-b]pyridines and 1H-pyrazolo[3,4-b]pyridines (named as pyrazolo[5,4-b]pyridines) with the —C(O)—NR⁴—C(O)—NR⁵R⁶ group as the 5-substituent and optionally substituted at the 1-, 3-, 4-, and/or 6-positions. Bicyclic heterocyclic compounds with a —C(O)NH₂ substituent instead of the —C(O)—NR⁴—C(O)—NR⁵R⁶ substituent are alleged to be disclosed in WO 02/060900 as intermediates in the synthesis of the —C(O)—NR⁴—C(O)—NR⁵R⁶ substituted compounds. See also WO 02/081463 A1 for similar MCP-1 antagonists.

WO 00/15222 (Bristol-Myers Squibb) discloses inter alia pyrazolo[3,4-b]pyridines having inter alia a C(O)—X₁ group at the 5-position and a group E₁ at the 4-position of the ring system. Amongst other things, X₁ can for example be —OR₉, —N(R₉)(R₁₀) or —N(R₅)(-A₂-R₂), and E₁ can for example be —NH-A₁-cycloalkyl, —NH-A₁-substituted cycloalkyl, or —NH-A₁-heterocyclo; wherein A₁ is an alkylene or substituted alkylene bridge of 1 to 10 carbons and A₂ can for example be a direct bond or an alkylene or substituted alkylene bridge of 1 to 10 carbons. The compounds are disclosed as being useful as inhibitors of cGMP phosphodiesterase, especially PDE type V, and in the treatment of various cGMP-associated conditions such as erectile dysfunction. Compounds with a cycloalkyl or heterocyclo group directly attached to —NH— at the 4-position of the pyrazolo[3,4-b]pyridine ring system and/or having PDE4 inhibitory activity do not appear to be disclosed in WO 00/15222.

H. de Mello, A. Echevarria, et al., J. Med. Chem., 2004, 47(22), 5427-5432, discloses 3-methyl or 3-phenyl 4-anilino-1H-pyrazolo[3,4-b]pyridine 5-carboxylic esters as potential anti-Leishmania drugs.

WO 2004/056823 A1 (PCT/EP2003/014867, filed on 19 Dec. 2003, published on 8 Jul. 2004, Glaxo Group Limited), and incorporated herein by reference in its entirety as though fully set forth, discloses and claims pyrazolo[3,4-b]pyridine compounds or salts thereof with a 4-NR³R^{3a} group (R^{3a} is preferably H) and with a group Het at the 5-position of the pyrazolo[3,4-b]pyridine, wherein Het is usually a 5-membered optionally substituted heteroaryl group. WO 2004/056823 A1 also discloses the use of these compounds as PDE4 inhibitors and for the treatment and/or prophylaxis of inter alia COPD, asthma or allergic rhinitis.

WO 2004/024728 A2 (PCT/EP2003/011814, filed on 12 Sep. 2003, published on 25 Mar. 2004, Glaxo Group Limited), and incorporated herein by reference in its entirety as though fully set forth, discloses pyrazolo[3,4-b]pyridine compounds or salts thereof with a 4-NHR³ group and a 5-C(O)—X group, according to the following formula:

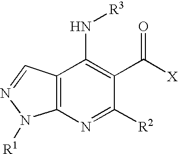

wherein:
R¹ is C₁₋₄alkyl, C₁₋₃fluoroalkyl, —CH₂CH₂OH or —CH₂CH₂CO₂C₁₋₂alkyl;
R² is a hydrogen atom (H), methyl or C₁fluoroalkyl;
R³ is optionally substituted C₃₋₈cycloalkyl or optionally substituted mono-unsaturated-C₅₋₇cycloalkenyl or an optionally substituted heterocyclic group of sub-formula (aa), (bb) or (cc); or R³ is a bicyclic group (dd) or (ee):

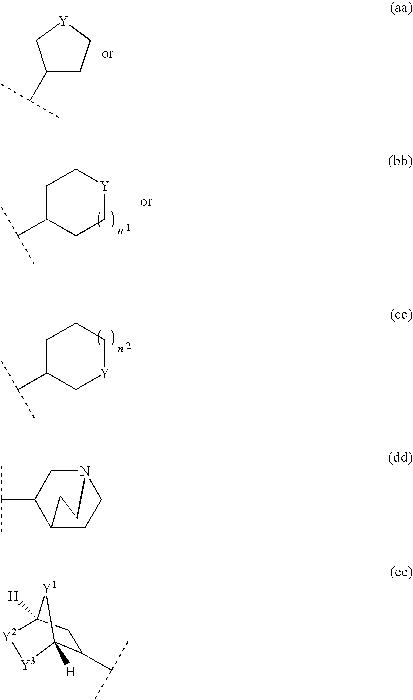

in which n¹ and n² independently are 1 or 2; and in which Y is O, S, SO₂, or NR¹⁰;
and wherein X is NR⁴R⁵ or OR^{5a}.

In WO 2004/024728 A2, R⁴ is a hydrogen atom (H); C₁₋₆alkyl; C₁₋₃fluoroalkyl; or C₂₋₆alkyl substituted by one substituent R¹¹.

In WO 2004/024728 A2, R⁵ can be: a hydrogen atom (H); C₁₋₈alkyl; C₁₋₈ fluoroalkyl; optionally substituted C₃₋₈cycloalkyl; optionally substituted —(CH₂)ₙ⁴—C₃₋₈cycloalkyl wherein n⁴ is 1, 2 or 3; substituted C₂₋₆alkyl; —(CH₂)ₙ¹¹—C(O)R¹⁶; —(CH₂)ₙ¹²—C(O)NR¹²R¹³; —CHR¹⁹—C(O)NR¹²R¹³; —(CH₂)ₙ¹²—C(O)OR¹⁶; —(CH₂)ₙ¹²—C(O)OH; —CHR¹⁹—C(O)OR¹⁶; —CHR¹⁹—C(O)OH; —(CH₂)ₙ¹²—SO₂—NR¹²R¹³; —(CH₂)ₙ¹²—SO₂R¹⁶; or —(CH₂)ₙ¹²—CN; —(CH₂)ₙ¹³—Het; or optionally substituted phenyl; wherein n¹¹ and n¹³ are 0, 1, 2, 3 or 4 and n¹² is 1, 2, 3 or 4.

Alternatively, in WO 2004/024728 A2, R⁵ can have the sub-formula (x), (y), (y1) or (z):

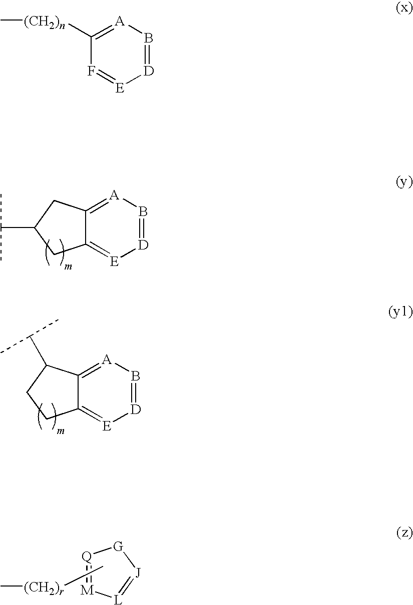

wherein in sub-formula (x), n=0, 1 or 2; in sub-formula (y) and (y1), m=1 or 2; and in sub-formula (z), r=0, 1 or 2; and wherein in sub-formula (x) and (y) and (y1), none, one or two of A, B, D, E and F are independently nitrogen or nitrogen-oxide (N⁺—O—) provided that no more than one of A, B, D, E and F is nitrogen-oxide, and the remaining of A, B, D, E and F are independently CH or CR⁶; and provided that when n is 0 in sub-formula (x) then one or two of A, B, D, E and F are independently nitrogen or nitrogen-oxide (N⁺—O—) and no more than one of A, B, D, E and F is nitrogen-oxide.

In WO 2004/024728 A2, the pyrazolo[3,4-b]pyridine compounds of formula (I) and salts thereof disclosed therein are disclosed as being inhibitors of phosphodiesterase type IV (PDE4), and as being useful for the treatment and/or prophylaxis of a variety of diseases/conditions, especially inflammatory and/or allergic diseases in mammals such as humans, for example: asthma, chronic obstructive pulmonary disease (COPD), atopic dermatitis, urticaria, allergic rhinitis, allergic conjunctivitis, vernal conjunctivitis, eosinophilic granuloma, psoriasis, rheumatoid arthritis, septic shock, ulcerative colitis, Crohn's disease, reperfusion injury of the myocardium and brain, chronic glomerulonephritis, endotoxic shock, adult respiratory distress syndrome, multiple sclerosis, cognitive impairment, depression, or pain. WO 2004/024728 A2 states that the compounds of formula (I) and/or their pharmaceutical compositions may be administered by oral, parenteral, inhaled (topical to the lung), or nasal administration.

WO 2004/024728 has been reviewed, and WO 2004/056823 mentioned, in Expert Opin. Ther. Patents, 2005 (January edition), 15(1), 111-114.

WO 2005/058892 A1 (PCT/EP2004/014490, filed on 17 Dec. 2004, published on 30 Jun. 2005, Glaxo Group Limited), and incorporated herein by reference in its entirety as though fully set forth, discloses pyrazolo[3,4-b]pyridine compounds or salts thereof with a 4-NHR³ group and a 5-C(O)—NH—C(R⁴)(R⁵)—Ar group, wherein at least one of R⁴ and R⁵ are not a hydrogen atom, and discloses the use of these compounds as PDE4 inhibitors and/or for the treatment and/or prophylaxis of inflammatory and/or allergic diseases such as COPD, asthma, rheumatoid arthritis, allergic rhinitis or atopic dermatitis.

Further pyrazolo[3,4-b]pyridine compounds or salts thereof, and their use as PDE4 inhibitors, are disclosed in copending patent applications WO 2005/090353 A1 (PCT/GB2005/000976), WO 2005/090348 A1 (PCT/GB2005/000983), WO 2005/090354 A1 (PCT/GB2005/000987), and WO 2005/090352 A1 (PCT/EP2005/003038) (all Glaxo Group Limited). PCT/EP2005/003038, PCT/GB2005/000987 and PCT/GB2005/000983, all filed on 15 Mar. 2005, are incorporated herein by reference in their entirety as though fully set forth.

### THE INVENTION

We have now found new dimeric pyrazolo[3,4-b]pyridine compounds which inhibit phosphodiesterase type IV (PDE4). In these compounds, two pyrazolo[3,4-b]pyridine moieties, which may be the same or different and which each have a —CH₂—NR^{4/4a}—C(O)— substituent at the 5-position of the pyrazolo[3,4-b]pyridine ring system, are linked by a linker Q via the 5-position substituent.

One or more specific compounds within the presently invented compounds may be suitable for use as PDE4 inhibitors via an inhaled route of administration. Preliminary tests appear to indicate that one or more specific compounds within the presently invented compounds may exhibit a reasonable level of efficacy and/or duration of action as measured by an intratracheal (i.t.) rat LPS-induced neutrophilia model.

The present invention therefore provides a compound of formula (I) or a salt thereof (in particular, a pharmaceutically acceptable salt thereof):

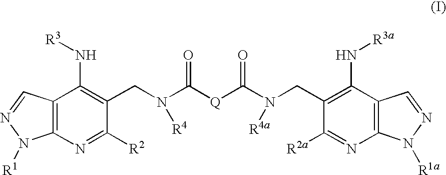

wherein:
Q is —(CH₂)ₘ¹—Ar¹—(CH₂)ₘ²—; —(CMe₂)—Ar²—(CMe₂)-; —(CHMe)—Ar³—(CHMe)-;
—(CH₂)ₘ¹—Ar⁴—O—CH₂—;

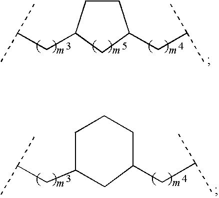

—(CH₂)ₘ⁶—X¹—(CH₂)ₘ⁷—; —(CMe₂)—X²—(CMe₂)-; or —(CHMe)—X³—(CHMe)-;
m¹ is 0, 1 or 2 (such as 0); m² is 0 or 1 (such as 0); m³ is 0 or 1 (such as 0); m⁴ is 0 or 1 (such as 0); and m⁵ is 1 or 2;
m⁶ is 0, 1, 2, 3, 4 or 5 (such as 1 or 2); and m⁷ is 0, 1, 2, 3, 4 or 5 (such as 1 or 2);
X¹ is —CH₂—, —CMe₂-, —CHMe-, O, S(O)₂, or NR⁵ wherein R⁵ is H or C₁₋₃alkyl such as methyl; provided that when X¹ is O or S(O)₂ then m⁶ and m⁷ independently are 1, 2, 3, 4 or 5 (such as 1 or 2);
X² and X³ are independently: a bond, —CH₂—, —(CH₂)₂—, or —(CH₂)₃—;
and Ar¹, Ar², Ar³ and Ar⁴ independently have the sub-formula (x1), (x2), (x3), (x4), (x5), (x6), (x7), (x8), (x9), (x10), (x11), (x12), (x13), (x14), (x15), (x16), (x17), (x18), (x19), (x20), (x21), (x22), (x23), (x24), (x25), (x26), (x27), (x28) or (x29):

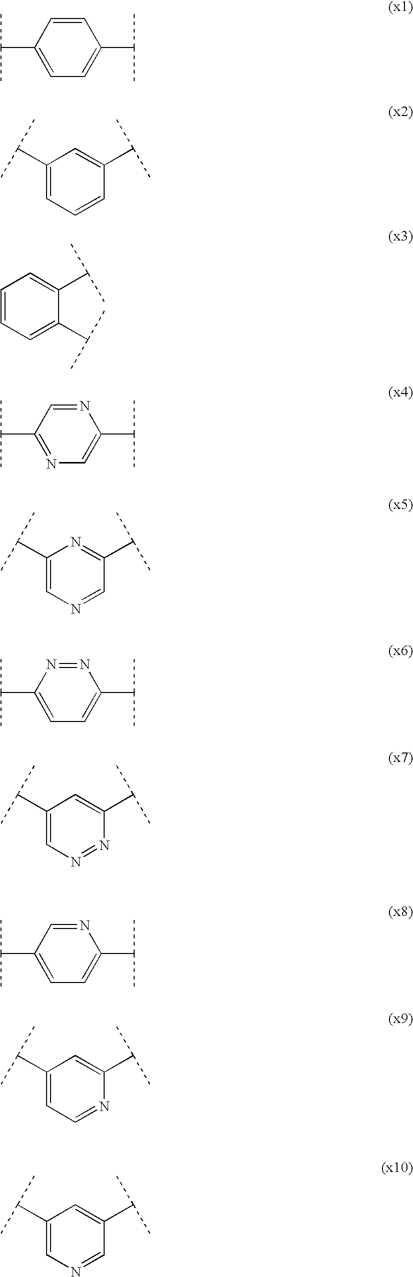

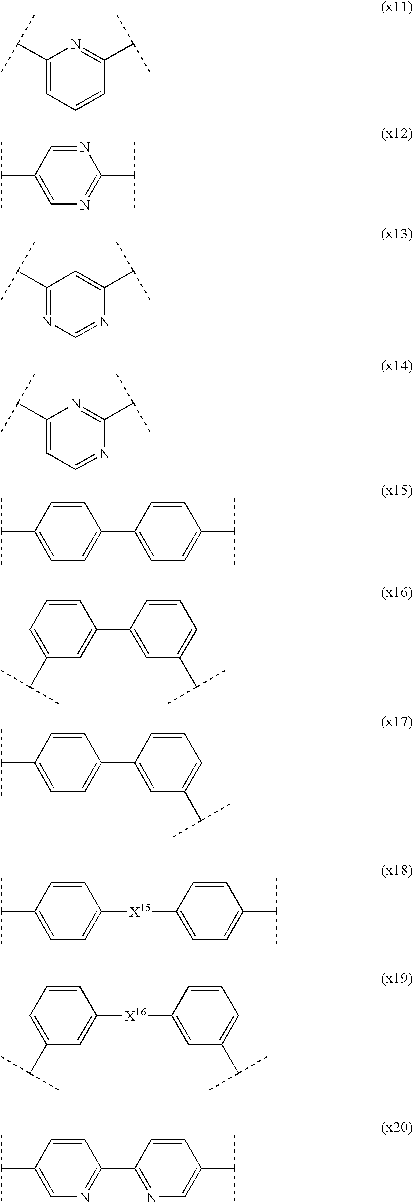

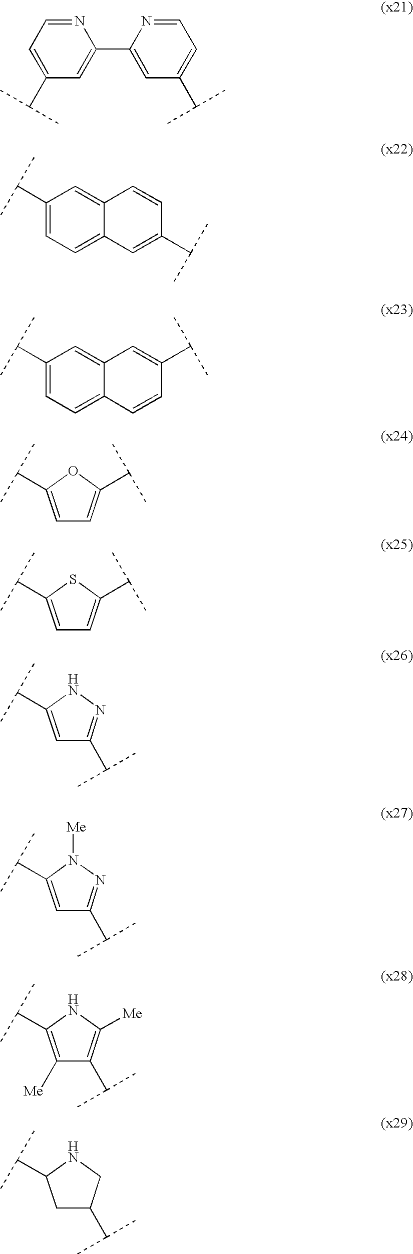

wherein X¹⁵ and X¹⁶ are independently: —CH₂—, —CMe₂-, —CHMe-, —CF₂—, O, C(O), or CHOH;
and wherein:
R¹ and R^{1a} independently are C₁₋₃alkyl, C₁₋₃fluoroalkyl, or —CH₂CH₂OH;
R² and R^{2a} independently are a hydrogen atom (H), methyl, ethyl, n-propyl, isopropyl, n-butyl, C₁₋₂fluoroalkyl, cyclopropyl, cyclobutyl, or (cyclopropyl)methyl-;
R⁴ and R^{4a} independently are a hydrogen atom (H), methyl or ethyl;
R³ and R^{3a} independently are: optionally substituted C₄₋₇cycloalkyl, or optionally substituted mono-unsaturated-C₅₋₇cycloalkenyl, or an optionally substituted heterocyclic group of sub-formula (aa), (bb) or (cc), or a bicyclic group of sub-formula (ee);

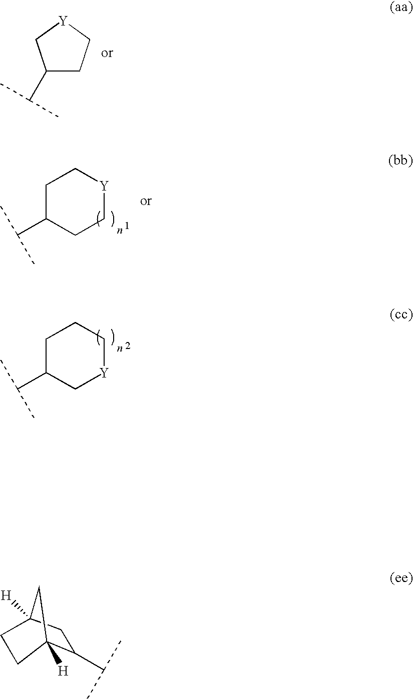

in which n¹ and n² independently are 1 or 2; and in which Y is O, S, SO₂, or NR¹⁰; where R¹⁰ is a hydrogen atom (H), methyl, C(O)NH₂, C(O)-methyl, or C(O)—C₁fluoroalkyl;
and wherein, when R³ and/or R^{3a} is or are optionally substituted C₄₋₇cycloalkyl, then R³ and/or R^{3a} is or are C₄₋₇cycloalkyl optionally substituted on a ring carbon with one or two substituents independently being: oxo (═O); OH; methoxy; C₁fluoroalkoxy; NH₂; C₁₋₂alkyl; C₁fluoroalkyl; —CH₂OH; —CH(Me)OH; —CH₂CH₂OH; —CH₂NH₂; —C(O)OH; —C(O)NHR²⁴ wherein R²⁴ is H or methyl; —C(O)R²⁵ wherein R²⁵ is methyl; fluoro; hydroxyimino (═N—OH); or (C₁₋₂alkoxy)imino (═N—OR²⁶ where R²⁶ is C₁₋₂alkyl); and wherein any OH, methoxy, fluoroalkoxy or NH₂ substituent is not substituted at the R³ and/or R^{3a} ring carbon attached (bonded) to the —NH— group of formula (I);
and wherein, when R³ and/or R^{3a} is or are the optionally substituted heterocyclic group of sub-formula (aa), (bb) or (cc), then R³ and/or R^{3a} is or are the heterocyclic group of sub-formula (aa), (bb) or (cc) optionally substituted on a ring carbon with one or two substituents independently being oxo (═O), OH or methyl; and wherein any OH substituent is not substituted at the R³ and/or R^{3a} ring carbon attached (bonded) to the —NH— group of formula (I) and is not substituted at either R³ and/or R^{3a} ring carbon bonded to the Y group of the heterocyclic group (aa), (bb) or (cc);
and wherein, when R³ and/or R^{3a} is optionally substituted mono-unsaturated-C₅₋₇cycloalkenyl, then the cycloalkenyl is optionally substituted on a ring carbon with one substituent being fluoro or methyl, and the R³ and/or R^{3a} ring carbon bonded to the —NH— group of formula (I) does not partake in the cycloalkenyl double bond;
provided that:
when R³ and/or R^{3a} is or are the heterocyclic group of sub-formula (aa) and Y is NR¹⁰, then R¹⁰ is not C(O)-methyl, or C(O)—C₁fluoroalkyl; and
when R³ and/or R^{3a} is or are the heterocyclic group of sub-formula (bb), and Y is NR¹⁰, then R¹⁰ is not methyl; and
when R³ and/or R^{3a} is or are the heterocyclic group of sub-formula (cc), then Y is O, S, SO₂ or NR¹⁰ wherein R¹⁰ is H or methyl;
and wherein:
when R³ and/or R^{3a} is or are optionally substituted C₄₋₇cycloalkyl, then any —C(O)NHR²⁴ or —C(O)R²⁵ substituent on a ring carbon is: at the 3-position of a R³ and/or R^{3a} cyclobutyl ring; or at the 3- or 4-position of a R³ and/or R^{3a} cyclopentyl ring; or at the 4-position of a R³ and/or R^{3a} cyclohexyl ring; or at the 3-, 4-, 5- or 6-position of a R³ and/or R^{3a} cycloheptyl ring (wherein, in this connection, the 1-position of the R³ and/or R^{3a} cycloalkyl ring is deemed to be the connection point to the —NH— in formula (I), that is the ring atom connecting to the —NH— in formula (I));
and wherein:
when R³ and/or R^{3a} is or are optionally substituted C₄₋₇cycloalkyl, then any OH, methoxy, fluoroalkoxy, —CH₂OH, —CH(Me)OH, —CH₂CH₂OH, —CH₂NH₂, or —C(O)OH substituent on a ring carbon is: at the 3-position of a R³ and/or R^{3a} cyclobutyl ring; or at the 3- or 4-position of a R³ and/or R^{3a} cyclopentyl ring; or at the 3-, 4- or 5-position of a R³ and/or R^{3a} cyclohexyl ring; or at the 3-, 4-, 5- or 6-position of a R³ and/or R^{3a} cycloheptyl ring; and
and wherein:
when R³ and/or R^{3a} is or are the heterocyclic group of sub-formula (aa), (bb) or (cc), then any OH substituent on a ring carbon is: at the 5-position of a six-membered R³ and/or R^{3a} heterocyclic group of sub-formula (cc) wherein n² is 1; or at the 5- or 6-position of a seven-membered R³ and/or R^{3a} heterocyclic group of sub-formula (cc) wherein n² is 2; or at the 6-position of a seven-membered R³ and/or R^{3a} heterocyclic group of sub-formula (bb) wherein n¹ is 2 (wherein, in this connection, the 1-position of the R³ and/or R^{3a} heterocyclic ring is deemed to be the connection point to the —NH— in formula (I), that is the ring atom connecting to the —NH— in formula (I), and the remaining positions of the ring are then numbered so that the ring heteroatom takes the lowest possible number).

In compounds, for example in the compounds of formula (I), an “alkyl” group or moiety may be straight-chain or branched. Alkyl groups, for example C₁₋₈alkyl or C₁₋₆alkyl or C₁₋₄alkyl or C₁₋₃alkyl or C₁₋₂alkyl, which may be employed include C₁₋₆alkyl or C₁₋₄alkyl or C₁₋₃alkyl or C₁₋₂alkyl such as methyl, ethyl, n-propyl, n-butyl, n-pentyl, or n-hexyl or any branched isomers thereof such as isopropyl, t-butyl, sec-butyl, isobutyl, 3-methylbutan-2-yl, 2-ethylbutan-1-yl, or the like.

A corresponding meaning is intended for “alkoxy”, “alkylene”, and like terms derived from alkyl. For example, “alkoxy” such as C₁₋₆alkoxy or C₁₋₄alkoxy or C₁₋₂alkoxy includes methoxy, ethoxy, propyloxy, and oxy derivatives of the alkyls listed above. “Alkylsulfonyl” such as C₁₋₄alkylsulfonyl includes methylsulfonyl (methanesulfonyl), ethylsulfonyl, and others derived from the alkyls listed above. “Alkylsulfonyloxy” such as C₁₋₄alkylsulfonyloxy includes methanesulfonyloxy (methylsulfonyloxy), ethanesulfonyloxy, et al.

“Cycloalkyl”, for example C₃₋₈cycloalkyl (e.g. C₄₋₇cycloalkyl), includes cyclopropyl, cyclobutyl, cyclopentyl, cyclohexyl, cycloheptyl, cyclooctyl, and the like. Suitably, a C₃₋₈cycloalkyl group can be C₃₋₆cycloalkyl or C₅₋₆cycloalkyl or C₄₋₇cycloalkyl or C₆₋₇cycloalkyl, that is contains a 3-6 membered or 5-6 membered or 4-7 membered or 6-7 membered carbocyclic ring.

“Fluoroalkyl” includes alkyl groups with one, two, three, four, five or more fluorine substituents, for example C₁₋₄fluoroalkyl or C₁₋₃fluoroalkyl or C₁₋₂fluoroalkyl such as monofluoromethyl, difluoromethyl, trifluoromethyl, pentafluoroethyl, 2,2,2-trifluoroethyl (CF₃CH₂—), 2,2-difluoroethyl (CHF₂CH₂—), 2fluoroethyl (CH₂FCH₂—), etc. “Fluoroalkoxy” includes C₁₋₄fluoroalkoxy or C₁₋₂fluoroalkoxy such as trifluoromethoxy, pentafluoroethoxy, monofluoromethoxy, difluoromethoxy, etc. “Fluoroalkylsulfonyl” such as C₁₋₄fluoroalkylsulfonyl includes trifluoromethanesulfonyl, pentafluoroethylsulfonyl, etc.

A halogen atom (“halo”) present in compounds, for example in the compounds of formula (I), means a fluorine, chlorine, bromine or iodine atom (“fluoro”, “chloro”, “bromo” or “iodo”), for example fluoro, chloro or bromo.

When the specification states that atom or moiety A is “bonded” or “attached” to atom or moiety B, it means that atom/moiety A is directly bonded to atom/moiety B usually by means of a covalent bond or a double covalent bond, and excludes A being indirectly attached to B via one or more intermediate atoms/moieties (e.g. excludes A-C-B); unless it is clear from the context that another meaning is intended.

Q can for example be —(CH₂)ₘ¹—Ar¹—(CH₂)ₘ²—; —(CH₂)ₘ¹—Ar⁴—O—CH₂—;

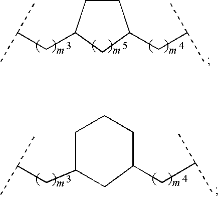

—(CH₂)ₘ⁶—X¹—(CH₂)ₘ⁷—; or —(CHMe)—X³—(CHMe)-.

Q can for example be —(CH₂)ₘ¹—Ar¹—(CH₂)ₘ²—; —(CH₂)ₘ¹—Ar⁴—O—CH₂—;

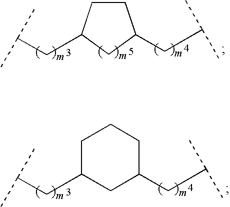

or —(CH₂)ₘ⁶—X¹—(CH₂)ₘ⁷—.

Suitably, Q is —(CH₂)ₘ¹—Ar¹—(CH₂)ₘ²- or —(CH₂)ₘ⁶—X¹—(CH₂)ₘ⁷—.

Preferably, Q is —(CH₂)ₘ¹—Ar¹—(CH₂)ₘ²—.

In one embodiment, either m¹ is 0, 1 or 2 (for example 0 or 1) and m² is 0, or m¹ is 1 and m² is 1.

m¹ can for example be 0 or 1, such as 0. m² can for example be 0.

Suitably, m¹ and m² are 0.

m³ can for example be 0. m⁴ can for example be O, Suitably, m³ and m⁴ are 0.

m⁶ can for example be 1 or 2. m⁷ can for example be 1 or 2. m⁶ can for example be the same as m⁷. Suitably, m⁶ and m⁷ are 1.

Suitably, X¹, X² and/or X³, in particular X¹, is or are independently: —CH₂—, —CMe₂-, —CHMe-, O or S(O)₂. Suitably, X¹, X² and/or X³, in particular X¹, is or are independently: —CH₂—, —CMe₂-, O or S(O)₂. Preferably, X¹, X² and/or X³, in particular X¹, is or are independently: —CH₂—, —CMe₂- or S(O)₂.

R⁵ can for example be H or methyl, e.g. methyl.

Suitably, X¹⁵ and/or X¹⁶ independently is or are: —CH₂—, —CMe₂-, —CHMe-, —CF₂— or 0; such as —CH₂—, —CMe₂- or 0, for example —CH₂— or O.

Suitably, Ar¹, Ar², Ar³ and/or Ar⁴, in particular Ar¹ and/or Ar⁴, independently have the sub-formula (x1), (x2), (x3), (x4), (x15), (x16), (x18), (x19), (x20), (x21), (x22), (x24), (x25), (x26), (x27), (x28), or (x29).

Suitably, Ar¹, Ar², Ar³ and/or Ar⁴, in particular Ar¹ and/or Ar⁴, independently have the sub-formula (x1), (x2), (x3), (x4), (x15), (x16), (x18), (x20), (x21), (x22), (x24), or (x25).

Suitably, Ar¹, Ar², Ar³ and/or Ar⁴, in particular Ar¹ and/or Ar⁴, independently have the sub-formula (x1), (x2), (x3), (x4), (x15), (x16), (x18), (x20), (x21) or (x24); for example (x1), (x2), (x3), (x4), (x15), (x16), (x18) or (x24).

Preferably, Ar¹, Ar², Ar³ and/or Ar⁴, in particular Ar¹ and/or Ar⁴, independently have the sub-formula (x15), (x16) or (x24).

In one preferable embodiment, Ar¹, Ar², Ar³ and/or Ar⁴, in particular Ar¹ and/or Ar⁴, independently have the sub-formula (x15) or (x16).

In one preferable embodiment, Ar¹, Ar², Ar³ and/or Ar⁴, in particular Ar¹ and/or Ar⁴, independently have the sub-formula (x24).

Suitably, Q is —(CH₂)ₘ⁸- wherein m⁸ is 2, 3, 4, 5, 6, 7 or 8 (such as 2, 3, 4 or 5, e.g. 2 or 3), or Q is —CHMe-CHMe-, or Q is —CH₂—X¹—CH₂— wherein X¹ is —CMe₂-, O, S(O)₂ or

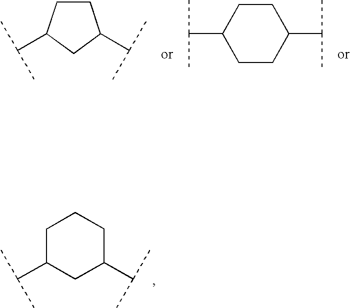

NMe (e.g. X¹ can be —CMe₂-, O or S(O)₂), or Q is

or Q has the sub-formula (q1a), (q1b), (q1c), (q1d), (q2a), (q2b), (q3a), (q3b), (q4), (q15), (q16), (q18a), (q18b), (q20), (q21), (q22), (q24) or (q25):

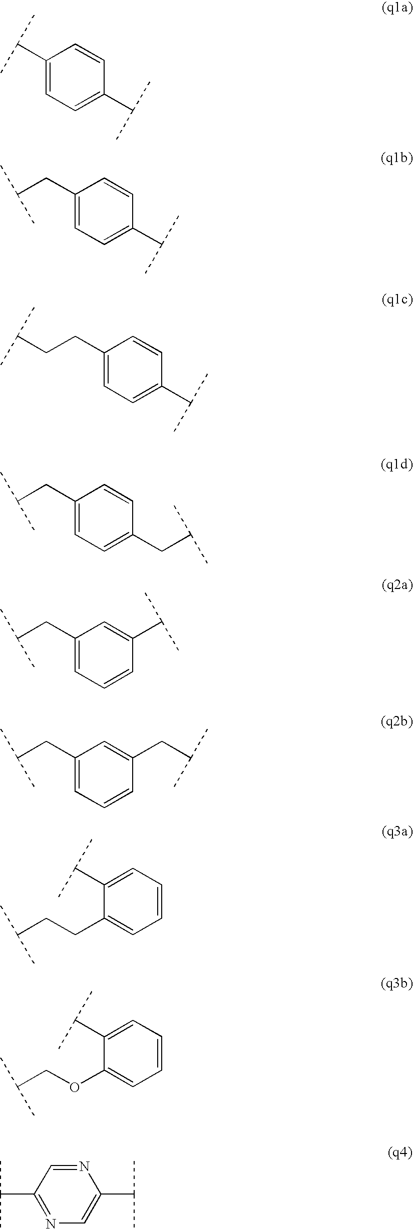

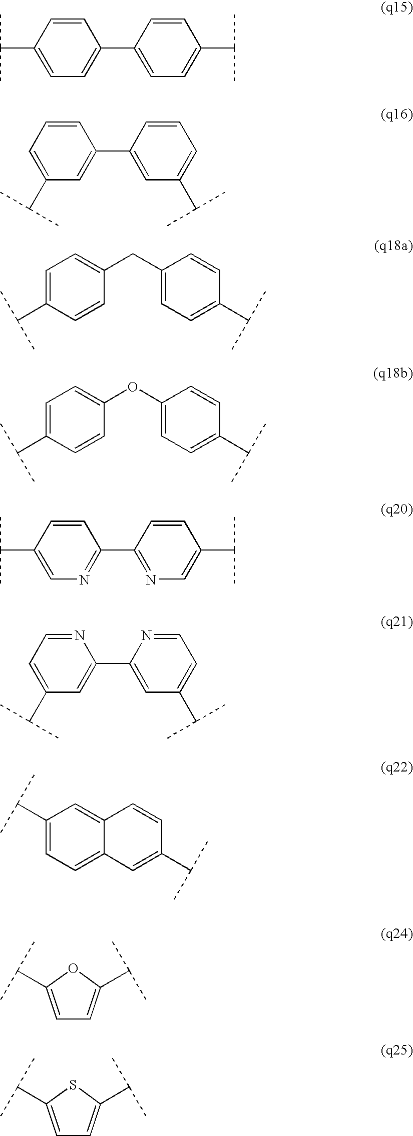

Suitably, Q has the sub-formula (q1a), (q1b), (q1c), (q1d), (q2a), (q2b), (q3a), (q3b), (q4), (q15), (q16), (q18a), (q18b), (q20), (q21), (q22), (q24) or (q25).

Preferably, Q has the sub-formula (q1b), (q1c), (q1d), (q2a), (q2b), (q3a), (q3b), (q4), (q15), (q16), (q18a), (q18b), (q20), (q21) or (q24); for example (q1b), (q1c), (q1d), (q2a), (q2b), (q3a), (q3b), (q4), (q15), (q16), (q18a), (q18b) or (q24).

More preferably, Q has the sub-formula (q15), (q16) or (q24).

In one preferable embodiment, Q has the sub-formula (q15) or (q16). According to an alternative separate embodiment, Q does not have the sub-formula (q15) or (q16).

In one preferable embodiment, Q has the sub-formula (q24). According to an alternative separate embodiment, Q does not have the sub-formula (q24).

In one preferred embodiment, the compound of formula (I) or the salt thereof is a compound of formula (II) or a salt thereof (e.g. a compound of formula (II) or a pharmaceutically acceptable salt thereof:

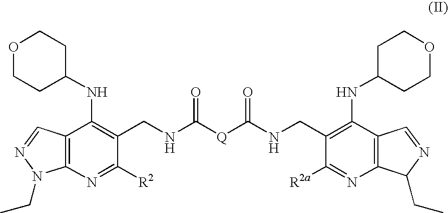

wherein Q has the sub-formula (q15) or (q16):

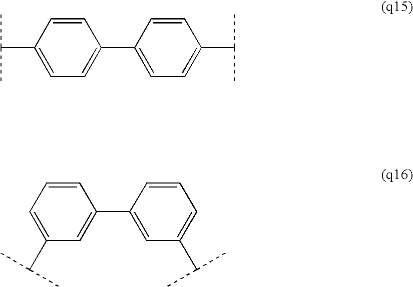

and wherein R² and R^{2a} independently are methyl or ethyl.

In formula (II), R² and R^{2a} are suitably the same and are methyl or ethyl.

In formula (II), R² and R^{2a} can be ethyl.

One aspect of the invention therefore provides a compound of formula (II) or a salt thereof (e.g. a compound of formula (II) or a pharmaceutically acceptable salt thereof.

The compound of formula (II) or the salt thereof can be for inhaled administration e.g. to a mammal such as a human.

In one particularly preferred embodiment, the compound of formula (II) or the salt thereof is (or another aspect of the invention provides): N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide, which is,

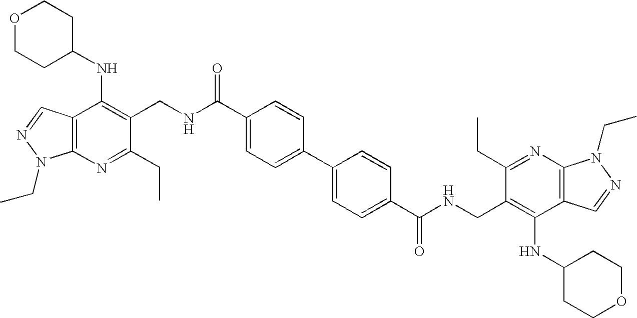

or a salt thereof; such as the compound or a pharmaceutically acceptable salt thereof, for example the compound or a 1,5-naphthalenedisulfonate, 1,2,4-benzenetricarboxylate, para-toluenesulfonate (tosylate, e.g. di-para-toluenesulfonate=di-tosylate), methanesulfonate (e.g. di-methanesulfonate), hydroxyethylidene-1,1-diphosphonate, hydrochloride (e.g. dihydrochloride), maleate or sulphate salt thereof. See for example Example 23 disclosed herein. This compound or the salt thereof can be for inhaled administration e.g. to a mammal such as a human. Preferably, the compound or salt is a 1,5-naphthalenedisulfonate, 1,2,4-benzenetricarboxylate, or para-toluenesulfonate (tosylate, e.g. di-para-toluenesulfonate=di-tosylate) salt of N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide.

In an alternative preferred embodiment, the compound of formula (II) or the salt thereof is (or another aspect of the invention provides): N,N′-bis{[1-ethyl-6-methyl-4-(tetrahydro-2H-pyran-4-ylamino)-1H-pyrazolo[3,4-b]pyridin-5-yl]methyl}-3,3′-biphenyldicarboxamide, which is,

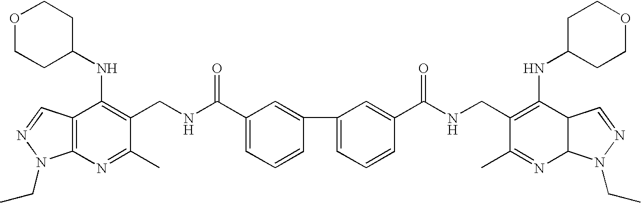

or a salt thereof (e.g. the compound or a pharmaceutically acceptable salt thereof. See for example Example 29 disclosed herein. This compound or the salt thereof can be for inhaled administration e.g. to a mammal such as a human.

In an alternative separate embodiment, the compound of formula (I) or the salt thereof does not include a compound of formula (II) or a salt thereof. Therefore, another alternative aspect of the invention provides a compound of formula (I) or a salt thereof which is not a compound of formula (II) or a salt thereof.

In one preferred embodiment of the invention, the compound of formula (I) or the salt thereof is a compound of formula (III), which is N,N′-bis{[1,6-diethyl-4-(tetrahydro-2H-pyran-4-ylamino)-1H-pyrazolo[3,4-b]pyridin-5-yl]methyl}-2,5furandicarboxamide, or a salt thereof, for example a compound of formula (III) or a pharmaceutically acceptable salt thereof:

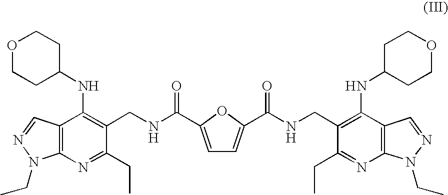

See for example Example 35 disclosed herein. This compound or the salt thereof can be for inhaled administration e.g. to a mammal such as a human.

One aspect of the invention therefore provides a compound of formula (III) or a salt thereof (e.g. a compound of formula (III) or a pharmaceutically acceptable salt thereof.

In an alternative separate embodiment, the compound of formula (I) or the salt thereof does not include a compound of formula (III) or a salt thereof. Therefore, another alternative aspect of the invention provides a compound of formula (I) or a salt thereof which is not a compound of formula (III) or a salt thereof.

R¹ and R^{1a} can be the same or different. R¹ and R^{1a} are preferably the same (for example for convenience and/or simplicity e.g. regarding compound synthesis).

When R¹ and/or R^{1a} is or are C₁₋₃alkyl or C₁₋₃fluoroalkyl, it or they can independently be straight-chained or branched. When R¹ and/or R^{1a} is or are C₁₋₃alkyl then it or they can independently be methyl, ethyl, n-propyl, or isopropyl. When R¹ and/or R^{1a} is or are C₁₋₃fluoroalkyl: then R¹ and/or R^{1a} can for example independently be C₁fluoroalkyl such as monofluoromethyl, difluoromethyl, trifluoromethyl; or R¹ and/or R^{1a} can independently be C₂fluoroalkyl such as pentafluoroethyl or more preferably C₁fluoroalkyl-CH₂— such as 2,2,2-trifluoroethyl (CF₃CH₂—), 2,2-difluoroethyl (CHF₂CH₂—), or 2fluoroethyl (CH₂FCH₂—).

R¹ and R^{1a} independently are C₁₋₃alkyl (e.g. methyl, ethyl or n-propyl), C₁₋₃fluoroalkyl or —CH₂CH₂OH. R¹ and/or R^{1a} can for example independently be C₁₋₃alkyl, C₁₋₂fluoroalkyl, or —CH₂CH₂OH. Suitably, R¹ and/or R^{1a} independently is or are C₂₋₃alkyl (e.g. ethyl or n-propyl), C₂fluoroalkyl (e.g. C₁fluoroalkyl-CH₂— such as CF₃—CH₂—) or —CH₂CH₂OH.

Preferably, R¹ and/or R^{1a} independently is or are ethyl, n-propyl or —CH₂CH₂OH. More preferably, R¹ and/or R^{1a} independently is or are ethyl. Most preferably, R¹ and R^{1a} are ethyl.

R² and R^{2a} can be the same or different. R² and R^{2a} are preferably the same (for example for convenience and/or simplicity e.g. regarding compound synthesis).

R² and/or R^{2a} can for example independently be a hydrogen atom (H), methyl, ethyl, n-propyl, isopropyl, C₁fluoroalkyl (such as CF₃ or CHF₂ or CH₂F), C₂fluoroalkyl such as C₂F₅ or C₁fluoroalkyl-CH₂— [e.g. 2,2,2-trifluoroethyl (CF₃CH₂—), 2,2-difluoroethyl (CHF₂CH₂—) or 2fluoroethyl (CH₂FCH₂—)], or cyclopropyl.

R² and/or R^{2a} can for example independently be methyl, ethyl, C₁fluoroalkyl (such as CF₃ or CHF₂ or CH₂F), or C₂fluoroalkyl such as C₂F₅ or C₁fluoroalkyl-CH₂— [e.g. 2,2,2-trifluoroethyl (CF₃CH₂—), 2,2-difluoroethyl (CHF₂CH₂—) or 2fluoroethyl (CH₂FCH₂—)].

R² and/or R^{2a} can for example independently be a hydrogen atom (H), methyl, ethyl or C₁fluoroalkyl (such as CF₃ or CHF₂ or CH₂F). Suitably, R² and/or R^{2a} independently is or are a hydrogen atom (H), methyl or ethyl. Alternatively, suitably, R² and/or R^{2a} independently is or are methyl, ethyl or C₁fluoroalkyl (such as CF₃ or CHF₂ or CH₂F).

Preferably, R² and/or R^{2a} independently is or are methyl or ethyl. Preferably, R² and R^{2a} are the same and are methyl or ethyl. More preferably, R² and/or R^{2a} independently are ethyl; in particular R² and R^{2a} can be ethyl.

Throughout this specification, unless where indicated otherwise, R³ and R^{3a} can be the same or different (i.e. are independent of each other). However, R³ and R^{3a} are preferably the same (for example for convenience and/or simplicity e.g. regarding compound synthesis).

Preferably, in R³ and/or R^{3a} there is one substituent or no substituent on a ring carbon.

In one preferable embodiment, R³ and/or R^{3a} is the optionally substituted C₄₋₇cycloalkyl or the optionally substituted heterocyclic group of sub-formula (aa), (bb) or (cc).

In one optional embodiment, when R³ and/or R^{3a} is optionally substituted C₄₋₇cycloalkyl, it is not unsubstituted C₅cycloalkyl, i.e. not unsubstituted cyclopentyl. In this case, suitably, R³ and/or R^{3a} is optionally substituted C₆₋₇cycloalkyl or optionally substituted cyclobutyl.

When R³ and/or R^{3a} is optionally substituted C₄₋₇cycloalkyl, it is suitably optionally substituted C₆₋₇cycloalkyl or optionally substituted cyclobutyl, preferably optionally substituted C₆cycloalkyl (i.e. optionally substituted cyclohexyl).

When R³ and/or R^{3a} is optionally substituted C₄₋₇cycloalkyl, then R³ and/or R^{3a} is C₄₋₇cycloalkyl (e.g. C₆₋₇cycloalkyl or cyclobutyl) optionally substituted on a ring carbon with one or two substituents independently being (e.g. one substituent being): oxo (═O); OH; methoxy; C₁fluoroalkoxy (e.g. trifluoromethoxy or difluoromethoxy); NH₂; C₁₋₂alkyl such as methyl; C₁fluoroalkyl such as —CH₂F or —CHF₂; —CH₂OH; —CH(Me)OH; —CH₂CH₂OH; —CH₂NH₂; —C(O)OH; —C(O)NHR²⁴ wherein R²⁴ is H or methyl (preferably H); —C(O)R²⁵ wherein R²⁵ is methyl; fluoro; hydroxyimino (═N—OH); or (C₁₋₂alkoxy)imino (═N—OR²⁶ where R²⁶ is C₁₋₂alkyl); and wherein any OH, methoxy, fluoroalkoxy or NH₂ substituent is not substituted at the R³ and/or R^{3a} ring carbon attached (bonded) to the —NH— group of formula (I).

When R³ and/or R^{3a} is optionally substituted C₄₋₇cycloalkyl, then R³ and/or R^{3a} can suitably be C₄₋₇cycloalkyl (e.g. C₆₋₇cycloalkyl or cyclobutyl) optionally substituted on a ring carbon with one or two substituents independently being (e.g. one substituent being): oxo (═O); OH; NH₂; C₁₋₂alkyl such as methyl; C₁fluoroalkyl such as —CH₂F or —CHF₂; —CH₂OH; —CH(Me)OH; —C(O)NHR²⁴ wherein R²⁴ is H or methyl (preferably H); —C(O)R²⁵ wherein R²⁵ is methyl; fluoro; hydroxyimino (═N—OH); or (C₁₋₂alkoxy)imino (═N—OR²⁶ where R²⁶ is C₁₋₂alkyl).

Preferably, when R³ and/or R^{3a} is optionally substituted C₄₋₇cycloalkyl, then R³ and/or R^{3a} is C₄₋₇cycloalkyl (e.g. C₆₋₇cycloalkyl or cyclobutyl) optionally substituted on a ring carbon with one or two substituents independently being (e.g. one substituent being): oxo (═O); OH; methyl; —CH₂F; —CHF₂; —CH₂OH; —C(O)NHR²⁴ wherein R²⁴ is H; fluoro; hydroxyimino (═N—OH); or methoxyimino (═N—OR²⁶ where R²⁶ is methyl).

More preferably, when R³ and/or R^{3a} is optionally substituted C₄₋₇cycloalkyl, then R³ and/or R^{3a} is C₄₋₇cycloalkyl (e.g. C₆₋₇cycloalkyl or cyclobutyl) optionally substituted on a ring carbon with one or two substituents independently being (e.g. one substituent being): oxo (═O); OH; methyl; —C(O)NHR²⁴ wherein R²⁴ is H; fluoro; or hydroxyimino (═N—OH).

Still more preferably, when R³ and/or R^{3a} is optionally substituted C₄₋₇cycloalkyl, then R³ and/or R^{3a} is C₄₋₇cycloalkyl (e.g. C₆₋₇cycloalkyl or cyclobutyl) optionally substituted on a ring carbon with one or two substituents independently being (e.g. one substituent being): oxo (═O); OH; —C(O)NHR²⁴ wherein R²⁴ is H; or hydroxyimino (═N—OH).

In one optional embodiment, in R³ and/or R^{3a}, the C₄₋₇cycloalkyl can be unsubstituted.

When R³ and/or R^{3a} is optionally substituted C₄₋₇cycloalkyl or optionally substituted C₅₋₇cycloalkenyl, e.g. optionally substituted (C₆₋₇cycloalkyl or cyclobutyl or C₅₋₇cycloalkenyl), such as optionally substituted C₆cycloalkyl (optionally substituted cyclohexyl) or optionally substituted cyclohexenyl, the one or two optional substituents on a ring carbon if present suitably can comprise a substituent (for example is or are substituent(s)) at the 3-, 4- and/or 5-position(s), e.g. at the 3- and/or 4-position(s), of the R³ and/or R^{3a} cycloalkyl or cycloalkenyl ring.

(In this connection and generally herein, the 1-position of the R³ and/or R^{3a} ring, e.g. of the R³ and/or R^{3a} cycloalkyl or cycloalkenyl ring, is deemed to be the connection point to the —NH— in formula (I)=the ring atom connecting to the —NH— in formula (I)).

Suitably, for R³ and/or R^{3a}, and in particular when R³ and/or R^{3a} is optionally substituted C₄₋₇cycloalkyl or optionally substituted C₅₋₇cycloalkenyl, R³ and/or R^{3a} is not substituted (other than optionally by alkyl or fluoroalkyl) at the ring atom connecting to the —NH— in formula (I), and R³ and/or R^{3a} is not substituted (other than optionally by alkyl, fluoroalkyl or NHR²¹) at the two ring atoms either side of (bonded to) the connecting atom. For example, suitably, for R³ and/or R^{3a}, and in particular when R³ and/or R^{3a} is optionally substituted C₄₋₇cycloalkyl or optionally substituted C₅₋₇cycloalkenyl, R³ and/or R^{3a} is not substituted at the ring atom connecting to the —NH— in formula (I), and R³ and/or R^{3a} is not substituted at the two ring atoms either side of (bonded to) the connecting atom.

Suitably, for R³ and/or R^{3a}, and in particular when R³ and/or R^{3a} is optionally substituted C₄₋₇cycloalkyl or optionally substituted C₅₋₇cycloalkenyl, the one or two optional R³ and/or R^{3a} ring-carbon substituents if present can comprise a substituent (for example is or are substituent(s)):

(a) at the 3-position of a R³ and/or R^{3a} cyclobutyl ring, or
(b) at the 3- and/or 4-position(s) of a R³ and/or R^{3a} cyclopentyl or cyclopentenyl ring, or
(c) at the 3-, 4- and/or 5-position(s) of a R³ and/or R^{3a} cyclohexyl or cyclohexenyl ring, or
(d) at the 3-, 4-, 5- and/or 6-position(s) of a R³ and/or R^{3a} cycloheptyl or cycloheptenyl ring, and/or
(f) at the 1-, 2- and/or highest-numbered-position(s) of a R³ and/or R^{3a} cycloalkyl or cycloalkenyl ring, for alkyl or fluoroalkyl substituent(s), and/or
(g) at the 2- and/or highest-numbered-position(s) of a R³ and/or R^{3a} cycloalkyl or cycloalkenyl ring, for NH₂ or fluoro substituent(s).

When R³ and/or R^{3a} is optionally substituted C₄₋₇cycloalkyl, then any OH, methoxy, fluoroalkoxy, —CH₂OH, —CH(Me)OH, —CH₂CH₂OH, —CH₂NH₂, or —C(O)OH substituent on a ring carbon is: at the 3-position of a R³ and/or R^{3a} cyclobutyl ring; or at the 3- or 4-position of a R³ and/or R^{3a} cyclopentyl ring; or at the 3-, 4- or 5-position of a R³ and/or R^{3a} cyclohexyl ring (such as at the 3- or 5-position of a R³ and/or R^{3a} cyclohexyl ring especially for any OH substituent); or at the 3-, 4-, 5- or 6-position (e.g. 4- or 5-position) of a R³ and/or R^{3a} cycloheptyl ring. Suitably, when R³ and/or R^{3a} is optionally substituted C₄₋₇cycloalkyl, then any OH, methoxy, fluoroalkoxy, —CH₂OH, —CH(Me)OH, —CH₂CH₂OH or —CH₂NH₂, or —C(O)OH substituent (or any OH substituent) on a ring carbon is at the 3- or 4-position of a R³ and/or R^{3a} cyclopentyl ring; or more suitably at the 3-, 4- or 5-position, such as at the 3- or 5-position, of a R³ and/or R^{3a} cyclohexyl ring.

When R³ and/or R^{3a} is optionally substituted C₄₋₇cycloalkyl, then any —C(O)NHR²⁴ or —C(O)R²⁵ substituent on a ring carbon is: at the 3-position of a R³ and/or R^{3a} cyclobutyl ring; or at the 3- or 4-position of a R³ and/or R^{3a} cyclopentyl ring; or at the 4-position of a R³ and/or R^{3a} cyclohexyl ring; or at the 3-, 4-, 5- or 6-position (e.g. 4- or 5-position) of a R³ and/or R^{3a} cycloheptyl ring. When R³ and/or R^{3a} is optionally substituted C₄₋₇cycloalkyl, then any —C(O)NHR²⁴ or —C(O)R²⁵ substituent, or any —C(O)NHR²⁴ substituent, on a ring carbon is suitably at the 3-position of a R³ and/or R^{3a} cyclobutyl ring or at the 4-position of a R³ and/or R^{3a} cyclohexyl ring. When R³ and/or R^{3a} is optionally substituted C₄₋₇cycloalkyl, it is preferable for any —C(O)NHR²⁴ substituent to be at the 4-position of a R³ and/or R^{3a} cyclohexyl ring.

When R³ and/or R^{3a} is optionally substituted C₄₋₇cycloalkyl, any NH₂ substituent on a ring carbon is at any position other than the 1-position (the ring atom connecting to the —NH— in formula (I)), e.g. at the 2-, 3-, 4-, 5-, 6- or 7-position. Suitably, any NH₂ substituent is at the 2-, 3-, 4-, 5- or 6-position, for example at the 3-, 4- or 5-position or at the 3- or 5-position, of a R³ and/or R^{3a} cyclohexyl ring.

When R³ and/or R^{3a} is optionally substituted C₄₋₇cycloalkyl or optionally substituted C₅₋₇cycloalkenyl, any alkyl or fluoroalkyl substituent on a ring carbon can for example be at the 1-, 2-, 3-, 4-, 5-, 6- or 7-position, for example at the 1-, 2-, 3-, 5- or 6-position, e.g. the 1-position, of the R³ and/or R^{3a} ring. Preferably, any such alkyl or fluoroalkyl substituent on a ring carbon is at the 1-, 2-, 3-, 5- or 6-position, or more preferably at the 1-, 3- or 5-position, of a R³ and/or R^{3a} cyclohexyl or cyclohexenyl ring.

When R³ and/or R^{3a} is optionally substituted C₄₋₇cycloalkyl or optionally substituted C₅₋₇cycloalkenyl, any fluoro substituent on a ring carbon can for example be at the 1-, 2-, 3-, 4-, 5-, 6- or 7-position, for example at the 2-, 3-, 4-, 5- or 6-position, such as at the 3- or 4-position, of the R³ and/or R^{3a} ring. Suitably, any fluoro substituent on a ring carbon is at the 3-, 4- or 5-position, in particular at the 4-position, of a R³ and/or R^{3a} cyclohexyl or cyclohexenyl ring.

When R³ and/or R^{3a} is optionally substituted C₄₋₇cycloalkyl, any oxo (═O), hydroxyimino (═N—OH); or (C₁₋₂alkoxy)imino (═N—OR²⁶) substituent on a ring carbon can for example be at the 3-, 4- or 5-position, e.g. at the 4-position, of the R³ and/or R^{3a} cycloalkyl (e.g. C₆₋₇cycloalkyl e.g. cyclohexyl, or cyclobutyl) ring. Any such substituent can for example be at the 3-position of a R³ and/or R^{3a} cyclobutyl ring or at the 4-position of a R³ and/or R^{3a} cyclohexyl ring. Preferably, any such substituent is at the 4-position of a R³ and/or R^{3a} cyclohexyl ring.

When R³ and/or R^{3a} is optionally substituted C₄₋₇cycloalkyl (e.g. C₆₋₇cycloalkyl or cyclobutyl, optionally substituted), then R³ and/or R^{3a} is suitably cyclohexyl (i.e. unsubstituted); or cycloheptyl (i.e. unsubstituted); or cyclohexyl substituted on a ring carbon by one substituent being oxo (═O), OH, NH₂, C₁₋₂alkyl, C₁fluoroalkyl such as —CH₂F or —CHF₂, —CH₂OH, —CH(Me)OH, —C(O)NHR²⁴ wherein R²⁴ is H or methyl (preferably H), —C(O)R²⁵, fluoro, hydroxyimino (═N—OH), or (C₁₋₂alkoxy)imino (═N—OR²⁶ wherein R²⁶ is C₁₋₂alkyl); or cyclohexyl substituted by two fluoro substituents; or cyclobutyl (i.e. unsubstituted); or cyclobutyl substituted on a ring carbon with one substituent being oxo (═O), OH, methyl, —CH₂F, —CHF₂, —CH₂OH, —C(O)NHR²⁴ wherein R²⁴ is H or methyl (preferably H), fluoro, hydroxyimino (═N—OH), or methoxyimino (═N—OR²⁶ where R²⁶ is methyl). Preferably, when R³ and/or R^{3a} is optionally substituted C₄₋₇cycloalkyl (e.g. C₆₋₇cycloalkyl or cyclobutyl, optionally substituted), then R³ and/or R^{3a} is cyclohexyl (i.e. unsubstituted); or cycloheptyl (i.e. unsubstituted); or cyclohexyl substituted on a ring carbon by one substituent being oxo (═O), OH, NH₂, C₁₋₂alkyl, C₁fluoroalkyl such as —CH₂F or —CHF₂, —CH₂OH, —C(O)NHR²⁴ wherein R²⁴ is H, fluoro, hydroxyimino (═N—OH), or (methoxy)imino (═N—OR²⁶ wherein R²⁶ is methyl); or cyclohexyl substituted by two fluoro substituents; or cyclobutyl (i.e. unsubstituted); or cyclobutyl substituted on a ring carbon with one substituent being oxo (═O), OH, methyl, —CH₂F, —CHF₂, —CH₂OH, —C(O)NHR²⁴ wherein R²⁴ is H, fluoro, hydroxyimino (═N—OH), or methoxyimino (═N—OR²⁶ where R²⁶ is methyl). More preferably, when R³ and/or R^{3a} is optionally substituted C₄₋₇cycloalkyl (e.g. C₆₋₇cycloalkyl or cyclobutyl, optionally substituted), then R³ and/or R^{3a} is cyclohexyl (i.e. unsubstituted); or cyclohexyl substituted on a ring carbon by one oxo (═O), hydroxyimino (═N—OH), —C(O)NH₂, methyl or OH substituent; or cyclobutyl substituted on a ring carbon by one —C(O)NHR²⁴ substituent wherein R²⁴ is H. The optional substituent can for example be at the 3- or 4-position of the R³ and/or R^{3a} cyclohexyl ring. Preferably, when R³ and/or R^{3a} is optionally substituted C₄₋₇cycloalkyl (e.g. C₆₋₇cycloalkyl or cyclobutyl, optionally substituted), then any OH substituent on a ring carbon is preferably at the 3-position of a R³ and/or R^{3a} cyclohexyl ring, and/or any oxo (═O), hydroxyimino (═N—OH), or (C₁₋₂alkoxy)imino (═N—OR²⁶) substituent on a ring carbon is preferably at the 4-position of a R³ and/or R^{3a} cyclohexyl ring or at the 3-position of a R³ and/or R^{3a} cyclobutyl ring, and/or any alkyl or fluoroalkyl substituent is preferably at the 1-, 3- or 5-position of a R³ and/or R^{3a} cyclohexyl ring.

When R³ and/or R^{3a} is optionally substituted cyclobutyl, then R³ and/or R^{3a} can preferably be cyclobutyl (i.e. unsubstituted) or more preferably 3-(aminocarbonyl)cyclobutyl (i.e. 3-(aminocarbonyl)cyclobutan-1-yl) (e.g. in a cis or trans configuration, preferably cis).

When R³ and/or R^{3a} is optionally substituted cyclopentyl, R³ and/or R^{3a} can for example be cyclopentyl (i.e. unsubstituted) or more suitably 3-hydroxy-cyclopentyl.

When R³ and/or R^{3a} is optionally substituted C₄₋₇cycloalkyl (e.g. optionally substituted C₆₋₇cycloalkyl or optionally substituted cyclobutyl), R³ and/or R^{3a} can for example be cyclobutyl (i.e. unsubstituted), 4-hydroxy-cyclohexyl (i.e. 4-hydroxycyclohexan-1-yl) (e.g. racemic or in a cis or trans configuration), 4-methylcyclohexyl (e.g. racemic), 2-aminocyclohexyl (e.g. racemic or in a cis or trans configuration, preferably trans), 4-aminocyclohexyl (e.g. racemic or in a cis or trans configuration, preferably racemic or cis), 3-oxocyclohexyl, 4-acetylcyclohexyl (e.g. racemic or in a cis or trans configuration, preferably racemic or cis), 4-(1-hydroxyethyl)cyclohexyl (e.g. racemic or in a cis or trans configuration with respect to the ring, preferably racemic or cis), or 3-(hydroxymethyl)cyclohexyl (e.g. racemic or in a cis or trans configuration).

However, when R³ and/or R^{3a} is optionally substituted C₄₋₇cycloalkyl (e.g. optionally substituted C₆₋₇cycloalkyl or optionally substituted cyclobutyl), R³ and/or R^{3a} is more preferably cyclohexyl (i.e. unsubstituted), cycloheptyl (i.e. unsubstituted), 3-hydroxy-cyclohexyl (i.e. 3-hydroxycyclohexan-1-yl) (e.g. racemic or in a cis or trans configuration, preferably racemic or cis), 4-oxo-cyclohexyl (i.e. 4-oxocyclohexan-1-yl), 4-(hydroxyimino)cyclohexyl (i.e. 4-(hydroxyimino)cyclohexan-1-yl), 4-(C₁₋₂alkoxyimino)cyclohexyl, 4-(aminocarbonyl)cyclohexyl (i.e. 4-(aminocarbonyl)cyclohexan-1-yl) (e.g. racemic or in a cis or trans configuration, preferably racemic or cis), 1-methylcyclohexyl (e.g. racemic), 3-methylcyclohexyl (e.g. racemic), 4,4-(difluoro)cyclohexyl, 3-aminocyclohexyl (e.g. racemic or in a cis or trans configuration), 4-(hydroxymethyl)cyclohexyl (e.g. racemic or in a cis or trans configuration), or 3-(aminocarbonyl)cyclobutyl (i.e. 3-(aminocarbonyl)cyclobutan-1-yl) (e.g. racemic or in a cis or trans configuration, preferably cis).

A “cis configuration” in general includes mixtures of configurations wherein the cis configuration is the major component.

When R³ and/or R^{3a} is optionally substituted C₄₋₇cycloalkyl (e.g. optionally substituted C₆₋₇cycloalkyl or optionally substituted cyclobutyl), R³ and/or R^{3a} is still more preferably cyclohexyl (i.e. unsubstituted), 3-hydroxy-cyclohexyl (i.e. 3-hydroxycyclohexan-1-yl) (preferably racemic or in a cis configuration), 4-oxo-cyclohexyl (i.e. 4-oxocyclohexan-1-yl), 4-(hydroxyimino)cyclohexyl (i.e. 4-(hydroxyimino)cyclohexan-1-yl), 4-(aminocarbonyl)cyclohexyl (i.e. 4-(aminocarbonyl)cyclohexan-1-yl) (preferably racemic or in a cis configuration), or 3-(aminocarbonyl)cyclobutyl (i.e. 3-(aminocarbonyl)cyclobutan-1-yl) (preferably racemic or in a cis configuration).

When R³ and/or R^{3a} is optionally substituted mono-unsaturated-C₅₋₇cycloalkenyl, suitably it is optionally substituted mono-unsaturated-C₅₋₆cycloalkenyl, preferably optionally substituted mono-unsaturated-C₆cycloalkenyl (i.e. optionally substituted mono-unsaturated-cyclohexenyl=optionally substituted cyclohexenyl). For example, the R³ and/or R^{3a} cyclohexenyl can be optionally substituted cyclohex-3-en-1-yl.

When R³ and/or R^{3a} is optionally substituted mono-unsaturated-C₅₋₇cycloalkenyl, suitably the R³ and/or R^{3a} cycloalkenyl (e.g. cyclohexenyl) is substituted on a ring carbon with one fluoro substituent or is unsubstituted. For example, the R³ and/or R^{3a} optionally substituted cycloalkenyl can be cyclohex-3-en-1-yl (i.e. unsubstituted) or 4-fluoro-cyclohex-3-en-1-yl.

For R³ and/or R^{3a} cycloalkenyl, the optional substituent(s) on a ring carbon can for example be at the 1-, 2-, 3-, 4-, 5- or 6-position(s) of the cycloalkenyl ring.

When R³ and/or R^{3a} is the heterocyclic group of sub-formula (aa), (bb) or (cc), then Y is suitably O or NR¹⁰. When R³ and/or R^{3a} is the heterocyclic group of sub-formula (aa) or (bb), then Y is preferably O or N—C(O)—NH₂.

R¹⁰ can for example be a hydrogen atom (H), methyl, ethyl, C(O)NH₂, C(O)-methyl or C(O)—C₁ fluoroalkyl.

Suitably, R¹⁰ is not methyl.

Suitably, R¹⁰ is a hydrogen atom (H), C(O)NH₂, C(O)-methyl or C(O)—C₁fluoroalkyl (e.g. C(O)—CF₃). More suitably, R¹⁰ is H, C(O)NH₂ or C(O)-methyl; in particular C(O)NH₂.

When R³ and/or R^{3a} is the heterocyclic group of sub-formula (aa), (bb) or (cc), then it is preferable that R³ and/or R^{3a} is the heterocyclic group of sub-formula (aa) or (bb), more preferably of sub-formula (bb).

In sub-formula (bb), n¹ is preferably 1. In sub-formula (cc), n² is preferably 1. That is, six-membered rings are preferred in the R³ and/or R^{3a} heterocyclic group.

When R³ and/or R^{3a} is the optionally substituted heterocyclic group of sub-formula (aa), (bb) or (cc), then R³ and/or R^{3a} is the heterocyclic group of sub-formula (aa), (bb) or (cc) optionally substituted on a ring carbon with one or two substituents independently being (e.g. one substituent being) oxo (═O), OH or methyl; and wherein any OH substituent is not substituted at the R³ and/or R^{3a} ring carbon attached (bonded) to the —NH— group of formula (I) and is not substituted at either R³ and/or R^{3a} ring carbon bonded to the Y group of the heterocyclic group (aa), (bb) or (cc).

Preferably, the R³ and/or R^{3a} heterocyclic group of sub-formula (aa), (bb) or (cc) is optionally substituted on a ring carbon with one or two substituents independently being (e.g. one substituent being) oxo (═O) or methyl. More preferably, the R³ and/or R^{3a} heterocyclic group of sub-formula (aa), (bb) or (cc) is optionally substituted on a ring carbon with one or two (e.g. one) substituents being oxo (═O).

It is generally preferable that, in R³ and/or R^{3a}, the heterocyclic group of sub-formula (aa), (bb) or (cc) is not substituted on a ring carbon. (In this connection, where Y is NR¹⁰, R¹⁰ is not a substituent on a ring carbon).

In the R³ and/or R^{3a} heterocyclic group of sub-formula (aa), (bb) or (cc), any oxo (═O) substituent on a ring carbon is suitably on a carbon atom bonded (adjacent) to Y. In one embodiment, any oxo (═O) substituent on a ring carbon can be on a carbon atom bonded (adjacent) to Y only when Y is O or NR¹⁰.

In the R³ and/or R^{3a} heterocyclic group of sub-formula (aa), (bb) or (cc), any oxo (═O) substituent on a ring carbon can suitably be at the 2-, 3-, 4-, 5- or 6-position of the R³ and/or R^{3a} heterocyclic ring. For example any ring-carbon oxo (═O) substituent(s) can be: at the 2-, 4- or 5-position(s) (e.g. 2-position or 4-position, or two oxo substituents at 2- and 4-positions) of a R³ and/or R^{3a} heterocyclic group of sub-formula (aa), at the 2-, 4-, 5- or 6-position(s) (e.g. 4-position) of a six-membered R³ and/or R^{3a} heterocyclic group of sub-formula (cc) wherein n² is 1, at the 2-, 3-, 5-, 6- or 7-position(s) (e.g. 5-position) of a seven-membered R³ and/or R^{3a} heterocyclic group of sub-formula (bb) wherein n¹ is 2, or at the 2-, 4-, 5-, 6- or 7-position(s) (e.g. 2-position) of a seven-membered R³ and/or R^{3a} heterocyclic group of sub-formula (cc) wherein n² is 2.

(In this connection and generally herein, the 1-position of the R³ and/or R^{3a} heterocyclic ring is deemed to be the connection point to the —NH— in formula (I)=the ring atom connecting to the —NH— in formula (I), and the remaining positions of the ring are then numbered so that the ring heteroatom takes the lowest possible number).

In the R³ and/or R^{3a} heterocyclic group of sub-formula (aa), (bb) or (cc), any methyl substituent on a ring carbon can for example be at the 1-, 2-, 3-, 4-, 5- or 6-position, e.g. the 1-position, of the R³ and/or R^{3a} heterocyclic ring, in particular at the 1-, 3- or 5-position of a six-membered R³ and/or R^{3a} heterocyclic ring which is of sub-formula (bb) wherein n¹ is 1 or which is of sub-formula (cc) wherein n² is 1.

In the R³ and/or R^{3a} heterocyclic group of sub-formula (aa), (bb) or (cc), then any OH substituent on a ring carbon is: at the 5-position of a six-membered R³ and/or R^{3a} heterocyclic group of sub-formula (cc) wherein n² is 1; at the 5- or 6-position of a seven-membered R³ and/or R^{3a} heterocyclic group of sub-formula (cc) wherein n² is 2; or at the 6-position of a seven-membered R³ and/or R^{3a} heterocyclic group of sub-formula (bb) wherein n¹ is 2.

Therefore, in the R³ and/or R^{3a} heterocyclic group of sub-formula (aa), (bb) or (cc), only methyl or oxo (═O) substitution or no substitution is allowed on a ring carbon, independently at each of the 2- and highest-numbered-positions of the R³ and/or R^{3a} heterocyclic ring (e.g. at each of the 2- and 6-positions of a six-membered R³ and/or R^{3a} heterocyclic ring); and only methyl substitution or no substitution is allowed at the 1-position ring-carbon of the R³ and/or R^{3a} heterocyclic ring.

However, it is generally preferable that, in R³ and/or R^{3a}, the heterocyclic group of sub-formula (aa), (bb) or (cc) is not substituted on a ring carbon. (In this connection, where Y is NR¹⁰, R¹⁰ is not a substituent on a ring carbon).

When R³ and/or R^{3a} is the heterocyclic group of sub-formula (aa) and Y is NR¹⁰, then R¹⁰ is not C(O)-methyl, or C(O)—C₁fluoroalkyl.

In one preferable embodiment, when R³ and/or R^{3a} is the heterocyclic group of sub-formula (aa), then Y is O, S, SO₂, NH or NC(O)NH₂ (in particular Y can be O, S, NH or NC(O)NH₂, such as NC(O)NH₂).

When R³ and/or R^{3a} is the heterocyclic group of sub-formula (bb), and Y is NR¹⁰ (e.g. when NHR³ and/or NHR^{3a} is

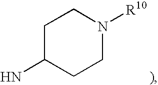

then R¹⁰ is not methyl.

Therefore, when R³ and/or R^{3a} is the heterocyclic group of sub-formula (bb), then Y is O, S, SO₂ or NR¹⁰ wherein R¹⁰ is H, C(O)NH₂, C(O)-methyl or C(O)—C₁fluoroalkyl (e.g. C(O)—CF₃). When R³ and/or R^{3a} is the heterocyclic group of sub-formula (bb), then R¹⁰ is preferably H, C(O)NH₂ or C(O)-methyl, for example C(O)NH₂ or C(O)-methyl, more preferably C(O)NH₂.

When R³ and/or R^{3a} is the heterocyclic group of sub-formula (cc), then Y is O, S, SO₂ or NR¹⁰ wherein R¹⁰ is H or methyl.

Suitably, when R³ and/or R^{3a} is the heterocyclic group of sub-formula (cc), then: either Y is O, S, SO₂ or NR¹⁰ wherein R¹⁰ is H, or NHR³ and/or NHR^{3a} is of sub-formula (m4):

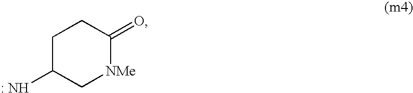

wherein the —NH-connection point of the NHR³ and/or NHR^{3a} group to the 4-position of the pyrazolopyridine of formula (I) is underlined.

Suitably, when R³ and/or R^{3a} is the heterocyclic group of sub-formula (cc), then Y is O, S, SO₂ or NR¹⁰ wherein R¹⁰ is H, or Y is O or NR¹⁰ wherein R¹⁰ is H.

Optionally, for sub-formula (bb) and/or for sub-formula (cc), Y is O or NR¹⁰.

When R³ and/or R^{3a} is optionally substituted C₄₋₇cycloalkyl (e.g. optionally substituted C₆₋₇cycloalkyl or optionally substituted cyclobutyl) or optionally substituted mono-unsaturated-C₅₋₇cycloalkenyl or an optionally substituted heterocyclic group of sub-formula (aa), (bb) or (cc), then a substituent on a ring carbon can be racemic or in the cis or trans configuration with respect to the —NH— group of formula (I) to which R³ and/or R^{3a} is attached (bonded). A cis or trans configuration includes mixtures of configurations wherein the stated configuration is the major component. In this context, “racemic” refers to a mixture of isomers containing substantially equal amounts of the cis and trans configurations with respect to a substituent and the —NH— group on the R³ and/or R^{3a} ring, and in this context “racemic” does not refer to isomerism at atoms other than R³ and/or R^{3a} ring carbon atoms. For example, an OH or —C(O)NHR²⁴ substituent on C₆₋₇cycloalkyl or cyclobutyl can for example be in the cis configuration and/or a NH₂ substituent on C₆₋₇cycloalkyl can for example be racemic or in the cis or trans configuration, with respect to the —NH— group of formula (I) to which R³ and/or R^{3a} is attached (bonded), including mixtures of configurations wherein the stated configuration is the major component.

When R³ and/or R^{3a} is a bicyclic group of sub-formula (ee), then NHR³ and/or NHR^{3a} can be of sub-formula (c6) or (c7):

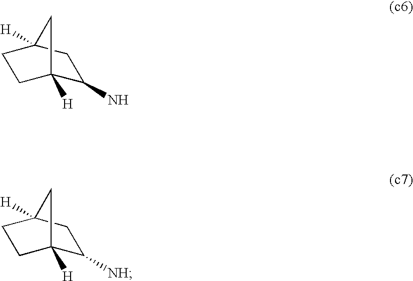

wherein the —NH-connection point of the NHR³ and/or NHR^{3a} group to the 4-position of the pyrazolopyridine of formula (I) is underlined.

Preferably, NHR³ and/or NHR^{3a} independently is or are of sub-formula (a1), (b), (c), (c 1), (c 2), (c 3), (c 4), (c 5), (c 6), (c 7), (d), (e), (f), (g), (g2), (g4), (h), (i), (j), (k), (k1), (k2), (k3), (L), (m), (m1), (m3), (m4), (n), (o), (o1), (o2), (o3), (p), (p1), (p2), (p3), (p5), (p6), (p9), (p10), (p12), (p13), (p14), (p15), or (q):

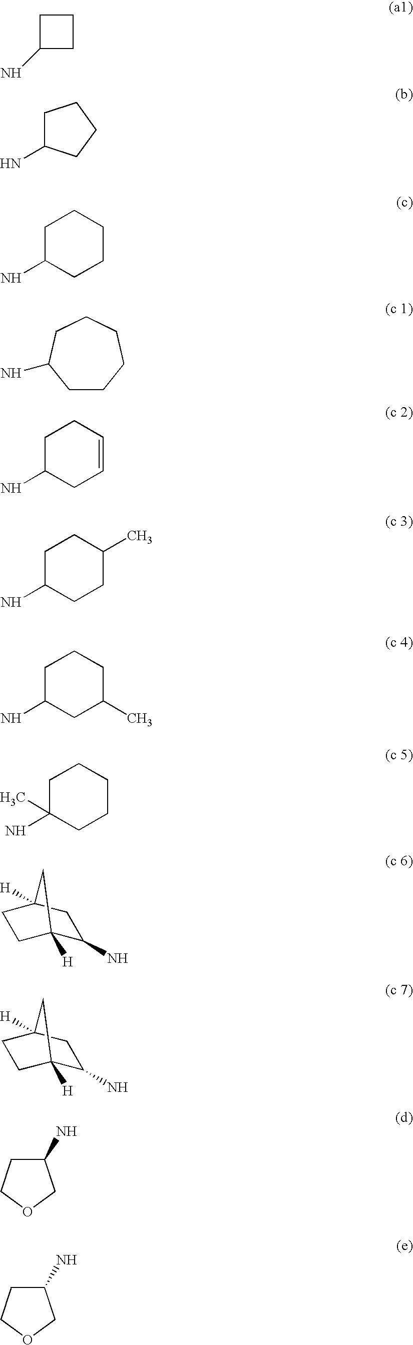

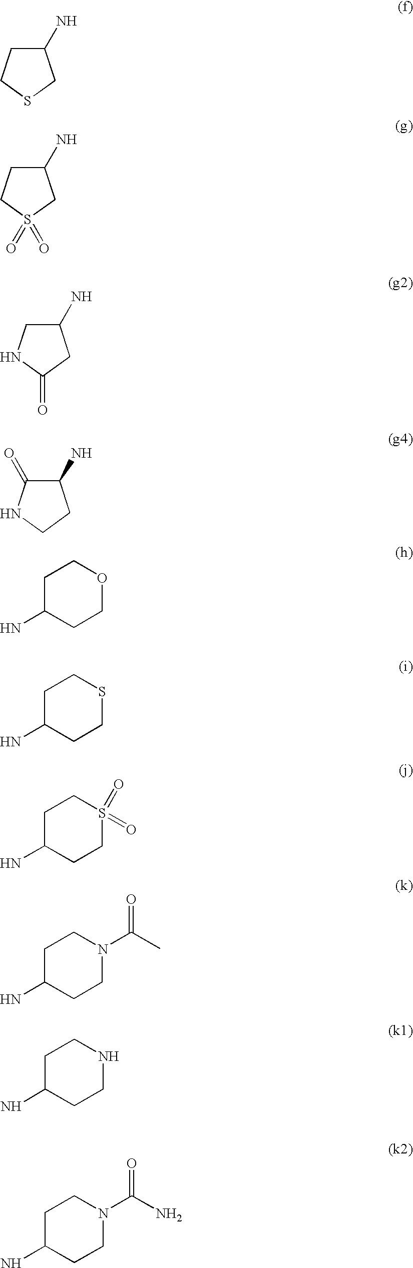

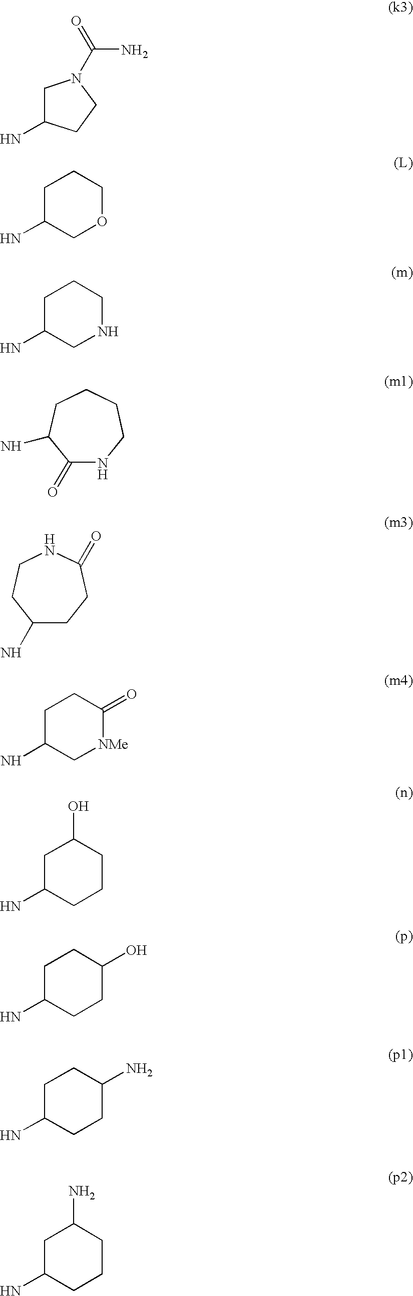

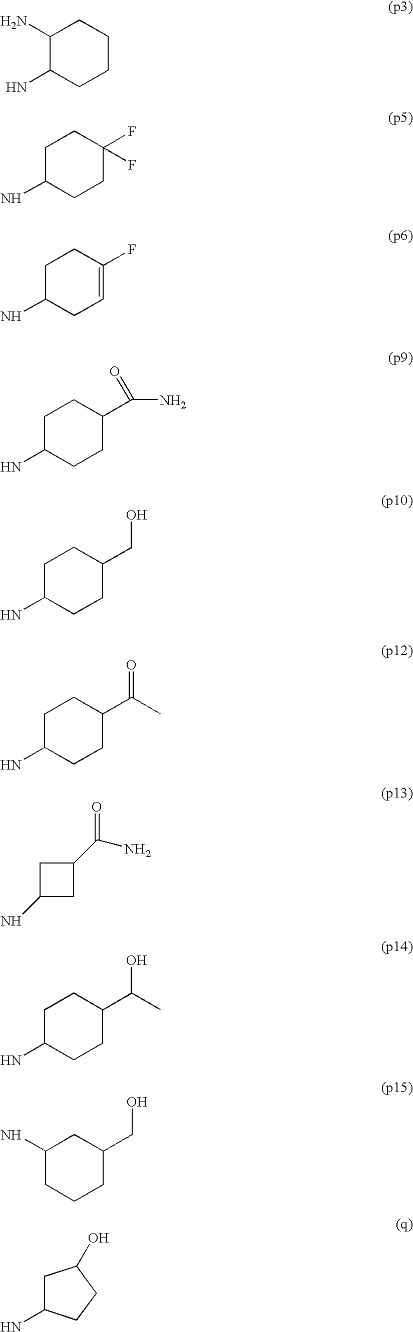

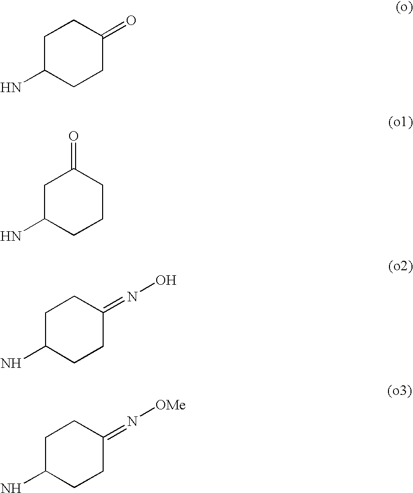

In the sub-formulae (al) to (q) etc above, the —NH— connection point of the NHR³ and/or NHR^{3a} group to the 4-position of the pyrazolopyridine of formula (I) is underlined.

Preferably, NHR³ and/or NHR^{3a} independently is or are of sub-formula (c), (c1), (c 2), (c 3), (c 4), (c 5), (c 6), (c 7), (d), (e), (f), (g4), (h), (i), (j), (k), (k1), (k2), (k3), (L), (m), (m1), (m3), (m4), (n), (o), (o1), (o2), (o3), (p), (p2), (p5), (p6), (p9), (p10), (p12), (p13), (p14), (p15) or (q); or preferably NHR³ and/or NHR^{3a} independently is or are of sub-formula (a1), (c), (c1), (c 2), (c 3), (c 4), (c 5), (c 6), (c 7), (d), (e), (f), (g4), (h), (i), (j), (k), (k1), (k2), (k3), (L), (m), (m1), (m3), (m4), (n), (o), (o1), (o2), (o3), (p), (p1), (p2), (p5), (p6), (p9), (p10), (p12), (p13), (p14), (p15) or (q).

More preferably, NHR³ and/or NHR^{3a} independently is or are of sub-formula (c), (c1), (c 4), (c 5), (h), (i), (k), (k2), (k3), (m1), (n), (o), (o2), (o3), (p2), (p5), (p6), (p9), (p10), (p13) or (p15).

NHR³ and/or NHR^{3a} independently is or are more preferably of sub-formula (c), (h), (k), (k2), (k3), (n), (o), (o2), (p9) or (p13); still more preferably NHR³ and/or NHR^{3a} independently is or are (c), (h), (k2), (k3), (n), (o), (o2), (p9) or (p13).

Most preferably, R³ and/or R^{3a} independently is or are tetrahydro-2H-pyran-4-yl or 1-(aminocarbonyl)-4-piperidinyl; that is NHR³ and/or NHR^{3a} independently is or are most preferably of sub-formula (h) or (k2), as shown above, in particular of sub-formula (h).

When NHR³ and/or NHR^{3a} independently is or are of sub-formula (n), then it can be in the trans configuration. But preferably it is in the cis configuration, i.e. preferably it is a cis-(3-hydroxycyclohexan-1-yl)amino group (including mixtures of configurations wherein the cis configuration is the major component), or it is racemic.

When NHR³ and/or NHR^{3a} independently is or are of sub-formula (p9), then it can be in the trans configuration. But preferably it is in the cis configuration, i.e. preferably it is a cis-[4-(aminocarbonyl)cyclohexan-1-yl]amino group (including mixtures of configurations wherein the cis configuration is the major component), or it is racemic.

When NHR³ and/or NHR^{3a} independently is or are of sub-formula (p12), then it can be in the trans configuration. But, preferably, it is in the cis configuration, i.e. preferably NHR³ and/or NHR^{3a} independently is or are a cis-[4-acetylcyclohexan-1-yl]amino group (including mixtures of configurations wherein the cis configuration is the major component), or it is racemic.

When NHR³ and/or NHR^{3a} independently is or are of sub-formula (p13), then it can be in the trans configuration. But, preferably, it is in the cis configuration, i.e. preferably NHR³ and/or NHR^{3a} independently is or are a cis-[3-(aminocarbonyl)cyclobutan-1-yl]amino group (including mixtures of configurations wherein the cis configuration is the major component), or it is racemic.

The NHR³ and/or NHR^{3a} group of sub-formula (p10), (p14) or (p15), independently, can for example be racemic; or it can be in the cis configuration with respect to the ring (including mixtures of configurations wherein the cis configuration is the major component).

R⁴ and/or R^{4a} can suitably independently be a hydrogen atom (H), methyl or ethyl.

R⁴ and/or R^{4a} can suitably independently be a hydrogen atom (H) or methyl.

Preferably, R⁴ and/or R^{4a} independently is or are a hydrogen atom (H). More preferably, R⁴ and R^{4a} are a hydrogen atom (H).

The compound of formula (I) or the salt thereof can suitably be:
N-{[1,6-diethyl-4-(tetrahydro-2H-pyran-4-ylamino)-1H-pyrazolo[3,4-b]pyridin-5-yl]methyl}
4-[3-({[1,6-diethyl-4-(tetrahydro-2H-pyran-4-ylamino)-1H-pyrazolo[3,4-b]pyridin-5-yl]methyl}amino)-3-oxopropyl]benzamide,
N,N′-bis{[1,6-diethyl-4-(tetrahydro-2H-pyran-4-ylamino)-1H-pyrazolo[3,4-b]pyridin-5-yl]methyl}pentanediamide,
N,N′-bis{[1,6-diethyl-4-(tetrahydro-2H-pyran-4-ylamino)-1H-pyrazolo[3,4-b]pyridin-5-yl]methyl}pentanediamide,
N,N′-bis{[1,6-diethyl-4-(tetrahydro-2H-pyran-4-ylamino)-1H-pyrazolo[3,4-b]pyridin-5-yl]methyl}-3,3-dimethylpentanediamide,
N,N′-bis{[1,6-diethyl-4-(tetrahydro-2H-pyran-4-ylamino)-1H-pyrazolo[3,4-b]pyridin-5-yl]methyl}-2,3-dimethylbutanediamide,
4,4′-methanediylbis(N-{[1,6-diethyl-4-(tetrahydro-2H-pyran-4-ylamino)-1H-pyrazolo[3,4-b]pyridin-5-yl]methyl}benzamide),
2,2′-benzene-1,4-diylbis(N-{[1,6-diethyl-4-(tetrahydro-2H-pyran-4-ylamino)-1H-pyrazolo[3,4-b]pyridin-5-yl]methyl}acetamide),
N-{[1,6-diethyl-4-(tetrahydro-2H-pyran-4-ylamino)-1H-pyrazolo[3,4-b]pyridin-5-yl]methyl}
2-[3-({[1,6-diethyl-4-(tetrahydro-2H-pyran-4-ylamino)-1H-pyrazolo[3,4-b]pyridin-5-yl]methyl}amino)-3-oxopropyl]benzamide,
2,2′-benzene-1,3-diylbis(N-{[1,6-diethyl-4-(tetrahydro-2H-pyran-4-ylamino)-1H-pyrazolo[3,4-b]pyridin-5-yl]methyl}acetamide),
2,2′-oxybis(N-{[1,6-diethyl-4-(tetrahydro-2H-pyran-4-ylamino)-1H-pyrazolo[3,4-b]pyridin-5-yl]methyl}acetamide),
2,2′-oxybis(N-{[1,6-diethyl-4-(tetrahydro-2H-pyran-4-ylamino)-1H-pyrazolo[3,4-b]pyridin-5-yl]methyl}acetamide),
N,N′-bis{[1,6-diethyl-4-(tetrahydro-2H-pyran-4-ylamino)-1H-pyrazolo[3,4-b]pyridin-5-yl]methyl}-3,3′-biphenyldicarboxamide,
N,N′-bis[(4-{[1-(aminocarbonyl)-4-piperidinyl]amino}-1,6-diethyl-1H-pyrazolo[3,4-b]pyridin-5-yl)methyl]-1,4-cyclohexanedicarboxamide,
4,4′-{sulfonylbis[(1-oxo-2,1-ethanediyl)iminomethanediyl(1,6-diethyl-1H-pyrazolo[3,4-b]pyridine-5,4-diyl)imino]}di(1-piperidinecarboxamide),
4-({5-[({[3-({[(4-{[1-(aminocarbonyl)-4-piperidinyl]amino}-1,6-diethyl-1H-pyrazolo[3,4-b]pyridin-5-yl)methyl]amino}carbonyl)phenyl]acetyl}amino)methyl]-1,6-diethyl-1H-pyrazolo[3,4-b]pyridin-4-yl}amino)-1-piperidinecarboxamide,
4-({5-[({3-[4-({[(4-{[1-(aminocarbonyl)-4-piperidinyl]amino}-1,6-diethyl-1H-pyrazolo[3,4-b]pyridin-5-yl)methyl]amino}carbonyl)phenyl]propanoyl}amino)methyl]-1,6-diethyl-1H-pyrazolo[3,4-b]pyridin-4-yl}amino)-1-piperidinecarboxamide,
4-({5-[({[4-({[(4-{[1-(aminocarbonyl)-4-piperidinyl]amino}-1,6-diethyl-1H-pyrazolo[3,4-b]pyridin-5-yl)methyl]amino}carbonyl)phenyl]acetyl}amino)methyl]-1,6-diethyl-1H-pyrazolo[3,4-b]pyridin-4-yl}amino)-1-piperidinecarboxamide,
4-[(5-{[({[2-({[(4-{[1-(aminocarbonyl)-4-piperidinyl]amino}-1,6-diethyl-1H-pyrazolo[3,4-b]pyridin-5-yl)methyl]amino}carbonyl)phenyl]oxy}acetyl)amino]methyl}-1,6-diethyl-1H-pyrazolo[3,4-b]pyridin-4-yl)amino]-1-piperidinecarboxamide,
N,N′-bis[(4-{[1-(aminocarbonyl)-4-piperidinyl]amino}-1,6-diethyl-1H-pyrazolo[3,4-b]pyridin-5-yl)methyl]butanediamide,
4,4′-{methanediylbis[benzene-4,1-diyl(oxomethanediyl)iminomethanediyl(1,6-diethyl-1H-pyrazolo[3,4-b]pyridine-5,4-diyl)imino]}di(1-piperidinecarboxamide),
N,N′-bis[(4-{[1-(aminocarbonyl)-4-piperidinyl]amino}-1,6-diethyl-1H-pyrazolo[3,4-b]pyridin-5-yl)methyl]-3,3-dimethylpentanediamide,
N,N′-bis[(4-{[1-(aminocarbonyl)-4-piperidinyl]amino}-1,6-diethyl-1H-pyrazolo[3,4-b]pyridin-5-yl)methyl]-1,3-cyclopentanedicarboxamide,
4,4′-{oxybis[(1-oxo-2,1-ethanediyl)iminomethanediyl (1,6-diethyl-1H-pyrazolo[3,4-b]pyridine-5,4-diyl)imino]}di(1-piperidinecarboxamide),
N,N′-bis{[1,6-diethyl-4-(tetrahydro-2H-pyran-4-ylamino)-1H-pyrazolo[3,4-b]pyridin-5-yl]methyl}-1,4-benzenedicarboxamide,
N, N-bis[(4-{[1-(aminocarbonyl)-4-piperidinyl]amino}-1,6-diethyl-1H-pyrazolo[3,4-b]pyridin-5-yl)methyl]-1,3-cyclohexanedicarboxamide,
N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide,
4,4′-oxybis(N-{[1,6-diethyl-4-(tetrahydro-2H-pyran-4-ylamino)-1H-pyrazolo[3,4-b]pyridin-5-yl]methyl}benzamide),
N,N′-bis[(4-{[1-(aminocarbonyl)-4-piperidinyl]amino}-1,6-diethyl-1H-pyrazolo[3,4-b]pyridin-5-yl)methyl]-1,4-benzenedicarboxamide,
N,N′-bis[(4-{[1-(aminocarbonyl)-4-piperidinyl]amino}-1,6-diethyl-1H-pyrazolo[3,4-b]pyridin-5-yl)methyl]-4,4′-biphenyldicarboxamide,
4,4′-{oxybis[benzene-4,1-diyl(oxomethanediyl)iminomethanediyl(1,6-diethyl-1H-pyrazolo[3,4-b]pyridine-5,4-diyl)imino]}di(1-piperidinecarboxamide),
N,N′-bis{[1-ethyl-6-methyl-4-(tetrahydro-2H-pyran-4-ylamino)-1H-pyrazolo[3,4-b]pyridin-5-yl]methyl}-4,4′-biphenyldicarboxamide,
N,N′-bis{[1-ethyl-6-methyl-4-(tetrahydro-2H-pyran-4-ylamino)-1H-pyrazolo[3,4-b]pyridin-5-yl]methyl}-3,3′-biphenyldicarboxamide,
N,N′-bis{[1-ethyl-6-methyl-4-(tetrahydro-2H-pyran-4-ylamino)-1H-pyrazolo[3,4-b]pyridin-5-yl]methyl}-2,6-naphthalenedicarboxamide,
N,N′-bis{[1,6-diethyl-4-(tetrahydro-2H-pyran-4-ylamino)-1H-pyrazolo[3,4-b]pyridin-5-yl]methyl}-2,2′-bipyridine-4,4′-dicarboxamide,
N,N′-bis{[1,6-diethyl-4-(tetrahydro-2H-pyran-4-ylamino)-1H-pyrazolo[3,4-b]pyridin-5-yl]methyl}-2,5-pyrazinedicarboxamide,
2,2′-(methylimino)bis(N-{[1,6-diethyl-4-(tetrahydro-2H-pyran-4-ylamino)-1H-pyrazolo[3,4-b]pyridin-5-yl]methyl}acetamide),
N,N′-bis{[1,6-diethyl-4-(tetrahydro-2H-pyran-4-ylamino)-1H-pyrazolo[3,4-b]pyridin-5-yl]methyl}-2,2′-bipyridine-5,5′-dicarboxamide,
N,N′-bis{[1,6-diethyl-4-(tetrahydro-2H-pyran-4-ylamino)-1H-pyrazolo[3,4-b]pyridin-5-yl]methyl}-2,5furandicarboxamide, or
N,N′-bis{[1,6-diethyl-4-(tetrahydro-2H-pyran-4-ylamino)-1H-pyrazolo[3,4-b]pyridin-5-yl]methyl}-2,5-thiophenedicarboxamide;
   or a salt thereof such as a pharmaceutically acceptable salt thereof.

The structures of the above-listed specific compounds or salts, or embodiments (e.g. specific salts) thereof, are given in Examples 1 to 36 hereinafter.

In one preferred embodiment, is still preferred that the compound of formula (I) or the salt thereof is a compound of Example 23, 29 or 35 (in particular Example 23), as defined by the structures and/or chemical names described herein, or a (any) salt thereof; e.g. the compound or salt can be a compound or a pharmaceutically acceptable salt thereof. The structures and names of these Examples are described in the Examples section and/or in the compounds list disclosed herein. The compound of Example 23, 29 or 35 or the salt thereof can suitably be for inhaled administration e.g. to a mammal such as a human. The compound of Example 23, 29 or 35, or a pharmaceutically acceptable salt thereof, can suitable be contained/comprised in a pharmaceutical composition suitable and/or adapted for inhaled administration, e.g. for inhaled administration to a mammal such as a human, monkey, or rodent (e.g. rat or mouse), in particular to a human.

In one particularly preferred embodiment, the compound of formula (I) or (II) or the salt thereof is: N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide, which is,

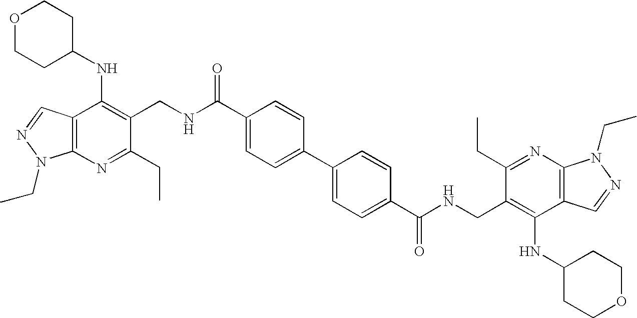

or a salt thereof (e.g. the compound or a pharmaceutically acceptable salt thereof). See for example Example 23 disclosed herein. This compound or the salt thereof can be for inhaled administration e.g. to a mammal such as a human.

In particular, the compound or salt can be N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide or a 1,5-naphthalenedisulfonate, 1,2,4-benzenetricarboxylate, para-toluenesulfonate (tosylate, e.g. di-para-toluenesulfonate=di-tosylate), methanesulfonate (e.g. di-methanesulfonate), hydroxyethylidene-1,1-diphosphonate, hydrochloride (e.g. dihydrochloride), maleate or sulphate salt thereof. This compound or the salt thereof can be for inhaled administration e.g. to a mammal such as a human. Preferably, the compound or salt is a 1,5-naphthalenedisulfonate, 1,2,4-benzenetricarboxylate, or para-toluenesulfonate (tosylate, e.g. di-para-toluenesulfonate=di-tosylate) salt of N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide.

In an alternative separate embodiment, the compound of formula (I) or the salt thereof does not include N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide

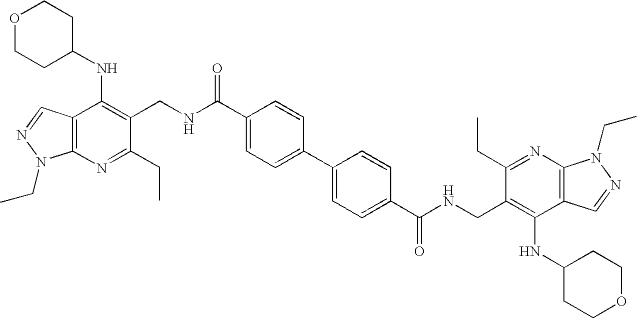

or a salt thereof.

In one preferred embodiment, the compound of formula (I) or (II) or the salt thereof is: N,N′-bis{[1-ethyl-6-methyl-4-(tetrahydro-2H-pyran-4-ylamino)-1H-pyrazolo[3,4-b]pyridin-5-yl]methyl}-3,3′-biphenydicarboxamide, which is,

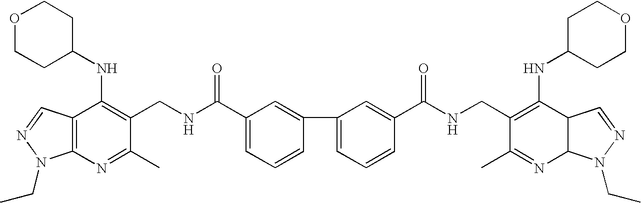

or a salt thereof (e.g. the compound or a pharmaceutically acceptable salt thereof). See for example Example 29 disclosed herein. This compound or the salt thereof can be for inhaled administration e.g. to a mammal such as a human.

In one preferred embodiment of the invention, the compound of formula (I) or the salt thereof is a compound of formula (III), which is N,N′-bis{[1,6-diethyl-4-(tetrahydro-2H-pyran-4-ylamino)-1H-pyrazolo[3,4-b]pyridin-5-yl]methyl}-2,5furandicarboxamide, or a salt thereof, for example a compound of formula (III) or a pharmaceutically acceptable salt thereof:

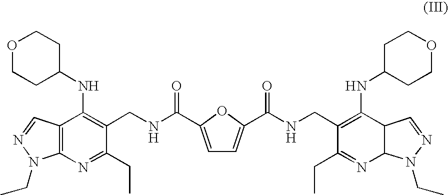

See for example Example 35 disclosed herein. This compound or the salt thereof can be for inhaled administration e.g. to a mammal such as a human.

In an alternative separate embodiment, the compound of formula (I) or the salt thereof does not include a compound of formula (III) or a salt thereof.

### Salts, Solvates, Isomers, Tautomeric Forms, Molecular Weights, Etc.

Because of their potential use in medicine, the salts of the compounds of formula (I) are preferably pharmaceutically acceptable. Suitable pharmaceutically acceptable salts can include acid addition salts, or less commonly (e.g. if a C(O)OH group is present in the compound) base addition salts.

In one embodiment, a pharmaceutically acceptable acid addition salt is optionally formed by reaction of a compound of formula (I) with a suitable inorganic or organic acid

(such as hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration. A suitable inorganic or organic acid can e.g. be hydrobromic, hydrochloric, sulfuric, nitric, phosphoric, maleic, para-toluenesulfonic, benzenesulfonic, methanesulfonic, ethanesulfonic, naphthalenesulfonic such as 2-naphthalenesulfonic acid, naphthalenedisulfonic acid such as 1,5-naphthalenedisulfonic acid, 1,2,4-benzenetricarboxylic acid, or hydroxyethylidene-1,1-diphosphonic acid. A pharmaceutically acceptable acid addition salt of a compound of formula (I) can comprise or be for example a hydrobromide, hydrochloride (e.g. dihydrochloride), sulfate, nitrate, phosphate, succinate, maleate, formate, acetate, propionate, fumarate, citrate, tartrate, lactate, benzoate, salicylate, glutamate, aspartate, para-toluenesulfonate (e.g. di-para-toluenesulfonate), benzenesulfonate, methanesulfonate (e.g. di-methanesulfonate), ethanesulfonate, naphthalenesulfonate (e.g. 2-naphthalenesulfonate), naphthalenedisulfonate (e.g. 1,5-naphthalenedisulfonate), 1,2,4-benzenetricarboxylate, hydroxyethylidene-1,1-diphosphonate, or hexanoate salt.

In one embodiment, a pharmaceutically acceptable base addition salt is optionally formed by reaction of a compound of formula (I) with a suitable inorganic or organic base (e.g. triethylamine, ethanolamine, triethanolamine, choline, arginine, lysine or histidine), optionally in a suitable solvent such as an organic solvent, to give the base addition salt which is usually isolated for example by crystallisation and filtration.

Other suitable pharmaceutically acceptable salts include pharmaceutically acceptable metal salts, for example pharmaceutically acceptable alkali-metal or alkaline-earth-metal salts such as sodium, potassium, calcium or magnesium salts; in particular pharmaceutically acceptable metal salts of one or more carboxylic acid moieties that may be present in the compound of formula (I).

Other non-pharmaceutically acceptable salts, e.g. oxalates, may be used, for example in the isolation of compounds of the invention, and are included within the scope of this invention.

The invention includes within its scope all possible stoichiometric and non-stoichiometric forms of the salts of the compounds of formula (I).

Also included within the scope of the invention are all solvates, hydrates and complexes of compounds and salts of the invention.

Certain groups, substituents, compounds or salts included in the present invention may be present as isomers. The present invention includes within its scope all such isomers, including racemates, enantiomers and mixtures thereof.

In the compounds or salts, pharmaceutical compositions, uses, methods of treatment/prophylaxis, methods of preparing, etc. according to the present invention, where a defined isomeric configuration e.g. stereochemical configuration is described or claimed, the invention includes a mixture comprising (a) a major component of the compound or salt which is in the described or claimed configuration, together with (b) one or more minor components of the compound or salt which is/are not in the described or claimed configuration. Preferably, in such a mixture, the major component of the compound or salt which is in the described or claimed configuration represents 70% or more, or 75% or more, more preferably 85% or more, still more preferably 90% or more, yet more preferably 95% or more, yet more preferably 98% or more, of the total amount of compound or salt present in the mixture on a molarity basis.

The percentage of one isomeric/stereochemical component in a mixture of different isomeric/stereochemical components, and if appropriate enantiomeric and/or diastereomeric excesses, can be measured using techniques known in the art. Such methods include the following:

(1) Measurement using NMR (e.g. ¹H NMR) spectroscopy in the presence of chiral agent. One can measure a nuclear magnetic resonance (NMR) spectrum (preferably a ¹H NMR spectrum, and/or a solution-phase NMR spectrum e.g. in CDCl₃ or D6-DMSO solvent) of the compound/salt mixture in the presence of a suitable chiral agent which “splits” the NMR peaks of a given atom in different isomers into different peak positions. The chiral agent can be: i) an optically pure reagent which reacts with the compound/salt e.g. to form a mixture of diastereomers, ii) a chiral solvent, iii) a chiral molecule which forms a transient species (e.g. diastereomeric species) with the compound/salt, or iv) a chiral shift reagent. See e.g. J. March, “Advanced Organic Chemistry”, 4th edn., 1992, pages 125-126 and refs. 138-146 cited therein. A chiral shift reagent can be a chiral lanthanide shift reagent such as tris[3-trifluoroacetyl-d-camphorato]europiijm-(III) or others as described in Morrill, “Lanthanide Shift Reagents in Stereochemical Analysis”, VCH, New York, 1986. Whatever the chiral agent is that is used, usually, the relative integrals (intensities) for the NMR peaks of a given atom or group in different isomers can provide a measurement of the relative amounts of each isomer present.

(2) Measurement using chiral chromatography, especially on an analytical scale. A suitable chiral column which separates the different isomeric components can be used to effect separation, e.g. using gas or liquid chromatography such as HPLC, and/or e.g. on an analytical scale. The peaks for each isomer can be integrated (area under each peak); and a comparison or ratio of the integrals for the different isomers present can give a measurement of the percentage of each isomeric component present. See for example: “Chiral Chromatography”, Separation Science Series Author: T. E. Beesley and R. P. W. Scott, John Wiley & Sons, Ltd., Chichester, UK, 1998, electronic Book ISBN: 0585352690, Book ISBN: 0471974277.

(3) Separation of pre-existing diastereomeric mixtures which are compounds/salts of the invention can be achieved (usually directly, without derivatisation) using separation techniques such as gas or liquid chromatography. Diastereomeric ratios and/or excesses can thereby be derived e.g. from the relative peak areas or relative separated masses.

(4) Conversion with a chiral/optically-active agent and subsequent separation of the resulting isomers, e.g. diastereomers. Conversion can be via derivatisation of a derivatizable group (e.g. —OH, —NHR) on the compound/salt with an optically-active derivatizing group (e.g. optically active acid chloride or acid anhydride); or can be via formation of an acid or base addition salt of the compound by treatment of the compound with an optically-active acid or base, such as + or − di-para-toluoyl tartaric acid. After derivatisation, separation of the resulting isomers e.g. diastereomers, can be using gas or liquid chromatography (usually non-chiral); or (especially with isomeric salts) can be by selective crystallisation of a single isomeric e.g. diastereoisomeric salt. Determination of isomeric ratios and/or excesses can be using chromatography peak areas or measurement of mass of each separated isomer.

See e.g. J. March, “Advanced Organic Chemistry”, 4th edn., 1992, pages 120-121 and 126, and refs. 105-115 and 147-149 cited therein.

(5) Measurement of optical activity [alpha] of mixture and comparison with optical activity of pure isomer [alpha]ₘₐₓ if available (e.g. see J. March, “Advanced Organic Chemistry”, 4th edn., 1992, page 125 and refs. 138-139 cited therein). This assumes a substantially linear relationship between [alpha] and concentration.

Certain of the groups, e.g. heteroaromatic ring systems, included in compounds of formula (I) or their salts may exist in one or more tautomeric forms. The present invention includes within its scope all such tautomeric forms, including mixtures.

The compound of formula (I) can optionally have a molecular weight of 1500 or less, for example 1200 or less, in particular 900 or less. Molecular weight here refers to that of the unsolvated “free base” compound, that is excluding any molecular weight contributed by any addition salts, solvent (e.g. water) molecules, etc.

### Synthetic Process Routes

The following non-limiting processes can generally be used to prepare the compounds of the invention:

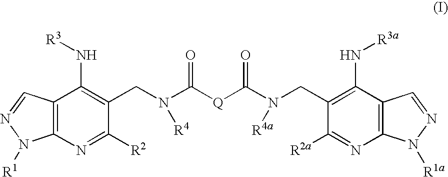

Some of the following synthetic processes may be exemplified for compounds of Formula (I), (II) or (III) with particular substitution patterns. However, at least some of these processes can be used with appropriate modification, e.g. of starting materials and reagents, for making other compounds of Formula (I).

### Process A

In the embodiment “Process A”, to prepare a compound of formula (I), (II) or (III), an amine of formula (IVa) or a salt thereof, and optionally also an amine of formula (IVb) (when different to the amine of formula (IVa)) or a salt thereof, can be reacted with a compound of formula (V) which is X^{1a}—C(O)-Q-C(O)—X^{1a}, wherein X^{1a} is a leaving group which is substitutable by the NHR⁴ moiety of the compound of formula (IVa) (and when (IVb) is used X^{1a} is a leaving group which is also substitutable by the NHR^{4a} amine moiety of the compound of formula (IVb)):

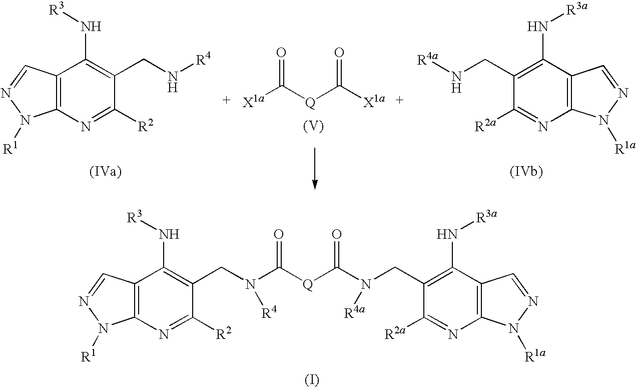

In the typical circumstance that R¹=R^{1a}, R²=R^{2a}, R³=R^{3a}, and R⁴=R^{4a}, then the amine of formula (IVa) or a salt thereof is reacted with a compound of formula (V) in the process; and an additional different amine of formula (IVb) is not required.

The compound of formula (V) which is X^{1a}—C(O)-Q-C(O)—X^{1a} is typically an activated derivative of the di-carboxylic acid of formula (VI), shown below. To form a compound of formula (V) which is X^{1a}—C(O)-Q-C(O)—X^{1a} a di-carboxylic acid of formula (VI)

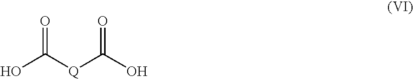

can be converted into the compound of formula (V).

In one preferred embodiment, the compound X^{1a}—C(O)-Q-C(O)—X^{1a} of formula (V) is an activated derivative of the di-carboxylic acid of formula (VI) in which the leaving group X^{1a} is

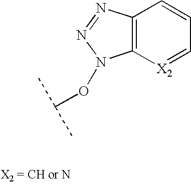

This activated compound X^{1a}—C(O)-Q-C(O)—X^{1a}, where X^{1a} is as shown above, is typically formed from the di-carboxylic acid of formula (VI) by the following reaction (a). In optional reaction (a), the di-carboxylic acid of formula (VI) is reacted with (i), (ii), (iii) or (iv):

(i) O-(7-azabenzotriazol-1-yl)-N,N,N′,N′-tetramethyluronium hexafluorophosphate (HATU) (when X₂ is N), or
(ii) O-(benzotriazol-1-yl)-N,N,N′,N′-tetramethyluronium hexafluorophosphate (HBTU) (when X₂ is CH), or
(iii) 2-(1H-benzotriazol-1-yl)-1,1,3,3-tetramethyluronium tetrafluoroborate (TBTU) (when X₂ is CH), or
(iv) benzotriazol-1-yl-oxy-trispyrrolidinophosphonium hexafluorophosphate (PyBOP). In one embodiment, reaction (a) is carried out in the presence of a tertiary amine base such as diisopropylethylamine (ⁱPr₂NEt=DIPEA), and/or in the presence of a non-aqueous non-alcohol organic solvent (e.g. anhydrous solvent) such as dimethyl formamide (DMF, e.g. dry DMF) or acetonitrile e.g. dry acetonitrile. In one embodiment, reaction (a) is carried out at room temperature (e.g. about 18 to about 25° C.), for example for from 1 hour to 4 days, for example from 2 hours to 3 days. For example reaction (a) can optionally be carried out under anhydrous conditions.

In an alternative embodiment, the di-carboxylic acid of formula (VI) is reacted with a suitable organic di-substituted carbodiimide, such as 1-(3-dimethylaminopropyl)-3-ethylcarbodiimide or a salt thereof (EDC) e.g. the hydrochloride salt, or such as dicyclohexylcarbodiimide (DCC), optionally also in the presence of 1-hydroxybenzotriazole (HOBT); and the resulting carbodiimide-diacid adduct or HOBT-diacid adduct of formula (V) for example then reacts with the amine of formula (IVa) or a salt thereof (and optionally if appropriate also an amine of formula (IVb)). In one embodiment, this reaction is carried out in the presence of a non-aqueous non-alcohol organic solvent (e.g. anhydrous solvent) such as dimethyl formamide (DMF) or acetonitrile and/or e.g. at room temperature and/or e.g. under anhydrous conditions. In one embodiment, the reaction is carried out in the presence of a tertiary amine base such as diisopropylethylamine (ⁱPr₂NEt=DIPEA).

In another alternative embodiment, the compound of formula (V) which is X^{1a}—C(O)-Q-C(O)—X^{1a} a can for example be the acid chloride Cl—C(O)-Q-C(O)—Cl (X^{1a}=Cl). The acid chloride Cl—C(O)-Q-C(O)—Cl within formula (V) can for example be formed from the corresponding di-carboxylic acid (VI) by reaction with thionyl chloride, either in an organic solvent such as chloroform or without solvent. When Cl—C(O)-Q-C(O)—Cl is used to prepare the compound of formula (I), (II) or (III), the reaction with amine (IVa), and optionally also with amine (IVb), is in one embodiment carried out in the presence of a tertiary amine base such as diisopropylethylamine (ⁱPr₂NEt=DIPEA) and/or in a suitable non-aqueous non-alcohol organic solvent such as acetonitrile (e.g. anhydrous), for example at room temperature (e.g. about 18 to about 25° C.).

Amine compounds of formula (IVa) or (IVb) wherein R⁴ (or R^{4a}) is H can generally be prepared by hydrogenation of an azide compound of formula (VII):

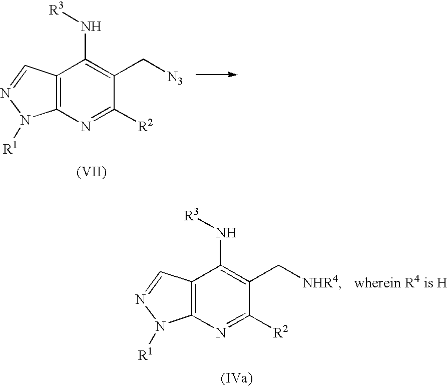

Typical hydrogenation conditions can include H₂/palladium on carbon. See for example Intermediates 8, 13 and 18.

In an alternative embodiment, to prepare amine compounds of formula (IVa) or (IVb) wherein R⁴ (or R^{4a}) is not H, an amine of formula (IVa) or (IVb) wherein R⁴ (or R^{4a}) is H, or a salt thereof, is optionally reacted with a reagent suitable for adding CF₃C(O)— to the primary-amine nitrogen atom (such as CF₃C(O)OEt); and then alkylation of the said nitrogen with R⁴ (e.g. using alkylating agent R⁴—X^{6a} wherein X^{6a} is a suitable leaving group such as an iodine atom); followed by removal of the CF₃C(O)— group from the NR⁴ nitrogen e.g. using NaOH:

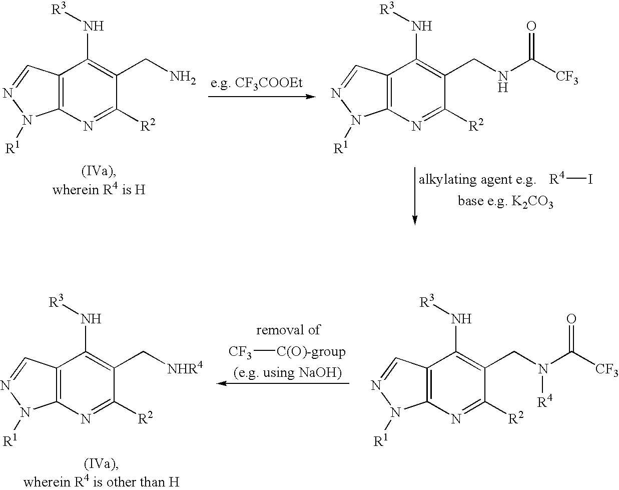

Azide compounds of formula (VII) can generally or sometimes be prepared by reaction of a compound of formula (VIII), wherein X^{3b} is a leaving group displaceable by azide, with a metal azide such as sodium azide, lithium azide or potassium azide:

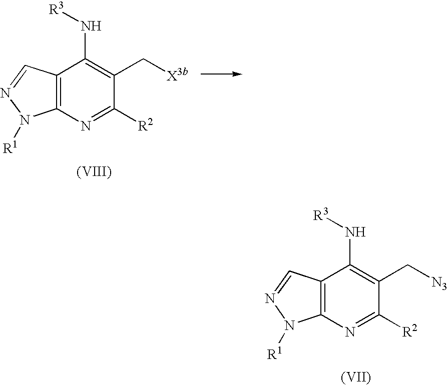

Typical conditions for the (VIII) to (VII) reaction, e.g. with sodium azide or lithium azide, can e.g. include DMSO solvent (e.g. dry) and/or reaction at room temperature. See for example Intermediates 7 and 17. In one embodiment, X^{3b} is a chlorine atom (Cl) or an organic sulfonate such as methanesulfonate, trifluoromethanesulfonate or p-toluenesulfonate, in particular a chlorine atom.

Compounds of formula (VIII), in particular wherein X^{3b} is Cl or an organic sulfonate, can generally or sometimes be prepared by conversion (e.g. chlorination) of an alcohol compound of formula (IX), e.g. by reaction with SOCl₂ (thionyl chloride) for chlorination (when X^{3b} is Cl), or by reaction with methanesulfonyl chloride (when X^{3b} is methanesulfonate) or p-toluenesulfonyl chloride (when X^{3b} is p-toluenesulfonate). The chlorination reaction with thionyl chloride may require heating, e.g. to about 60 to about 90° C., for example at about 85° C.

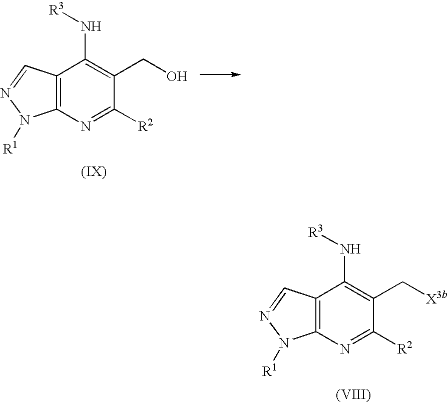

It is noted that when R³ includes a urea functionality, e.g. when NHR³ is of sub-formula (k2) or (k3), then this is unlikely to tolerate thionyl chloride.

In an alternative method, azide compounds of formula (VII) can be prepared directly from the alcohol compound of formula (IX). For example, reacting compounds of formula (IX) with an azide, e.g. sodium azide, in the presence of carbon tetrabromide and triphenylphosphine can give compounds of formula (VII) (e.g. see Toyota et. al. Journal of Organic Chemistry, 2000, 65(21), 7110-7113); see for example Intermediate 11.

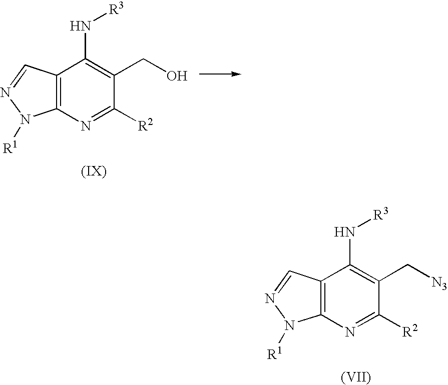

In another alternative method, the amine compound of formula (IVa) can be prepared directly from the compound of formula (VIII) (for example wherein X^{3b} is or comprises a chlorine atom), without first converting (VIII) to the azide compound of formula (VII):

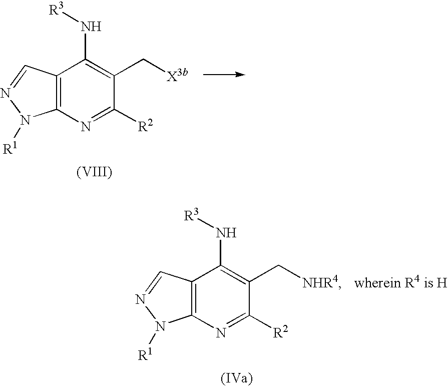

For example, this reaction of (VIII) to (IVa) can generally or sometimes be carried out by reaction of the compound of formula (VIII) (for example wherein X^{3b} is or comprises a chlorine atom) with an aminating agent such as lithium hexamethyldisilazide, in a suitable non-aqueous non-alcohol organic solvent (e.g. anhydrous solvent) such as tetrahydrofuran, for example at a suitable temperature of for example about 50 to about 60° C. The reaction can optionally be followed by treatment with an acid such as 5M aqueous hydrochloric acid at a suitable temperature such as room temperature.

Compounds of formula (IX) can generally or sometimes be prepared by reduction of an ester compound of formula (X), wherein R^{e} is C₁₋₄alkyl such as ethyl, in the presence of a reducing agent. The reducing agent can for example be diisobutylaluminum hydride (for which the reduction can e.g. be in dichloromethane solvent) or lithium borohydride (LiBH₄):

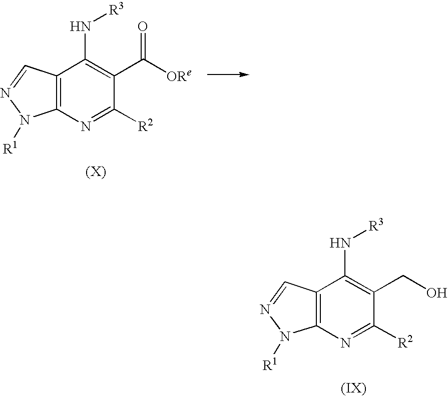

Compounds of formula (X) can generally or sometimes be prepared by reaction of a compound of formula (XI) with an amine of formula R³NH₂ (or with an R³-protected derivative of R³NH₂ as appropriate, for which see later for when NHR³ is of sub-formula (k2) or (k3)). The reaction is for example carried out in the presence of a tertiary amine base such as triethylamine or N,N-diisopropylethylamine, and/or in an organic solvent such as ethanol, dioxane, 1-methyl-2-pyrrolidinone (NMP) or acetonitrile. The reaction may comprise heating e.g. heating to ca. 60-180° C., for example ca. 60-100° C. (e.g. ca. 80-90° C.) or ca. 110-160° C., for example depending on the reflux temperature or boiling point of the solvent(s) used. For an example process, see Intermediates 5 or 15 herein, or see the process shown in Yu et. al. in J. Med. Chem., 2001, 44, 1025-1027 (see steps c+d of Scheme 1 therein):

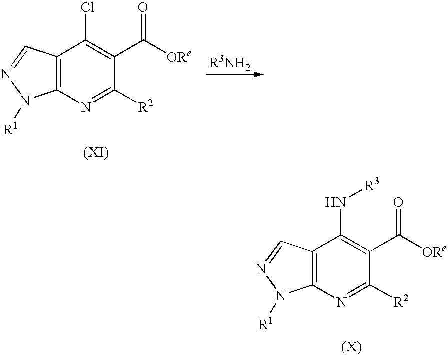

A slightly varied process for preparing the 4-amino 5-ester pyrazolopyridine compound of formula (X) involves the use of a protecting group within R³, deprotection, and derivatisation of R³. This process can sometimes be used for example when R³ contains a C(O)NH₂ ring-carbon substituent or when R³ is a heterocyclic group of sub-formula (bb) wherein R¹⁰ is C(O)NH₂. For example, this can be when R³ is a heterocyclic group of sub-formula (bb) wherein n¹ is 1 or 0 respectively and wherein R¹⁰ is C(O)NH₂, such as when NHR³ is of sub-formula (k2) or (k3) [i.e. R³ is a N-aminocarbonyl-piperidinyl or N-aminocarbonyl-pyrrolidinyl group respectively].

In a typical example wherein NHR³ is of sub-formula (k2) or (k3), the corresponding 4-amino 5-ester pyrazolopyridine compound of formula (X) can generally or sometimes be prepared by reacting a compound of formula (Xa), wherein R¹, R² and R^{e} are as defined above and n¹ is 1 or 0 respectively, or a salt thereof (e.g. a hydrochloride salt thereof) with a urea-forming reagent capable of converting the (4-piperidinyl)amino or (3-pyrrolidinyl)amino group in the compound of formula (Xa) into a [(1-aminocarbonyl)-4-piperidinyl]amino group or [(1-aminocarbonyl)-3-pyrrolidinyl]amino group (as in the formula (X) embodiment below) respectively:

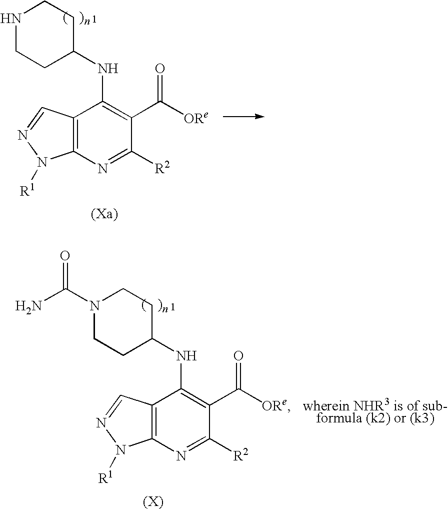

The urea-forming reagent may be benzyl isocyanate (followed later by debenzylation e.g. reductive debenzylation), or preferably the urea-forming reagent is tri(C₁₋₄alkyl)silyl isocyanate such as a tri(C₁₋₂alkyl)silyl isocyanate, preferably trimethylsilyl isocyanate. The conversion of the compound (Xa) or salt thereof to the compound (X) may be carried out in the presence of a suitable base such as N,N-diisopropylethylamine, in a suitable solvent such as dichloromethane or chloroform, at a suitable temperature such as at room temperature or at the reflux temperature of the solvent.

Compound (Xa) or the salt thereof is for example prepared from compound (Xb), wherein Prot is a nitrogen protecting group such as (tert-butyloxy)carbonyl, by removal of the nitrogen protecting group. For example, removal of the (tert-butyloxy)carbonyl group is effected under suitable acidic conditions, such as with hydrogen chloride (e.g. 4M) in a suitable solvent such as 1,4-dioxane:

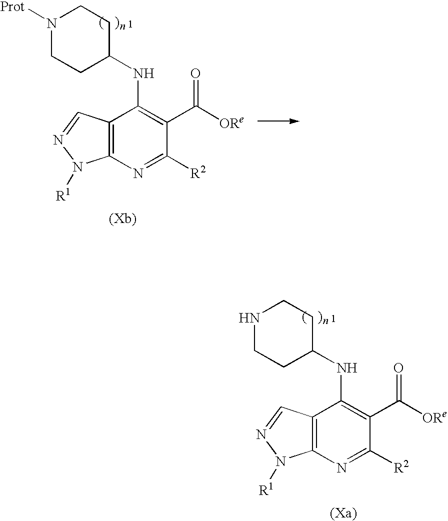

Compound (Xb), wherein R^{e} is ethyl and Prot is (tert-butyloxy)carbonyl, can usually be prepared by reaction of a compound of formula (XI), wherein R^{e} is ethyl, with 1,1-dimethylethyl 4-amino-1-piperidinecarboxylate (e.g. commercially available from AstaTech, Philadelphia, USA) or 1,1-dimethylethyl 3-amino-1-pyrrolidinecarboxylate (e.g. commercially available from Aldrich). The reaction is for example carried out in the presence of a tertiary amine base such as triethylamine or N,N-diisopropylethylamine, and/or in a suitable organic solvent such as ethanol, dioxane, 1-methyl-2-pyrrolidinone (NMP) or acetonitrile. The reaction may comprise heating e.g. heating to ca. 60-180° C., for example ca. 60-100° C. (e.g. ca. 80-90° C.) or ca. 110-160° C., for example depending on the reflux temperature or boiling point of the solvent(s) used. See for example Intermediate 9 herein:

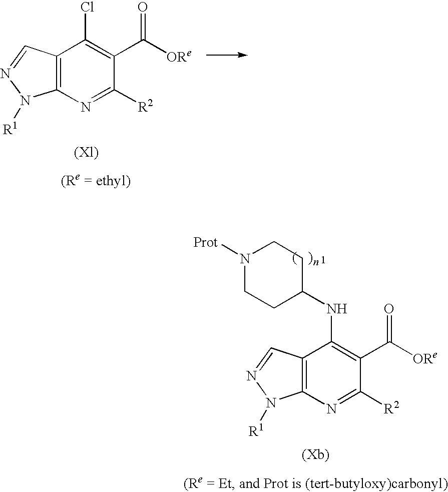

In one embodiment, a compound of formula (XI) is prepared by reaction of a compound of formula (XII) with (R²)(OEt)C═C(CO₂R^{e})₂ of formula (XIII) or (R²)(Cl)C═C(CO₂R^{e})₂ of formula (XIV), followed by reaction with phosphorous oxychloride (POCl₃). The compound (R²)(OEt)C═C(CO₂R^{e})₂ or (R²)(Cl)C═C(CO₂R^{e})₂ can for example be:

diethyl (ethoxymethylene)malonate (wherein R² is H and R^{e} is Et, formula (XIII)), or

diethyl 2-(1-ethoxyethylidene)malonate (formula (XIII)) or diethyl (1-chloroethylidene)propanedioate (formula (XIV)) (both having R²=Me and R^{e}=Et), or

diethyl (1-chloropropylidene)propanedioate (wherein R² is Et and R^{e} is Et, formula (XIV)):

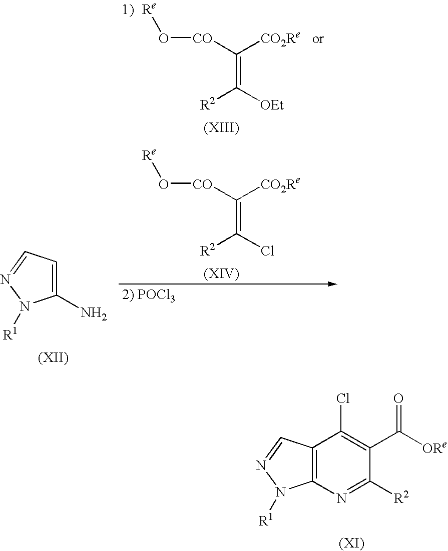

In one embodiment of the (XII) to (XI) process, the compound of formula (XII) is reacted with a dialkyl (1-chloroallylidene)propanedioate of formula (XIV) with heating, for example in a suitable organic solvent such as toluene, and for example in the presence of a suitable base such as triethylamine, e.g. at a suitable temperature such as the reflux temperature of the solvent. Suitable conditions for the reaction of the intermediate, formed from (XII) and (XIV), with phosphorous oxychloride (POCl₃) include heating, e.g. heating at the reflux temperature of phosphorous oxychloride.

For examples of the compound (XII) to compound (XI) process, see for example:

(i) Intermediate 4 synthesis hereinafter, wherein R² is ethyl, and R¹ and R^{e} are ethyl, and using diethyl (1-chloropropylidene)propanedioate within formula (XIV);
(ii) Intermediate 14 synthesis hereinafter, wherein R² is methyl, and R¹ and R^{e} are ethyl, and using (1-chloroethylidene)propanedioate within formula (XIV);
(iii) G. Yu et. al., J. Med. Chem., 2001, 44, 1025-1027 (see steps a+b of Scheme 1 therein), wherein R² is H, and R¹ and R^{e} are ethyl, and using diethyl (ethoxymethylene)malonate within formula (XIII);
(iv) WO 2004/024728 (Intermediate 25 on page 98 therein), wherein R² is H, R¹ is methyl and R^{e} is ethyl (i.e. reaction of 5-amino-1-methylpyrazole with diethyl (ethoxymethylene)malonate within formula (XIII)).

In one embodiment, the compound of formula (XIV), (R²)(Cl)C═C(CO₂R^{e})₂, is prepared by reaction of a compound of formula (XV), with phosphorus oxychloride (POCl₃) in the presence of a suitable base such as tributylamine, at a suitable temperature such as ca. 80-130° C., for example ca. 100-120° C.

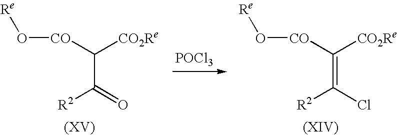

In one embodiment, a compound of formula (XV) is prepared by reaction of a dialkyl malonate of formula (XVI) with magnesium chloride and a suitable base such as triethylamine, in a suitable solvent such as acetonitrile, at a suitable temperature such as ca. 5-10° C., followed by addition of an acid chloride of formula (XVII), for example propanoyl chloride when R² is ethyl, at a suitable temperature such as between 10° C. and room temperature.

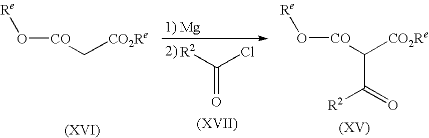

Compounds of formulae (XVI) and (XVII) are either known compounds or may be prepared by conventional means. For example compounds of formulae (XVI) and (XVII) where R^{e} and R² respectively represent ethyl are available from Aldrich.

Where the desired amino pyrazole of formula (XII) is not commercially available, preparation of the amino pyrazole (XII) can sometimes be achieved, for example, by reaction of cyanoethyl hydrazine of formula (XVIII) with a suitable aldehyde of formula R⁴⁰CHO in a solvent such as ethanol, with heating, followed by reduction, for example reduction with sodium in a solvent such as t-butanol. (See for example the method(s) described by Dorgan et. al. in J. Chem. Soc., Perkin Trans. 1, (4), 938-42; 1980.) R⁴⁰ should be chosen so as to contain one less carbon atom than R¹, so that for example R⁴⁰=methyl will afford R¹=ethyl.

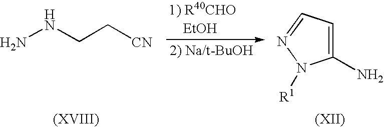

Alternatively, e.g. where the desired amino pyrazole of Formula (XII) is not commercially available, preparation of a 4-amino 5-ester pyrazolopyridine compound of Formula (X) can sometimes be achieved from a 4-chloro 5-ester pyrazolopyridine compound of Formula (X¹) having a different R¹ group, using a generalised version of the reaction scheme shown below. In this method:

the 4-chloro 5-ester pyrazolopyridine compound of Formula (X¹) is optionally converted to the 4-alkoxy (e.g. C₁₋₄alkoxy such as ethoxy)pyrazolopyridine;

the old non-desired R¹ group is removed (e.g. using N-bromosuccinimide (NBS) and preferably base e.g. Na₂CO₃);

the 4-amino NHR³ group is inserted by displacing the 4-chloro or 4-alkoxy group by reaction with R³NH₂;

and the resulting pyrazolopyridine is alkylated at N−1 by reacting it with R¹—X⁴¹, wherein R¹ is the new desired R¹ group, and wherein X⁴¹ is a group displaceable by the N−1 nitrogen of the pyrazolopyridine, in order to re-insert the desired R¹ group [i.e. to prepare the desired 4-amino 5-ester pyrazolopyridine compound of Formula (X)]. X⁴¹ can for example be a halogen, e.g. Cl, Br or I; or X⁴¹ can be —O—S(O)₂—R⁴¹ where R⁴¹ is C₁₋₄alkyl, C₁₋₂fluoroalkyl, or phenyl optionally substituted by C₁₋₂alkyl. The N−1 alkylation reaction with R¹—X⁴¹ is optionally carried out in the presence of a suitable base.

The scheme below shows a suitable example of a route and conditions for this R¹ removal and re-insertion route, for removal of R¹=ethyl, and for insertion of R¹=n-propyl and R³=tetrahydro-2H-pyran-4-yl, when R²=a hydrogen atom (H):

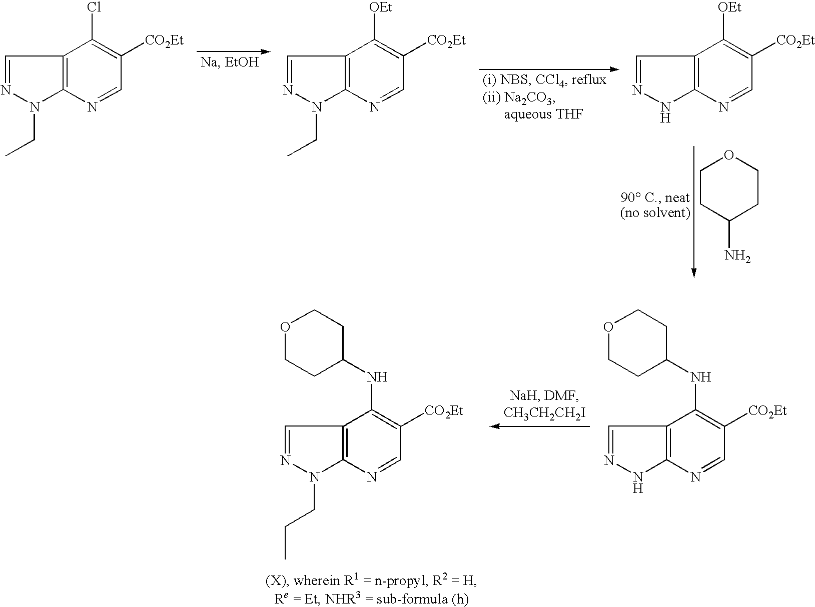

In the final alkylation step of the process exemplified above, in generalised terms a 4-amino 5-ester pyrazolopyridine compound of formula (X) can sometimes be prepared by reaction of a compound of formula (XIX) with an alkylating agent of formula R¹—X³, where X³ is a leaving group displaceable by the 1-position pyrazolopyridine nitrogen atom of the compound of formula (XIX):

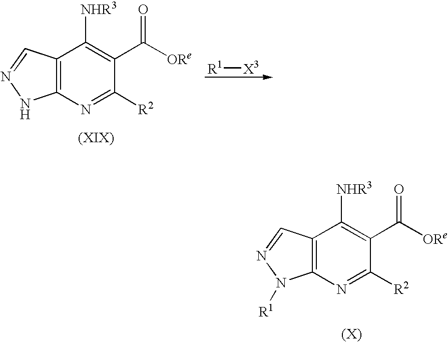

A suitable alkylating agent of formula R¹—X³ can be used. For example, X³ can be a halogen atom such as a chlorine atom or more preferably a bromine or iodine atom, or X³ can be —O—S(O)₂—R³⁶ wherein R³⁶ is C₁₋₈alkyl (e.g. C₁₋₄alkyl or C₁₋₂alkyl such as methyl), C₁₋₆fluoroalkyl (e.g. C₁₋₄fluoroalkyl or C₁₋₂fluoroalkyl such as CF₃ or C₄F₉), or phenyl wherein the phenyl is optionally substituted by one or two of independently C₁₋₂alkyl, halogen or C₁₋₂alkoxy (such as phenyl or 4-methyl-phenyl). The alkylation reaction of (XIX) to (X) is for example carried out in the presence of a base capable of deprotonating the 1-position pyrazolopyridine nitrogen atom of the compound of formula (XIX); the base can for example comprise or be sodium hydride, potassium hydride, potassium t-butoxide, lithium diisopropylamide (LDA), or a suitable basic resin or polymer such as polymer-bound 2-tert-butylimino-2-diethylamino-1,3-dimethyl-perhydro-1,3,2-diazaphosphorine. The reaction is for example carried out in the presence of a solvent, e.g. an organic solvent such as DMF; the solvent is for example anhydrous.

Compounds of formula (XIX) can sometimes be prepared, using a method analogous to that used for the preparation of compounds of formula (X) from compounds of formula (XI), by reaction of a compound of formula (XX) (which is the same as compound of formula (XI) but wherein R¹ is replaced by H) with an amine of formula R³NH₂. The reaction is for example carried out in the presence of a suitable base such as triethylamine or N,N-diisopropylethylamine, and/or in an organic solvent such as ethanol, dioxane or acetonitrile. The reaction may e.g. comprise heating e.g. to ca. 60-100° C., for example ca. 80-90° C.:

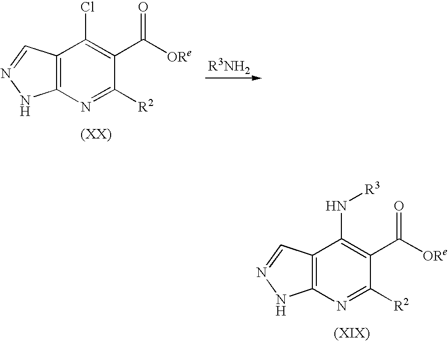

Alternatively, in formula (XX), the 4-chloro is optionally replaced by 4-C₁₋₄alkoxy such as 4-ethoxy; these modified compounds, of formula (XXa), is optionally made similarly to method(s) described herein.

In an alternative to the reaction of compound (XI) to prepare a 4-amino 5-ester pyrazolopyridine compound of Formula (X), instead of the 4-chloro substituent in the compound of formula (XI), a different non-fluorine halogen atom such as a bromine atom, or another suitable leaving group which is displaceable by an amine of formula R³NH₂, can be used at the 4-position of the pyrazolopyridine. The leaving group displaceable by the amine can for example be R^{LA}, in the compound of formula (XIa) shown below, wherein R^{LA} is a bromine atom or an alkoxy group OR³⁵ such as OC₁₋₄alkyl (in particular OEt) or a group —O—S(O)₂—R³⁷. Here, R³⁷ is C₁₋₈alkyl (e.g. C₁₋₄-alkyl or C₁₋₂alkyl such as methyl), C₁₋₆fluoroalkyl (e.g. C₁₋₄fluoroalkyl or C₁₋₂fluoroalkyl such as CF₃ or C₄F₉), or phenyl wherein the phenyl is optionally substituted by one or two of independently C₁₋₂alkyl, halogen or C₁₋₂alkoxy (such as phenyl or 4-methyl-phenyl). The reaction of the compound of formula (XIa) with the amine of formula R³NH₂ is optionally carried out with or without solvent, and e.g. may comprise heating:

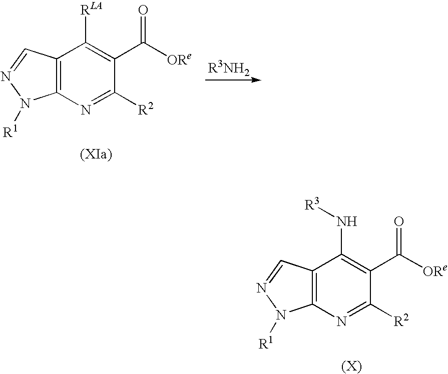

In one embodiment, in order to prepare compounds of formula (I) wherein R² is C₁fluoroalkyl (e.g. CF₃ or CHF₂, or CH₂F), the following processes are optionally used, generally illustrated for non-limiting examples wherein NHR³ is of sub-formula (h), and R¹=Et, (and R^{e}=Et within the compound of formula (X)). In one example, compounds wherein R² is CF₃ are optionally prepared as follows:

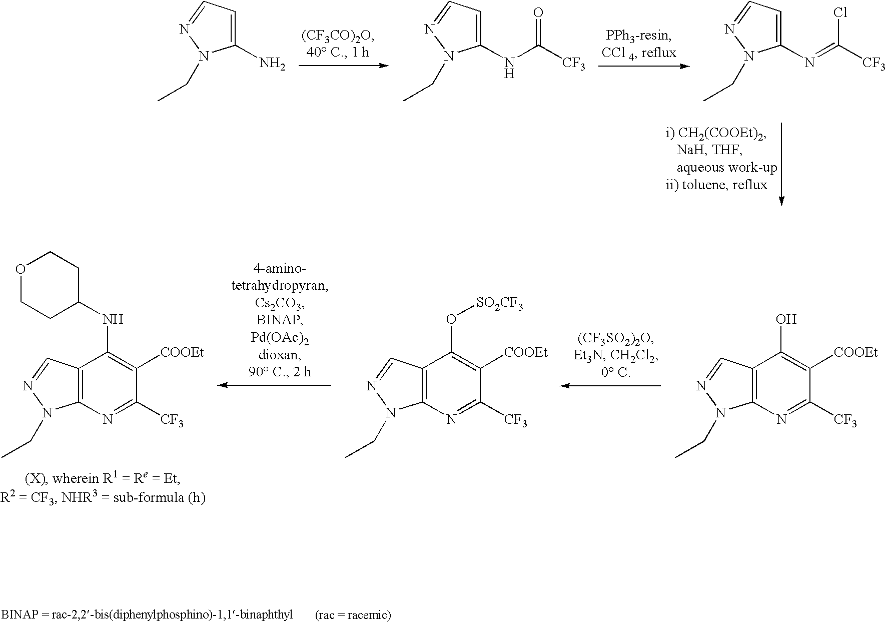

In another example, compounds wherein R² is CHF₂ or CH₂F are optionally prepared as follows:

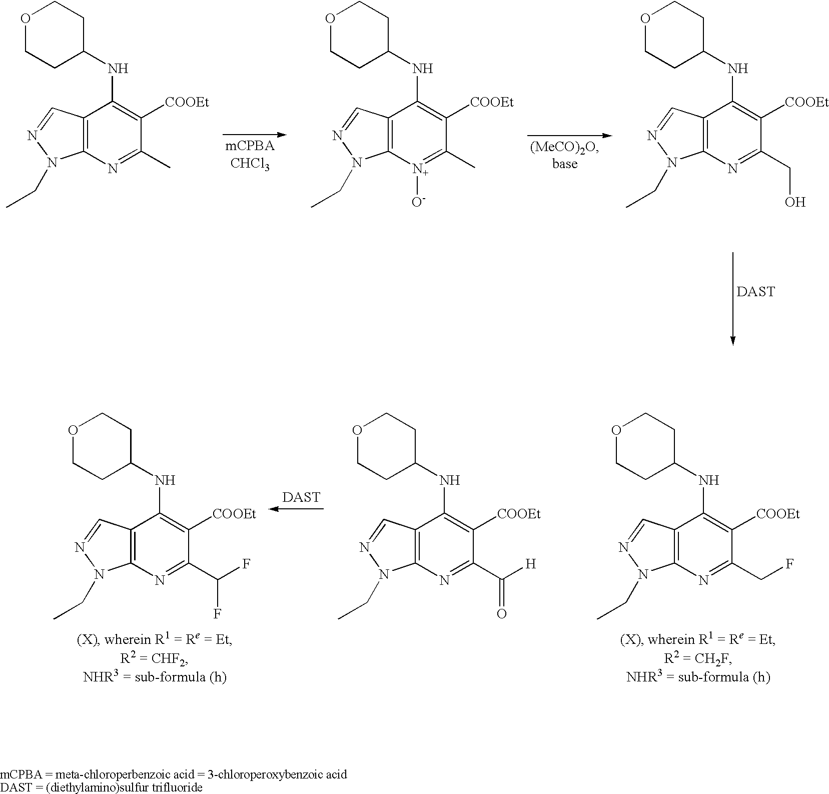

### Process B

Process B is an alternative version of process A and, without limitation, it is thought that it may be suitable, for example, for where the two amines of formula (IVa) and (IVb) [used within Process A to form the active compound (I)] are different (i.e. where it is not the case that R¹=R^{1a}, R²=R^{2a}, R³=R^{3a}, and R⁴=R^{4a}). In process B, the compound of formula (I) can be prepared by reacting an amine of formula (IVa) with an activated carboxylic acid derivative of formula (XXI), e.g. using amide coupling conditions, wherein X^{1a} is a leaving group substitutable by the NHR⁴ moiety of the amine of formula (IVa):

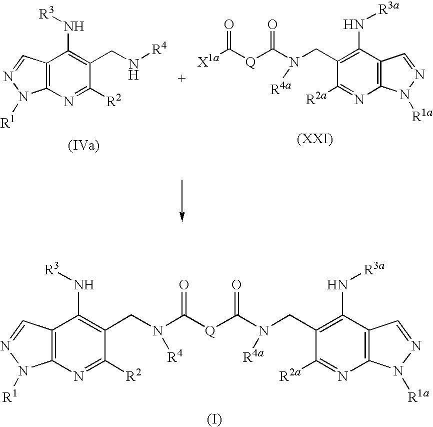

It is thought that the compound of formula (XXI) can optionally be prepared as follows, generally or sometimes. A a dicarboxylic acid compound of formula (VI) which is HO—C(O)-Q-C(O)—OH is optionally converted to a monoprotected dicarboxylic acid of formula Prot¹—O—C(O)-Q-C(O)—OH using a suitable protecting agent (e.g. an esterification agent where the Prot¹—O—C(O)— moiety is an ester). An amine of formula (IVb) (see Process A) is optionally coupled with the monoprotected dicarboxylic acid of formula Prot¹—O—C(O)-Q-C(O)—OH or an activated derivative thereof of formula Prot¹—O—C(O)-Q-C(O)—X^{1a}, e.g. using amide coupling conditions, to form Prot¹—C(O)-Q-C(O)—N(R^{4a})—CH₂-[substituted-pyrazolopyridine]. The monoprotected dicarboxylic acid or its derivative can for example be one in which Prot¹—O—C(O)— is an ester (e.g. Prot¹ can be alkyl or alkaryl or aryl) such as a t-butyl ester (Prot¹=t-butyl) or a benzyl ester (Prot¹=benzyl). The protecting group Prot¹ is optionally then removed, e.g. using ester hydrolysis conditions where Prot¹—O—C(O)— is an ester, to generate the unprotected carboxylic acid of the following formula HO—C(O)-Q-C(O)—N(R^{4a})—CH₂-[substituted-pyrazolopyridine]. Activation of the carboxylic acid moiety of this compound optionally forms the compound of formula (XXI), above.

### Process C

In process C, a compound of formula (I), in which R⁴ and R^{4a} are not a hydrogen atom (H), is optionally prepared, either by di-alkylation of the two amide nitrogen atoms of a compound of formula (Ib), or by mono-alkylation of one amide nitrogen atom of a compound of formula (Ic), in the presence of an alkylating agent R⁴—X^{c} (optionally also R^{4a}—X^{c}) which is or are suitable for such a di-alkylation or mono-alkylation. X^{c} is a leaving group suitable for such an alkylation such as an iodine atom. Where compound (Ib) is di-alkylated by R⁴—X^{c} then typically R⁴ is the same as R^{4a}. In one embodiment, the (di)alkylation reaction is optionally carried out in the presence of a suitable base such as potassium carbonate or sodium carbonate.

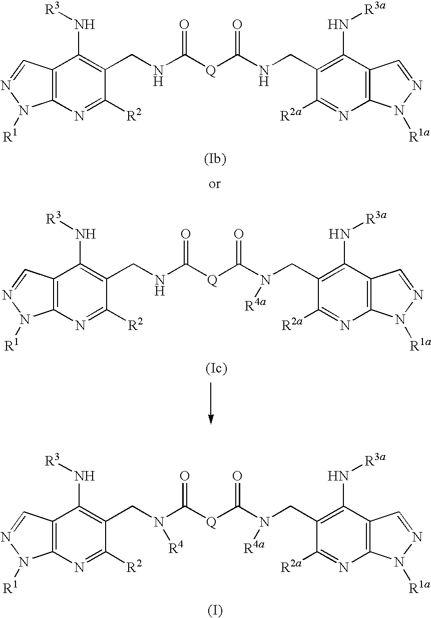

The compounds of formula (Ib) and (Ic) are optionally prepared as otherwise described herein for compounds of formula (I), e.g. via process A or otherwise.

### Process D

It is possible that compounds of formula (I), wherein R³=R^{3a}, might also be preparable by reaction of a compound of formula (XXII) with an amine of formula R³NH₂. This is Process D. In the compound of formula (XXII), R^{LD} is a leaving group which is displaceable by the amine of formula R³NH₂. R^{LD} can e.g. be a bromine atom (Br) or more particularly a chlorine atom (Cl), or alternatively R^{LD} can be an alkoxy group OR³⁵ such as OC₁₋₄alkyl (in particular OEt) or a group —O—S(O)₂—R³⁷. Here, R³⁷ is C₁₋₈alkyl (e.g. C₁₋₄alkyl or C₁₋₂alkyl such as methyl), C₁₋₆fluoroalkyl (e.g. C₁₋₄fluoroalkyl or C₁₋₂fluoroalkyl such as CF₃ or C₄F₉), or phenyl wherein the phenyl is optionally substituted by one or two of independently C₁₋₂alkyl, halogen or C₁₋₂alkoxy (such as phenyl or 4-methyl-phenyl). The reaction of (XXII) to (I) is optionally carried out in the presence of a base, such as triethylamine or N,N-diisopropylethylamine, and/or in an organic solvent such as ethanol, THF, dioxane or acetonitrile. The reaction may require heating, e.g. to ca. 60-100° C. or ca. 80-90° C., for example for 8-48 hours such as 12-24 hours:

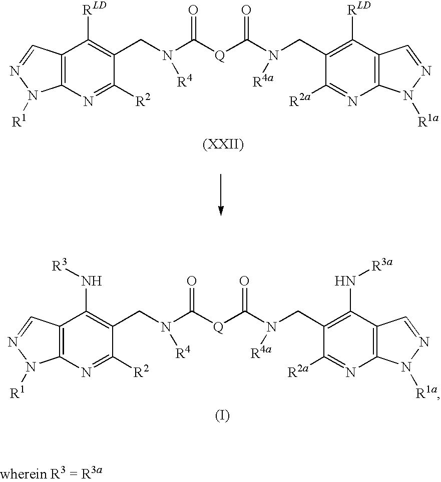

Process E

In optional Process E, a compound of formula (I), wherein R¹=R^{1a}, is e.g. prepared by reaction of a compound of formula (XXIII) with an alkylating agent of formula R¹—X³, where X³ is a leaving group displaceable by the 1-position pyrazolopyridine nitrogen atom of the compound of formula (XXIII):

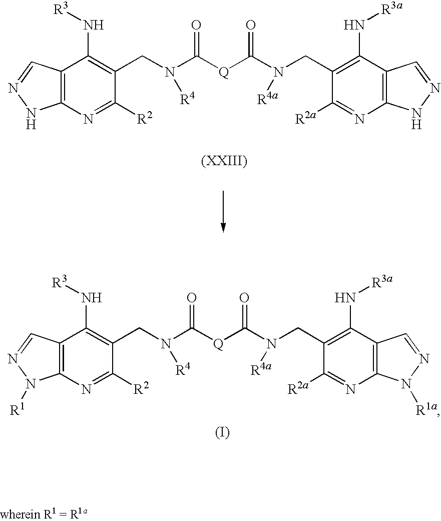

For example, in one embodiment, X³ is e.g. a halogen atom such as a chlorine atom or more preferably a bromine or iodine atom, or X³ can be —O—S(O)₂—R³⁶ wherein R³⁶ is C₁₋₈alkyl (e.g. C₁₋₄alkyl or C₁₋₂alkyl such as methyl), C₁₋₆fluoroalkyl (e.g. C₁₋₄fluoroalkyl or C₁₋₂fluoroalkyl such as CF₃ or C₄F₉), or phenyl wherein the phenyl is optionally substituted by one or two of independently C₁₋₂alkyl, halogen or C₁₋₂alkoxy (such as phenyl or 4-methyl-phenyl). The reaction is optionally carried out in the presence of a suitable base capable of deprotonating the 1-position pyrazolopyridine nitrogen atom of the compound of formula (XXIII); the base can for example comprise or be sodium hydride, potassium hydride, potassium t-butoxide, lithium diisopropylamide (LDA), or a suitable basic resin or polymer such as polymer-bound 2-tert-butylimino-2-diethylamino-1,3-dimethyl-perhydro-1,3,2-diazaphosphorine. The reaction is optionally carried out in the presence of a solvent, e.g. an organic solvent such as DMF; the solvent can be anhydrous.

Process F: Conversion of One Compound of Formula (I), or a Salt Thereof into Another Compound of Formula (I) or a Salt Thereof

In optional Process F, one compound of formula (I) or salt thereof (or a protected version thereof, such as an N-protected version thereof e.g. BOC-N-protected or benzyloxycarbonyl-N-protected version; or a derivative thereof such as an ester derivative thereof) is optionally converted into another compound of formula (I) or a salt thereof. This conversion reaction can for example occur within the R³ group. This conversion optionally comprises or is one or more of the following processes F1 to F10:

F1. Conversion of a ketone into the corresponding oxime.
F2. An oxidation process. For example, the oxidation process can comprise or be oxidation of an alcohol to a ketone (e.g. using Jones reagent) or oxidation of an alcohol or a ketone to a carboxylic acid.
F3. A reduction process, for example reduction of a ketone or a carboxylic acid to an alcohol.
F4. Acylation, for example acylation of an amine (e.g. see Examples 329-349 and Example 353 of WO 2004/024728 A2, incorporated herein by reference, for suitable process details), or acylation of a hydroxy group.
F5. Alkylation, for example alkylation of an amine or of a hydroxy group.
F6. Hydrolysis, e.g. hydrolysis of an ester to the corresponding carboxylic acid or salt thereof (e.g. see Examples 351, 488, 489, 650, 651 of WO 2004/024728 A2, incorporated herein by reference, for suitable process details).
F7. Deprotection, e.g. deprotection of (e.g. deacylation of, or t-butyloxycarbonyl (BOC) removal from, or benzyloxycarbonyl removal from) an amine group. BOC deprotection is usually carried out under acidic conditions e.g. using hydrogen chloride in an organic solvent such as dioxan. Benzyloxycarbonyl deprotection is optionally carried out by hydrogenation.
F8. Formation of an ester or amide, for example from the corresponding carboxylic acid.
and/or
F9. Beckmann rearrangement of one compound of formula (I) into another compound of formula (I), for example using cyanuric chloride (2,4,6-trichloro-1,3,5-triazine) together with a formamide such as DMF, e.g. at room temperature (see L. D. Luca, J. Org. Chem., 2002, 67, 6272-6274). The Beckmann rearrangement can for example comprise conversion of a compound of formula (I) wherein NHR³ is of sub-formula (o2)

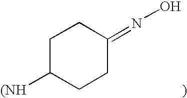

into a compound of formula (I) wherein NHR³ is of sub-formula (m3)

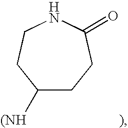

and process details can be for example as illustrated in Examples 658 and 659 of WO 2004/024728 A2, incorporated herein by reference.

### Synthetic Process Summary

The present invention therefore also provides a process for preparing a compound of formula (I) or a salt (e.g. pharmaceutically acceptable salt) thereof:

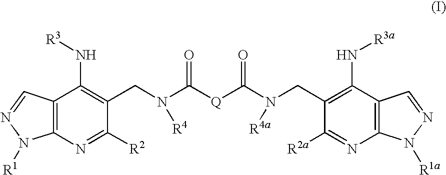

the process comprising:
(A) reacting an amine of formula (IVa) or a salt thereof, and optionally also an amine of formula (IVb) (when different to the amine of formula (IVa)) or a salt thereof,

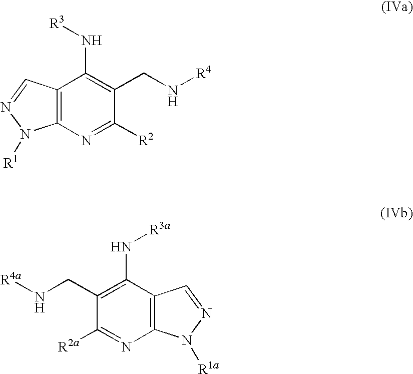

with a compound of formula (V):

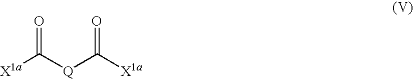

wherein X^{1a} is a leaving group which is substitutable by the NHR⁴ moiety of the compound of formula (IVa) (and when (IVb) is used X^{1a} is a leaving group which is also substitutable by the NHR^{4a} amine moiety of the compound of formula (IVb));
or
(B) reacting an amine of formula (IVa) with an activated carboxylic acid derivative of formula (XXI), wherein X^{1a} is a leaving group substitutable by the NHR⁴ moiety of the amine of formula (IVa):

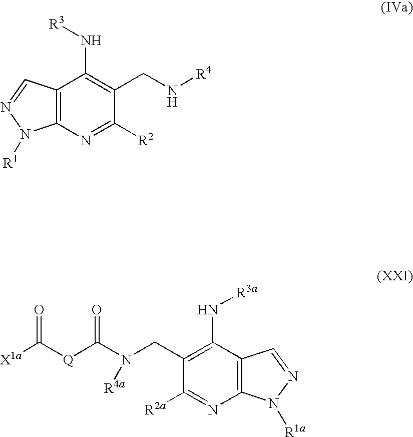

or
(C) to prepare a compound of formula (I) in which R⁴ and R^{4a} are not a hydrogen atom (H), either di-alkylation of the two amide nitrogen atoms of a compound of formula (Ib), or mono-alkylation of one amide nitrogen atom of a compound of formula (Ic), in the presence of an alkylating agent R⁴—X^{c} (optionally also R^{4a}—X^{c}) which is or are suitable for such a di-alkylation or mono-alkylation:

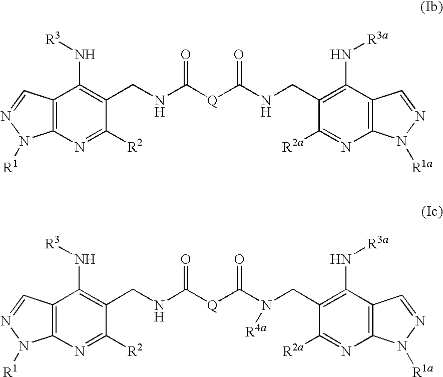

or
(D) to prepare a compound of formula (I) in which R³=R^{3a}, reacting a compound of formula (XXII) with an amine of formula R³NH₂:

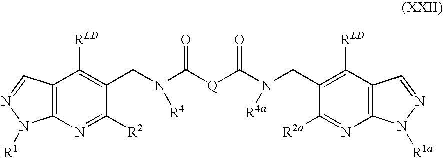

or
(E) to prepare a compound of formula (I) in which R¹=R^{1a}, reacting a compound of formula (XXIII) with an alkylating agent of formula R¹—X³, where X³ is a leaving group displaceable by the 1-position pyrazolopyridine nitrogen atom of the compound of formula (XXIII):

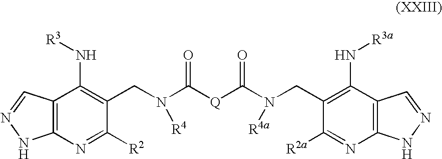

or
(F) converting one compound of formula (I) or a salt thereof, or a protected version thereof or a derivative (e.g. ester derivative) thereof, into another compound of formula (I) or a salt thereof;
and, in the case of (A), (B), (C), (D), (E) or (F), optionally converting the compound of formula (I) into a salt thereof such as a pharmaceutically acceptable salt thereof;
or
(G) to prepare a salt of a compound of formula (I), converting a compound of formula (I) into a salt thereof such as a pharmaceutically acceptable salt thereof.
Preferred, suitable or optional features of steps (A), (B), (C), (D), (E), or (F), independently of each other, can be as described above for Processes A, B, C, D, E, or F, with all necessary changes being made.

The present invention also provides: (G) a process for preparing a pharmaceutically acceptable salt of a compound of formula (I) comprising conversion of the compound of formula (I) or a salt thereof into the desired pharmaceutically acceptable salt thereof. Such processes can for example be as described herein, e.g. as described in the Salts section above. For example, a pharmaceutically acceptable salt can be an acid addition salt, or less commonly (e.g. if a C(O)OH group is present in the compound) a base addition salt. In one embodiment, a pharmaceutically acceptable acid addition salt is optionally prepared by reaction of a compound of formula (I) with a suitable inorganic or organic acid (e.g. as described hereinabove).

The present invention also provides a compound of formula (I) or a salt thereof, prepared by (or obtainable by) a method as defined herein.

### Medical Uses

The present invention also provides a compound of formula (I) or a pharmaceutically acceptable salt thereof for use as an active therapeutic substance in a mammal such as a human. The compound or salt can be for use in the treatment and/or prophylaxis of any of the diseases/conditions described herein (e.g. for use in the treatment and/or prophylaxis of an inflammatory and/or allergic disease in a mammal such as a human) and/or can be for use as a phosphodiesterase 4 (PDE4) inhibitor. “Therapy” may include treatment and/or prophylaxis.

Also provided is the use of a compound of formula (I) or a pharmaceutically acceptable salt thereof in the manufacture of a medicament (e.g. pharmaceutical composition) for the treatment and/or prophylaxis of any of the diseases/conditions described herein in a mammal such as a human, e.g. for the treatment and/or prophylaxis of an inflammatory and/or allergic disease in a mammal such as a human.

Also provided is a method of treatment and/or prophylaxis of any of the diseases/conditions described herein in a mammal (e.g. human) in need thereof, e.g. a method of treatment and/or prophylaxis of an inflammatory and/or allergic disease in a mammal (e.g. human) in need thereof, which method comprises administering to the mammal (e.g. human) a therapeutically effective amount of a compound of formula (I) as herein defined or a pharmaceutically acceptable salt thereof.

Phosphodiesterase 4 inhibitors are thought to be useful in the treatment and/or prophylaxis of a variety of diseases/conditions, especially inflammatory and/or allergic diseases, in a mammal such as a human, for example: chronic obstructive pulmonary disease (COPD) (e.g. chronic bronchitis and/or emphysema), asthma, rhinitis (e.g. allergic rhinitis), rheumatoid arthritis, atopic dermatitis, psoriasis, urticaria, allergic conjunctivitis, vernal conjunctivitis, eosinophilic granuloma, septic shock, inflammatory bowel disease (e.g. ulcerative colitis and/or Crohn's disease), reperfusion injury of the myocardium and/or brain, chronic glomerulonephritis, endotoxic shock, or adult respiratory distress syndrome, in a mammal such as a human; in particular: chronic obstructive pulmonary disease (COPD) (e.g. chronic bronchitis and/or emphysema), asthma, rhinitis (e.g. allergic rhinitis), atopic dermatitis, psoriasis, urticaria, allergic conjunctivitis, vernal conjunctivitis, eosinophilic granuloma, inflammatory bowel disease (e.g. ulcerative colitis and/or Crohn's disease), or adult respiratory distress syndrome, in a mammal such as a human. Ulcerative colitis and/or Crohn's disease are collectively often referred to as inflammatory bowel disease.

In the treatment and/or prophylaxis using the compound of formula (I) or the pharmaceutically acceptable salt thereof, the inflammatory and/or allergic disease can for example be chronic obstructive pulmonary disease (COPD), asthma, rhinitis (e.g. allergic rhinitis), atopic dermatitis or psoriasis, in a mammal (e.g. human). Preferably, the compound of formula (I) or the pharmaceutically acceptable salt thereof is for the treatment and/or prophylaxis of COPD, asthma or rhinitis (e.g. allergic rhinitis), in a mammal (e.g. human).

PDE4 inhibitors, for example cilomilast and roflumilast, are thought to be effective in the treatment of COPD. For example, see S. L. Wolda, Emerging Drugs, 2000, 5(3), 309-319; Z. Huang et al., Current Opinion in Chemical Biology, 2001, 5: 432-438; H. J. Dyke et al., Expert Opinion on Investigational Drugs, January 2002, 11(1), 1-13; C. Burnouf et al., Current Pharmaceutical Design, 2002, 8(14), 1255-1296; A. M. Doherty, Current Opinion Chem. Biol., 1999, 3(4), 466-473; A. M. Vignola, Respiratory Medicine, 2004, 98, 495-503; D. Spina, Drugs, 2003, 63(23), 2575-2594; and references cited in the aforementioned publications; G. Krishna et al., Expert Opinion on Investigational Drugs, 2004, 13(3), 255-267 (see especially pp. 259-261 and refs. 102-111 and 201 therein); and B. J. Lipworth, The Lancet, 2005, 365, 167-175.

The PDE4 inhibitor cilomilast (Ariflo™) at 15 mg orally twice daily appears to improve forced expiratory volume in 1 s (FEV₁) in COPD patients (C. H. Compton et al., The Lancet, 2001, vol. 358, 265-270), and appears to have antiinflammatory effects in COPD patients (E. Gamble et al., Am. J. Respir. Crit. Care Med., 2003, 168, 976-982). On cilomilast, see also R. D. Border et al., Chest, 2003, vol. 124 Suppl. 4, p. 170S (abstract) and J. D. Eddleston et al., Am. J. Respir. Crit. Care Med., 2001, 163, A277 (abstract). The PDE4 inhibitor roflumilast appears to show small improvements in FEV₁ in COPD patients (see B. J. Lipworth, The Lancet, 2005, 365, 167-175, and refs 49-50 therein).

COPD is often characterised by the presence of airflow obstruction due to chronic bronchitis and/or emphysema (e.g., see S. L. Wolda, Emerging Drugs, 2000, 5(3), 309-319).

For treatment and/or prophylaxis of COPD or asthma in a mammal (e.g. human), inhaled or parenteral administration to the mammal of the compound of formula (I) or a pharmaceutically acceptable salt thereof can be used, preferably inhaled administration.

PDE4 inhibitors are thought to be effective in the treatment and/or prophylaxis of asthma (e.g. see M. A. Giembycz, Drugs, February 2000, 59(2), 193-212; Z. Huang et al., Current Opinion in Chemical Biology, 2001, 5: 432-438; H. J. Dyke et al., Expert Opinion on Investigational Drugs, January 2002, 11(1), 1-13; C. Burnoufetal., Current Pharmaceutical Design, 2002, 8(14), 1255-1296; A. M. Doherty, Current Opinion Chem. Biol., 1999, 3(4), 466-473; P. J. Barnes, Nature Reviews—Drug Discovery, October 2004, 831-844; B. J. Lipworth, The Lancet, 2005, 365, 167-175; and references cited in the aforementioned publications).

In one optional embodiment, the compound of formula (I) or the pharmaceutically acceptable salt thereof is for the treatment and/or prophylaxis of rhinitis, such as allergic rhinitis (e.g. seasonal allergic rhinitis or perennial allergic rhinitis) or non-allergic rhinitis (e.g. vasomotor rhinitis), in a mammal such as a human. By way of example, for rhinitis such as allergic rhinitis, intranasal or parenteral administration of the compound of formula (I) or a pharmaceutically acceptable salt thereof is optionally used.

The PDE4 inhibitor roflumilast, given orally at 500 ug once daily for 9 days, is reported to be effective in improving rhinal airflow during the treatment period (compared to placebo), in humans with histories of allergic rhinitis but asymptomatic at screening, and who were challenged with intranasal allergen provocation (pollen extracts) daily beginning the third day of treatment and each time approx. 2 hours after study drug administration (B. M. Schmidt et al., J. Allergy & Clinical Immunology, 108(4), 2001, 530-536).

PDE4 inhibitors are thought to be effective in the treatment of rheumatoid arthritis (e.g. see H. J. Dyke et al., Expert Opinion on Investigational Drugs, January 2002, 11(1), 1-13; C. Burnouf et al., Current Pharmaceutical Design, 2002, 8(14), 1255-1296; and A. M. Doherty, Current Opinion Chem. Biol., 1999, 3(4), 466-473; and references cited in these publications). For rheumatoid arthritis, parenteral administration is optionally used.

PDE4 inhibition has been suggested for the treatment of inflammatory bowel disease (e.g. ulcerative colitis and/or Crohn's disease), see K. H. Banner and M. A. Trevethick, Trends Pharmacol. Sci., August 2004, 25(8), 430-436.

In one embodiment, the compound or salt can for example be for use in the treatment and/or prophylaxis of an inflammatory and/or allergic skin disease, such as atopic dermatitis or psoriasis, in a mammal such as a human.

In one optional embodiment, the treatment and/or prophylaxis is of atopic dermatitis in a mammal such as a human or pig, preferably in a human, in particular in a human aged 21 years or less, e.g. 18 years or less. For treatment and/or prophylaxis of atopic dermatitis in a mammal, external topical administration to the mammal of the compound of formula (I) or a pharmaceutically acceptable salt thereof (e.g. topical administration to the skin e.g. to skin affected by the atopic dermatitis) can be used, though alternatively oral or parenteral administration can be used. For treatment and/or prophylaxis of atopic dermatitis, inhaled administration is usually not suitable.

“Atopic dermatitis” has been proposed to include two general sub-classes: (1) an “allergic (extrinsic)” type of atopic dermatitis which generally occurs in the context of sensitization to environmental allergens and/or which is generally accompanied by elevated serum IgE levels; and (2) an “non-allergic (intrinsic)” type of atopic dermatitis generally with little or no detectable sensitization and/or generally with normal or low serum IgE levels (N. Novak et al., J. Allergy Clin. Immunol., 2003, 112, 252-262; and T. C. Roos et al., Drugs, 2004, 64(23), 2639-2666, see e.g. pages 2640-2641). The compound of formula (I) or the pharmaceutically acceptable salt thereof can therefore be for the treatment and/or prophylaxis of allergic (extrinsic) atopic dermatitis and/or non-allergic (intrinsic) atopic dermatitis in a mammal (e.g. human or pig, preferably human).

“External topical” administration means topical administration to an external body part (i.e. excluding, for example, the lung or mouth, but including the lips), preferably excluding the eye.

“External topical” administration preferably is topical administration to the skin, for example to the skin of an arm, hand, leg, foot, head (e.g. face), neck and/or torso of a mammal such as a human. External topical administration can for example be to those parts of a mammal's skin affected by or susceptible to atopic dermatitis. For the use of PDE4 inhibitors in atopic dermatitis, see for example:

J. M. Hanifin et al., “Type 4 phosphodiesterase inhibitors have clinical and in vitro anti-inflammatory effects in atopic dermatitis”, J. Invest. Dermatol., 1996, 107(1), 51-56; which reports reductions of inflammatory parameters in atopic dermatitis patients treated with PDE4 inhibitor CP80,633 (0.5% ointment, twice daily topical application);

C. E. M. Griffiths et al., “Randomized comparison of the type 4 phosphodiesterase inhibitor cipamfylline cream, cream vehicle and hydrocortisone 17-butyrate cream for the treatment of atopic dermatitis”, Br. J. Dermatol., 2002, 147(2), 299-307, which reports that cipamfylline (0.15%) cream is significantly more effective than vehicle, but significantly less effective than hydrocortisone 17-butyrate (0.1%) cream, in the treatment of atopic dermatitis patients;

T. C. Roos et al., “Recent advances in treatment strategies for atopic dermatitis”, Drugs, 2004, 64(23), 2639-2666 (see e.g. page 2657 and refs. 201-209 therein);

A. M. Doherty, Current Opinion Chem. Biol., 1999, 3(4), 466-473 (e.g. see p. 470); and

H. J. Dyke et al., Expert Opinion Invest. Drugs, 2002, 11(1), 1-13 (e.g. see p. 7 and refs. 74, 75 and 76 cited therein);

and references cited in the above references.

For the use of the PDE4 inhibitors SB 207499 (cilomilast) and AWD 12-281 in mouse models of the allergic type of dermatitis, see W. Bäumer et al., Eur. J. Pharmacol., 2002, 446, 195-200 and W. Bäumer et al., J. Pharmacy Pharmacol., 2003, 55, 1107-1114.

### Pharmaceutical Compositions, Routes of Administration, and Dosage Regimens

For use in medicine, the compounds or salts of the present invention are usually administered as a pharmaceutical composition.

The present invention therefore provides in a further aspect a pharmaceutical composition comprising a compound of formula (I) or a pharmaceutically acceptable salt thereof and one or more pharmaceutically acceptable carriers and/or excipients.

The pharmaceutical composition can be for use in the treatment and/or prophylaxis of any of the conditions described herein, for example chronic obstructive pulmonary disease (COPD), asthma, rhinitis (e.g. allergic rhinitis), atopic dermatitis or psoriasis in a mammal (e.g. human).

The invention also provides a method of preparing a pharmaceutical composition comprising a compound of formula (I), as herein defined, or a pharmaceutically acceptable salt thereof, and one or more pharmaceutically acceptable carriers and/or excipients,
the method comprising mixing the compound or salt with the one or more pharmaceutically acceptable carriers and/or excipients.

The invention also provides a pharmaceutical composition prepared by said method.

The compounds of formula (I) or salts thereof and/or the pharmaceutical composition may be administered, for example, by inhaled, intranasal, external topical (e.g. skin topical), parenteral (e.g. intravenous, subcutaneous, or intramuscular), or oral administration, for example to a mammal such as a human. Inhaled administration involves topical administration to the lung e.g. by aerosol or dry powder composition.

Accordingly, the pharmaceutical composition can be suitable for (e.g. adapted for) inhaled, intranasal, external topical (e.g. skin topical), parenteral (e.g. intravenous, subcutaneous, or intramuscular), or oral administration, e.g. to a mammal such as a human. The pharmaceutical composition can for example be suitable for inhaled, intranasal or external topical (e.g. skin topical) administration, e.g. to a mammal such as a human.

Inhaled or intranasal administration, in particular inhaled administration, is generally a preferred route of administration, and in particular is preferred for N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide or a salt thereof (e.g. Example 23, 23a, 23b or 23c).

Oral administration is generally not a preferred route of administration, and in particular is not preferred for N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide or a salt thereof (e.g. Example 23, 23a, 23b or 23c).

The pharmaceutical composition can optionally be in unit dose form. The unit dose form can for example be:

(a) a rupturable or peel-openable sealed dose container containing a dry powder inhalable pharmaceutical composition (e.g. a plurality of which are usually disposed inside a suitable inhalation device);

(b) a vial, ampoule or filled syringe for parenteral administration e.g. comprising a solution or suspension of the compound or pharmaceutically acceptable salt in a suitable carrier such as an aqueous carrier or e.g. containing a lyophilised parenteral pharmaceutical composition (the vial or ampoule can optionally be manufactured using a blow-fill-seal process); or

(c) a tablet or capsule for oral administration e.g. for oral administration to a human.

Alternatively, the composition can be in a form adapted for the administration of varying amounts of composition as desired by the user, such as a spreadable or sprayable external topical composition such as a cream, an ointment, a gel, or a liquid.

### Pharmaceutical Compositions Suitable For Inhalable or Intranasal Administration, and Particle-Size Reduction

Pharmaceutical compositions suitable for (e.g. adapted for) intranasal or inhaled administration may conveniently be formulated as aerosols, drops, gels or dry powders.

Aerosol formulations, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomizing device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it can contain a suitable propellant under pressure such as compressed air, carbon dioxide, or an organic propellant such as a chlorofluorocarbon (CFC) or hydrofluorocarbon (HFC). Suitable CFC propellants include dichlorodifluoromethane, trichlorofluoromethane and dichlorotetrafluoroethane. Suitable HFC propellants include 1,1,1,2,3,3,3-heptafluoropropane and 1,1,1,2-tetrafluoroethane. The aerosol dosage forms can also take the form of a pump-atomiser.

### Particle Size Reduction of Compound of Formula (I) or Salt Thereof.

In, for example, pharmaceutical compositions suitable (e.g. adapted for) inhaled administration, the compound or salt of formula (I) can be in a particle-size-reduced form. The size-reduced form can for example be obtained or obtainable by micronization. Micronization usually involves subjecting the compound/salt to collisional and/or abrasional forces in a fast-flowing circular or spiral/vortex-shaped airstream often including a cyclone component. The particle size of the size-reduced (e.g. micronised) compound or salt can be defined by a D50 value of about 0.5 to about 10 microns, e.g. about 1 to about 7 microns or about 1 to about 5 microns (e.g. as measured using laser diffraction). For example, the compound or salt of formula (I) can have a particle size defined by: a D10 of about 0.3 to about 3 microns (e.g. about 0.4 to about 2 microns, or about 0.5 to about 1 microns), and/or a D50 of about 0.5 to about 10 microns or about 1 to about 7 microns or (e.g. about 1 to about 5 microns or about 1.5 to about 5 microns or about 1.5 to about 4 microns), and/or a D90 of about 1 to about 30 microns or about 2 to about 20 microns or about 2 to about 15 microns or about 3 to about 15 microns (e.g. about 2 to about 10 microns or about 4 to about 10 microns); for example as measured using laser diffraction.

In particle size measurements, D90, D50 and D10 respectively mean that 90%, 50% and 10% of the material is less than the micron size specified. D50 is the median particle size. DV90, DV50 and DV10 respectively mean that 90%, 50% and 10% by volume of the material is less than the micron size specified. DM90, DM50 and DM10 respectively mean that 90%, 50% and 10% by weight of the material is less than the micron size specified.

Laser diffraction measurement of particle size can use a dry method (wherein a suspension of the compound/salt in an airflow crosses the laser beam) or a wet method [wherein a suspension of the compound/salt in a liquid dispersing medium, such as isooctane or ca. 0.05% lecithin in isooctane or (e.g. if compound is soluble in isooctane) 0.1% Tween 80 in water, crosses the laser beam]. With laser diffraction, particle size is preferably calculated using the Fraunhofer calculation; and/or preferably a Malvern Mastersizer or Sympatec apparatus is used for measurement. For example, particle size measurement and/or analysis by laser diffraction can use any or all of (e.g. all of) the following apparatus and/or conditions: a Malvern Mastersizer 2000 version apparatus, a dispersing medium of isooctane or ca. 0.05% lecithin in isooctane or ca. 0.1% Tween 80 in water, a stirring speed of ca. 1500-2500 rpm, ca. 30 seconds to ca. 3 mins (e.g. ca. 30 seconds) sonification prior to final dispersion and analysis, a 300 RF (Reverse Fourier) lens, and/or the Fraunhofer calculation with Malvern software. In another example, particle size measurement and/or analysis by laser diffraction can use any or all of (e.g. all of) the following apparatus and/or conditions: a Malvern Mastersizer longbed version apparatus, a dispersing medium of ca. 0.1% Tween 80 in water, a stirring speed of ca. 1500 rpm, ca. 3 mins sonification prior to final dispersion and analysis, a 300 RF (Reverse Fourier) lens, and/or the Fraunhofer calculation with Malvern software.

An illustrative non-limiting example of a small-scale micronization process is now given:

### Micronization Example Micronization of a Compound or Salt of One of the Examples

Purpose: To micronise a compound or salt of one of the Examples (described hereinafter), e.g. in an amount of approximately 600-3000 mg thereof, using a Jetpharma MC1 micronizer.
The parent (unmicronised) and micronised materials are analyzed for particle size by laser diffraction and crystallinity by PXRD.

### Micronization Example General Equipment and Material

| Equipment/material | Description and specification |
|---|---|
| Jetpharma MC1 Micronizer | Nitrogen supply: Air tank e.g. with 275 psi |
| | rate tubing |
| Analytical balance | can e.g. be Sartorius Analytical |
| Top loader balance | can e.g. be Mettler PM400 |
| Digital Caliper | can e.g. be VWR Electronic caliper |
| Materials to be micronised | a compound or salt of one of the Examples |

The Jetpharma MC1 Micronizer comprises a horizontal disc-shaped milling housing having: a tubular compound inlet (e.g. angled at ca. 30 degrees to the horizontal) for entry of a suspension of unmicronised compound of formula (I) or salt in a gasflow, a separate gas inlet for entry of gases, a gas outlet for exit of gases, and a collection vessel (micronizer container) for collecting micronised material. The milling housing has two chambers: (a) an outer annular chamber in gaseous connection with the gas inlet, the chamber being for receiving pressurised gas (e.g. air or nitrogen), and (b) a disc-shaped inner milling chamber within and coaxial with the outer chamber for micronising the input compound/salt, the two chambers being separated by an annular wall. The annular wall (ring R) has a plurality of narrow-bored holes connecting the inner and outer chambers and circumferentially-spaced-apart around the annular wall. The holes opening into the inner chamber are directed at an angle (directed part-way between radially and tangentially), and in use act as nozzles directing pressurised gas at high velocity from the outer chamber into the inner chamber and in an inwardly-spiral path (vortex) around the inner chamber (cyclone). The compound inlet is in gaseous communication with the inner chamber via a nozzle directed tangentially to the inner chamber, within and near to the annular wall/ring R. Upper and lower broad-diameter exit vents in the central axis of the inner milling chamber connect to (a) (lower exit) the collection vessel which has no air outlet, and (b) (upper exit) the gas outlet. Inside and coaxial with the tubular compound inlet and longitudinally-movable within it is positioned a venturi inlet (V) for entry of gases. The compound inlet also has a bifurcation connecting to an upwardly-directed material inlet port for inputting material.

In use, the narrow head of the venturi inlet (V) is preferably positioned below and slightly forward of the material inlet port, so that when the venturi delivers pressurised gas (e.g. air or nitrogen) the feed material is sucked from the material inlet port into the gas stream through the compound inlet and is accelerated into the inner milling chamber tangentially at a subsonic speed. Inside the milling chamber the material is further accelerated to a supersonic speed by the hole/nozzle system around the ring (R) (annular wall) of the milling chamber. The nozzles are slightly angled so that the acceleration pattern of the material is in the form of an inwardly-directed vortex or cyclone. The material inside the milling chamber circulates rapidly and particle collisions occur during the process, causing larger particles to fracture into smaller ones. “Centrifugal” acceleration in the vortex causes the larger particles to remain at the periphery of the inner chamber while progressively smaller particles move closer to the centre until they exit the milling chamber, generally through the lower exit, at low pressure and low velocity. The particles that exit the milling chamber are heavier than air and settle downward through the lower exit into the collection vessel (micronizer container), while the exhaust gas rises (together with a minority of small particles of micronised material) and escapes into the atmosphere at low pressure and low velocity.

### Micronization Example: General Procedure:

The micronizer is assembled. The narrow head of the venturi inlet is positioned below and slightly forward of the material inlet port and is measured with a micro-caliper to make sure that it is inserted correctly. The ring (R) and venturi (V) pressures are adjusted according to the values specified in the experimental design (refer to experimental section below) by adjusting the valves on the pressure gauges on the micronizer. The setup is checked for leakage by observing if there is any fluctuation in the reading of the pressure gauges.

Note that the venturi (V) pressure is kept at least about 2 bars greater than the ring (R) pressure to prevent regurgitation of material, e.g. outwardly from the material inlet port.

Balance performance is checked with calibration weights. Specified amount of the parent material is fed into the input container of the micronizer using a spatula. The input container plus material is weighed. The equipment pressure is monitored during the micronization process.

Upon completion of the micronising run, the nitrogen supply is shut off and the micronised material is allowed to settle into the micronizer container. The micronised powder in the micronizer container (collection vessel) and the cyclone (above the recovery vessel) are collected together into a pre-weighed and labelled collection vial. The weight of the micronised material is recorded. The input container is re-weighed in order to calculate the amount of input material by difference. The micronizer is disassembled and residual PDE4 compound on the micronizer inner surface is rinsed with 70/30 isopropyl alcohol/water and collected into a flask. The micronizer is then thoroughly cleaned in a Lancer washing machine and dried before subsequent runs are performed.

In one embodiment, one example of suitable micronization conditions is: Material input amount about 0.7 g to about 2 g; Venturi Pressure (V) about 4 to about 10 bar; Ring Pressure (R) about 2 to about 6 bar. Material feed rate can optionally be from about 70 to about 200 mg/min.

% yield=[(Material from collection vessel+Material from cyclone)/Material input amount]×100.

### Micronization Examples Carried Out

### Micronization Example 1

N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide 1,5-naphthalenedisulfonate (e.g. Example 23a) was micronised, using a general procedure and/or general apparatus generally similar or analogous to those described above, and generally using substantially the following experimental parameters:

Equipment Used JetPharma MC-One microniser
Quantity micronised: about 1.8571 g
Venturi Pressure (V): about 4 bar
Ring (grind) pressure (R): about 2 bar
Pre-micronization screening: not done
Quantity recovered: about 1.4901 g (about 80.7% yield)
Micronization process time: about 23mins 38 secs.
Process Observations Input material appeared to be cohesive. Feed funnel of microniser was monitored for blockage. Blockage was found on one occasion which was cleared using a spatula.

### Micronization Example 2

N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide 1,2,4-benzenetricarboxylate (e.g. Example 23b) was micronised, using a general procedure and/or general apparatus generally similar or analogous to those described above, and generally using substantially the following experimental parameters:

Equipment Used JetPharma MC-One microniser
Pre-micronization screening: It was noted during the process that the input active pharmaceutical ingredient (API) material contained some hard aggregates. Therefore most or all of the unmicronised material was screened through a 600 micron screen or sieve before micronization.
Initial quantity of material dispensed for micronising: about 1.3024 g (before screening)
Quantity of screened material micronised: about 0.7953 g
Venturi Pressure used (V): about 3 bar to about 5 bar
Ring (Grind) pressure (R) used: about 1.25 bar to about 2 bar
Quantity recovered: about 0.3419 g (about 26.3% of initial dispensed quantity or about 43% of quantity after screening)
Process Observations During processing blockage of the feed funnel was observed; hence the remainder of the material was screened to de-aggregate it as described above. Also there was exhaust of some micronised API through the filter sock in the exhaust stream and there was subsequent blockage of the filter sock; this required an in-process change of the filter sock and reduction of the pressures through the process to try to counter these problems.

Particle size measurement/analysis was done by laser diffraction using the following conditions/apparatus: a Malvern Mastersizer 2000 version apparatus, ca. 30 seconds sonification prior to final dispersion and analysis, a “wet cell method” using a dispersing medium of 0.05% lecithin in isooctane, a stirring speed of ca. 2500 rpm, and a 300 RF (Reverse Fourier) lens. Possibly, the Fraunhofer calculation with Malvern software was used.

The results of the particle size analysis of the Micronization Example 2 product were: DV10=about 0.63 microns, DV50=about 1.90 microns, DV90=about 5.21 microns.

### Dry Powder Inhalable Compositions

For pharmaceutical compositions suitable (e.g. adapted for) inhaled administration, the pharmaceutical composition may for example be a dry powder inhalable composition. Such a composition can comprise a powder base such as lactose or starch, the compound of formula (I) or salt thereof (suitably in particle-size-reduced form, e.g. in micronised form), and optionally a ternary agent such as L-leucine, mannitol, trehalose, magnesium stearate, calcium stearate and/or cellobiose octaacetate (e.g. alpha-D-isomer of cellobiose octaacetate, e.g. available from Aldrich). For cellobiose octaacetate and storage stability, see WO 03/088943.

The dry powder inhalable composition can comprise a dry powder blend of lactose and the compound of formula (I) or salt thereof. The lactose can be lactose hydrate e.g. lactose monohydrate and/or can be inhalation-grade and/or fine-grade lactose. The particle size of the lactose can for example be defined by 90% or more (by weight or by volume) of the lactose particles being less than 1000 microns (micrometers) (e.g. 10-1000 microns e.g. 30-1000 microns) in diameter, and/or 50% or more of the lactose particles being less than 500 microns (e.g. 10-500 microns) in diameter. The particle size of the lactose can for example be defined by 90% or more of the lactose particles being less than 300 microns (e.g. 10-300 microns e.g. 50-300 microns) in diameter, and/or 50% or more of the lactose particles being less than 100 microns in diameter. Optionally, the particle size of the lactose can be defined by 90% or more of the lactose particles being less than 100-200 microns in diameter, and/or 50% or more of the lactose particles being less than 40-70 microns in diameter. It can be about 3 to about 30% (e.g. about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 JD Zwolle, Netherlands).

In the dry powder inhalable composition the compound of formula (I) or salt thereof can for example be present in about 0.1% to about 70% (e.g. about 1% to about 50%, e.g. about 5% to about 40%, e.g. about 20 to about 30%) by weight of the composition.

An illustrative non-limiting example of a dry powder inhalable composition follows:

Dry Powder Formulation Example—Dry powder Lactose Blend Preparation

Using a size-reduced e.g. micronised form of the compound of formula (I) or salt thereof (e.g. as prepared in the Micronization Example herein), the dry powder blend is, for example, prepared by mixing the required amount of the compound/salt (e.g. 10 mg, 1% w/w) with inhalation-grade lactose containing 10% fines (e.g. 990 mg, 99% w/w) in a Teflon™ (polytetrafluoroethene) pot in a Mikro-dismembrator ball-mill (but without a ball bearing) at % speed (ca. 2000-2500 rpm) for about 4 hours at each blend concentration. The Mikro-dismembrator (available from B. Braun Biotech International, Schwarzenberger Weg 73-79, D-34212 Melsungen, Germany; www.bbraunbiotech.com) comprises a base with an upwardly-projecting and sidewardly-vibratable arm to which is attached the Teflon™ pot. The vibration of the arm achieves blending.

Other blends can include: 10% w/w compound/salt (50 mg)+90% w/w lactose (450 mg, inhalation-grade lactose containing 10% fines).

Serial dilution of the 1% w/w blend can achieve e.g. 0.1% and 0.3% w/w blends.

### Dry Powder Inhalation Devices

Optionally, in particular for dry powder inhalable compositions, a pharmaceutical composition for inhaled administration can be incorporated into a plurality of sealed dose containers (e.g. containing the dry powder composition) mounted longitudinally in a strip or ribbon inside a suitable inhalation device. The container can be rupturable or peel-openable on demand and the dose, e.g. of the dry powder composition, can be administered by inhalation via a device such as the DISKUS™ device, marketed by GlaxoSmithKline. The DISKUS™ inhalation device can e.g. be substantially as described in GB 2,242,134 A. In such device at least one container for the pharmaceutical composition in powder form (the at least one container preferably being a plurality of sealed dose containers mounted longitudinally in a strip or ribbon) is defined between two members peelably secured to one another; the device comprises: means defining an opening station for the said at least one container; means for peeling the members apart at the opening station to open the container; and an outlet, communicating with the opened container, through which a user can inhale the pharmaceutical composition in powder form from the opened container.

### Pharmaceutical Compositions Suitable for External Topical Administration

The pharmaceutical composition of the invention can for example be suitable for (e.g. adapted for) external topical (e.g. skin topical) administration, for example to a mammal such as a human. The pharmaceutical composition suitable for external topical administration can suitably be for the treatment and/or prophylaxis of atopic dermatitis in a mammal such as a human.

“External topical administration” is defined above under the “medical uses” section. External topical administration can for example be to those parts of the skin-affected by or susceptible to the disease or condition e.g. atopic dermatitis, in particular in a mammal (e.g. human) suffering from or susceptible to atopic dermatitis.

An external-topical pharmaceutical composition, e.g. skin topical pharmaceutical composition, can for example be an ointment, a cream (usually an oil-in-water or water-in-oil pharmaceutical composition, usually an emulsion), an aqueous gel, or a microemulsion. The pharmaceutical composition can alternatively be a DMSO-containing solution such as a DMSO/acetone solution or DMSO/water solution (DMSO=dimethyl sulfoxide); a DMSO-containing solution can be used for experimental animal tests, but due to likely or possible skin irritancy is not usually desirable for use in humans.

In the external-topical pharmaceutical composition, e.g. an ointment or an oil-in-water or water-in-oil composition, the compound of formula (I) or the pharmaceutically acceptable salt thereof can be present in 0.1% to 10%, such as 0.2% to 5%, or 0.5% to 5%, or 1% to 5%, or 0.5% to 3% (e.g. about 1% or about 2%), by weight of the composition (w/w).

In one optional embodiment, the compound of formula (I) or the pharmaceutically acceptable salt thereof can optionally be in a particle-size-reduced form, for example obtained or obtainable by micronization. This can be, for example, for use in a pharmaceutical composition suitable for (e.g. adapted for) external topical (e.g. skin topical) administration. See the Particle size reduction sub-section below, within the Inhalable pharmaceutical compositions section, for more details.

Aqueous solubility: A preliminary screen, which can aim to estimate roughly the aqueous solubility of a compound or salt of the invention, can include (as an approximate summary): (i) creating a ca. 10 mM solution of the compound in DMSO, (ii) diluting a portion of this DMSO solution by mixing about 19 parts by volume of pH 7.4 aqueous phosphate buffered saline (PBS) buffer with 1 part by volume of the ca. 10 mM DMSO solution, (iii) “filtering” the mixture with the aid of centrifugation, and then (iv) measuring the concentration of the dissolved compound in the “filtrate”. Although some DMSO (about 5% by volume) is usually present in this solubility screen “filtrate”, the results can be a very approximate estimate of aqueous solubility, e.g. at room temperature.

Lipophilicity: The clogP (calculated log of the octanol/water partition coefficient (P)) of a particular compound or salt of the invention can estimate the lipophilicity of the compound or salt.

Solubilising and/or skin-penetration-enhancing agents: In one embodiment, an external-topical pharmaceutical composition, e.g. an ointment or an oil-in-water cream or water-in-oil cream, suitably includes an agent which acts as a skin-penetration enhancer for and/or a solubiliser of the compound of formula (I) or the salt thereof. The skin-penetration-enhancing- and/or solubilising-agent can for example be propylene glycol, diethylene glycol monoethyl ether (e.g. TRANSCUTOL™) and/or caprylocaproyl macrogolglycerides (e.g. LABRASOL™), preferably propylene glycol. The solubiliser and/or skin-penetration enhancer suitably does not comprise DMSO. The solubiliser and/or skin-penetration enhancer is preferably both a solubiliser and skin-penetration enhancer, and/or can for example be present in 0.5% to 50%, suitably 5% to 50%, more suitably 7% to 30%, for example 7% to 25%, such as about 10% to about 20% (e.g. about 10% or about 20%), by weight of the composition (w/w).

The skin-penetration enhancer is for delivery of the compound of formula (I) or salt thereof (“active agent” or “drug”) through the skin. Solubilization of the drug also helps.

The solubilising and/or skin-penetration-enhancing agents should ideally (a) be safe and/or tolerable, (b) have as low a potential for skin irritancy as possible consistent with being an effective skin penetration enhancer, and (c) be compatible with the active pharmaceutical ingredient. Note that the agent preferably functions both as a solubilising agent and a skin-penetration-enhancing agent.

Surfactants: An external-topical pharmaceutical composition, e.g. an ointment or in particular an oil-in-water cream or water-in-oil cream, can include a surfactant (e.g. as an emulsifier), for example for achieving emulsification of compositions having two or more phases. The total surfactant content can for example be 0.3% to 20%, e.g. 0.5% to 15% or 0.5% to 12% or 0.5% to 10% or 1% to 12% or 3% to 10%, by weight of the composition (w/w). The surfactant can for example comprise a nonionic surfactant such as one or more of the following: a polyoxyl C₁₂₋₂₂alkyl ether (e.g. a polyoxyl C₁₂₋₁₈alkyl ether such as polyoxyl cetyl ether or polyoxyl stearyl ether or polyoxyl lauryl ether) (e.g. present at 0.5% to 10% w/w, e.g. 2.5% to 10% w/w such as about 5% to about 8% w/w), glycerol monostearate (e.g. Arlacel 165™) (e.g. present at 0.5% to 10% w/w, e.g. about 2% w/w), sorbitan monostearate (e.g. Span 60™) (e.g. present at 0.05% to 10% w/w, e.g. about 1% w/w), and cetyl alcohol and/or stearyl alcohol (e.g. cetostearyl alcohol, e.g. wherein the total of any cetyl alcohol and any stearyl alcohol present is 0.5% to 15% w/w, e.g. 1% to 10% w/w such as 2% to 10% w/w or 5% to 10% w/w). Polyoxyl stearyl ether (steareth) can e.g. be a polyoxyl 2-21 stearyl ether, such as polyoxyl 2 stearyl ether (steareth-2), polyoxyl 10 stearyl ether (steareth-10), polyoxyl 20 stearyl ether (steareth-20) or polyoxyl 21 stearyl ether (steareth-21). Polyoxyl cetyl ether (ceteth) can e.g. be a polyoxyl 2-20 cetyl ether such as ceteth-2, ceteth-10 or ceteth-20. Polyoxyl alkyl ethers are also named polyoxyethylene alkyl ethers. Alternatively or additionally, the surfactant can for example comprise an ionic surfactant such as sodium dodecyl sulfate (SDS)=sodium lauryl sulfate (e.g. SDS present at 0.3% to 2% w/w such as 0.5% to 1.5% w/w).

Ointments and creams (and oil phase): An external-topical pharmaceutical composition can be an ointment or an oil-in-water cream or water-in-oil cream. The ointment or cream typically contains an oil phase (oily ointment base). The oil phase (ointment base) typically comprises an oil and/or a fat, preferably of a consistency suitable for skin-spreadability.

Preferably, an oil comprising or being white soft paraffin (white petrolatum) and/or a mineral oil (such as liquid paraffin) can be used. (Mineral oil can also be used as a solubiliser and/or emollient). The white soft paraffin (white petrolatum) can be of various grades, for example (for Penreco supplier) Penreco Regent White grade, Penreco Snow White grade, or Penreco Ultima White grade, in particular high melting point white soft paraffin (e.g. of Penreco Ultima White grade). Microcrystalline wax or beeswax or beeswax substitute can be used as an oil/fat in the oil phase.

Alternatively or additionally, one or more fats like straight or branched chain mono- or dialkyl esters such as isopropyl myristate, isopropyl palmitate, diisopropyl adipate, isocetyl stearate, isostearyl isostearate, decyl oleate, butyl stearate, 2-ethylhexyl palmitate, propylene glycol diester of coconut fatty acids, or a mixed ester of 2-ethyl hexanoic acid with a blend of cetyl or stearyl alcohols (e.g. known as Crodamol CAP), may be used in the oil phase (some of these are also solubilisers and/or surfactants). These may be used singly or in combination depending on the properties required.

The oil phase (oily ointment base) can for example be present at:

30% to 99.8% w/w (e.g. 50% to 99.5% w/w, e.g. 50% to 95% w/w, e.g. 60% to 95% w/w, e.g. 60% to 90% w/w) in an ointment (e.g. emulsion or homogeneous single phase);

25% to 85% w/w (e.g. 35% to 70% w/w) in a water-in-oil cream (e.g. emulsion); or

5% to 60% w/w or 8% to 55% w/w (e.g. 10% to 45% w/w or 12% to 30% w/w) in an oil-in-water cream (e.g. emulsion).

Note that the w/w percentages for the oil phase (oily ointment base), mentioned above or in the example formulations below or generally herein, exclude the amount of any surfactant(s) present (except for compound(s) listed herein as fats which also have surfactant properties), and exclude the amount of any non-oil non-fat solubilising and/or skin-penetration-enhancing agents present.

Example ointments: As an example, an external-topical pharmaceutical composition can be an ointment comprising:

the compound of formula (I) or pharmaceutically acceptable salt thereof present at 0.1% to 10% w/w (e.g. 0.2% to 5% w/w, or 0.5% to 5% w/w, or 0.5% to 3% w/w); and

an oil phase (oily ointment base) present at 30% to 99.8% w/w or 50% to 99.5% w/w or 50% to 95% w/w or 60% to 95% w/w or 60% to 90% w/w (i.e. by weight of the composition).

For example, in the above example ointment, the oil phase or composition can suitably comprise white petrolatum present at 25% to 99.5% w/w or 45% to 99% w/w or 55% to 85% w/w (i.e. by weight of the composition). Optionally, additionally or alternatively, the oil phase or composition can comprise mineral oil (e.g. as solubiliser and emollient) present at 2.5% to 25% w/w such as 4% to 20% w/w (i.e. by weight of the composition)].

In the above example ointment, the ointment can optionally comprise one or more surfactants (e.g. polyoxyl stearyl ether, polyoxyl cetyl ether or cetostearyl alcohol) present in total at 0.5% to 10% w/w or 3% to 10% w/w.

In the above example ointment, the ointment can optionally comprise one or more agents acting as a skin-penetration enhancer (preferably acting as both a solubiliser and skin-penetration enhancer and/or preferably hydrophilic such as propylene glycol) present in total at 0.5% to 50% w/w, such as 5% to 50% w/w or 7% to 30% w/w.

In the above example ointment, the ointment can optionally comprise (a) one or more antioxidants (e.g. butylated hydroxyanisole), e.g. present in total at 0.001% to 2% w/w such as 0.02% to 2% w/w; and/or (b) one or more preservatives, e.g. present in total at 0.01% to 4% w/w such as 0.05% to 1% w/w (e.g. methylparaben present at 0.05% to 2% w/w and/or propylparaben present at 0.01% to 2% w/w).

The above example ointment composition, including the oil “phase” and an optional penetration enhancer, can optionally be a homogeneous single phase. However, in one embodiment of the above example ointment composition, e.g. when using propylene glycol or another hydrophilic solubiliser and penetration enhancer, the oil phase (oily ointment base) and a hydrophilic phase containing the hydrophilic solubiliser and penetration enhancer (e.g. propylene-glycol-containing phase) have been emulsified to form an ointment emulsion.

Ointment compositions having two phases can optionally be prepared using an emulsification process whereby the hydrophilic phase (e.g. propylene-glycol-containing phase) and oil phase are first prepared in separate vessels. The hydrophilic phase can optionally contain a penetration enhancer such as propylene glycol, and optionally some or all of the compound of formula (I) or salt thereof. The oil phase can optionally contain a surfactant. Temperatures of both phases are maintained at elevated temperatures, such as about 55-90° C. or preferably from above 70 to 90° C., the oil phase temperature being sufficiently high (e.g. from above 70 to 90° C.) to melt the oil phase. While hot, one phase is added to another while mixing, e.g. using a high shear mixer, to effect emulsification, preferably keeping the temperature above 70° C. such as from above 70 to 90° C. The resulting ointment emulsion is allowed to cool, e.g. to about 15-35° C. such as to about 18-30° C., preferably while the agitation continues e.g. at lower speeds. The ointment emulsion can then optionally be dispensed from the manufacturing vessel and filled into primary packaging, for example tubes or sachets.

Optionally, an ointment can comprise a polyethylene glycol base, e.g. present at 25% to 99% w/w such as 50% to 98% w/w, instead of or as well as an oily ointment base.

Creams: An external-topical pharmaceutical composition can be a cream, e.g. a water-in-oil cream or an oil-in-water cream. Creams can sometimes be more fluid than ointments, can sometimes provide more moisture, and hence may in principle in certain cases allow for improved and/or good efficacy in patients with atopic dermatitis.

Water-in-oil creams: These usually have an increased aqueous content compared to ointments. Preferably, the water-in-oil cream is a water-in-oil cream emulsion. That is, preferably, in the water-in-oil cream, an oil phase and an aqueous phase have been emulsified to form a water-in-oil cream emulsion.

As an example, an external-topical pharmaceutical composition can be a water-in-oil cream (e.g. cream emulsion) comprising:

the compound of formula (I) or pharmaceutically acceptable salt thereof present at 0.1% to 10% w/w (e.g. 0.2% to 5% w/w, or 0.5% to 5% w/w, or 0.5% to 3% w/w);

an oil phase (oily ointment base) present at 25% to 85% w/w or 35% to 70% w/w [for example: comprising white petrolatum present at 25% to 75% w/w or 30% to 65% w/w (i.e. by weight of the composition), and/or comprising mineral oil (e.g. as solubiliser and emollient) present at 2.5% to 20% w/w or 4% to 15% w/w (i.e. by weight of the composition)];

water present in 2% to 30% w/w, e.g. 5% to 25% or 10% to 22% w/w;

one or more surfactants (e.g. polyoxyl stearyl ether) present in total at 0.5% to 12% w/w, such as 3% to 10% w/w; and

preferably, one or more agents acting as a skin-penetration enhancer (preferably acting as both a solubiliser and skin-penetration enhancer and/or preferably hydrophilic such as propylene glycol) present in total at 0.5% to 50% w/w, such as 5% to 50% w/w or 7% to 30% w/w; and

optionally, one or more antioxidants (e.g. butylated hydroxyanisole), e.g. present in total at 0.001 to 2% w/w such as 0.02 to 2% w/w; and

optionally, one or more preservatives, e.g. present in total at 0.01 to 4% w/w such as 0.05 to 1% w/w (e.g. methylparaben present at 0.05 to 2% w/w and/or propylparaben present at 0.01 to 2% w/w).

Oil-in-water creams: These usually have an increased aqueous content compared to ointments and water-in-oil creams. Preferably, the oil-in-water cream is a oil-in-water cream emulsion. That is, preferably, in the oil-in-water cream, an oil phase and an aqueous phase have been emulsified to form a oil-in-water cream emulsion.

Preferable oil-in-water creams are high-occlusion creams, wherein, after topical administration to the skin, moisture loss from the skin and/or from the cream is reduced or limited by means of sufficiently high coverage of the skin and/or by providing a sufficient barrier at the site of application.

Preferably, the oil-in-water cream contains one or more emollients (hydrating agents), such as silicones (e.g. dimethicone, e.g. dimethicone 360 or dimethicone 20), a high-viscosity wax such as microcrystalline wax, and/or mineral oil. A sufficiently high water content is also preferred, for example wherein the water is present in 15% to 60% w/w, 20% to 50% w/w, or 25% to 40% w/w.

As an example, an external-topical pharmaceutical composition can be an oil-in-water cream (e.g. cream emulsion) comprising:

the compound of formula (I) or pharmaceutically acceptable salt thereof present at 0.1% to 10% w/w (e.g. 0.2% to 5% w/w, or 0.5% to 5% w/w, or 0.5% to 3% w/w);

an oil phase (oily ointment base), preferably containing one or more ingredients capable of acting as emollients, the oil phase being present at 5% to 60% w/w or 8% to 55% w/w or preferably 10% to 45% w/w or 12% to 30% w/w;

water present in 7% to 75% w/w or 7% to 60% w/w or 10% to 60% w/w, preferably 15% to 50% w/w or 20% to 40% w/w;

one or more surfactants present in total at 0.5% to 20% w/w, e.g. 3% to 15% w/w or 3% to 10% w/w; and

preferably, one or more agents acting as a skin-penetration enhancer (preferably acting as both a solubiliser and skin-penetration enhancer and/or preferably hydrophilic such as propylene glycol) present in total at 0.5% to 50% w/w, preferably 5% to 50% w/w or 7% to 25% w/w; and

optionally, one or more solubilisers (e.g. isopropyl myristate), e.g. present at 0.5% to 20% w/w, e.g. 3 to 12% w/w; and

optionally, one or more buffers (e.g. citric acid and/or dibasic sodium phosphate), e.g. present in total at 0.05 to 5% w/w.

In the above example oil-in-water cream composition, the oil phase preferably comprises mineral oil (e.g. as emollient and solubiliser) present at 15% to 50% w/w or 20% to 45% w/w (i.e. by weight of the composition), and/or comprises a high-viscosity wax such as microcrystalline wax (e.g. as emollient) present at 5% to 25% w/w such as 8% to 15% w/w, and/or comprises a silicone (such as dimethicone e.g. dimethicone 360 or dimethicone 20, e.g. as emollient) present at 0.5% to 20% such as 0.5% to 10% or 1% to 5% w/w.

In the above example oil-in-water cream composition, the one or more surfactants preferably comprise: glycerol monostearate present at 0.5% to 10% w/w, and/or sorbitan monostearate present at 0.05% to 10% w/w, and/or [cetyl alcohol and/or stearyl alcohol] present in total at 0.1% to 15% or 1 to 10% w/w.

Cream emulsions, e.g. water-in-oil or oil-in-water cream emulsions, can be prepared by a process in which an aqueous phase is prepared, e.g. prepared before emulsification. The aqueous phase usually contains water and a solubiliser and/or skin-penetration enhancer such as propylene glycol, and optionally contains some or all of the compound of formula (I) or salt thereof, and/or optionally contains surfactant. The oil phase, e.g. containing white petrolatum and/or mineral oil, and/or optionally containing surfactant, can be prepared in a separate vessel. Temperatures of both phases are maintained at elevated temperatures, such as about 55-90° C. or preferably from above 70 to 90° C., the oil phase temperature being sufficiently high (e.g. from above 70 to 90° C.) to melt the oil phase. While hot, one phase is added to another while mixing, e.g. using a high shear mixer, to effect emulsification, preferably keeping the temperature above 70° C. such as from above 70 to 90° C. The resulting emulsion is allowed to cool, e.g. to about 15-35° C. such as to about 18-30° C., preferably while the agitation continues e.g. at lower speeds. The cream emulsion can then optionally be dispensed from the manufacturing vessel and filled into primary packaging, for example tubes or sachets.

Typically, a pharmaceutical composition of the invention suitable for external topical administration can be administered once daily, twice daily or more than twice daily, to external body part(s), e.g. on the skin such as at a site of diseased skin, e.g. skin suffering from atopic dermatitis.

### Pharmaceutical Compositions Suitable For Parenteral or Oral Administration

A pharmaceutical composition suitable for (e.g. adapted for) parenteral (e.g. intravenous, subcutaneous, or intramuscular) administration can comprise a solution or suspension of the compound or pharmaceutically acceptable salt in a sterile pharmaceutically and parenterally acceptable aqueous liquid carrier (e.g. sterile water or a sterile aqueous solution) or in a parenterally acceptable oil. Alternatively, an aqueous solution can be lyophilised to prepare the parenteral composition. A lyophilised pharmaceutical composition suitable for (e.g. adapted for) parenteral administration may, in use, optionally be reconstituted with a suitable solvent, e.g. sterile water or a sterile parenterally acceptable aqueous solution, just prior to administration. A pharmaceutical composition suitable for (e.g. adapted for) parenteral administration may optionally comprise a preservative.

As mentioned above, oral administration is generally not thought to be a preferred route of administration. However, in the event that oral administration is to be used, a pharmaceutical composition suitable for oral administration can be liquid or solid; for example it can be a syrup, suspension or emulsion, a tablet, a capsule or a lozenge. A liquid formulation (e.g. oral) can generally consist of a suspension or solution of the compound or pharmaceutically acceptable salt in a suitable pharmaceutically acceptable liquid carrier(s), for example an aqueous solvent such as water, aqueous ethanol or aqueous glycerine, or a non-aqueous solvent, such as polyethylene glycol or an oil. The formulation may also contain a suspending agent, preservative, flavouring and/or colouring agent.

In one embodiment, the pharmaceutical composition is in unit dose form, such as a tablet or capsule for oral administration, e.g. for oral administration to a human.

A pharmaceutical composition suitable for oral administration being a tablet can comprise one or more pharmaceutically acceptable carriers and/or excipients suitable for preparing tablet formulations. The carrier can for example be or include lactose, cellulose (for example microcrystalline cellulose), or mannitol. The tablet can also or instead contain one or more pharmaceutically acceptable excipients, for example a binding agent such as hydroxypropylmethylcellulose or povidone (polyvinylpyrrolidone), a lubricant e.g. an alkaline earth metal stearate such as magnesium stearate, and/or a tablet disintegrant such as sodium starch glycollate, croscarmellose sodium, or crospovidone (cross-linked polyvinylpyrrolidone). The pharmaceutical composition being a tablet can be prepared by a method comprising the steps of: (i) mixing the compound of formula (I), as herein defined, or a pharmaceutically acceptable salt thereof, with the one or more pharmaceutically acceptable carriers and/or excipients, (ii) compressing the resulting mixture (which is usually in powder form) into tablets, and (iii) optionally coating the tablet with a tablet film-coating material.

A pharmaceutical composition suitable for oral administration being a capsule can be prepared using encapsulation procedures. For example, pellets or powder containing the active ingredient can be prepared using a suitable pharmaceutically acceptable carrier and then filled into a hard gelatin capsule. Alternatively, a dispersion or suspension can be prepared using any suitable pharmaceutically acceptable carrier, for example an aqueous gum or an oil and the dispersion or suspension then filled into a soft gelatin capsule.

### Dosage Regimens

In a pharmaceutical composition suitable for (e.g. adapted for) external topical administration, e.g. an ointment or an oil-in-water or water-in-oil composition, the compound of formula (I) or the pharmaceutically acceptable salt thereof can be present in 0.1% to 10%, such as 0.2% to 5%, or 0.5% to 5%, or 0.5% to 3%, by weight of the composition (w/w). Typically, an external-topical pharmaceutical composition can be administered once daily, twice daily or more than twice daily, to external body part(s), e.g. to the skin such as at a site of diseased skin. The amount administered is usually such as substantially to cover the site(s) of diseased skin.

The pharmaceutical composition can optionally be in unit dose form. The unit dose form can for example be:

(a) a rupturable or peel-openable sealed dose container containing a dry powder inhalable pharmaceutical composition (e.g. a plurality of which are usually disposed inside a suitable inhalation device);
(b) a vial, ampoule or filled syringe for parenteral administration e.g. comprising a solution or suspension of the compound or pharmaceutically acceptable salt in a suitable carrier such as an aqueous carrier or e.g. containing a lyophilised parenteral pharmaceutical composition (the vial or ampoule can optionally be manufactured using a blow-fill-seal process); or
(c) a tablet or capsule for oral administration e.g. for oral administration to a human.

In the pharmaceutical composition of the invention, a or each dosage unit for inhaled or intranasal administration can for example contain from 0.001 to 10 mg, such as 0.005 to 7.5 mg, for example 0.02 to 1 mg or 0.05 to 0.25 mg, of a compound (e.g. of formula (I)) or a pharmaceutically acceptable salt thereof, calculated as the free base. A or each dosage unit for oral or parenteral administration can for example contain from 0.02 to 1000 mg, such as 0.2 to 350 mg, of a compound (e.g. of formula (I)) or a pharmaceutically acceptable salt thereof, calculated as the free base.

When an inhalable or intranasal composition is used, a pharmaceutically acceptable compound or salt of the invention can for example be administered to a mammal (e.g. human) in a daily inhaled or intranasal dose of: 0.0001 to 0.1 mg/kg body weight/day, e.g. 0.0003 to 0.015 mg/kg/day or 0.0007 to 0.004 mg/kg/day, of the compound (e.g. of formula (I)) or a pharmaceutically acceptable salt thereof, calculated as the free base.

A compound, e.g. of formula (I), or a pharmaceutically acceptable salt thereof of the invention can, for example, be administered to a human in a total daily inhaled or intranasal dose of: 0.001 to 10 mg per day, or 0.005 to 7.5 mg per day, or 0.02 to 1 mg per day, or 0.05 to 0.25 mg per day, of the compound (e.g. of formula (I)) or a pharmaceutically acceptable salt thereof, calculated as the free base. These total daily doses can be administered as a single dose once daily, or can represent the summation of two or more separate doses administered at different times of the day (e.g. two doses per day administered every ca. 12 hours). These total daily doses can e.g. be for administration to an adult human e.g. of 50-120 kg or 60-100 kg body weight.

When a parenteral or oral composition is used, a pharmaceutically acceptable compound or salt of the invention is optionally, for example, administered to a mammal (e.g. human) in a daily parenteral or oral dose of 0.0003 mg to 15 mg per kg body weight per day (mg/kg/day), for example 0.003 to 5 mg/kg/day, of the compound (e.g. of formula (I)) or a pharmaceutically acceptable salt thereof, calculated as the free base.

A compound, e.g. of formula (I), or pharmaceutically acceptable salt thereof of the invention is optionally, for example, administered to a human (e.g. adult human) in a total daily parenteral or oral dose of 0.02 mg to 1000 mg per day or 0.2 to 350 mg per day of the compound (e.g. of formula (I)) or a pharmaceutically acceptable salt thereof, calculated as the free base.

### Combinations

The compounds, salts and/or pharmaceutical compositions according to the invention may also be used in combination with another therapeutically active agent, for example, a β₂ adrenoreceptor agonist, an anticholinergic compound (e.g. muscarinic (M) receptor antagonist), an anti-histamine, an anti-allergic, an anti-inflammatory agent, an antiinfective agent or an immunosuppressant.

The invention thus provides, in a further aspect, a combination comprising a compound of formula (I) or a pharmaceutically acceptable salt thereof together with another therapeutically active agent, for example, a muscarinic (M) receptor antagonist, a 2-adrenoreceptor agonist (beta-2 adrenoreceptor agonist), an anti-histamine, an anti-allergic, an anti-inflammatory agent, an antiinfective agent or an immunosuppressant.

The invention also provides, in a further preferred aspect, a combination comprising a compound of formula (I) or a pharmaceutically acceptable salt thereof together with a muscarinic (M) receptor antagonist.

The muscarinic (M) receptor antagonist can be an M₁, M₂, M₁/M₂, or M₃ receptor antagonist, more preferably a M₃ receptor antagonist, still more preferably a M₃ receptor antagonist which selectively antagonizes (e.g. antagonizes 10 times or more strongly) the M₃ receptor over the M₁ and/or M₂ receptor.

For combinations of a muscarinic (M) receptor antagonist with PDE4 inhibitors, see for example WO 03/011274 A2 and WO 02/069945 A2/US 2002/0193393 A1 and US 2002/052312 A1, and some or all of these publications give examples of anticholinergic compounds/muscarinic (M) receptor antagonists which may be used with the compounds of formula (I) or salts, and/or suitable pharmaceutical compositions. For example, the muscarinic receptor antagonist can comprise or be an ipratropium salt (e.g. ipratropium bromide), an oxitropium salt (e.g. oxitropium bromide), or more preferably a tiotropium salt (e.g. tiotropium bromide); see e.g. EP 418 716 A1 for tiotropium.

Muscarinic antagonists which can be used in the combination of the present invention include a compound (including a pharmaceutically acceptable salt thereof) defined by claim 1, 2, 3 or 4 of WO 2005/037280 A1. These compounds are stated or implied as being muscarinic (e.g. M₃) acetylcholine receptor antagonists.

The muscarinic (M) receptor antagonist, e.g. M₃ receptor antagonist, is preferably for inhaled administration, more preferably in particle-size-reduced form e.g. as defined herein. More preferably, both the muscarinic (M) receptor antagonist and the compound of formula (I) or the pharmaceutically acceptable salt thereof are for inhaled administration. Preferably, the muscarinic receptor antagonist and the compound of formula (I) or salt are for simultaneous administration. The muscarinic receptor antagonist combination is preferably for treatment and/or prophylaxis of COPD.

In one embodiment, the combination includes a β₂-adrenoreceptor agonist (beta-2 adrenoreceptor agonist) being salmeterol (e.g. as racemate or a single enantiomer such as the R-enantiomer), salbutamol, formoterol, salmefamol, fenoterol or terbutaline, or a salt thereof (e.g. pharmaceutically acceptable salt thereof, for example the xinafoate salt of salmeterol, the sulphate salt or free base of salbutamol or the fumarate salt of formoterol. Long-acting β₂-adrenoreceptor agonists are preferred, especially those having a therapeutic effect over a 12-24 hour period such as salmeterol or formoterol. Preferably, the β₂-adrenoreceptor agonist is for inhaled administration, e.g. once per day and/or for simultaneous inhaled administration; and more preferably the β₂-adrenoreceptor agonist is in particle-size-reduced form e.g. as defined herein. Preferably, the β₂-adrenoreceptor agonist combination is for treatment and/or prophylaxis of COPD or asthma. Salmeterol or a pharmaceutically acceptable salt thereof, e.g. salmeterol xinofoate, can be administered to humans at an inhaled dose of 25 to 50 micrograms twice per day (measured as the free base).

Preferred long acting 2-adrenoreceptor agonists for use in the combination include those described in WO 02/066422A, WO 03/024439, WO 02/070490 and WO 02/076933.

Preferred long-acting β₂-adrenoreceptor agonists (beta-2 adrenoreceptor agonists) include compounds of formula (XX) (described in WO 02/066422):

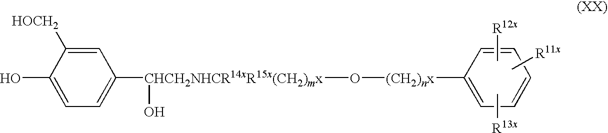

or a salt or solvate thereof, wherein in formula (XX):
m^{X} is an integer of from 2 to 8;
n^{X} is an integer of from 3 to 11,
with the proviso that m^{X}+n^{X} is 5 to 19,
R^{11X} is —XSO₂NR^{16X}R^{17X} wherein X is —(CH₂)p^{x}- or C₂₋₆ alkenylene;
R^{16X} and R^{17X} are independently selected from hydrogen, C₁₋₆alkyl, C₃₋₇cycloalkyl, C(O)NR^{18X}R^{19X}, phenyl, and phenyl (C₁₋₄alkyl)-,
or R^{16X} and R^{17X}, together with the nitrogen to which they are bonded, form a 5-, 6-, or 7-membered nitrogen containing ring, and R^{16X} and R^{17X} are each optionally substituted by one or two groups selected from halo, C₁₋₆alkyl, C₁₋₆haloalkyl, C₁₋₆alkoxy, hydroxy-substituted C₁₋₆alkoxy, —CO₂R^{18X}, —SO₂NR^{18X}R^{19X}, —CONR^{18X}R^{19X}, —NR^{18X}C(O)R^{19X}, or a 5-6- or 7-membered heterocylic ring;
R^{12X} and R^{19X} are independently selected from hydrogen, C₁₋₆alkyl, C₃₋₆cycloalkyl, phenyl, and phenyl (C₁₋₄alkyl)-; and
p^{X} is an integer of from 0 to 6, preferably from 0 to 4;

R^{12X} and R^{13X} are independently selected from hydrogen, C₁₋₆alkyl, C₁₋₆alkoxy, halo, phenyl, and C₁₋₆haloalkyl; and

R^{14X} and R^{15X} are independently selected from hydrogen and C₁₋₄alkyl with the proviso that the total number of carbon atoms in R^{14X} and R^{15X} is not more than 4.
Preferred β₂-adrenoreceptor agonists disclosed in WO 02/066422 include:
3-(4-{[6-({(2R)-2-hydroxy-2-[4-hydroxy-3-(hydroxymethyl)-phenyl]ethyl}amino)hexyl]oxy}butyl)benzenesulfonamide, or
3-(3-{[7-({(2R)-2-hydroxy-2-[4-hydroxy-3-hydroxymethyl)phenyl]ethyl}-amino)heptyl]oxy}propyl)benzenesulfonamide; or a salt thereof.

A preferred β₂-adrenoreceptor agonist disclosed in WO 03/024439 is:
4-{(1R)-2-[(6-{2-[(2,6-dichlorobenzyl)oxy]ethoxy}hexyl)amino]-1-hydroxyethyl}-2-(hydroxymethyl)phenol or a salt thereof.

An anti-histamine usable in a combination of a compound of formula (I) or salt can for example be for oral administration (e.g. this can be as a separately-administrable tablet), and can be for treatment and/or prophylaxis of allergic rhinitis. Examples of anti-histamines for oral administration include methapyrilene, or H1 antagonists such as cetirizine, loratadine (e.g. Clarityn™), desloratadine (e.g. Clarinex™) or fexofenadine (e.g. Allegra™).

An anti-histamine usable in a combination of a compound of formula (I) or salt can for example be for intranasal administration. An anti-histamine for intranasal administration can e.g. be azelastine or a salt thereof (e.g. azelastine hydrochloride, e.g. 0.1% w/v aqueous solution), or levocabastine or a salt thereof (e.g. levocabastine hydrochloride). The anti-histamine olopatadine (e.g. as olopatadine HCl) can be used e.g. as eye drops.

Other possible combinations include, for example, a combination comprising a compound of formula (I) or a pharmaceutically acceptable salt thereof together with another anti-inflammatory agent such as an anti-inflammatory corticosteroid; or a non-steroidal anti-inflammatory drug (NSAID) such as a leukotriene antagonist (e.g. montelukast), an iNOS inhibitor, a tryptase inhibitor, a elastase inhibitor, a beta-2 integrin antagonist, a adenosine 2a agonist, a CCR3 antagonist, or a 5-lipoxygenase inhibitor; or an antiinfective agent (e.g. an antibiotic or an antiviral). An iNOS inhibitor is optionally for oral administration. Examples of iNOS inhibitors (inducible nitric oxide synthase inhibitors) include those disclosed in WO 93/13055, WO 98/30537, WO 02/50021, WO 95/34534 and WO 99/62875.

In a combination comprising a compound of formula (I) or a pharmaceutically acceptable salt thereof together with an anti-inflammatory corticosteroid (which can for example be for treatment and/or prophylaxis of asthma, COPD, allergic rhinitis, psoriasis or atopic dermatitis), then the anti-inflammatory corticosteroid can be fluticasone propionate (e.g. see U.S. Pat. No. 4,335,121), beclomethasone 17-propionate ester, beclomethasone 17,21-dipropionate ester, dexamethasone or an ester thereof, mometasone or an ester thereof (e.g. mometasone furoate), betamethasone valerate (for external topical administration), clobetasol propionate (for external topical administration), ciclesonide, budesonide, flunisolide, or a compound as described in WO 02/12266 A1 (e.g. as claimed in any of claims 1 to 22 therein), or a pharmaceutically acceptable salt of any of the above. If the anti-inflammatory corticosteroid is a compound as described in WO 02/12266 A1, then it can be Example 1 therein {which is 6α,9α-difluoro-17α-[(2-furanylcarbonyl)oxy]-11β-hydroxy-16α-methyl-3-oxo-androsta-1,4-diene-17β-carbothioic acid S-fluoromethyl ester} or Example 41 therein {which is 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester}, or a pharmaceutically acceptable salt thereof. The anti-inflammatory corticosteroid can for example be for inhaled, intranasal or external topical administration. Fluticasone propionate can be used and is preferably for inhaled administration to a human either (a) at a dose of 250 micrograms once per day or (b) at a dose of 50 to 250 micrograms twice per day. In a combination comprising betamethasone valerate for external topical administration, the betamethasone valerate can be present at from about 0.025% to about 0.1% w/w in an externally-topically-administrable composition such as a cream or ointment. In a combination comprising clobetasol propionate for external topical administration, the clobetasol propionate can be present at about 0.0525% w/w or about 0.05% w/w in an externally-topically-administrable composition such as a cream or ointment.

Also provided is a combination comprising a compound of formula (I) or a pharmaceutically acceptable salt thereof together with 2-adrenoreceptor agonist and an anti-inflammatory corticosteroid, for example as described in WO 03/030939 A1.

Preferably this combination is for treatment and/or prophylaxis of asthma, COPD or allergic rhinitis. The β₂-adrenoreceptor agonist and/or the anti-inflammatory corticosteroid can be as described above and/or as described in WO 03/030939 A1. In this “triple” combination, the β₂-adrenoreceptor agonist can for example be salmeterol or a pharmaceutically acceptable salt thereof (e.g. salmeterol xinafoate), and the anti-inflammatory corticosteroid can for example be fluticasone propionate.

Other examples of combinations, in particular for external topical administration (e.g. versus atopic dermatitis), include, for example, a combination comprising a compound of formula (I) or a pharmaceutically acceptable salt thereof together with an immunosuppressant, e.g. a calcineurin inhibitor such as pimecrolimus or tacrolimus. The immunosuppressant can in particular be an externally-topically administrable immunosuppressant such as pimecrolimus (e.g. pimecrolimus at ca. 1% w/w concentration in a topical composition such as a cream, and/or e.g. Elidel™) or tacrolimus (e.g. tacrolimus at from about 0.03% to about 0.1% w/w concentration in a topical composition such as an ointment, and/or e.g. Protopic™). The externally-topically administrable immunosuppressant can be administered or administrable in a external-topical composition separately from the compound or salt of the invention, or it can be contained with the compound of formula (I) or pharmaceutically acceptable salt in a combined externally-topically-administrable composition.

For external topical administration, e.g. versus an inflammatory and/or allergic skin disease such as atopic dermatitis or psoriasis, in a combination of the compound or salt of the invention together with an anti-infective agent, the anti-infective agent can include (e.g. be) an externally-topically-administrable antibacterial, such as mupirocin or a salt thereof (e.g. mupirocin calcium salt) (e.g. Bactroban™) or such as an externally-topically-administrable pleuromutilin antibacterial (e.g. retapamulin or a salt thereof, which can be present in about 1% w/w by weight of an externally-topically-administrable pharmaceutical composition, such as an ointment). Alternatively or additionally, for external topical administration, the anti-infective agent can include an externally-topically-administrable antifungal such as clotrimazole (e.g. at about 1% to about 10% w/w or at about 1% to about 2% w/w in a topical composition), or ketoconazole, or terbinafine (e.g. as HCl salt and/or at about 1% w/w).

For external topical administration, e.g. versus atopic dermatitis, a combination with an anti-itch compound may optionally be used.

The combinations referred to above may be presented for use in the form of a pharmaceutical composition and thus a pharmaceutical composition comprising a combination as defined above together with one or more pharmaceutically acceptable carriers and/or excipients represent a further aspect of the invention.

The individual compounds of such combinations may be administered either sequentially or simultaneously in separate or combined pharmaceutical composition.

In one embodiment, the combination as defined herein can be for simultaneous inhaled administration and is disposed in a combination inhalation device. Such a combination inhalation device is another aspect of the invention. Such a combination inhalation device can comprise a combined pharmaceutical composition for simultaneous inhaled administration (e.g. dry powder composition), the composition comprising all the individual compounds of the combination, and the composition being incorporated into a plurality of sealed dose containers mounted longitudinally in a strip or ribbon inside the inhalation device, the containers being rupturable or peel-openable on demand; for example such inhalation device can be substantially as described in GB 2,242,134 A (DISKUS™) and/or as described above. Alternatively, the combination inhalation device can be such that the individual compounds of the combination are administrable simultaneously but are stored separately (or wholly or partly stored separately for triple combinations), e.g. in separate pharmaceutical compositions, for example as described in PCT/EP03/00598 filed on 22 Jan. 2003, published as WO 03/061743 (e.g. as described in the claims thereof e.g. claim 1).

The invention also provides a method of preparing a combination as defined herein,
the method comprising either
(a) preparing a separate pharmaceutical composition for administration of the individual compounds of the combination either sequentially or simultaneously, or
(b) preparing a combined pharmaceutical composition for administration of the individual compounds of the combination simultaneously,
wherein the pharmaceutical composition comprises the combination together with one or more pharmaceutically acceptable carriers and/or excipients.

The invention also provides a combination as defined herein, prepared by a method as defined herein.

### Biological Test Methods

### PDE 3, PDE 4B, PDE 4D, PDE 5, PDE 6 Primary Assay Methods

The biological activity of the compounds or salts of the invention can be measured in the assay methods shown below.

Some of the Examples disclosed herein and encompassed within the invention are selective PDE4 inhibitors, i.e. they inhibit PDE4 (e.g. PDE4B) more strongly than they inhibit PDE3 and/or more strongly than they inhibit PDE5 and/or more strongly than they inhibit PDE6. It is to be recognised that such selectivity is not essential to the invention.

Possible PDE Enzyme Sources and Literature References

Human recombinant PDE4B, in particular the 2B splice variant thereof (HSPDE4B2B), is disclosed in WO 94/20079 and also M. M. McLaughlin et al., “A low Km, rolipram-sensitive, cAMP-specific phosphodiesterase from human brain: cloning and expression of cDNA, biochemical characterisation of recombinant protein, and tissue distribution of mRNA”, J. Biol. Chem., 1993, 268, 6470-6476. For example, in Example 1 of WO 94/20079, human recombinant PDE4B is described as being expressed in the PDE-deficient yeast Saccharomyces cerevisiae strain GL62, e.g. after induction by addition of 150 uM CuSO₄, and 100,000×g supernatant fractions of yeast cell lysates are described for use in the harvesting of PDE4B enzyme.

Human recombinant PDE4D (HSPDE4D3A) is disclosed in P. A. Baecker et al., “Isolation of a cDNA encoding a human rolipram-sensitive cyclic AMP phosphodiesterase (PDE IV_{D})”, Gene, 1994, 138, 253-256.

Human recombinant PDE5 is disclosed in K. Loughney et al., “Isolation and characterisation of cDNAs encoding PDE5A, a human cGMP-binding, cGMP-specific 3′,5′-cyclic nucleotide phosphodiesterase”, Gene, 1998, 216, 139-147.

PDE3 can be purified from bovine aorta as described by H. Coste and P. Grondin, “Characterisation of a novel potent and specific inhibitor of type V phosphodiesterase”, Biochem. Pharmacol., 1995, 50, 1577-1585.

PDE6 can be purified from bovine retina as described by: P. Catty and P. Deterre, “Activation and solubilization of the retinal cGMP-specific phosphodiesterase by limited proteolysis”, Eur. J. Biochem., 1991, 199, 263-269; A. Tar et al. “Purification of bovine retinal cGMP phosphodiesterase”, Methods in Enzymology, 1994, 238, 3-12; and/or D. Srivastava et al. “Effects of magnesium on cyclic GMP hydrolysis by the bovine retinal rod cyclic GMP phosphodiesterase”, Biochem. J., 1995, 308, 653-658.

### Inhibition of PDE 3, PDE 4B, PDE 4D, PDE 5 or PDE 6 Activity: Radioactive Scintillation Proximity Assay (SPA)

The ability of compounds to inhibit catalytic activity at PDE4B or 4D (human recombinant), PDE3 (from bovine aorta), PDE5 (human recombinant) or PDE6 (from bovine retina) can optionally be determined by Scintillation Proximity Assay (SPA) in a 96-well format.

Test compounds (as a solution in DMSO, preferably about 2 microliter (ul) volume of DMSO solution) are preincubated at ambient temperature (room temperature, e.g. 19-23° C.) in Wallac Isoplates (code 1450-514) with PDE enzyme in 50 mM Tris-HCl buffer pH 7.5, 8.3 mM MgCl₂, 1.7 mM EGTA, 0.05% (w/v) bovine serum albumin for 10-30 minutes (usually 30 minutes). The enzyme concentration is adjusted so that no more than 20% hydrolysis of the substrate defined below occurs in control wells without compound, during the incubation. For the PDE3, PDE4B and PDE4D assays, [5′,8-3H]Adenosine 3′,5′-cyclic phosphate (Amersham Pharmacia Biotech, code TRK.559; or Amersham Biosciences UK Ltd, Pollards Wood, Chalfont St Giles, Buckinghamshire HP8 4SP, UK) is added to give 0.05 uCi per well and about 10 nM final concentration. For the PDE5 and PDE6 assays, [8-³H]Guanosine 3′,5′-cyclic phosphate (Amersham Pharmacia Biotech, code TRK.392) is added to give 0.05 uCi per well and about 36 nM final concentration. Plates containing assay mixture, preferably approx. 100 ul volume of assay mixture, are mixed on an orbital shaker for 5 minutes and incubated at ambient temperature for 1 hour. Phosphodiesterase SPA beads (Amersham Pharmacia Biotech, code RPNQ 0150) are added (about 1 mg per well) to terminate the assay. Plates are sealed and shaken and allowed to stand at ambient temperature for 35 minutes to 1 hour (preferably 35 minutes) to allow the beads to settle. Bound radioactive product is measured using a WALLAC TRILUX 1450 Microbeta scintillation counter. For inhibition curves, 10 concentrations (e.g. 1.5 nM-30 uM) of each compound are assayed. Curves are analysed using ActivityBase and XLfit (ID Business Solutions Limited, 2 Ocean Court, Surrey Research Park, Guildford, Surrey GU2 7QB, United Kingdom) Results are expressed as pIC₅₀ values.

In an alternative to the above radioactive SPA assay, PDE4B or PDE4D inhibition can be measured in the following Fluorescence Polarisation (FP) assay:

### Inhibition of PDE4B or PDE4D Activity: Fluorescence Polarisation (FP) Assay

The ability of compounds to inhibit catalytic activity at PDE4B (human recombinant) or PDE4D (human recombinant) can optionally be determined by IMAP Fluorescence Polarisation (FP) assay (IMAP Explorer kit, available from Molecular Devices Corporation, Sunnydale, Calif., USA; Molecular Devices code: R8062) in 384-well format.

The IMAP FP assay is able to measure PDE activity in an homogenous, non-radioactive assay format. The FP assay uses the ability of immobilised trivalent metal cations, coated onto nanoparticles (tiny beads), to bind the phosphate group of FI-AMP that is produced on the hydrolysis of fluorescein-labelled (FI) cyclic adenosine mono-phosphate (FI-cAMP) to the non-cyclic FI-AMP form. FI-cAMP substantially does not bind. Binding of FI-AMP product to the beads (coated with the immobilised trivalent cations) slows the rotation of the bound FI-AMP and leads to an increase in the fluorescence polarisation ratio of parallel to perpendicular light. Inhibition of the PDE reduces/inhibits this signal increase.

Test compounds (small volume, e.g. ca. 0.5 to 1 microliters (ul), preferably ca. 0.5 ul, of solution in DMSO) are preincubated at ambient temperature (room temperature, e.g. 19-23° C.) in black 384-well microtitre plates (supplier: NUNC, code 262260) with PDE enzyme in 10 mM Tris-HCl buffer pH 7.2, 1 mM MgCl₂, 0.1% (w/v) bovine serum albumin, and 0.05% NaN₃ for 10-30 minutes. The enzyme level is set by experimentation so that reaction is linear throughout the incubation. Fluorescein adenosine 3′,5′-cyclic phosphate (from Molecular Devices Corporation, Molecular Devices code: R7091) is added to give about 40 nM final concentration (final assay volume usually ca. 20-40 ul, preferably ca. 20 ul). Plates are mixed on an orbital shaker for 10 seconds and incubated at ambient temperature for 40 minutes. IMAP binding reagent (as described above, from Molecular Devices Corporation, Molecular Devices code: R7207) is added (60 ul of a 1 in 400 dilution in binding buffer of the kit stock solution) to terminate the assay. Plates are allowed to stand at ambient temperature for 1 hour. The Fluorescence Polarisation (FP) ratio of parallel to perpendicular light is measured using an Analyst™ plate reader (from Molecular Devices Corporation). For inhibition curves, 10 concentrations (e.g. 1.5 nM-30 uM) of each compound are assayed. Curves are analysed using ActivityBase and XLfit (ID Business Solutions Limited, 2 Ocean Court, Surrey Research Park, Guildford, Surrey GU2 7QB, United Kingdom). Results are expressed as pIC₅₀ values.

In the FP assay, reagents can be dispensed using Multidrop™ (available from Thermo Labsystems Oy, Ratastie 2, PO Box 100, Vantaa 01620, Finland).

For a given PDE4 inhibitor, the PDE4B (or PDE4D) inhibition values measured using the SPA and FP assays can differ slightly. However, in a regression analysis of 100 test compounds (not necessarily compounds of the invention), the pIC₅₀ inhibition values measured using SPA and FP assays have been found generally to agree within about 0.5 log units, for each of PDE4B and PDE4D (linear regression coefficient 0.966 for PDE4B and 0.971 for PDE4D; David R. Mobbs et al., “Comparison of the IMAP Fluorescence Polarisation Assay with the Scintillation Proximity Assay for Phosphodiesterase Activity”, poster presented at 2003 Molecular Devices UK & Europe User Meeting, 2 Oct. 2003, Down Hall, Harlow, Essex, United Kingdom).

Biological Data obtained for some of the Examples (PDE4B inhibitory activity, either as one reading (n=1) or as an average of 2 or more readings (n=2 or more) are generally as follows, based on measurements only, generally (without any warranty) using SPA and/or FP assay(s) generally as described above or generally similar or generally analogous to those described above. In each of the SPA and FP assays, absolute accuracy of measurement is not possible, and the readings given are generally thought to be accurate only up to very approximately ±0.5 of a log unit, depending on the number of readings made and averaged:

| | PDE4B pIC₅₀ |
|---|---|
| | (generally in FP |
| | assay) |
| Example numbers | (±about 0.5) |
| 2, 2b, 6, 8, 9, 9b, 13, 17, 22, 24, 32, 34 | about 9.0 to |
| | about 9.9 |
| 1, 3, 5, 7, 11, 12, 14, 15, 16, | about 10.0 to |
| 18, 19, 20, 25, 26, 27, 31 | about 10.8 |
| 10 | about 10.0 |
| 23 | about 9.8 |
| 28 | about 10.6 |
| 29 | about 10.0 |
| 35 | about 10.4 |

A large majority or substantially all of the Examples have been tested for PDE4B inhibition, mostly or all using the FP assay generally as described above or a generally similar or generally analogous assay. A large majority or substantially all of the Examples tested have PDE4B inhibitory activities in the range of pIC₅₀=about 8.4 (±about 0.5) to about 10.8 (± about 0.5).

Some or all of the PDE4B-tested Examples have also been tested, on an optional basis, for PDE3 and/or PDE5 inhibition using the above-described assays or generally similar or generally analogous assays or other assays.

Emesis: Some known PDE4 inhibitors can cause emesis and/or nausea to greater or lesser extents, especially after systemic exposure e.g. after oral administration (e.g. see Z. Huang et al., Current Opinion in Chemical Biology, 2001, 5: 432-438, see especially pages 433-434 and refs cited therein). Therefore, it would be preferable, but not essential, if a PDE4 inhibitory compound or salt of the invention were to cause only limited or manageable emetic side-effects, e.g. after inhaled or parenteral or external-topical administration. Emetic side-effects can for example be measured by the emetogenic potential of the compound or salt when administered to ferrets or monkeys; for example the time to onset, extent, frequency and/or duration of vomiting, retching and/or writhing in ferrets or monkeys is optionally measured, after intratracheal or parenteral or intraperitoneal (or oral) administration of the PDE4 inhibitor compound or salt. See for example A. Robichaud et al., “Emesis induced by inhibitors of [PDE IV] in the ferret”, Neuropharmacology, 1999, 38, 289-297, erratum Neuropharmacology, 2001, 40, 465-465.

Other side effects: Some known PDE4 inhibitors can cause other side effects such as headache and/or other central nervous sytem (CNS—) mediated side effects; and/or gastrointestinal (GI) tract disturbances. Therefore, it would be preferable but not essential if a particular PDE4 inhibitory compound or salt of the invention were to cause only limited or manageable side-effects in one or more of these side-effect categories.

### Other Optional In Vitro Assays:

### Inhibition of TNF-α (TNF-alpha) Production in Human PBMC (Peripheral Blood Mononuclear Cell) Assay (MSD Technology)

This is an optional supplementary test, e.g. for potentially inhalably-administrable PDE4 inhibitors.

A 96-well plate (96 MicroWell™ Plates Nunclon™Δ-High Flange Design, Fisher Scientific UK, Bishop Meadow Road, Loughborough LE 11 5 RG, Leicestershire, UK) is prepared by initially adding to column 1 ca. 10 mM of test compound dissolved in DMSO. For a more potent compound, a more diluted solution in DMSO may be used. The compound is further diluted with DMSO into columns 2 to 9 by 8 successive 3fold dilutions using the Biomek® FX Laboratory Automation Workstation (Beckman Coulter, Inc., 4300 N. Harbor Boulevard, P.O. Box 3100, Fullerton, Calif. 92834-3100 USA). Column 10 is used as a DMSO negative control (High Signal, 0% response), whilst column 11, which contains 10 mM of the PDE4 inhibitor roflumilast, is used as a positive control (Low Signal, 100% response). About 1 μl (about 1 ul) of compound is transferred to the compound plate using the Biomek® FX.

PBMC cells (peripheral blood mononuclear cells) are prepared from heparinised human blood (using 1% v/v Heparin Sodium 10001 U/ml Endotoxin Free, Leo Laboratories Ltd., Cashel Road, Dublin 12. Ireland, Cat No: PL0043/0149) from normal volunteers using the Accuspin™ System-Histopaque®-1077 essentially (Sigma-Aldrich Company Ltd., The Old Brickyard New Rd, Gillingham Dorset SP8 4XT). About 20 ml of blood is overlaid onto 15 ml Histopaque® in Accuspin™ tubes. The tube is then centrifuged at about 800 g for ca. 20 minutes. The cells are collected from the interface, washed by centrifugation (ca. 1300 g, ca. 10 minutes) and resuspended in RPMI1640 medium (Low endotoxin RPMI1640 medium, Cat No: 31870-025, Invitrogen Corporation Invitrogen Ltd, 3 Fountain Drive, Inchinnan Business Park, Paisley PA4 9RF, UK) containing 10% foetal calf serum, 1% L-glutamine (Invitrogen Corporation, Cat No: 25030024) and 1% penicillin/streptomycin (Invitrogen Corporation, Cat No: 15140-122). Viable cells are counted by trypan blue staining and diluted to 1×10⁶ viable cells/ml. About 50 μl (about 50 ul) of diluted cells and about 75 μl (about 75 ul) of LPS (ca. 1 ng/ml final; Sigma Cat No: L-6386) are added to the compound plate, which is then incubated at 37° C., 5% CO₂, for 20 hours.

The supernatant is removed and the concentrations of TNF-α are determined by electrochemiluminescence assay using the Meso Scale Discovery (MSD) technology (Meso Scale Discovery, 9238 Gaither Road, Gaithersburg, Md. 20877, USA). See the “TNF-α (TNF-alpha) MSD Assay” described below for typical details.

Results can be expressed as pIC50 values for inhibition of TNF-α (TNF-alpha) production in PBMCs, and it should be appreciated that these results can be subject to a large variability or error.

### Inhibition of TNF-α (TNF-alpha) Production in Human PBMC (Peripheral Blood Mononuclear Cell) Assay (IGEN Technology)

This is an optional supplementary test, e.g. for potentially inhalably-administrable PDE4 inhibitors.

Test compounds are prepared as a ca. 10 mM stock solution in DMSO and a dilution series prepared in DMSO with 8 successive 3fold dilutions, either directly from the 10 mM stock solution or from a more dilute solution in DMSO. The compound is added to assay plates using a Biomek Fx liquid handling robot.

PBMC cells (peripheral blood mononuclear cells) are prepared from heparinised human blood from normal volunteers by centrifugation on histopaque at ca. 1000 g for ca. 30 minutes. The cells are collected from the interface, washed by centrifugation (ca. 1300 g, ca. 10 minutes) and resuspended in assay buffer (RPMI 1640 containing 10% foetal calf serum, 1% L-glutamine and 1% penicillin/streptomycin) at 1×10⁶ cells/ml. Ca. 50 μl (ca. 50 ul) of cells are added to microtitre wells containing ca. 0.5 or ca. 1.0 μl (ul) of an appropriately diluted compound solution. Ca. 75 μl (ul) of LPS (lipopolysaccharide) (ca. 1 ng/ml final) is added and the samples are incubated at 37° C., 5% CO₂, for 20 hours.

The supernatant is removed and the concentrations of TNF-α are determined by electrochemiluminescence assay using the IGEN technology or by ELISA (see below).

Results can be expressed as pIC50 values for inhibition of TNF-α (TNF-alpha) production in PBMCs, and it should be appreciated that these results can be subject to a large variability or error.

### PBMC Assay Results:

For the following Examples, which are compounds of formula (I) or salts thereof, and generally (without any warranty) when using the one of the above assays, or a generally similar or generally analogous assay, the measured and/or mean pIC50 values for inhibition of TNF-α (TNF-alpha) production in PBMCs are generally as follows (subject to a possibly large variability or error):

| | PBMC pIC₅₀ | |
|---|---|---|
| Example | (MSD assay) | PBMC pIC₅₀ |
| numbers | (n = no. of measurements) | (IGEN assay) |
| 10 | about 10.7 (n = ca. 4) | |
| 23 | about 9.9 (n = ca. 233) | about 10.3 (n = about 4) |
| 28 | about 9.9 (n = ca. 4) | |
| 29 | about 9.1 (n = ca. 15) | |
| 35 | about 10.0 (n = ca. 8) | |

### Inhibition of TNF-α (TNF-Alpha) Production in Human Whole Blood

This is an optional supplementary test, e.g. for potentially orally-administrable PDE4 inhibitors. Also, as the assay may measure the effect of PDE4 inhibitors after loss by protein binding, it might possibly also be relevant to externally-topically-administrable PDE4 inhibitors as protein-binding-loss of compound is possible during transport through the skin.

Test compounds are prepared as a ca. 10 mM stock solution in DMSO and a dilution series prepared in DMSO with 8 successive 3fold dilutions, either directly from the 10 mM stock solution or from a more dilute solution in DMSO. The compound is added to assay plates using a Biomek Fx liquid handling robot.

Heparinised blood drawn from normal volunteers is dispensed (ca. 100 μl=ca. 100 ul) into microtitre plate wells containing ca. 0.5 or ca. 1.0 μl (ul, microlitres) of an appropriately diluted test compound solution. After ca. 1 hr incubation at ca. 37° C., 5% CO₂, ca. 25 μl (ca. 25 ul) of LPS (lipopolysaccharide) solution (S. typhosa) in RPMI 1640 (containing 1% L-glutamine and 1% Penicillin/streptomycin) is added (ca. 50 ng/ml final). The samples are incubated at ca. 37° C., 5% CO₂, for ca. 20 hours, and ca. 100 μl (ca. 100 ul) physiological saline (0.138% NaCl) is added, and diluted plasma is collected using a Platemate or Biomek FX liquid handling robot after centrifugation at ca. 1300 g for ca. 10 min. Plasma TNF-α content is determined by electrochemiluminescence assay using the MSD technology (see below), the IGEN technology (see below) or by enzyme linked immunosorbant assay (ELISA) (see below).

Results can be expressed as pIC50 values for inhibition of TNF-α (TNF-alpha) production in Human Whole Blood, and it should be appreciated that these results can be subject to a large variability or error.

### TNF-α (TNF-Alpha) MSD Assay

Using the Biomek FX, 25 μl (25 ul) of MSD Human Serum Cytokine Assay Diluent (Meso Scale Discovery, 9238 Gaither Road, Gaithersburg, Md. 20877) is added to a 96-well High-Bind MSD plate pre-coated with anti-hTNF alpha capture antibody (MA6000) and then incubated for 24 hours at 4° C. to prevent non-specific binding. About 20 μl (ul) of supernatant from the PBMC plate or about 40 μl (μl) of supernatant from the whole blood (WB) plate are then transferred from columns 1-11 to columns 1-11 of the MSD plate using the Biomek FX. About 20 μl (ul) of TNF-α standard (Cat No. 210-TA; R&D Systems Inc., 614 McKinley Place Nebr., Minneapolis, Minn. 55413, USA) are added to column 12 of the MSD plate to generate a standard calibration curve (about 0 to 30000 pg/ml final).

For the Whole Blood assay, plates are washed after 2 hours shaking with a Skanwasher 300 version B (Skatron Instruments AS. PO Box 8, N-3401 Lier, Norway). About 40 μl (ul) of diluted sulfo-TAG antibody (ca. 1 μg/ml final) is added, the plates are shaken at room temperature for 1 hours, and the plates washed again as above. About 150 μl (ul) of Read Buffer T (2×) is added to the plates, which are then read on a MSD Sector 6000.

For the PBMC assay, about 20 μl (ul) of diluted sulfo-TAG antibody (ca. 1 μg/ml final) is added to each well, and the plates/wells are shaken at room temperature for 2 hours. Finally, about 90 μl (ul) of MSD Read Buffer P (diluted to 2.5 times with distilled water) is added and the plates are read on a MSD Sector 6000.

### Data Analysis

Data analysis is performed with ActivityBase/XC50 module (ID Business Solutions Ltd., 2 Occam Court, Surrey Research Park, Guildford, Surrey, GU2 7QB UK). Data are normalized and expressed as % inhibition using the formula 100*((U-C1)/(C2-C1)) where U is the unknown value, C1 is the average of the high signal (0%) control wells (column 10), and C2 is the average of the low signal (100%) control wells (column 11). Curve fitting is performed with the following equation: y=A+((B−A)/(1+(10̂×/10̂C) ̂D)), where A is the minimum response, B is the maximum response, C is the log 10(IC50), and D is the Hill slope. The results for each compound are recorded as pIC50 values (—C in the above equation).

### TNF-α (TNF-Alpha) IGEN Assay

Ca. 50 μl supernatant from either whole blood or PBMC assay plates is transferred to a 96 well polypropylene plate. Each plate also contains a TNF-α standard curve (ca. 0 to 30000 pg/ml: R+D Systems, 210-TA). Ca. 50 μl (ul) of streptavidin/biotinylated anti-TNF-α antibody mix, ca. 25 μl ruthenium tagged anti-TNF-α monoclonal and ca. 100 μl PBS containing 0.1% bovine serum albumin are added to each well and the plates are sealed and shaken for ca. 2 hours before being read on an IGEN instrument.

### TNF-α (TNF-Alpha) ELISA Assay (Enzyme Linked Immunosorbant Assay)

Human TNF-α can be assayed using a commercial ELISA assay kit (AMS Biotechnology, 211-90-164-40) according to the manufacturers' instructions but with TNF-α calibration curves prepared using Pharmingen TNF-α (cat. No. 555212).

### In Vivo Biological Assays

The in vitro enzymatic PDE4B inhibition assay(s) described above or generally similar or analogous assays should be regarded as being the primary test(s) of biological activity. However, some additional in vivo biological tests, which are optional only, and which are not an essential measure of activity, efficacy or side-effects, and which have not necessarily been carried out, are described below.

### In Vivo Assay 1. LPS Induced Pulmonary Neutrophilia in Rats: Effect of Intratracheally Administered PDE4 Inhibitors

This assay is an animal model of inflammation in the lung—specifically neutrophilia induced by lipopolysaccharide (LPS)— and allows the study of putative inhibition of such neutrophilia (anti-inflammatory effect) by intratracheally (i.t.) administered PDE4 inhibitors. The PDE4 inhibitors are preferably in dry powder or wet suspension form. I.t. administration is one model of inhaled administration, allowing topical delivery to the lung.

Animals: Male CD (Sprague Dawley Derived) rats supplied by Charles River, Raleigh, N.C., USA or Charles River, United Kingdom are housed in groups of 5 rats per cage, acclimatized after delivery for at least 5 days with bedding/nesting material regularly changed, fed on SDS diet R1 pelleted food given ad lib, and supplied with daily-changed pasteurized animal grade drinking water.

Device for dry powder administration: Disposable 3-way tap between dosing needle and syringe. The intratracheal dosing device (a 3-way sterile tap, Vycon 876.00; or Penn Century dry powder insufflator, DP-4) is weighed, the drug blend or inhalation grade lactose (vehicle control) is then added to the tap, the tap is closed to prevent loss of drug, and the tap is re-weighed to determine the weight of drug in the tap. After dosing, the tap is weighed again to determine the weight of drug that had left the tap. The needle, a Sigma Z21934-7 syringe needle 19-gauge 152 mm (6 inches) long with luer hub, is cut by engineering to approximately 132 mm (5.2 inches), a blunt end is made to prevent them damaging the rat's trachea, and the needle is weighed prior to and after drug delivery to confirm that no drug is retained in the needles after dosing.

Device for wet suspension administration: This is similar to the above but a blunt dosing needle, whose forward end is slightly angled to the needle axis, is used, with a flexible plastic portex canula inserted into the needle.

Drugs and Materials: Lipopolysaccharide (LPS) (Serotype:0127:B8) is dissolved in phosphate-buffered saline (PBS). PDE4 inhibitors are preferably used in size-reduced (e.g. micronised) form, for example according to the Micronization Example(s) given herein.

For dry powder administration of the drug, the Dry Powder Formulation Example given herein, comprising drug and inhalation-grade lactose, can optionally be used. One suitable inhalation-grade lactose that can be used has 10% fines (10% of material under 15 um (15 micron) particle size measured by Malvern particle size).

Wet suspensions of the drug (aqueous) can be prepared by adding the required volume of vehicle to the drug; the vehicle used can for example be saline alone or a mixture of saline/tween (e.g. 0.2% tween 80). The wet suspension is usually sonicated for ca. 10 minutes prior to use.

Preparation, and dosing with PDE 4 inhibitor: Rats are anaesthetized by placing the animals in a sealed Perspex chamber and exposing them to a gaseous mixture of isoflurane (4.5%), nitrous oxide (3 litres.minute⁻¹) and oxygen (1 litre.minute⁻¹). Once anaesthetized, the animals are placed onto a stainless steel i.t. dosing support table. They are positioned on their back at approximately a 35° angle. A light is angled against the outside of the throat to highlight the trachea. The mouth is opened and the opening of the upper airway visualised. The procedure varies for wet suspension and dry powder administration of PDE4 inhibitors as follows:

Dosing with a Wet suspension: A portex cannula is introduced via a blunt metal dosing needle that has been carefully inserted into the rat trachea. The animals are intratracheally dosed with vehicle or PDE4 inhibitor via the dosing needle with a new internal canula used for each different drug group. The formulation is slowly (ca. 10 seconds) dosed into the trachea using a syringe attached to the dosing needle.

Dosing with a Dry Powder: The intratracheal dosing device (a three-way sterile tap device, Vycon 876.00; or Penn Century dry powder insufflator, DP-4) and needle are inserted into the rat trachea up to a pre-determined point established to be located approximately 1 cm above the primary bifurcation. Another operator holds the needle at the specified position whilst 2×4 ml of air (using 3-way tap device) is delivered through the three-way tap by depressing the syringes (ideally coinciding with the animal inspiring), aiming to expel the entire drug quantity from the tap. (Alternatively, 2×3 ml of air is delivered using Penn Century dry powder insufflator device.) After dosing, the needle and tap or device are removed from the airway, and the tap closed off to prevent any retained drug leaving the tap.

After dosing with either wet suspension or dry powder, the animals are then removed from the table and observed constantly until they have recovered from the effects of anaesthesia. The animals are returned to the holding cages and given free access to food and water; they are observed and any unusual behavioural changes noted.

Exposure to LPS: About 2 hours after i.t. dosing with vehicle control or the PDE4 inhibitor, the rats are placed into sealed Perspex containers and exposed to an aerosol of LPS (nebuliser concentration ca. 150 μg.ml⁻¹=ca. 150 ug/ml) for ca. 15 minutes. Aerosols of LPS are generated by a nebuliser (DeVilbiss, USA) and this is directed into the Perspex exposure chamber. Following the 15-minute LPS-exposure period, the animals are returned to the holding cages and allowed free access to both food and water.

In an alternative embodiment, the rats can be exposed to LPS less than 2 hours (e.g. about 30 minutes) after i.t. dosing.

In another alternative embodiment, the rats can be exposed to LPS more than 2 hours (e.g. ca. 4 hours to ca. 36 hours, such as 4 hours, 6 hours, 12 hours, 18 hours, 24 hours or 36 hours, in particular 12 hours) after i.t. dosing by vehicle or PDE4 inhibitor, to test whether or not the PDE4 inhibitor has a long duration of action (which is not essential).

Bronchoalveolar lavage: About 4 hours after LPS exposure the animals are killed by overdose of sodium pentobarbitone (i.p.). The trachea is cannulated with polypropylene tubing and the lungs are lavaged (washed out) with 3×5 mis of heparinised (25 units.ml⁻¹) phosphate buffered saline (PBS).

Neutrophil cell counts: The Bronchoalveolar lavage (BAL) samples are centrifuged at ca. 1300 rpm for ca. 7 minutes. The supernatant is removed and the resulting cell pellet resuspended in ca. 1 ml PBS. A cell slide of the resuspension fluid is prepared by placing ca. 100 μl (ca. 100 ul) of resuspended BAL fluid into cytospin holders and then is spun at ca. 5000 rpm for ca. 5 minutes. The slides are allowed to air dry and then stained with Leishmans stain (ca. 20 minutes) to allow differential cell counting. The total cells are also counted from the resuspension. From these two counts, the total numbers of neutrophils in the BAL are determined. For a measure of PDE4-inhibitor-induced inhibition of neutrophilia, a comparison of the neutrophil count in rats treated with vehicle and rats treated with PDE4 inhibitors is conducted.

By varying the dose of the PDE4 inhibitor used in the dosing step (e.g. 0.2 or 0.1 mg of PDE4 inhibitor per kg of body weight, down to e.g. 0.01 mg/kg), a dose-response curve can be generated, and thence an ED₅₀ value.

Results: In an assay(s), generally involving LPS-induced pulmonary neutrophilia in rats and the effect of intratracheally administered PDE4 inhibitors to those rats, which assay(s) is or are generally similar to or generally analogous to the above-mentioned assay, the following results were measured:

Example 23 showed an ED₅₀ of about 50 micrograms/kg body weight, for inhibition of neutrophilia, when administered intratracheally (i.t.) 2 hours prior to LPS challenge as a wet suspension in saline/tween.

Example 29 showed about 40% inhibition of the neutrophilia when administered intratracheally (i.t.) 2 hours prior to LPS challenge as a wet suspension in saline/tween.

Example 35 showed about 43% inhibition when administered intratracheally (i.t.) 2 hours prior to LPS challenge as a wet suspension in saline/tween.

It is noted that these results might be subject to a possibly significant margin of error; and generally results may vary as the assay or measurement method varies.

### In Vivo Assay A:

### Activity of Topically-Applied Compounds in a Pig Model of Atopic Dermatitis: Effect of Compounds, Applied by Skin Topical Administration, on the Dinitrofluorobenzene (DNFB)-Induced Delayed Type Hypersensitivity (DTH) Response in Pigs

### General Study Design:

The pig DTH (delayed type hypersensitivity) model of contact hypersensitivity utilizes the Th2-mediated inflammatory response in pig skin to mimic the pathology of atopic dermatitis in humans. The model measures the potential anti-inflammatory effect of compounds, topically-applied to the skin, on the acute DTH (delayed type hypersensitivity) response in castrated male Yorkshire pigs.

In general in the assay, pigs (domestic Yorkshire pigs, 15-18 kg at time of sensitization, castrated males) are first sensitized by topical application of ca. 10% (w/v) dinitrofluorobenzene (DNFB) dissolved in DMSO:acetone:olive oil (ca. 1:5:3) (ca. 40 mg DNFB, 400 microliter solution total) to the ears (outer) and groin (inner). The pigs are then challenged 12 days later with ca. 0.6% (w/v) DNFB applied to randomized sites on the shaved back of the pigs (ca. 90 micrograms/site; sites are identified and numbered by grid made with marking pen).

On the day of challenge, the treatments are performed at the challenge sites at about 2 hours prior to and about 6 hours after challenge (for DMSO/acetone solutions/suspensions containing the PDE4 inhibitor, to maximize exposure to drug), or at about 30 minutes after and about 6 hours after challenge (for topical ointments or creams containing the PDE4 inhibitor, representing a more clinically relevant treatment protocol).

One day (about 24 hrs) after challenge, and optionally again at ca. 48 hrs post challenge, test sites are visually evaluated for intensity and extent of erythema by measuring the diameter of the reaction at its widest point and assigning scores of 0 to 4 for each of erythema intensity and erythema extent. Induration (a measure of swelling) is also scored 0 to 4. Scores for erythema intensity, erythema extent and induration are assigned according to the following criteria: Intensity of Erythema: 0=normal, 1=minimal, barely visible, 2=mild, 3=moderate, 4=severe. Extent of Erythema (not raised): 0=no edema, 1=macules of pin head size, 2=lentil sized macules, 3=confluent macules, 4=diffuse over entire site. Induration (palpable): 0=normal, 1=nodules of pin head size, 2=doughy lentil sized nodules, 3=confluent firm nodules, 4=diffuse hard lesion. The summed visual score at ca. 24 hours includes the individual scores for erythema intensity, erythema extent, and induration; so the maximal summed score for each site would be 12. High summed scores can generally indicate a high inflammatory response. Visual scores are subject to some inaccuracy/error.

Differences in the summed score between adjacent control (placebo) and treatment sites on the grids are calculated. This difference value is then used to determine the percent inhibition compared to the summed score for the control (placebo) sites. The more negative the difference value, the greater the calculated inhibition. Percent inhibition of (percent inhibition compared to) the mean summed score can be calculated.

About 24 hours after challenge, treatment sites can optionally also be visually evaluated for lesion area.

All publications, including but not limited to patents and patent applications, cited in this specification are herein incorporated by reference as if each individual publication were specifically and individually indicated to be incorporated by reference herein as though fully set forth.

### EXAMPLES

The various aspects of the invention will now be described by reference to the following examples. These examples are merely illustrative and are not to be construed as a limitation of the scope of the present invention.

In this section, “Intermediates” can represent syntheses of intermediate compounds intended for use in the synthesis of one or more of the “Examples”, and/or “Intermediates” can represent syntheses of intermediate compounds which can possibly be used in the synthesis of compounds of formula (I) or salts thereof. “Examples” are generally examples of compounds or salts of the invention, for example compounds of formula (I) or salts thereof.

Abbreviations used herein:
AcOH acetic acid
Ac₂O acetic anhydride
BEMP 2-t-butylimino-2-diethylamino-1,3-dimethylperhydro-1,3,2-diazaphosphazine
BOC₂O di tert-butyl carbonate
DMSO dimethyl sulfoxide
DCC N,N′-dicyclohexylcarbodiimide
DCM dichloromethane
DMF dimethyl formamide
DIPEA diisopropylethyl amine (ⁱPr₂NEt)
EDC 1-(3-Dimethylaminopropyl)-3-ethylcarbodiimide hydrochloride
EtOAc ethyl acetate
Et₂O diethyl ether
Et₃N triethylamine
EtOH ethanol
HATU O-(7-Azabenzotriazol-1-yl)-N,N,N′,N′-tetramethyluronium hexafluorophosphate
HBTU O-(Benzotriazol-1-yl)-N,N,N′,N′-tetramethyluronium hexafluorophosphate
HOBT hydroxybenzotriazole=1-hydroxybenzotriazole
IPA isopropanol (isopropyl alcohol)
Lawesson's reagent 2,4-bis(4-methoxyphenyl)-1,3-dithia-2,4-diphosphetane-2,4-disulphide
LPS lipopolysaccharide
MeCN acetonitrile
MeOH methanol
MDAP mass directed autoprep HPLC
NMP 1-methyl-2-pyrrolidinone (also named 1-methyl-2-pyrrolidone)
PyBOP Benzotriazole-1-yl-oxy-trispyrrolidinophosphonium hexafluorophosphate
TMS tetramethylsilane
THF Tetrahydrofuran
TFA Trifluoroacetic acid
HPLC high performance liquid chromatography
h hours
min minutes
LCMS liquid chromatography/mass spectroscopy
NMR nuclear magnetic resonance (in which: s=singlet, d=doublet, t=triplet, q=quartet, dd=doublet of doublets, m=multiplet, H=no. of protons)
SPE solid phase extraction. Unless otherwise specified, the solid phase is usually silica gel. Aminopropyl SPE typically refers to a silica SPE column with aminopropyl residues immobilised on the solid phase (e.g. IST Isolute™ columns). It is thought that compounds isolated by SPE are usually isolated as the free base.
TLC thin layer chromatography
T_{RET} or R_{T} retention time (e.g. from LCMS)
Room temperature (ambient temperature): this is usually in the range of about 18 to about 25° C.

### General Experimental Details

### Machine Methods Used Herein:

### LCMS (Liquid Chromatography/Mass Spectroscopy)

Waters ZQ mass spectrometer operating in positive ion electrospray mode, mass range 100-1000 amu.

UV wavelength: 215-330 nM
Column: 3.3 cm×4.6 mm ID, 31m ABZ+PLUS
Flow Rate: 3 ml/min

### Injection Volume: 5 μl

Solvent A: 95% acetonitrile+0.05% formic acid
Solvent B: 0.1% formic acid+10 mMolar ammonium acetate
Gradient: 0% A/0.7 min, 0-100% A/3.5 min, 100% A/1.1 min, 100-0% A/0.2 min

It should be noted that retention times (T_{RET}) quoted herein are inherently variable (e.g. the variability can be about +/−0.2 min.). Variability can arise e.g. when samples are run on different Waters machines, or on the same Waters machine at different times of day or under slightly different conditions, even when the same type of column and identical flow rates, injection volumes, solvents and gradients are used.

### Mass Directed Autoprep HPLC

The preperative HPLC column generally used is a Supelcosil ABZplus (10 cm×2.12 cm) (usually 10 cm×2.12 cm×5 μm). A mass spectrometer attached to the end of the column can detect peaks arising from eluted compounds.

UV wavelength: 200-320 nM
Flow: 20 ml/min
Injection Volume: 1 ml; or more preferably 0.5 ml
Gradient systems: mixtures of Solvent A and Solvent B are used according to a choice of 5 generic gradient profiles (expressed as % Solvent B in the mixture), ranging from a start of 0 to 50% Solvent B, with all finishing at 100% Solvent B to ensure total elution. Generally, two alternative solvent systems have been used, Method 1 and Method 2:

### Method 1

Solvent A: 0.1% formic acid
Solvent B: 95% acetonitrile+5% formic acid; or more usually 99.95% acetonitrile+0.05% formic acid

It is thought that compounds isolated by this method can sometimes be isolated as formate salts.

### Method 2

Solvent A: water+0.1% trifluoroacetic acid
Solvent B: acetonitrile+0.1% trifluoroacetic acid

It is thought that compounds isolated by this method can sometimes be isolated as trifluoroacetate salts.

### Intermediates and Examples

Reagents not detailed in the text below are usually commercially available from chemicals suppliers, e.g. established suppliers such as Sigma-Aldrich. The addresses and/or contact details of the suppliers for some of the starting materials mentioned in the Intermediates and Examples below or the Assays above, or suppliers of chemicals in general, are as follows:

AB Chem, Inc., 547 Davignon, Dollard-des-Ormeaux, Quebec, H₉B1Y4, Canada

ABCR GmbH & CO. KG, P.O. Box 21 01 35, 76151 Karlsruhe, Germany

ACB Blocks Ltd; Kolokolnikov Per, 9/10 Building 2, Moscow, 103045, Russia

Aceto Color Intermediates (catalogue name), Aceto Corporation, One Hollow Lane, Lake Success, N.Y., 11042-1215, USA

Acros Organics, A Division of Fisher Scientific Company, 500 American Road, Morris Plains, N.J. 07950, USA

Aldrich (catalogue name), Sigma-Aldrich Company Ltd., Dorset, United Kingdom, telephone: +44 1202 733114; Fax: +44 1202 715460; ukcustsv@eurnotes.sial.com; or

Aldrich (catalogue name), Sigma-Aldrich Corp., P.O. Box 14508, St. Louis, Mo. 63178-9916, USA; telephone: +1-314-771-5765; fax: +1-314-771-5757; custserv@sial.com; or

Aldrich (catalogue name), Sigma-Aldrich Chemie GmbH, Munich, Germany; telephone: +49 89 6513 0; Fax: +49 89 6513 1169; deorders@eurnotes.sial.com.

Alfa Aesar, A Johnson Matthey Company, 30 Bond Street, Ward Hill, Mass. 01835-8099, USA

Amersham Biosciences UK Ltd, Pollards Wood, Chalfont St Giles, Buckinghamshire HP8 4SP, United Kingdom

Apin Chemicals Ltd., 82 C Milton Park, Abingdon, Oxon OX14 4RY, United Kingdom

Apollo Scientific Ltd., Unit 1A, Bingswood Industrial Estate, Whaley Bridge, Derbyshire SK23 7LY, United Kingdom

Arch Corporation, 100 Jersey Avenue, Building D, New Brunswick, N.J. 08901, USA

Array Biopharma Inc., 1885 33rd Street, Boulder, Colo. 80301, USA

Asinex-Reag.

AstaTech, Inc., 8301 Torresdale Ave., 19C, Philadelphia, Pa. 19136, USA

Austin Chemical Company, Inc., 1565 Barclay Blvd., Buffalo Grove, Ill. 60089, USA

Avocado Research, Shore Road, Port of Heysham Industrial Park, Heysham, Lancashire LA3 2XY, United Kingdom
Bayer A G, Business Group Basic and Fine Chemicals, D-51368 Leverkusen, Germany

Berk Univar pic, Berk House, P.O. Box 56, Basing View, Basingstoke, Hants RG21 2E6, United Kingdom

Bionet Research Ltd; Highfield Industrial Estate, Camelford, Cornwall PL32 9QZ UK

Butt Park Ltd., Braysdown Works, Peasedown St. John, Bath BA2 8LL, United Kingdom

Chemical Building Blocks (catalogue name), Ambinter, 46 quai Louis Bleriot, Paris, F-75016, France

ChemBridge Europe, 4 Clark's Hill Rise, Hampton Wood, Evesham, Worcestershire WR11 6FW, United Kingdom

ChemService Inc., P.O. Box 3108, West Chester, Pa. 19381, USA

CiventiChem, PO Box 12041, Research Triangle Park, N.C. 27709, USA

Combi-Blocks Inc., 7949 Silverton Avenue, Suite 915, San Diego, Calif. 92126, USA

Dynamit Nobel GmbH, Germany; also available from: Saville Whittle Ltd (UK agents of Dynamit Nobel), Vickers Street, Manchester M40 8EF, United Kingdom

E. Merck, Germany; or E. Merck (Merck Ltd), Hunter Boulevard, Magna Park, Lutterworth, Leicestershire LE17 4XN, United Kingdom

Esprit Chemical Company, Esprit Plaza, 7680 Matoaka Road, Sarasota, Fla. 34243, USA

Exploratory Library (catalogue name), Ambinter, 46 quai Louis Bleriot, Paris, F-75016, France

Fluka Chemie A G, Industriestrasse 25, P.O. Box 260, CH-9471 Buchs, Switzerland

Fluorochem Ltd., Wesley Street, Old Glossop, Derbyshire SK13 7RY, United Kingdom

Heterocyclic Compounds Catalog (Florida Center for Heterocyclic Compounds, University of Florida, PO Box 117200, Gainsville, Fla. 32611-7200 USA

ICN Biomedicals, Inc., 3300 Hyland Avenue, Costa Mesa, Calif. 92626, USA

Interchim Intermediates (catalogue name), Interchim, 213 Avenue Kennedy, BP 1140, Montlucon, Cedex, 03103, France

Key Organics Ltd., 3, Highfield Industrial Estate, Camelford, Cornwall PL32 9QZ, United Kingdom

Lancaster Synthesis Ltd., Newgate, White Lund, Morecambe, Lancashire LA3 3DY, United Kingdom

Manchester Organics Ltd., Unit 2, Ashville Industrial Estate, Sutton Weaver, Runcorn, Cheshire WA7 3 PF, United Kingdom

Matrix Scientific, P.O. Box 25067, Columbia, S.C. 29224-5067, USA

Maybridge Chemical Company Ltd., Trevillett, Tintagel, Cornwall PL34 0 HW, United Kingdom

Maybridge Combichem (catalogue name), Maybridge Chemical Company Ltd., Trevillett, Tintagel, Cornwall PL34 0 HW, United Kingdom

Maybridge Reactive Intermediates (catalogue name), Maybridge Chemical Company Ltd., Trevillett, Tintagel, Cornwall PL34 0 HW, United Kingdom

MicroChemistry Building Blocks (catalogue name), MicroChemistry-RadaPharma, Shosse Entusiastov 56, Moscow, 111123, Russia

Miteni S.p.A., Via Mecenate 90, Milano, 20138, Italy

Molecular Devices Corporation, Sunnydale, Calif., USA

N.D. Zelinsky Institute, Organic Chemistry, Leninsky prospect 47, 117913 Moscow B-334, Russia

Oakwood Products Inc., 1741, Old Dunbar Road, West Columbia, S.C., 29172, USA

OmegaChem. Inc., 8800, Boulevard de la Rive Sud, Levis, PQ, G6V 9H1, Canada

Optimer Building Block (catalogue name), Array BioPharma, 3200 Walnut Street, Boulder,

### Colo. 80301, USA

Peakdale Molecular Ltd., Peakdale Science Park, Sheffield Road, Chapel-en-le-Frith, High Peak

SK23 0 PG, United Kingdom

Pfaltz & Bauer, Inc., 172 East Aurora Street, Waterbury, Conn. 06708, USA

Qualigens Fine Chemicals

Rare Chemicals (catalogue name), Rare Chemicals GmbH, Schulstrasse 6, 24214 Gettorf, Germany

Rieke

SALOR (catalogue name) (Sigma Aldrich Library of Rare Chemicals), Aldrich Chemical Company Inc, 1001 West Saint Paul Avenue, Milwaukee, Wis. 53233, USA

Sigma (catalogue name), Sigma-Aldrich Corp., P.O. Box 14508, St. Louis, Mo. 63178-9916, USA; see “Aldrich” above for other non-US addresses and other contact details

SIGMA-RBI, One Strathmore Road, Natick, Mass. 01760-1312, USA

Spectrochem

Synchem OHG Heinrich-Plett-Strasse 40, Kassel, D-34132, Germany

Syngene International Pvt Ltd, Hebbagodi, Hosur Road, Bangalore, India.

TCI America, 9211 North Harborgate Street, Portland, Oreg. 97203, USA

TimTec Building Blocks A or B, TimTec, Inc., P 0 Box 8941, Newark, Del. 19714-8941, USA

TimTec Overseas Stock, TimTec Inc., 100 Interchange Blvd. Newark, Del. 19711, USA

TimTec Stock Library, TimTec, Inc., P 0 Box 8941, Newark, Del. 19714-8941, USA

Trans World Chemicals, Inc., 14674 Southlawn Lane, Rockville, Md. 20850, USA

Tyger

Ubichem PLC, Mayflower Close, Chandlers Ford Industrial Estate, Eastleigh, Hampshire SO53 4AR, United Kingdom

Ultrafine (UFC Ltd.), Synergy House, Guildhall Close, Manchester Science Park, Manchester M15 6SY, United Kingdom

INTERMEDIATES

| Intermediate No. | Name |
|---|---|
| 1 | 1-ethyl-1H-pyrazol-5-amine |
| 2 | diethyl (1-chloropropylidene)propanedioate |
| 3 | diethyl (1-chloroethylidene)propanedioate |
| 4 | Ethyl 4-chloro-1,6-diethyl-1H-pyrazolo[3,4-b]pyridine-5-carboxylate |
| 5 | ethyl 1,6-diethyl-4-(tetrahydro-2H-pyran-4-ylamino)-1H-pyrazolo[3,4- |
| | b]pyridine-5-carboxylate |
| 6 | [1,6-diethyl-4-(tetrahydro-2H-pyran-4-ylamino)-1H-pyrazolo[3,4- |
| | b]pyridin-5-yl]methanol |
| 7 | 5-(azidomethyl)-1,6-diethyl-N-(tetrahydro-2H-pyran-4-yl)-1H- |
| | pyrazolo[3,4-b]pyridin-4-amine |
| 8 | 5-(aminomethyl)-1,6-diethyl-N-(tetrahydro-2H-pyran-4-yl)-1H- |
| | pyrazolo[3,4-b]pyridin-4-amine |
| 9 | ethyl 4-[(1-{[(1,1-dimethylethyl)oxy]carbonyl}-4-piperidinyl)amino]- |
| | 1,6-diethyl-1H-pyrazolo[3,4-b]pyridine-5-carboxylate |
| 10 | 1,1-dimethylethyl 4-{[1,6-diethyl-5-(hydroxymethyl)-1H-pyrazolo[3,4- |
| | b]pyridin-4-yl]amino}-1-piperidinecarboxylate |
| 11 | 1,1-dimethylethyl 4-{[5-(azidomethyl)-1,6-diethyl-1H-pyrazolo[3,4- |
| | b]pyridin-4-yl]amino}-1-piperidinecarboxylate |
| 12 | 4-{[5-(azidomethyl)-1,6-diethyl-1H-pyrazolo[3,4-b]pyridin-4-yl]amino}- |
| | 1-piperidinecarboxamide |
| 13 | 4-{[5-(aminomethyl)-1,6-diethyl-1H-pyrazolo[3,4-b]pyridin-4- |
| | yl]amino}-1-piperidinecarboxamide |
| 14 | ethyl 4-chloro-1-ethyl-6-methyl-1H-pyrazolo[3,4-b]pyridine-5- |
| | carboxylate |
| 15 | ethyl 1-ethyl-6-methyl-4-(tetrahydro-2H-pyran-4-ylamino)-1H- |
| | pyrazolo[3,4-b]pyridine-5-carboxylate |
| 16 | 1-ethyl-6-methyl-4-(tetrahydro-2H-pyran-4-ylamino)-1H-pyrazolo[3,4- |
| | b]pyridin-5-yl]methanol |
| 17 | 5-(azidomethyl)-1-ethyl-6-methyl-N-(tetrahydro-2H-pyran-4-yl)-1H- |
| | pyrazolo[3,4-b]pyridin-4-amine |
| 18 | 5-(aminomethyl)-1-ethyl-6-methyl-N-(tetrahydro-2H-pyran-4-yl)-1H- |
| | pyrazolo[3,4-b]pyridin-4-amine |

### Intermediate 1

### 1-ethyl-1H-pyrazol-5-amine

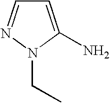

Acrylonitrile (e.g. available from E. Merck, 536 g, 10.11 moles) was added dropwise to hydrazine hydrate (e.g. available from Qualigens Fine Chem., 511 g, 10.22 moles) maintaining the reaction temperature between 30-35° C. (little exothermicity was observed). After completion of the addition the reaction mixture was stirred at the same temperature for an additional hour. Water from the mixture was removed at 45-50° C. under reduced pressure to give the Michael addition product (835 g) as pale yellow oil, which was used in the next step without further purification.

A solution of acetaldehyde (e.g. available from Sigma-Aldrich, 561 ml, 9.99 moles) in ethanol (1008 ml) was added dropwise to a stirred solution of the above 2-cyanoethylhydrazine (835 g, 9.82 moles) in ethanol (700 ml) at such a rate that the temperature did not rise above 35° C. The batch was heated under reflux for 1 hour and ethanol removed under reduced pressure to afford the Schiff's base (1097 g) as pale yellow oil, which was directly used in the subsequent step.

The above Schiffs base was added to a stirred mixture of sodium tert-butoxide (908 g, 9.46 moles) in tert-butanol and the reaction mixture was heated under reflux for 12 hours. The cooled reaction mixture was then poured into water (23 L), stirred for 0.5 hours and extracted with diethyl ether (1×10 L and 3×6 L). The combined organic layer was dried (Na₂SO₄) and the solution evaporated to dryness. The residue was dissolved in DCM (1.5 L) to remove impurities and the solvent evaporated to dryness to give the title compound (385 g) as a red oil which was used without further purification. 1H NMR (200 MHz, chloroform-d) δ (delta) ppm 1.38 (3H, t, J=7.3 Hz), 3.65 (2H, br s), 3.92 (2H, q, J=7.3 Hz), 5.49 (1H, s), 7.23 (1H, s).

### Intermediate 2

### diethyl (1-chloropropylidene)propanedioate

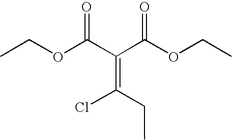

To a solution of diethyl malonate (e.g. available from Spectrochem, 704 g) in acetonitrile (3.8 L) was added anhydrous magnesium chloride (e.g. available from Lancaster, 419 g). Triethylamine (1222 ml) was added dropwise maintaining the temperature at 5-10° C., followed by the dropwise addition of propionyl chloride (406 g), maintaining the temperature below 30° C. Stirring was continued for 1 hour at 10-15° C. and then the mixture was allowed to reach room temperature. Hydrochloric acid (1M) was added to the reaction mixture until the pH of the mixture was about 2.0 (approx. 4.8 L was required). The mixture was extracted with diethyl ether (3×800 ml). The combined ethereal extracts were washed with aqueous hydrochloric acid (1M, 2×1000 ml) followed by water (2×1000 mL) and finally with brine (2×1000 mL). Evaporation of the solvent under reduced pressure afforded diethyl propanoylpropanedioate (845 g) as a pale yellow oil.

Tri-n-butylamine was added dropwise to the above keto-diester derivative (300 g) in phosphorus oxychloride (POCl₃, 3.1 L) at room temperature. The reaction mixture was heated under reflux for 7 hrs. After cooling, excess phosphorus oxychloride was removed under reduced pressure. The reaction mixture was extracted with a 1:2 mixture of hexane and diethyl ether (3×1.2 L). The combined organic extracts were washed with aqueous hydrochloric acid (1M, 1×1 L), aqueous sodium hydroxide solution (0.1M, 2×500 ml), and brine (2×500 ml), and dried. Evaporation of the solvent under reduced pressure afforded the title compound (245 g) as a red oil which was used without further purification.

### Intermediate 3

### diethyl (1-chloroethylidene)propanedioate

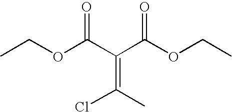

To a cooled (10° C.) suspension of diethyl malonate (200 g, 1.41 mol) in dry acetonitrile (300 mL) was added anhydrous magnesium chloride (e.g. available from Lancaster, 119.37 g, 1.26 mol), maintaining the temperature below 20° C. Triethylamine (348 ml) was added dropwise to the slurry followed by the dropwise addition of a solution of acetyl chloride (98.12 g, 1.25 mol) in acetonitrile (100 ml), maintaining the temperature at 10-15° C. Stirring was continued for 1 hour at 10-15° C., and the mixture was allowed to warm to room temperature overnight. Hydrochloric acid (1M) was added to the cooled reaction mixture (10° C.) until the pH of the mixture was about 1.0 (approx. 1.1 L was required). The mixture was extracted with diethyl ether (2×800 ml). The combined ethereal extracts were washed with hydrochloric acid (1M, 1×600 ml) and brine. Evaporation of the solvent under reduced pressure afforded diethyl acetylpropanedioate as the product (233.28 g) as an orange oil.

To the above keto-diester derivative (233 g) in phosphorus oxychloride (POCl₃, 2.2 L) was added tri-n-butylamine (250 ml) dropwise, and the solution was then heated at 120° C. for 7 hours. Excess phosphorus oxychloride was removed under reduced pressure; and the cooled reaction mixture was extracted with a 1:2 mixture of hexane and diethyl ether (3×1.2 L). The combined organic extracts were washed with hydrochloric acid (1M, 2×1 L), NaOH solution (0.1M, 2×1 L), with water (2×1 L), and brine (2×1 L), and dried (Na₂SO₄). Evaporation of the solvent under reduced pressure afforded the title compound (158 g) as a red oil which was used without further purification.

### Intermediate 4

### Ethyl 4-chloro-1,6-diethyl-1H-pyrazolo[3,4-b]pyridine-5-carboxylate

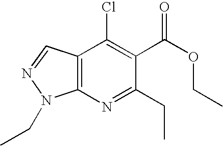

Triethylamine (230 ml) was added dropwise to a mixture of Intermediate 2 (208 g), Intermediate 1 (101 g) in toluene (2.65 L). The mixture was heated under reflux for 16 hours. The reaction mixture was cooled to room temperature, and the solid removed by filtration. The filtrate was evaporated under reduced pressure. The residue was heated under reflux in phosphorus oxychloride (POCl₃, 2.65 L) for 16 hrs. Excess phosphorus oxychloride was removed under reduced pressure and the cooled mixture was poured onto a mixture of saturated aqueous NaHCO₃ solution (4 L) and EtOAc (1.5 L). The organic layer was separated and the aqueous layer further extracted with ethyl acetate (2×1 L). The combined EtOAc extracts were washed with saturated aqueous NaHCO₃ solution (2×2 L) and dried (Na₂SO₄). Evaporation of solvent under reduced pressure afforded the crude product (202 g). The crude product was purified by chromatography (silica gel, 60-120 mesh, 3.5 kg), eluting with 3% EtOAc in hexane. Fractions containing the product were pooled and evaporated to give the title compound (133 g) as a pale yellow thick liquid, which solidified on standing. 1H NMR (200 MHz, chloroform-d) δ (delta) ppm 1.37 (3H, t, J=7.1 Hz), 1.45 (3H, t, J=7.1 Hz), 1.57 (3H, t, J=7.2 Hz), 2.91 (2H, q, J=7.1 Hz), 4.48 (2H, q, J=7.2 Hz), 4.57 (2H, q, J=7.1 Hz) 8.05 (1H, s).

### Intermediate 5

### ethyl 1,6-diethyl-4-(tetrahydro-2H-pyran-4-ylamino)-1H-Pyrazolo[3,4-b]pyridine-5-carboxylate

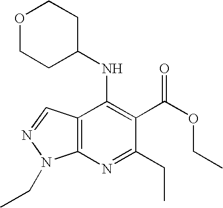

To a solution of Intermediate 4 (36 g, 127.8 mmol) in 1-methyl-2-pyrrolidinone (300 ml) was added DIPEA (44.5 ml, 255.6 mmol), resulting in a colour change from yellow to red/orange. Tetrahydro-2H-pyran-4-amine (e.g. available from Peakdale, 15.5 g, 153.3 mmol) was added and the reaction mixture heated at 115° C. with stirring overnight. The cooled mixture was poured into water (1200 ml), forming an oily orange mixture. This was extracted with EtOAc (4×250 ml), the organic extracts combined, washed with water (50 ml), 5% aqueous LiCl solution (50 ml), dried (MgSO₄), filtered and evaporated. The residue was purified by silica gel (1 kg) chromatography eluting with 2:1 cyclohexane:EtOAc (6000 ml) followed by 1:1 cyclohexane:EtOAc (3000 ml). The fractions containing product were pooled and evaporated to give the title compound as a mobile yellow oil (44.3 g) which solidified on standing. LCMS m/z 347 [MH⁺]; T_{RET}=3.01 min.

### Intermediate 6

### [1,6-diethyl-4-(tetrahydro-2H-pyran-4-ylamino)-1H-pyrazolo[3,4-b]pyridin-5-yl]methanol

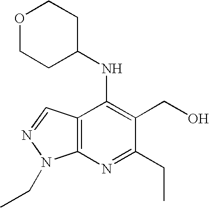

A solution of Intermediate 5 (17.9 g, 51.9 mmol) in DCM (150 ml) was cooled to 0° C. under an atmosphere of nitrogen and treated dropwise with di-iso-butylaluminum hydride

(100 ml of a 1.5M solution in toluene, 150 mmol) over 30 minutes. Stirring was continued for a further 30 minutes and the reaction quenched by the addition of saturated aqueous potassium sodium tartrate solution (120 ml) keeping the temperature below 5° C. The resulting mixture was diluted with EtOAc (300 ml) and filtered through Celite. During this filtration the sinter funnel imploded. Material spilt on the fumehood floor was soaked up and recovered as much as possible. The organic layer was separated and further extracted with EtOAc, dried (MgSO₄) and evaporated to give the title compound (14 g) as an off-white waxy solid. LCMS m/z 305 [MH⁺]; T_{RET}=1.86 min.

### Intermediate 6 Alternative Preparation

### [1,6-diethyl-4-(tetrahydro-2H-pyran-4-ylamino)-1H-pyrazolo[3,4-b]pyridin-5-yl]methanol

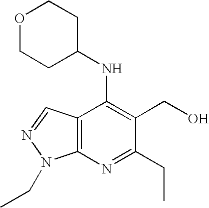

A solution of Intermediate 5 (60.4 g, 174 mmol) in dry THF (300 ml) was treated with dry methanol (28.3 ml, 698 mmol) followed by addition of lithium borohydride (LiBH₄, 2M solution in THF, 262 ml, 523 mmol) over 30 minutes. The mixture was heated as the lithium borohydride was added, reaching reflux about 20 minutes following the addition. Further aliquots of MeOH (14.1 ml) were added after 1 and 1.5 hours and the heating continued for a further 30 minutes. The reaction vessel was cooled (ice/water bath) and carefully treated with MeOH (100 ml) followed by cautious addition of water (200 ml) resulting a precipitate. Addition of more water (800 ml) produced a more homogenous suspension which was stirred for 1 h and was then extracted with DCM (about 1.5 L total volume). The organic extracts were combined, washed with water, brine, dried and evaporated to give the title compound as a white solid (49.84 g). LCMS m/z 305 [MH⁺]; T_{RET}=1.79 & 1.83 min (double peak).

### Intermediate 7

### 5-(azidomethyl)-1,6-diethyl-N-(tetrahydro-2H-pyran-4-yl)-1H-pyrazolo[3,4-b]pyridin-4-amine

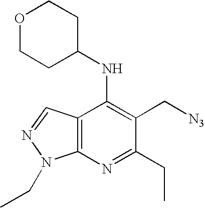

Thionyl chloride (SOCl₂, 51 ml, 69 mmol) was added to a suspension of Intermediate 6 (14 g, 46 mmol) in toluene (140 ml) at room temperature over 15 minutes. The mixture was heated to 85° C. for 4 hours, allowed to cool and then evaporated to dryness and azeotroped with three further portions of toluene. The residue was dissolved in dry DMSO (100 ml) and treated with sodium azide (4.5 g, 69 mmol) and the mixture stirred at room temperature for 18 hours. The mixture was poured into saturated aqueous sodium bicarbonate solution and was extracted with 3 portions of EtOAc. The combined organic extracts were washed with brine and water, and then were dried (Na₂SO₄) and evaporated. The residue was purified by flash chromatography eluting with 1:1 cyclohexane:EtOAc. Fractions containing product were pooled and evaporated to give the title compound (10.3 g). LCMS m/z 330 [MH⁺]; T_{RET}=2.20 min.

### Intermediate 8

### 5-(aminomethyl)-1,6-diethyl-N-(tetrahydro-2H-pyran-4-yl)-1H-pyrazolo[3,4-b]pyridin-4-amine

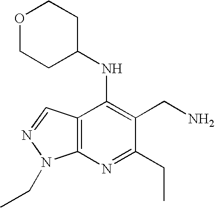

A suspension of Intermediate 7 (10.3 g, 34 mmol) in ethanol was added to 10% palladium on carbon (1 g) and stirred under an atmosphere of hydrogen for 3 hours. The catalyst was removed by filtration under nitrogen and was washed with ethanol. The filtrate was evaporated to give the title compound (9.4 g) as a dark oil. Ether was added and then evaporated to give a semi-solid. LCMS m/z 304 [MH⁺]; T_{RET}=1.67 min.

### Intermediate 8 (Alternative Procedure)

### 5-(aminomethyl)-1,6-diethyl-N-(tetrahydro-2H-pyran-4-yl)-1H-pyrazolo[3,4-b]pyridin-4-amine

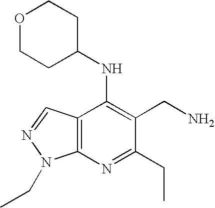

Ethanol (300 ml) was added to 10% palladium on carbon (9.9 g, 50% wet) followed by Intermediate 7 (49.6 g) in ethanol (900 ml). The mixture was hydrogenated at room temperature and pressure overnight. The catalyst was removed by filtration and the filtrate was evaporated. The residue was purified by chromatography eluting initially with 5% methanol in dichloromethane followed by 10% and finally 15% methanol in dichloromethane to give the title compound as an almost white solid (41.3 g). LCMS m/z 304 [MH⁺]; T_{RET}=1.65, 1.68 min (split peak).

### Intermediate 9

### ethyl 4-[(1-{[(1,1-dimethylethyl)oxy]carbonyl}-4-piperidinyl)amino]-1,6-diethyl-1H-pyrazolo[3,4-b]pyridine-5-carboxylate

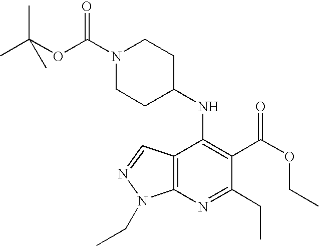

A solution of Intermediate 4 (14.8 g, 52.6 mmol) in 1-methyl-2-pyrrolidinone (140 ml) was treated with DIPEA (22.9 ml, 131 mmol) followed by 1,1-dimethylethyl 4-amino-1-piperidinecarboxylate (e.g. available from Astatech, 11.6 g, 57.9 mmol) and heated at 120° C. for 24 hours. The mixture was cooled and poured into aqueous lithium chloride solution (5% LiCl, 1 L) and the aqueous phase was extracted with EtOAc (3×250 ml). The combined organics were dried (Na₂SO₄) and the solvent evaporated. The residue was purified by silica gel chromatography (500 g) eluting with a gradient of from 4:1 hexane:EtOAc to 2:1 hexane:EtOAc. The fractions containing the product were pooled and evaporated to give the title compound (18.6 g) as an oil which solidified on standing. LCMS m/z 446 [MH⁺]; T_{RET}=3.68 min.

Intermediate 10

### 1,1-dimethylethyl 4-{[1,6-diethyl-5-(hydroxymethyl)-1H-pyrazolo[3,4-b]pyridin-4-yl]amino}-1-piperidinecarboxylate

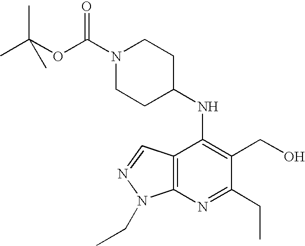

A solution of Intermediate 9 (18.3 g, 41.1 mmol) in THF (100 ml) was treated with lithium borohydride (LiBH₄, 2M in THF, 40 ml, 80 mmol) followed by MeOH (3.6 ml, 88.8 mmol) and heated under reflux for 3 hours. To the cooled mixture was added a further portion of lithium borohydride (2M in THF, 40 ml, 80 mmol) followed by MeOH (3.6 ml, 88.8 mmol). The mixture was heated under reflux for 2 hours and allowed to cool to room temperature. Solid lithium borohydride (1.6 g, 73.5 mmol) was added followed by MeOH (3.6 ml, 88.8 mmol) and the mixture heated under reflux for 2 hours and allowed to cool overnight. The mixture was cooled in an ice/water bath, and treated with MeOH (20 ml). After stirring for 10 min, water (10 ml) was added. When the effervescence ceased more water was added cautiously (500 ml total volume) and the mixture stirred for 45 minutes. The mixture was partitioned between water and DCM, the aqueous phase separated and extracted with DCM (x2). The combined organic extracts were washed with brine, dried (Na₂SO₄) and the solvent was evaporated. The residue was purified by chromatography (silica gel, 800 g) using chloroform to load the mixture and eluting with hexane:EtOAc (1:1, 800 ml), hexane:EtOAc (1:2, 1800 ml), and finally neat EtOAc. The fractions containing the product were combined and evaporated to give the title compound (12.4 g). LCMS m/z 404 [MH⁺]; T_{RET}=2.40 min.

### Intermediate 11

### 1,1-dimethylethyl 4-{[5-(azidomethyl)-1,6-diethyl-1H-pyrazolo[3,4-b]pyridin-4-yl]amino}-1-piperidinecarboxylate

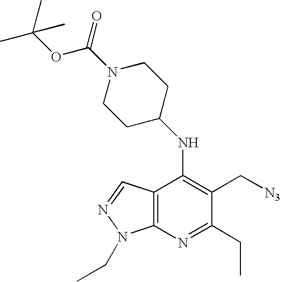

A suspension of Intermediate 10 (14 g, 34.7 mmol) in DMF (110 ml) was treated with sodium azide (4.6 g, 70.6 mmol) and carbon tetrabromide (23.1 g, 69.7 mmol). The stirred suspension was cooled in an ice/water bath and a solution of triphenylphosphine (18.6 g, 70.9 mmol) in DMF (75 ml) added dropwise over 30 minutes. The resulting yellow solution was allowed to warm to room temperature and stirred for a further 3.5 hours. The suspension was concentrated to about ⅓ original volume and partitioned between water (500 ml) and EtOAc (600 ml). The organic phase was separated and washed with water (x2). The aqueous washings were back extracted with EtOAc. The combined organic extracts were then washed with 5% aqueous lithium chloride solution, dried (Na₂SO₄) and evaporated to dryness. The residue was purified by chromatography (silica gel, 800 g) loading with DCM and eluting with 1:1 hexane:EtOAc. The fractions containing the product were combined and evaporated to give the title compound (12.6 g). LCMS m/z 429 [MH⁺]; T_{RET}=2.92 min.

### Intermediate 12

### 4-[5-(azidomethyl)-1,6-diethyl-1H-pyrazolo[3,4-b]pyridin-4-yl]amino}-1-piperidinecarboxamide

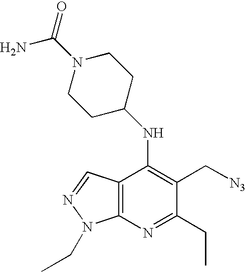

A solution of Intermediate 11 (8.9 g, 20.8 mmol) in 1,4-dioxane (50 ml) was treated with a 4M solution of hydrogen chloride in 1,4-dioxane (125 ml). After 2 hours the solvent was evaporated and the residue partitioned between DCM and saturated aqueous NaHCO₃ solution. The organic phase was collected and washed with saturated aqueous NaHCO₃ solution. The aqueous phases were back-extracted with DCM and then the combined organic phases were dried (Na₂SO₄) and evaporated to give an oil (LCMS showed MH⁺=329; T_{RET}=1.75 min). The oil was dissolved in DCM (100 ml), treated with DIPEA (6.1 ml, 35.0 mmol) followed by trimethylsilyl isocyanate (e.g. available from Aldrich, 3.6 ml, 26.6 mmol) and stirred at room temperature overnight. Additional DIPEA (6.1 ml, 35.0 mmol) and trimethylsilyl isocyanate (Aldrich, 3.6 ml, 26.6 mmol) was added. After stirring for a further 7 h additional trimethylsilyl isocyanate (3.6 ml) was added and stirring continued over the weekend (about 62 hours). Water (100 ml) was added and the mixture stirred for 1 hour and the white solid collected by filtration, washed with water followed by diethyl ether and then dried to give the title compound (2.21 g). LCMS m/z 372 [MH⁺]; T_{RET}=2.08 min.

### Alternative preparation of 4-{[5-(azidomethyl)-1,6-diethyl-1H-pyrazolo[3,4-b]pyridin-4-yl]amino}-1-piperidinecarboxamide

1,1-dimethylethyl 4-{[5-(azidomethyl)-1,6-diethyl-1H-pyrazolo[3,4-b]pyridin-4-yl]amino}-1-piperidinecarboxylate (5.14 g) is treated with a 4M solution of hydrogen chloride in dioxane (50 ml). The reaction mixture is left to stand for 2 hours. The solvent is evaporated and co-evaporated with dichloromethane (2×50 ml) then diethyl ether (2×50 ml) to give 5-(azidomethyl)-1,6-diethyl-N-4-piperidinyl-1H-pyrazolo[3,4-b]pyridin-4-amine, hydrochloride salt. This product is dissolved in dichloromethane (100 ml) and the solution is treated with DIPEA (6.3 ml) and trimethylsilyl isocyanate (1.8 ml), and then is left to stand at room temperature for 18 hours. Water (50 ml) is added and the precipitated solid is collected by filtration, washed with diethyl ether and dried to give 4-{[5-(azidomethyl)-1,6-diethyl-1H-pyrazolo[3,4-b]pyridin-4-yl]amino}-1-piperidinecarboxamide.

### Intermediate 13

### 4-{[5-(aminomethyl)-1,6-diethyl-1H-pyrazolo[3,4-b]pyridin-4-yl]amino}-1-piperidinecarboxamide

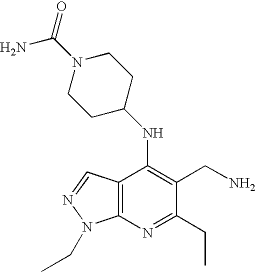

10% Palladium on carbon (0.65 g) was treated with water (about 2 ml), ethanol (20 ml) and a suspension of Intermediate 12 (4.1 g, 11.0 mmol) in ethanol (100 ml). Additional ethanol (200 ml) was used to wash in the precipitate. The suspension was stirred under an atmosphere of hydrogen for 21 hours. The mixture was filtered through celite and the filtrate was evaporated to dryness to give the title compound as a grey foam (3.8 g). LCMS m/z 346 [MH⁺]; T_{RET}=1.66 min.

### Intermediate 14

### ethyl 4-chloro-1-ethyl-6-methyl-1H-pyrazolo[3,4-b]pyridine-5-carboxylate

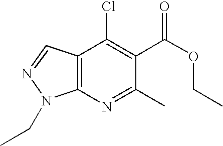

A mixture of Intermediate 3 (158 g, 0.72 mol), Intermediate I (79 g, 0.71 mol) and triethylamine (196 ml) in toluene (2 L) was heated under reflux for 16 hrs. The reaction mixture was then cooled to room temperature, filtered and the solid residue washed thoroughly with toluene. From the combined filtrate and the washings, toluene was removed by evaporation under reduced pressure. The residue was treated with phosphorus oxychloride (POCl₃, 2 L) and then heated under reflux for 16 hrs. Excess phosphorus oxychloride was removed under reduced pressure. The reaction mixture was diluted with EtOAc (1 L) and cooled to 10° C. Saturated aqueous sodium bicarbonate solution (800 ml) was added dropwise. The organic layer was separated, dried (Na₂SO₄) and evaporated to give an oil.

The crude product was purified by chromatography (silica gel, 60-120 mesh, 3 kg) eluting with 3% ethyl acetate in hexane. The desired fractions were combined and evaporated to give the title compound (76 g). 1H NMR (200 MHz, chloroform-d) δ (delta) ppm 1.42(3H, t, J=7.2 Hz), 1.50 (3H, t, J=7.3 Hz), 2.68 (3H, s), 4.47 (2H, q, J=7.3 Hz), 4.56 (2H, q, J=7.2 Hz), 8.04 (1H, s).

### Alternative preparation of ethyl 4-chloro-1-ethyl-6-methyl-1H-pyrazolo[3,4-b]pyridine-5-carboxylate

A mixture of 5-amino-1-ethylpyrazole (1.614 g, 14.5 mmol) and diethyl 2-(1-ethoxyethylidene)malonate [e.g. see J. Am. Chem. Soc., 1931, 53, 1836] (3.68 g) is heated at 150° C. under Dean Stark conditions for 5 hours. Phosphorous oxychloride (25 ml) is carefully added to the mixture and the resulting solution is heated at 130° C. under reflux for 18 hours. The mixture is concentrated in vacuo and the residual oil is carefully added, with cooling, to water (100 ml). The resulting mixture is extracted with dichloromethane (3×100 ml) and the combined organic extracts are dried over anhydrous sodium sulphate and concentrated in vacuo. The residual oil is purified by Biotage chromatography (silica; 90 g) eluting with 5% ethyl acetate in petroleum ether. Fractions containing the desired product are combined and concentrated in vacuo to give ethyl 4-chloro-1-ethyl-6-methyl-1H-pyrazolo[3,4-b]pyridine-5-carboxylate.

### Intermediate 15

### ethyl 1-ethyl-6-methyl-4-(tetrahydro-2H-pyran-4-ylamino)-1H-pyrazolo[3,4-b]pyridine-5-carboxylate

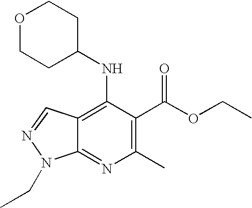

Intermediate 14 (58 g, 217 mmol) in 1-methyl-2-pyrrolidinone (380 ml) was treated with DIPEA (75 ml, 433 mmol) and a solution of tetrahydro-2H-pyran-4-amine (e.g. available from Peakdale, 26.2 g, 260 mmol) in 1-methyl-2-pyrrolidinone (100 ml), and was then heated at 115° C. overnight. The cooled mixture was poured into water (2500 ml) and extracted with EtOAc (6×250 ml). The combined organic extracts were washed with water and brine, dried and evaporated. The residue was suspended in diethyl ether (about 150 ml) and the solid collected by filtration to give the title compound (44.5 g) as a white solid. LCMS m/z 333 [MH⁺]; T_{RET}=2.89 min.

The filtrate was evaporated, dissolved in EtOAc, washed with water and brine, dried and evaporated. Treatment with diethyl ether as described above gave a further quantity of the title compound (9.6 g). A further repeat of this procedure on the filtrate gave an additional quantity of the title compound (2.16 g).

### Alternative preparation of ethyl 1-ethyl-6-methyl-4-(tetrahydro-2H-pyran-4-ylamino)-1H-pyrazolo[3,4-b]pyridine-5-carboxylate

4-Aminotetrahydro-2H-pyran hydrochloride (e.g. see Intermediate 8A of WO 2004/024728 A2, 0.413 g, 3.0 mmol) is added to a mixture of ethyl 4-chloro-1-ethyl-6-methyl-1H-pyrazolo[3,4-b]pyridine-5-carboxylate (0.268 g, 1.0 mmol) and DIPEA (0.87 ml, 5.0 mmol) in MeCN (3 ml). The resulting mixture is heated at 85° C. for 24 hours. Volatiles are removed in vacuo and the residue is dissolved in chloroform (1.5 ml) and applied to a SPE cartridge (silica, 5 g). The cartridge is eluted successively with Et₂O,

EtOAc and EtOAc-MeOH (9:1). Fractions containing the desired product (which might be contaminated with starting material) are combined and concentrated in vacuo. Further purification using a SPE cartridge (silica, 5 g) eluting with EtOAc-cyclohexane (1:3) affords ethyl 1-ethyl-6-methyl-4-(tetrahydro-2H-pyran-4-ylamino)-1H-pyrazolo[3,4-b]pyridine-5-carboxylate.

### Intermediate 16

### [1-ethyl-6-methyl-4-(tetrahydro-2H-pyran-4-ylamino)-1H-pyrazolo[3,4-b]pyridin-5-yl]methanol

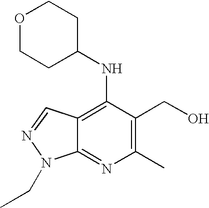

Intermediate 15 (26.9 g, 80.9 mmol) in dry DCM (270 ml) under an atmosphere of nitrogen was cooled to 0° C. and treated with di-iso-butyl aluminium hydride (162 ml of a 1.5M solution in toluene, 243 mmol). The addition took 27 minutes. The resulting pale yellow solution was stirred at 0° C. for 35 minutes and quenched by careful addition of about 20% aqueous potassium sodium tartrate solution (Rochelle's salt, 250 ml). This was exothermic and produced effervescence (temperature reached a maximum of 30° C.). After stirring for 20 min, the solids were removed by filtration and washed with DCM and EtOAc and the aqueous phase of the filtrate extracted firstly with DCM then EtOAc. The combined organics were washed with water then brine, dried and evaporated to give 3 g of material. The aqueous phase was treated with more potassium sodium tartrate, concentrated and exhaustively extracted with EtOAc. The EtOAc extracts were combined, dried and evaporated to give the title compound (17.9 g) as a cream solid. LCMS m/z 291 [MH⁺]; T_{RET}=1.74, 1.81 min (split peak).

### Intermediate 17

### 5-(azidomethyl)-1-ethyl-6-methyl-N-(tetrahydro-2H-pyran-4-yl)-1H-pyrazolo[3,4-b]pyridin-4-amine

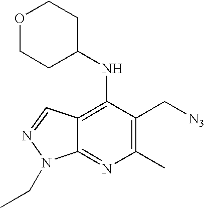

A suspension of Intermediate 16 (9.7 g, 33.4 mmol) in toluene (100 ml) was treated dropwise with thionyl chloride (SOCl₂, 36.6 ml, 500 mmol) over 10 minutes resulting in a gummy lump. The mixture was heated to 80° C. for 2.5 hours by which time the lump had broken down to be a pale brown suspension. The mixture was cooled, then evaporated and the residue was azeotroped with more toluene (about 20 ml). The resultant pale brown solid was suspended in DMSO (75 ml) and treated with sodium azide (3.25 g, 50.1 mmol); the mixture became darker and slightly more translucent. After 2 hours the mixture was partitioned between saturated aqueous sodium bicarbonate solution and EtOAc, the layers separated and the aqueous phase extracted with more EtOAc. The combined organics were washed with water then brine, dried and evaporated. The residue was purified by silica gel chromatography (800 g) eluting with 1:1 cyclohexane:EtOAc. The fractions containing the product were pooled and evaporated to give the title compound (7.6 g). 1H NMR (400 MHz, CDCl₃) δ (delta) ppm 1.51 (3H, t), 1.68 (2H, m), 2.16 (2H, m), 2.64 (3H, s), 3.61 (2H, m), 4.05 (2H, m), 4.13 (1H, m), 4.45 (2H, s), 4.47 (2H, q), 4.96 (1H, d), 7.88 (1H, s).

### Intermediate 18

### 5-(aminomethyl)-1-ethyl-6-methyl-N-(tetrahydro-2H-pyran-4-yl)-1H-pyrazolo[3,4-b]pyridin-4-amine

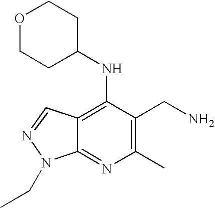

10% palladium on carbon (50% wet, 2.1 g) was treated with ethanol (20 ml) followed by a solution of Intermediate 17 (10.48 g) in ethanol (180 ml), and was hydrogenated at room temperature and pressure overnight. The catalyst was removed by filtration, washing through with more ethanol, and the solvents were removed under reduced pressure to give the title compound (9.0 g) as a grey foam: 1H NMR (400 MHz, d6 DMSO) δ (delta) ppm 1.36 (3H, t), 1.59 (2H, m), 2.0 (2H, m), 2.50 (3H, s), 3.37 (2H, br s), 3.62 (2H, m), 3.89 (2H, s), 3.91 (2H, m), 4.14 (1H, m), 4.33 (2H, q), 7.62 (1H, d), 8.02 (1H, s).

### Optional Intermediates

In this Optional Intermediates section, “Intermediates” generally represent syntheses of intermediate compounds which (in some cases) might theoretically be usable in the synthesis of compounds of formula (I) or salts thereof, but which have not necessarily been used to prepare specific compounds of formula (I) or salts thereof (and in most or all cases they have not been so used).

### Optional Intermediate 60

### [1-ethyl-4-(tetrahydro-2H-pyran-4-ylamino)-1H-pyrazolo[3,4-b]pyridin-5-yl]methanol

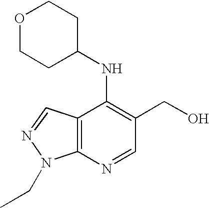

A solution of 1M diisobutylaluminum hydride in dichloromethane (80 ml) is added dropwise to a stirred solution of ethyl 1-ethyl-4-(tetrahydro-2H-pyran-4-ylamino)-1H-pyrazolo[3,4-b]pyridine-5-carboxylate [e.g. see Intermediate 32 and/or Example 3 of WO 2004/024728 A2] (13.8 g) in dichloromethane (75 ml) at 0° C. under nitrogen. The reaction mixture is maintained below 5° C. during the addition, and is then stirred for 0.5 h at 0° C. The mixture is then quenched by addition of aqueous potassium sodium tartrate (10% solution), diluted with water (150 ml) and the organic phase separated. The aqueous phase is extracted with ethyl acetate (2×250 ml) and the combined organics are dried (magnesium sulphate) and evaporated. The residue is purified by column chromatography on silica gel eluting with a gradient of 0-100% ethyl acetate in cyclohexane followed by 0-20% methanol in ethyl acetate to give Intermediate 60.

### Intermediate 61

### 5-(chloromethyl)-1-ethyl-N-(tetrahydro-2H-pyran-4-yl)-1H-pyrazolo[3,4-b]pyridin-4-amine

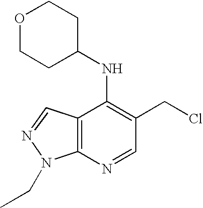

Intermediate 60 (80 mg) is treated with thionyl chloride (1 ml) and heated at 80° C. for 1 h then allowed to cool. The orange solution is evaporated to dryness and the residue azeotroped with toluene (2×5 ml) to give Intermediate 61.

### Optional Intermediate 62

### 5-(azidomethyl)-1-ethyl-N-(tetrahydro-2H-pyran-4-yl)-1H-pyrazolo[3,4-b]pyridin-4-amine

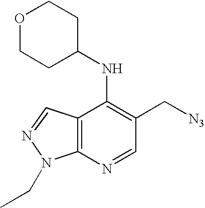

A solution of Intermediate 61 (50 mg) in anhydrous dimethylsulphoxide (0.2 ml) is treated with lithium azide (9 mg) and the solution is stirred at room temperature for 20 h. A further portion of lithium azide (15 mg) is then added, and after a further day stirring at room temperature, water (0.25 ml) is added. The solution is extracted with dichloromethane (2×5 ml) and the combined organic extracts are passed through a hydrophobic frit (6 ml) and then are blown to dryness. The residue is dissolved in dichloromethane (0.5 ml) and applied to an SPE cartridge (silica; 1 g). The cartridge is eluted with 50% ethyl acetate in cyclohexane and fractions containing the desired material are combined and blown to dryness to give Intermediate 62.

### Optional Intermediate 63

### 1-ethyl-N⁴-(tetrahydro-2H-pyran-4-yl)-1H-pyrazolo[3,4-b]pyridine-4,5-diamine

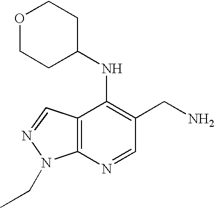

A solution of Intermediate 62 (0.351 g) in ethanol (30 ml) is added to palladium on carbon (5% wet, 0.050 g) and the mixture is stirred at room temperature for 20 hours under an atmosphere of hydrogen. The mixture is filtered through a glass fibre filter and through celite, which is then washed with ethanol (50 ml). The combined filtrates and washings are concentrated in vacuo to give Intermediate 63.

| | | One or two possible published |
|---|---|---|
| Optional | | reference(s) to (e.g. synthetic |
| Intermediate | | reference to), or one possible |
| Number | Structure of compound | source of, the compound |
| 100 | 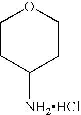 | Intermediate 8A of WO 2004/024728 A2 (Glaxo Group Limited) |
| 101 | 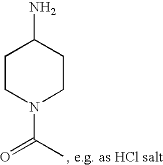 | Intermediate 6 of WO 2004/024728 A2 (Glaxo Group Limited), which refers to WO 00/42011 |
| 102 | 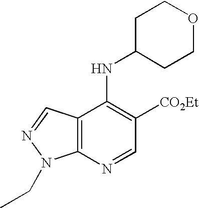 | Intermediate 32 and/or Example 3 of WO 2004/024728 A2 (Glaxo Group Limited); or Intermediate 4 of WO 2005/058892 A1 (Glaxo Group Limited) |
| 103 | 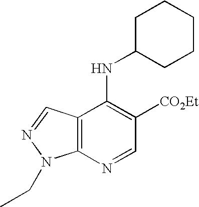 | Intermediate 5 of WO 2005/058892 A1 (Glaxo Group Limited) |
| 104 | 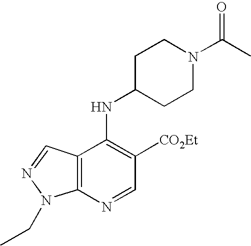 | Example 207 of WO 2004/024728 A2 (Glaxo Group Limited); or Intermediate 6 of WO 2005/058892 A1 (Glaxo Group Limited) |
| 105 | 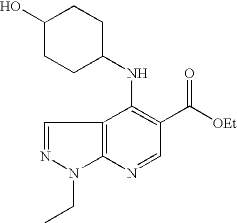 | Example 204 of WO 2004/024728 A2 (Glaxo Group Limited) |
| 106 | 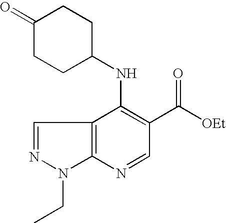 | Example 205 of WO 2004/024728 A2 (Glaxo Group Limited) |
| 107 | 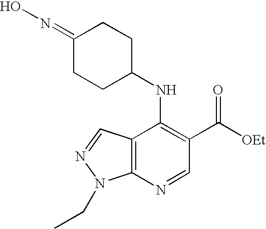 | Example 652 of WO 2004/024728 A2 (Glaxo Group Limited) |
| 108 | 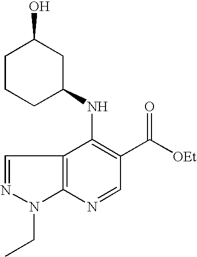 [cis-(3-hydroxycyclohex-1-yl)amino group, racemic] | Intermediate 12 of WO 2005/058892 A1 (Glaxo Group Limited) |
| 109 | 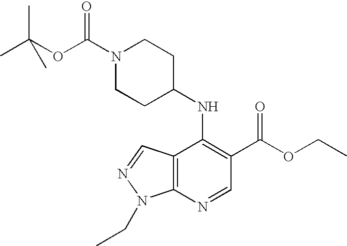 | Intermediate 102 of WO 2005/058892 A1 (Glaxo Group Limited) |
| 110 | 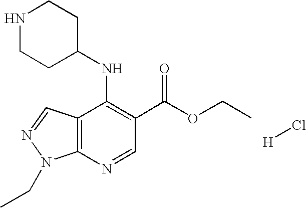 | Intermediate 103 of WO 2005/058892 A1 (Glaxo Group Limited) |
| 111 | 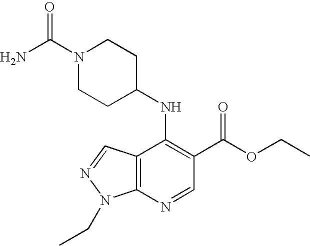 | Intermediate 104 of WO 2005/058892 A1 (Glaxo Group Limited) |
| 112 | 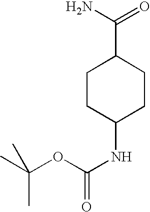 | Intermediate 112 of WO 2005/058892 A1 (Glaxo Group Limited) |
| 113 | 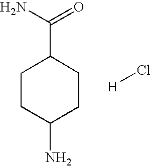 | Intermediate 113 of WO 2005/058892 A1 (Glaxo Group Limited) |
| 114 | 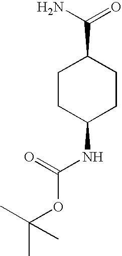 | Intermediate 114 of WO 2005/058892 A1 (Glaxo Group Limited) |
| 115 | 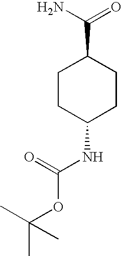 | Intermediate 115 of WO 2005/058892 A1 (Glaxo Group Limited) |
| 116 | 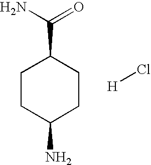 | Intermediate 116 of WO 2005/058892 A1 (Glaxo Group Limited) |
| 117 | 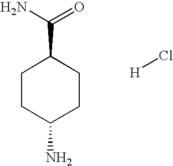 | Intermediate 117 of WO 2005/058892 A1 (Glaxo Group Limited) |
| 118 | 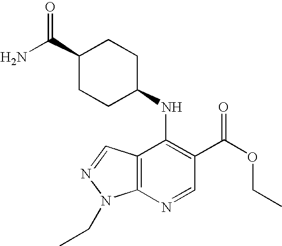 | Intermediate 118 of WO 2005/058892 A1 (Glaxo Group Limited) |
| 119 | 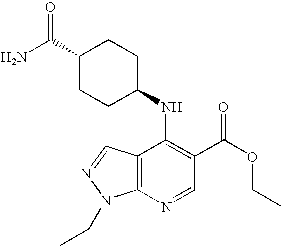 | Intermediate 119 of WO 2005/058892 A1 (Glaxo Group Limited) |
| 120 | 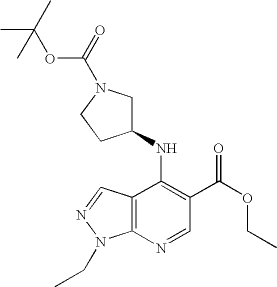 | Intermediate 146 of WO 2005/058892 A1 (Glaxo Group Limited) |
| 121 | 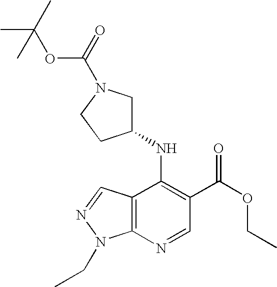 | Intermediate 147 of WO 2005/058892 A1 (Glaxo Group Limited) |
| 122 | 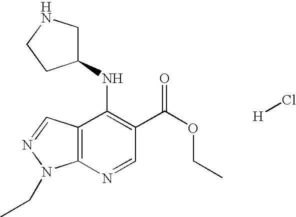 | Intermediate 148 of WO 2005/058892 A1 (Glaxo Group Limited) |
| 123 | 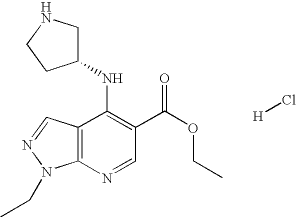 | Intermediate 149 of WO 2005/058892 A1 (Glaxo Group Limited) |
| 124 | 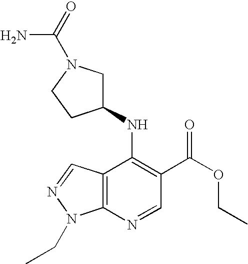 | Intermediate 150 of WO 2005/058892 A1 (Glaxo Group Limited) |
| 125 | 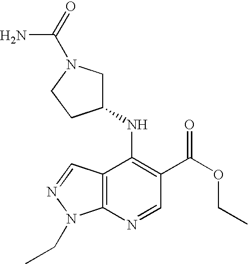 | Intermediate 151 of WO 2005/058892 A1 (Glaxo Group Limited) |
| 126 | 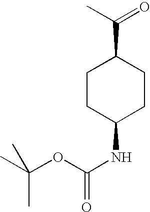 | Intermediate 155 of WO 2005/058892 A1 (Glaxo Group Limited) |
| 127 | 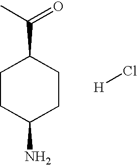 | Intermediate 156 of WO 2005/058892 A1 (Glaxo Group Limited) |
| 128 | 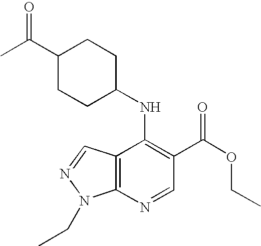 (mixture of cis and trans isomers) | Intermediate 157 of WO 2005/058892 A1 (Glaxo Group Limited) |
| 129 | 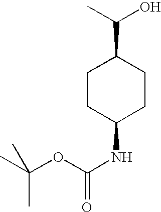 | Intermediate 159 of WO 2005/058892 A1 (Glaxo Group Limited) |
| 130 | 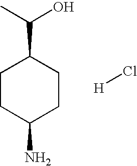 | Intermediate 160 of WO 2005/058892 A1 (Glaxo Group Limited) |
| 131 | 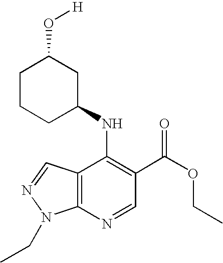 [contains trans-(3-hydroxycyclohex-1-yl)amino group, racemic] | Intermediate 161 of WO 2005/058892 A1 (Glaxo Group Limited) |
| 132 | 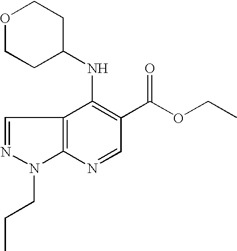 | Example 185 of WO 2004/024728 A2 (Glaxo Group Limited); or Intermediate 171 of WO 2005/058892 A1 (Glaxo Group Limited) |
| 133 | 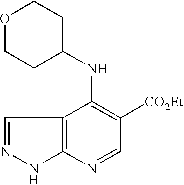 | Example 20 of WO 2004/024728 A2 (Glaxo Group Limited) |
| 134 | 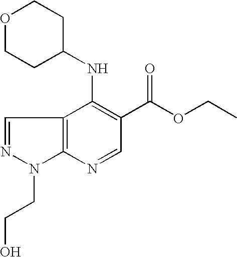 | Example 186 of WO 2004/024728 A2 (Glaxo Group Limited) |
| 135 | 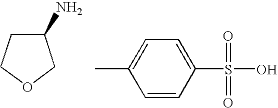 | Fluka Chemie AG, Germany (CAS 111769-27-8) |
| 136 | 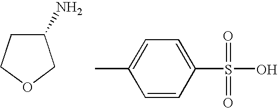 | E. Merck, Germany; or E. Merck (Merck Ltd), Hunter Boulevard, Magna Park, Lutterworth, Leicestershire LE17 4XN, United Kingdom (CAS 104530-80-5) |
| 137 | 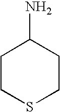 | Intermediate 11 of WO 2004/024728 A2, and optionally reference cited therein |
| 138 | 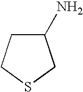 | Intermediate 12 of WO 2004/024728 A2, and optionally references cited therein |
| 139 | 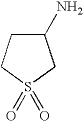 | Sigma Aldrich Library of Rare Chemicals (SALOR) (CAS-6338-70-1) |
| 140 | 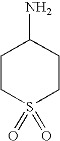 | Intermediate 14 of WO 2004/024728 A2, and optionally references cited therein |
| 141 | 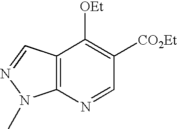 | Intermediate 3 of WO 2004/024728 A2 (Glaxo Group Limited) |
| 142 | 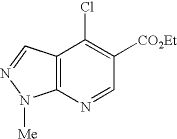 | Intermediate 25 of WO 2004/024728 A2 (Glaxo Group Limited) |
| 143 | 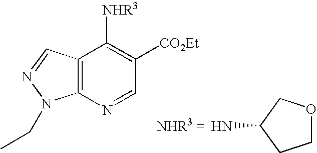 | Example 8 of WO 2004/024728 A2 (Glaxo Group Limited) |
| 144 | 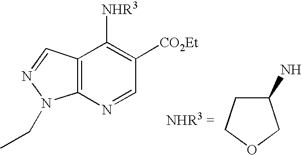 | Example 9 of WO 2004/024728 A2 (Glaxo Group Limited) |
| 145 | 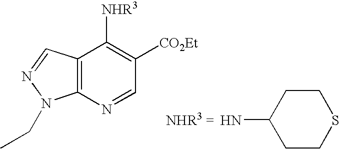 | Example 10 of WO 2004/024728 A2 (Glaxo Group Limited) |
| 146 | 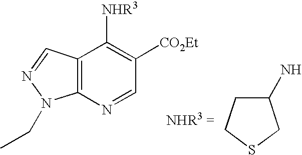 | Example 11 of WO 2004/024728 A2 (Glaxo Group Limited) |
| 147 | 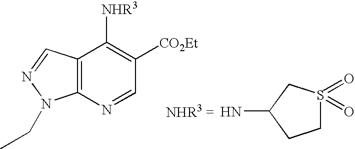 | Example 13 of WO 2004/024728 A2 (Glaxo Group Limited) |
| 148 | 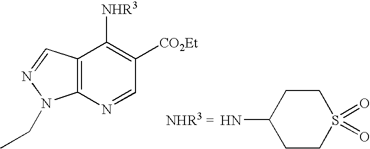 | Example 14 of WO 2004/024728 A2 (Glaxo Group Limited) |
| 149 | 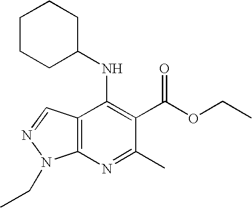 | Example 190 of WO 2004/024728 A2 (Glaxo Group Limited) |
| 150 | 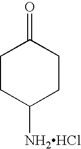 | Intermediate 54 of WO 2004/024728 A2 (Glaxo Group Limited) |
| 151 | 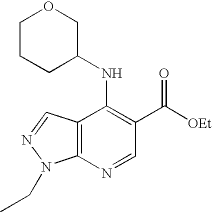 | Intermediate 58A of WO 2004/024728 A2 (Glaxo Group Limited) |
| 152 | 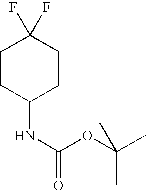 [sample containing 1,1-dimethylethyl (4-fluoro-3-cyclohexen-1- yl)carbamate as an impurity] | Intermediate 62 of WO 2004/024728 A2 (Glaxo Group Limited) |
| 153 | 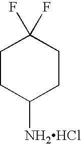 [sample containing 4-fluoro-3- cyclohexen-1-amine) as an impurity] | Intermediate 63 of WO 2004/024728 A2 (Glaxo Group Limited) |
| 154 | 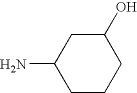 | AB Chem, Inc., Canada (mixture of cis and trans); or J. Chem. Soc., Perkin Trans. 1, 1994, 537 |
| 155 | as Intermediate 154, but racemic cis- isomer, i.e. racemic cis-(3-hydroxy- cyclohex-1-yl)-amine | J. Chem. Soc., Perkin Trans 1, 1994, 537 (discloses a 3.3:1 cis:trans mixture) |
| 156 | 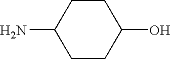 | Aldrich; or TCI-America |
| 157 | 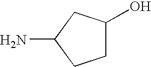 | U.S. Pat. No. 4,219,660 |
| 158 | 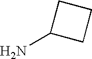 | Aldrich |
| 159 | 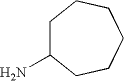 | Aldrich |
| 160 | 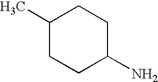 | Aldrich |
| 161 | 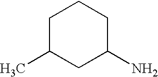 | Pfaltz-Bauer |
| 162 | 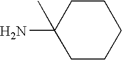 | J. Org. Chem., 1985, 50(11), 1859 |
| 163 | 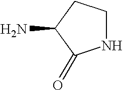 | WO 99/12933 |
| 164 | 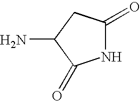 | EP 1188744 |
| 165 | 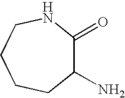 (3-Aminoazepan-2-one) | Sigma-Aldrich Company Ltd |
| 166* | 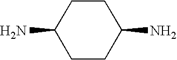 | J. Med. Chem., 1994, 37(17), 2360 |
| 167* | 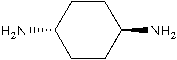 | Aldrich |
| 168* | 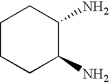 (trans isomer, e.g. optionally racemic) | Aldrich |
| 169* | 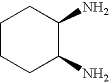 | Aldrich |
| 170* | 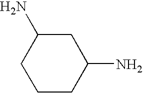 | Peakdale Molecular Ltd |
| 171 | 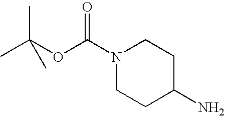 1,1-dimethylethyl 4-amino-1- piperidinecarboxylate | AstaTech |
| 172 | 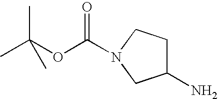 | |
| 173 | 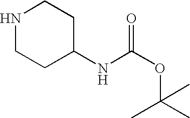 1,1-dimethyl 4- piperidinylcarbamate | Syngene or AstaTech |
| 174 | 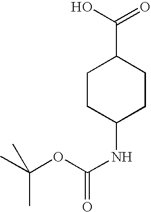 4-({[(1,1-dimethylethyl)oxy]carbon- yl}amino)cyclohexane carboxylic acid | Fluka |
| 175 | 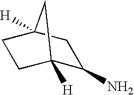 | Aldrich |
| 176 | 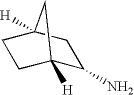 | Aldrich |

### Examples

Table of Examples

| Example No. | Example Name |
|---|---|
| 1 | N-{[1,6-diethyl-4-(tetrahydro-2H-pyran-4-ylamino)-1H- |
| | pyrazolo[3,4-b]pyridin-5-yl]methyl}-4-[3-({[1,6-diethyl-4- |
| | (tetrahydro-2H-pyran-4-ylamino)-1H-pyrazolo[3,4-b]pyridin-5- |
| | yl]methyl}amino)-3-oxopropyl]benzamide trifluoroacetate |
| 2 | N,N′-bis{[1,6-diethyl-4-(tetrahydro-2H-pyran-4-ylamino)-1H- |
| | pyrazolo[3,4-b]pyridin-5-yl]methyl}pentanediamide |
| | trifluoroacetate |
| 2b | N,N′-bis{[1,6-diethyl-4-(tetrahydro-2H-pyran-4-ylamino)-1H- |
| | pyrazolo[3,4-b]pyridin-5-yl]methyl}pentanediamide |
| 3 | N,N′-bis{[1,6-diethyl-4-(tetrahydro-2H-pyran-4-ylamino)-1H- |
| | pyrazolo[3,4-b]pyridin-5-yl]methyl}-3,3-dimethylpentanediamide |
| | trifluoroacetate |
| 4 | N,N′-bis{[1,6-diethyl-4-(tetrahydro-2H-pyran-4-ylamino)-1H- |
| | pyrazolo[3,4-b]pyridin-5-yl]methyl}-2,3-dimethylbutanediamide |
| | trifluoroacetate |
| 5 | 4,4′-methanediylbis(N-{[1,6-diethyl-4-(tetrahydro-2H-pyran-4- |
| | ylamino)-1H-pyrazolo[3,4-b]pyridin-5-yl]methyl}benzamide) |
| | trifluoroacetate |
| 6 | 2,2′-benzene-1,4-diylbis(N-{[1,6-diethyl-4-(tetrahydro-2H-pyran- |
| | 4-ylamino)-1H-pyrazolo[3,4-b]pyridin-5-yl]methyl}acetamide) |
| | trifluoroacetate |
| 7 | N-{[1,6-diethyl-4-(tetrahydro-2H-pyran-4-ylamino)-1H- |
| | pyrazolo[3,4-b]pyridin-5-yl]methyl}-2-[3-({[1,6-diethyl-4- |
| | (tetrahydro-2H-pyran-4-ylamino)-1H-pyrazolo[3,4-b]pyridin-5- |
| | yl]methyl}amino)-3-oxopropyl]benzamide trifluoroacetate |
| 8 | 2,2′-benzene-1,3-diylbis(N-{[1,6-diethyl-4-(tetrahydro-2H-pyran- |
| | 4-ylamino)-1H-pyrazolo[3,4-b]pyridin-5-yl]methyl}acetamide) |
| | trifluoroacetate |
| 9 | 2,2′-oxybis(N-{[1,6-diethyl-4-(tetrahydro-2H-pyran-4-ylamino)- |
| | 1H-pyrazolo[3,4-b]pyridin-5-yl]methyl}acetamide)trifluoroacetate |
| 9b | 2,2′-oxybis(N-{[1,6-diethyl-4-(tetrahydro-2H-pyran-4-ylamino)- |
| | 1H-pyrazolo[3,4-b]pyridin-5-yl]methyl}acetamide) |
| 10 | N,N′-bis{[1,6-diethyl-4-(tetrahydro-2H-pyran-4-ylamino)-1H- |
| | pyrazolo[3,4-b]pyridin-5-yl]methyl}-3,3′-biphenyldicarboxamide |
| | trifluoroacetate |
| 11 | N,N′-bis[(4-{[1-(aminocarbonyl)-4-piperidinyl]amino}-1,6-diethyl- |
| | 1H-pyrazolo[3,4-b]pyridin-5-yl)methyl]-1,4-cyclohexanedicarboxamide |
| | trifluoroacetate |
| 12 | 4,4′-{sulfonylbis[(1-oxo-2,1-ethanediyl)iminomethanediyl(1,6-diethyl- |
| | 1H-pyrazolo[3,4-b]pyridine-5,4-diyl)imino]}di(1-piperidinecarboxamide) |
| | trifluoroacetate |
| 13 | 4-({5-[({[3-({[(4-{[1-(aminocarbonyl)-4-piperidinyl]amino}-1,6- |
| | diethyl-1H-pyrazolo[3,4-b]pyridin-5- |
| | yl)methyl]amino}carbonyl)phenyl]acetyl}amino)methyl]-1,6- |
| | diethyl-1H-pyrazolo[3,4-b]pyridin-4-yl}amino)-1- |
| | piperidinecarboxamide trifluoroacetate |
| 14 | 4-({5-[({3-[4-({[(4-{[1-(aminocarbonyl)-4-piperidinyl]amino}-1,6- |
| | diethyl-1H-pyrazolo[3,4-b]pyridin-5- |
| | yl)methyl]amino}carbonyl)phenyl]propanoyl}amino)methyl]-1,6- |
| | diethyl-1H-pyrazolo[3,4-b]pyridin-4-yl}amino)-1- |
| | piperidinecarboxamide trifluoroacetate |
| 15 | 4-({5-[({[4-({[(4-{[1-(aminocarbonyl)-4-piperidinyl]amino}-1,6- |
| | diethyl-1H-pyrazolo[3,4-b]pyridin-5- |
| | yl)methyl]amino}carbonyl)phenyl]acetyl}amino)methyl]-1,6- |
| | diethyl-1H-pyrazolo[3,4-b]pyridin-4-yl}amino)-1- |
| | piperidinecarboxamide trifluoroacetate |
| 16 | 4-[(5-{[({[2-({[(4-{[1-(aminocarbonyl)-4-piperidinyl]amino}-1,6- |
| | diethyl-1H-pyrazolo[3,4-b]pyridin-5- |
| | yl)methyl]amino}carbonyl)phenyl]oxy}acetyl)amino]methyl}-1,6- |
| | diethyl-1H-pyrazolo[3,4-b]pyridin-4-yl)amino]-1- |
| | piperidinecarboxamide trifluoroacetate |
| 17 | N,N′-bis[(4-{[1-(aminocarbonyl)-4-piperidinyl]amino}-1,6-diethyl- |
| | 1H-pyrazolo[3,4-b]pyridin-5-yl)methyl]butanediamide trifluoroacetate |
| 18 | 4,4′-{methanediylbis[benzene-4,1- |
| | diyl(oxomethanediyl)iminomethanediyl(1,6-diethyl-1H- |
| | pyrazolo[3,4-b]pyridine-5,4-diyl)imino]}di(1- |
| | piperidinecarboxamide) trifluoroacetate |
| 18b | 4,4′-{methanediylbis[benzene-4,1- |
| | diyl(oxomethanediyl)iminomethanediyl(1,6-diethyl-1H- |
| | pyrazolo[3,4-b]pyridine-5,4-diyl)imino]}di(1- |
| | piperidinecarboxamide) |
| 19 | N,N′-bis[(4-{[1-(aminocarbonyl)-4-piperidinyl]amino}-1,6-diethyl- |
| | 1H-pyrazolo[3,4-b]pyridin-5-yl)methyl]-3,3- |
| | dimethylpentanediamide trifluoroacetate |
| 20 | N,N′-bis[(4-{[1-(aminocarbonyl)-4-piperidinyl]amino}-1,6-diethyl- |
| | 1H-pyrazolo[3,4-b]pyridin-5-yl)methyl]-1,3- |
| | cyclopentanedicarboxamide trifluoroacetate |
| 21 | 4,4′-{oxybis[(1-oxo-2,1-ethanediyl)iminomethanediyl(1,6- |
| | diethyl-1H-pyrazolo[3,4-b]pyridine-5,4-diyl)imino]}di(1- |
| | piperidinecarboxamide) trifluoroacetate |
| 21A | N,N′-bis[(4-{[1-(aminocarbonyl)-4-piperidinyl]amino}-1,6-diethyl- |
| | 1H-pyrazolo[3,4-b]pyridin-5-yl)methyl]-1,3- |
| | cyclohexanedicarboxamide trifluoroacetate |
| 22 | N,N′-bis{[1,6-diethyl-4-(tetrahydro-2H-pyran-4-ylamino)-1H- |
| | pyrazolo[3,4-b]pyridin-5-yl]methyl}-1,4-benzenedicarboxamide |
| 23 | N,N′-bis{[1,6-diethyl-4-(tetrahydro-2H-pyran-4-ylamino)-1H- |
| | pyrazolo[3,4-b]pyridin-5-yl]methyl}-4,4′-biphenyldicarboxamide |
| 23a | N,N′-bis{[1,6-diethyl-4-(tetrahydro-2H-pyran-4-ylamino)-1H- |
| | pyrazolo[3,4-b]pyridin-5-yl]methyl}-4,4′-biphenyldicarboxamide |
| | 1,5-naphthalenedisulfonate |
| 23b | N,N′-bis{[1,6-diethyl-4-(tetrahydro-2H-pyran-4-ylamino)-1H- |
| | pyrazolo[3,4-b]pyridin-5-yl]methyl}-4,4′-biphenyldicarboxamide |
| | 1,2,4-benzenetricarboxylate |
| 23c | N,N′-bis{[1,6-diethyl-4-(tetrahydro-2H-pyran-4-ylamino)-1H- |
| | pyrazolo[3,4-b]pyridin-5-yl]methyl}-4,4′-biphenyldicarboxamide |
| | para-toluenesulfonate |
| 24 | 4,4′-oxybis(N-{[1,6-diethyl-4-(tetrahydro-2H-pyran-4-ylamino)- |
| | 1H-pyrazolo[3,4-b]pyridin-5-yl]methyl}benzamide) |
| 25 | N,N′-bis[(4-{[1-(aminocarbonyl)-4-piperidinyl]amino}-1,6-diethyl- |
| | 1H-pyrazolo[3,4-b]pyridin-5-yl)methyl]-1,4-benzenedicarboxamide |
| 26 | N,N′-bis[(4-{[1-(aminocarbonyl)-4-piperidinyl]amino}-1,6-diethyl- |
| | 1H-pyrazolo[3,4-b]pyridin-5-yl)methyl]-4,4′-biphenyldicarboxamide |
| 27 | 4,4′-{oxybis[benzene-4,1- |
| | diyl(oxomethanediyl)iminomethanediyl(1,6-diethyl-1H- |
| | pyrazolo[3,4-b]pyridine-5,4-diyl)imino]}di(1- |
| | piperidinecarboxamide) |
| 28 | N,N′-bis{[1-ethyl-6-methyl-4-(tetrahydro-2H-pyran-4-ylamino)- |
| | 1H-pyrazolo[3,4-b]pyridin-5-yl]methyl}-4,4′-biphenyldicarboxamide |
| 29 | N,N′-bis{[1-ethyl-6-methyl-4-(tetrahydro-2H-pyran-4-ylamino)- |
| | 1H-pyrazolo[3,4-b]pyridin-5-yl]methyl}-3,3′-biphenyldicarboxamide |
| 30 | N,N′-bis{[1-ethyl-6-methyl-4-(tetrahydro-2H-pyran-4-ylamino)- |
| | 1H-pyrazolo[3,4-b]pyridin-5-yl]methyl}-2,6- |
| | naphthalenedicarboxamide |
| 31 | N,N′-bis{[1,6-diethyl-4-(tetrahydro-2H-pyran-4-ylamino)-1H- |
| | pyrazolo[3,4-b]pyridin-5-yl]methyl}-2,2′-bipyridine-4,4′-dicarboxamide |
| 32 | N,N′-bis{[1,6-diethyl-4-(tetrahydro-2H-pyran-4-ylamino)-1H- |
| | pyrazolo[3,4-b]pyridin-5-yl]methyl}-2,5-pyrazinedicarboxamide |
| 33 | 2,2′-(methylimino)bis(N-{[1,6-diethyl-4-(tetrahydro-2H-pyran-4- |
| | ylamino)-1H-pyrazolo[3,4-b]pyridin-5-yl]methyl}acetamide) |
| | (non-preferred name) |
| 34 | N,N′-bis{[1,6-diethyl-4-(tetrahydro-2H-pyran-4-ylamino)-1H- |
| | pyrazolo[3,4-b]pyridin-5-yl]methyl}-2,2′-bipyridine-5,5′-dicarboxamide |
| 35 | N,N′-bis{[1,6-diethyl-4-(tetrahydro-2H-pyran-4-ylamino)-1H- |
| | pyrazolo[3,4-b]pyridin-5-yl]methyl}-2,5-furandicarboxamide |
| 36 | N,N′-bis{[1,6-diethyl-4-(tetrahydro-2H-pyran-4-ylamino)-1H- |
| | pyrazolo[3,4-b]pyridin-5-yl]methyl}-2,5-thiophenedicarboxamide |

### Examples 1 to 10

The appropriate di-carboxylic acids (0.035 mmol) were treated with 0.2 ml of a solution of HATU (0.64 g, 1.68 mmol) in DMF (4.6 ml) followed by DIPEA (0.04 ml). After 5 minutes, 0.2 ml of a solution of Intermediate 8 (0.581 g, 1.9 mmol) in DMF (4.6 ml) was dispensed into each reaction. The reactions were shaken for 5 minutes then allowed to stand at room temperature for 3 days. The solvent was evaporated and the residues dissolved in CHCl₃ and purified by passing through amino-methyl SPE cartridges (0.5 g) eluting with CHCl₃ then EtOAc:MeOH. The solvent was evaporated and the residues further purified by MDAP to give the examples as trifluoroacetate (TFA) salts.

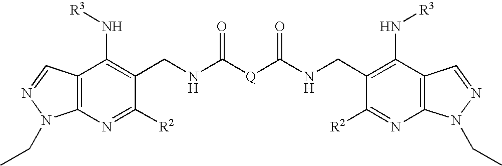

| Example Number | Starting material | R² | R³ | Di-acid starting material 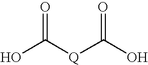 | One possible Source of Di- acid | LCMS m/z T_{RET} MH⁺ (min) | |
|---|---|---|---|---|---|---|---|
| 1 | Intermed- iate 8 | Et | 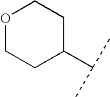 | 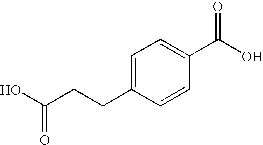 | Aldrich | 765 | 2.40 |
| 2 | Intermed- iate 8 | Et | 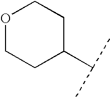 | 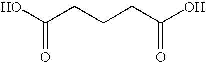 | Aldrich | 703 | 2.25 |
| 3 | Intermed- iate 8 | Et | 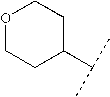 | 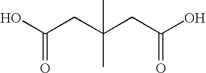 | Aldrich | 731 | 2.35 |
| 4 | Intermed- iate 8 | Et | 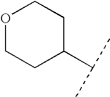 | 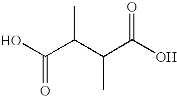 | Aldrich | 717 | 2.26 |
| 5 | Intermed- iate 8 | Et | 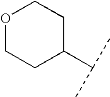 | 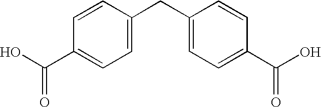 | ABCR | 827 | 2.71 |
| 6 | Intermed- iate 8 | Et | 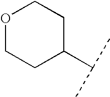 | 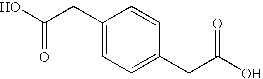 | Aldrich | 765 | 2.36 |
| 7 | Intermed- iate 8 | Et | 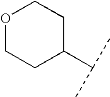 | 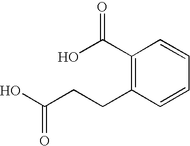 | Aldrich | 765 | 2.4 |
| 8 | Intermed- iate 8 | Et | 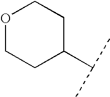 | 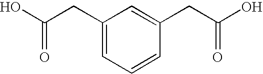 | Aldrich | 765 | 2.38 |
| 9 | Intermed- iate 8 | Et | 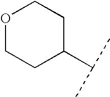 | 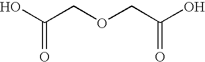 | Aldrich | 705 | 2.25 |
| 10 | Intermed- iate 8 | Et | 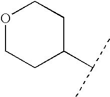 | 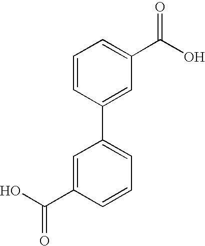 | Woods et al.; J. Am. Chem. Soc.; 72; 1950; 3221. | 813 | 2.74 |

### Example 2b Alternative Preparation

### N,N′-bis{[1,6-diethyl-4-(tetrahydro-2H-pyran-4-ylamino)-1H-pyrazolo[3,4-b]pyridin-5-yl]methyl}pentanediamide

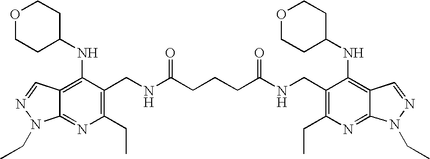

A solution of pentanedioic acid (130 mg, μmol) in DMF (10 ml) was treated with HATU (760 mg, 2 mmol) and DIPEA (1 ml, 6 mmol) and stirred at room temperature for 10 minutes. Solid Intermediate 8 (600 mg, 1.98 mmol) was added and the mixture stirred at room temperature for 16 hours. The mixture was added to a rapidly stirred mixture of saturated aqueous NaHCO₃ solution/water (1:1, 200 ml). The mixture was extracted with EtOAc (3×200 ml), the organic fractions combined, dried (Na₂SO₄) and evaporated. The residue was purified by recrystallisation from diethyl ether to give the title compound (462 mg). LCMS m/z 703 [MH⁺]; T_{RET}=2.16 min.

### Example 9b Alternative Preparation

### 2,2′-oxybis(N-{[1,6-diethyl-4-(tetrahydro-2H-pyran-4-ylamino)-1H-pyrazolo[3,4-b]pyridin-5-yl]methyl}acetamide)

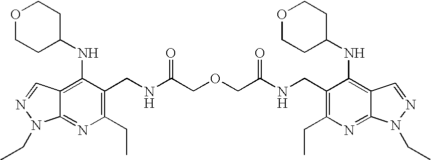

A solution of 2,2′-oxydiacetic acid (134 mg, 1 mmol) in DMF (10 ml) was treated with HATU (760 mg, 2 mmol) and DIPEA (1 ml, 6 mmol) and stirred at room temperature for 10 minutes. Solid Intermediate 8 (600 mg, 1.98 mmol) was added and the mixture stirred at room temperature for 16 hours. The mixture was added to a rapidly stirred mixture of saturated aqueous NaHCO₃ solution/water (1:1, 200 ml) and the mixture stirred for 20 minutes. The solid was collected by filtration and recrystallised from isopropanol to give the title compound (336 mg) as a white solid. LCMS showed M⁻=703; T_{RET}=2.14 min.

### Examples 11 to 21A

The appropriate di-carboxylic acids (0.035 mmol) were treated with 0.2 ml of a solution of HATU (0.64 g, 1.68 mmol) in DMF (4.6 ml) followed by DIPEA (0.04 ml). After 5 minutes, 0.2 ml of a solution of Intermediate 13 (0.662 g, 1.9 mmol) in DMF (4.6 ml) was dispensed into each reaction. The reactions were shaken for 5 minutes then allowed to stand at room temperature for 3 days. The solvent was evaporated and the residues dissolved in CHCl₃ and purified by passing through amino-methyl SPE cartridges (0.5 g) eluting with CHCl₃ then EtOAc:MeOH. The solvent was evaporated and the residues further purified by MDAP to give the examples as trifluoroacetate (TFA) salts.

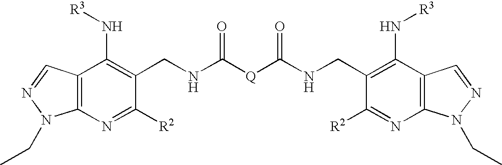

| Example Number | Starting material | R² | R³ | Di-acid starting material 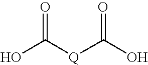 | One possible Source of Di- acid | LCMS m/z T_{RET} MH⁺ (min) | |
|---|---|---|---|---|---|---|---|
| 11 | Inter- mediate 13 | Et | 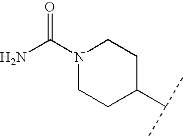 | 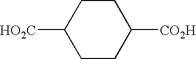 | Aldrich | 827 | 2.19 |
| 12 | Inter- mediate 13 | Et | 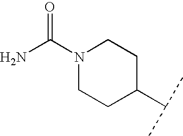 | 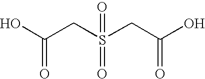 | ASINEX- REAG | 837 | 2.12 |
| 13 | Inter- mediate 13 | Et | 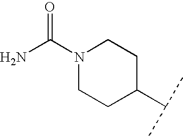 | 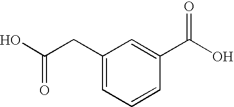 | H. Des Abbayes et. al., Tetrahed- ron Letters (1983), 24(37), 4005-8. | 835 | 2.18 |
| 14 | Inter- mediate 13 | Et | 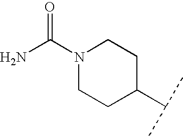 | 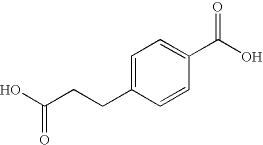 | Aldrich | 849 | 2.20 |
| 15 | Inter- mediate 13 | Et | 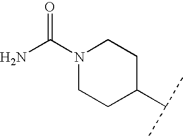 | 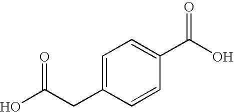 | Ubichem | 835 | 2.21 |
| 16 | Inter- mediate 13 | Et | 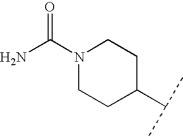 | 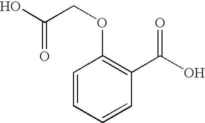 | Lan- caster | 851 | 2.23 |
| 17 | Inter- mediate 13 | Et | 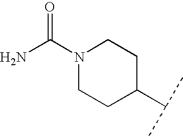 | 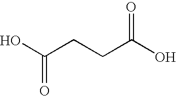 | Aldrich | 773 | 2.05 |
| 18 | Inter- mediate 13 | Et | 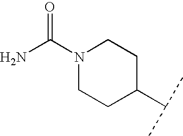 | 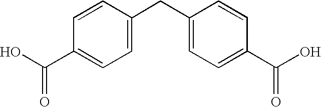 | ABCR | 456 [(M + 2H)²⁺/ 2] | 2.45 |
| 19 | Inter- mediate 13 | Et | 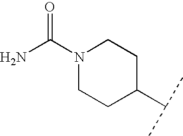 | 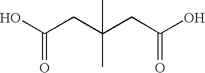 | Aldrich | 408 [(M + 2H)²⁺/ 2] | 2.16 |
| 20 | Inter- mediate 13 | Et | 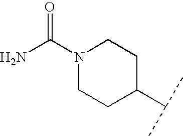 | 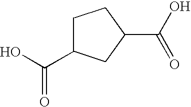 | Rieke | 407 [(M + 2H)²⁺/ 2] | 2.16 |
| 21 | Inter- mediate 13 | Et | 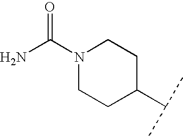 | 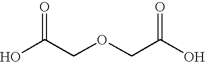 | Aldrich | 789 | 2.10 |
| 21A | Inter- mediate 13 | Et | 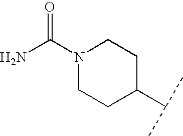 | 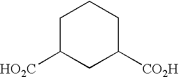 | Aldrich | 414 [(M + 2H)²⁺/ 2] | 2.19 |

### Example 18b

### 4,4′-{methanediylbis[benzene-4,1-diyl(oxomethanediyl)iminomethanediyl(1,6-diethyl-1H-pyrazolo[3,4-b]pyridine-5,4-diyl)imino]}di(1-piperidinecarboxamide)

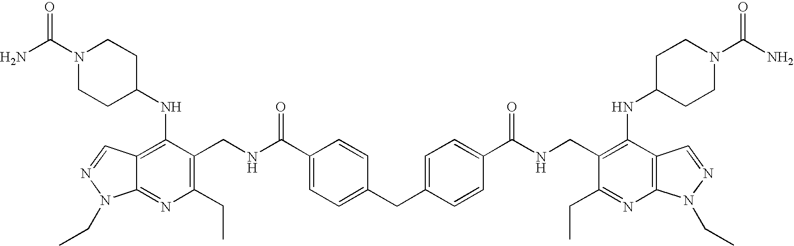

A solution of 4,4′-methanediyldibenzoic acid (555 mg, 2.17 mmol, e.g. available from Fluorochem) in DMF was treated with HATU (1.81 g, 4.77 mmol) followed by DIPEA (2.2 ml, 13.02 mmol) and allowed to stir at room temperature for 5 minutes. Intermediate 13 (1.5 g, 4.34 mmol) was added and the solution stirred for a further 2 h. The mixture was poured into a mixture of saturated aqueous NaHCO₃ solution/water (400 ml, 1:1). After stirring for 5 minutes the solid was collected by filtration and recrystallised from isopropanol and cyclohexane to give the title compound (989 mg) as an off-white solid. LCMS m/z 456 [(M+2H)²⁺/2]; T_{RET}=2.34 min.

### Examples 22, 23 and 24

A solution of HATU (61 mg, 0.16 mmol) in DMF (2 ml) was dispensed onto the appropriate solid dicarboxylic acids (0.08 mmol) followed by DIPEA (0.1 ml) and the mixtures were allowed to stand for 10 minutes. A solution of Intermediate 8 (50 mg, 0.16 mmol) was added and the mixtures were allowed to stand at room temperature for 16 hours. The solvent was evaporated and the residues were purified by MDAP.

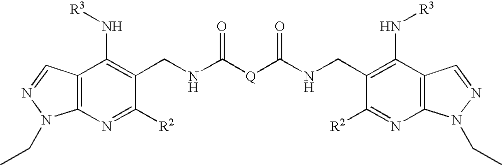

| Example No. | Starting material | R² | R³ | Di-acid starting material 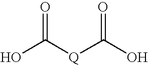 | One possible Source of Di-acid | LCMS m/z T_{RET} [(M + 2H)²⁺/2] (min) | |
|---|---|---|---|---|---|---|---|
| 22 | Intermediate 8 | Et | 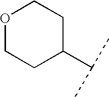 | 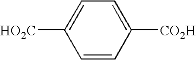 | Aldrich | 369 | 2.42 |
| 23 | Intermediate 8 | Et | 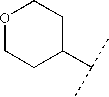 | 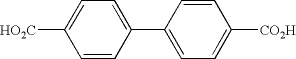 | Aldrich | 407 | 2.68 |
| 24 | Intermediate 8 | Et | 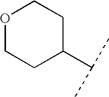 | 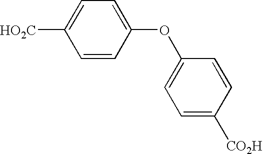 | Aldrich | 415 | 2.68 |

### Example 23

### Alternative Preparation No. 1

### N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide

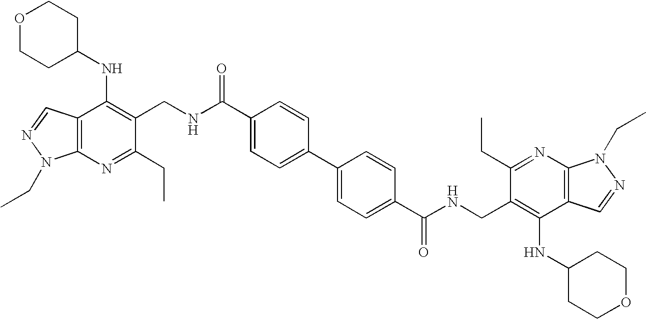

A solution of 4,4′-biphenyldicarboxylic acid (0.8 g, 3.3 mmol), DIPEA (3.4 ml, 19 mmol) in DMF (30 ml) was treated with HATU (3.0 g, 8.0 mmol) and stirred under nitrogen for 10 minutes. The resulting suspension was treated with Intermediate 8 (2.0 g, 6.6 mmol) and stirred at room temperature for 16 hours. The resulting suspension was added to a mixture of saturated aqueous NaHCO₃ solution/water (1:1, 350 ml) and the mixture was stirred for 5 minutes and the solid collected by filtration. The cream solid was washed with water (2×20 ml) and air dried. The damp solid was triturated with hot isopropanol (about 70 ml) followed by trituration with diethyl ether (20 ml) to give the title compound as a white powder (1.85 g). LCMS m/z 407 [(M+2H)²⁺/2]; T_{RET}=2.63 min.

### Example 23

### Alternative Preparation No. 2

### N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide

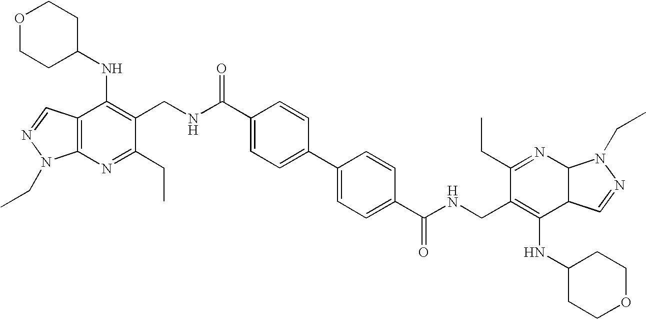

A solution of 4,4′-biphenyldicarboxylic acid (1.82 g, 7.5 mmol), DIPEA (7.7 ml, 43.2 mmol) in DMF (50 ml) was treated with HATU (6.82 g, 18.8 mmol) and stirred under nitrogen for 10 minutes. The resulting suspension was treated with Intermediate 8 (4.55 g, 15 mmol) and stirred at room temperature for 3 hours. The mixture was added to a mixture of saturated aqueous NaHCO₃ solution/water (1:1, 600 ml) and the mixture was stirred for 20 minutes and the solid collected by filtration. The solid was washed with water and diethyl ether, and then air dried. The solid was pre-absorbed onto silica and purified by flash chromatography eluting with DCM/MeOH (9:1). Fractions containing the product were combined and evaporated to dryness. The residue was triturated with diethyl ether, collected by filtration, washed with diethyl ether, and dried under vacuum to give the title compound (4.76 g). LCMS m/z 407 [(M+2H)²⁺/2]; T_{RET}=2.61 min. 1H NMR (400 MHz, CDCl₃) δ (delta) ppm 1.38 (6H, t), 1.48 (6H, t), 1.78 (4H, m), 2.09 (4H, m), 2.94 (4H, q), 3.58 (4H, m), 4.06 (6H, m), 4.45 (4H, q), 4.77 (4H, d), 6.40 (2H, t), 6.73 (2H, d), 7.66 (4H, d), 7.85 (4H, d), 7.86 (2H, s).

### Example 23

### Alternative Preparation No. 3

### N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide

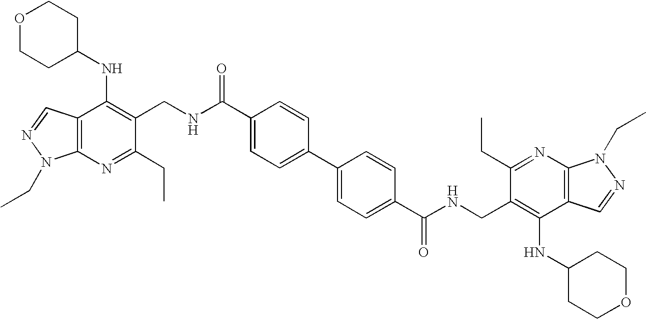

DIPEA (60 ml) was added to a suspension of 4,4′-biphenyldicarboxylic acid (14.6 g, 60.4 mmol) in dry DMF. HBTU (54.9 g, 145 mmol) was added and the mixture stirred for 10 minutes. Intermediate 8 (40.35 g, 133 mmol) was added with additional DMF (50 ml) and the mixture stirred overnight (e.g. at room temperature). The suspension was poured into a mixture of saturated aqueous sodium bicarbonate solution/water (1:1, 2.5 L), stirred for 20 minutes and then filtered. The solid was washed with a mixture of saturated aqueous sodium bicarbonate solution/water (1:1, 300 ml) and dried. It was then stirred in isopropanol for 2 h, filtered, washed with isopropanol followed by diethylether, and dried under vacuum to give the title compound (50.01 g). LCMS m/z 407 [(M+2H)²⁺/2]; T_{RET}=2.67 min.

### Example 23a

### N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide 1,5-naphthalenedisulfonate

MeOH (30 ml) was added to N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide (the “free base”, 2 g), and the resulting mixture was stirred at 70° C. for an hour. After an hour, the free base had not dissolved. 1,5-Naphthalenedisulfonic acid tetrahydrate (about 1.86 g, about 2.1 equivalents) was dissolved in MeOH (10 ml). This solution of the 1,5-naphthalenedisulfonic acid was then added to the undissolved free base/MeOH mixture slowly throughout the course of the day while the reaction was temperature cycling (0 to 40° C.). The reaction was seeded twice. The reaction was left to temperature cycle (0 to 40° C.) over the weekend. The solid product was isolated by filtration and washed with MeOH, was left on the filter paper for a while, and then was dried in a vacuum oven overnight at 40° C. Isolated yield=2.58 g.

¹H NMR [400 MHz, d6-DMSO (solvent), TMS (standard, 0.00 ppm)] δ (delta) ppm 1.31 (6H, t), 1.38 (6H, t), 1.69 (4H, m), 1.97 (4H, brd), 3.10 (4H, m), 3.63 (4H, t), 3.92 (4H, br d), 4.34 (2H, br m), 4.45 (4H, m), 4.60 (4H, br d), 7.39 (2H, t), 7.90 (6H, m), 8.00 (4H, d), 8.43 (2H, br m), 8.85 (2H, d), 9.04 (2H, br m), 9.26 (2H, br m), 13.24 (2H, br s). Stoich-iometry by ¹H NMR: very approximately 1:1 free base: 1,5-naphthalenedisulfonic acid.

The X-ray powder diffraction (XRPD) data of the solid product: The data were acquired on a PANalytical X'Pert Pro powder diffractometer, model PW3040/60, serial number DY1850 using an XCelerator detector. The acquisition conditions were approximately: radiation: Cu Kα (Cu K-alpha); generator tension: 40 kV; generator current: 45 mA; start angle: 2.0°. 2.θ; end angle: 40.0°. 2θE; step size: 0.0167° 2θ; time per step: 31.75 seconds. The sample was prepared by mounting a few milligrams of sample on a Si wafer (zero background) plate(s), resulting in a thin layer of powder. XRPD angles for certain XRPD peaks or characteristic XRPD peaks, and calculated d-spacings (lattice spacings) therefor, are generally as recorded in Table 1 below (peak positions were measured using Highscore software).

TABLE 1

| 2θ/° | d-spacing/A |
|---|---|
| (2 theta/degrees) | (Angstroms) |
| 8.4 | 10.5 |
| 8.7 | 10.2 |
| 10.4 | 8.5 |
| 11.9 | 7.5 |
| 12.9 | 6.9 |
| 13.8 | 6.4 |
| 14.0 | 6.3 |
| 14.8 | 6.0 |
| 15.6 | 5.7 |
| 17.0 | 5.2 |
| 17.7 | 5.0 |
| 19.1 | 4.7 |
| 20.4 | 4.3 |
| 20.8 | 4.3 |
| 21.3 | 4.2 |
| 22.6 | 3.9 |
| 22.9 | 3.9 |
| 26.6 | 3.3 |
| 28.1 | 3.2 |

### Example 23b

### N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide 1,2,4-benzenetricarboxylate

MeOH (25 ml) was added to N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide (the “free base”, 2 g, about 2.46×10⁻³ moles), and the resulting mixture was heated at 70° C. for an hour while stirring to try to dissolve the free base. After about half an hour, the temperature was lowered to 50° C. and MeOH (10 ml) was added as the slurry was thick. 1,2,4-Benzenetricarboxylic acid (1.086 g, about 2.1 equivalents) was dissolved in MeOH. This solution of the 1,2,4-benzenetricarboxylic acid was then added to the free base/MeOH mixture, dropwise over 2 hours at 50° C. After the solution of the acid was added, the reaction was left temperature cycling (0 to 40° C.) over the weekend. The solid was isolated from the resulting slurry by filtration, was washed with MeOH (about 2 ml), and was left on the filter paper for about 2 hours. The isolated solid product was then dried in a vacuum oven overnight at 40° C. Isolated yield=about 2.61 g. Stoichiometry by ¹H NMR: very approximately 1:0.905 free base: 1,2,4-benzenetricarboxylic acid.

### Example 23c

### N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide para-toluenesulfonate MeOH (22 ml) was added to N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide (the “free base”, 1600 mg (1.6 g), about 1.97×10⁻³ moles), and the resulting mixture was heated at 70° C. for an hour to try to dissolve the free base. MeOH (10 ml) was added to para-toluenesulfonic acid monohydrate (786 mg, about 2.1 equivalents). The solution of the para-toluenesulfonic acid was then added to the undissolved free base/MeOH mixture slowly over the course of a day. While the acid solution was being added, the reaction was temperature cycled (0 to 40° C.). The reaction was left to temperature cycle (0 to 40° C.) for about 2 days. The resulting white solid product was isolated by filtration and washed with MeOH, was left on the filter paper for a while, and then was dried in a vacuum oven overnight at 40° C. Isolated yield=about 1.98 g. Stoichiometry by ¹H NMR: very approximately 1:1.92 free base:para-toluenesulfonic acid. This particular solid product is thought to be a solvate (e.g. methanol solvate).

### Example 23c

### Alternative Preparation

N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide para-toluenesulfonate

MeOH (6 ml) was added to N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide (the “free base”, 300 mg, about 3.69×10⁻⁴ moles), and the resulting mixture was heated at 70° C. for an hour to try to dissolve the free base. Since the free base did not dissolve after an hour, para-toluenesulfonic acid monohydrate (147 mg, about 2.1 equivalents) was added. The reaction was then left to temperature cycle (0 to 40° C.) for about 3 days. The resulting solid product was isolated by filtration, washed with solvent (e.g. MeOH), was left on the filter paper for a while, and then was dried in a vacuum oven at 40° C. over the weekend. Isolated yield=about 336 mg. Stoichiometry by ¹H NMR: very approximately 1:2 free base:para-toluenesulfonic acid.

### Examples 25, 26 and 27

A solution of HATU (55 mg, 0.16 mmol) in DMF (2 ml) was dispensed onto the appropriate solid dicarboxylic acids (0.072 mmol) followed by DIPEA (0.1 ml), and the mixtures were allowed to stand for 10 minutes. A solution of Intermediate 13 (50 mg, 0.145 mmol) was added and the mixtures were allowed to stand at room temperature for 16 hours. The solvent was evaporated and the residues were purified by MDAP.

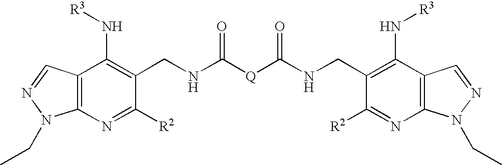

| Example No. | Starting material | R² | R³ | Di-acid starting material 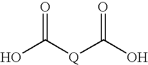 | One possible Source of Di-acid | LCMS m/z T_{RET} [(M + 2H)²⁺/2] (min) | |
|---|---|---|---|---|---|---|---|
| 25 | Intermediate 13 | Et | 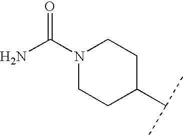 | 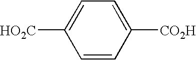 | Aldrich | 411 | 2.24 |
| 26 | Intermediate 13 | Et | 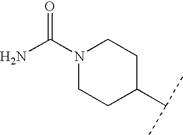 | 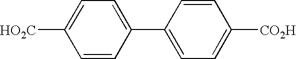 | Aldrich | 449 | 2.44 |
| 27 | Intermediate 13 | Et | 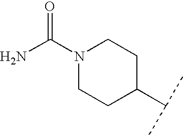 | 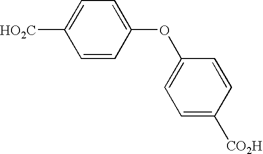 | Aldrich | 457 | 2.40 |

### Example 28

### N,N′-bis{[1-ethyl-6-methyl-4-(tetrahydro-2H-pyran-4-ylamino)-1H-pyrazolo[3,4-b]pyridin-5-yl]methyl}-4,4′-biphenyldicarboxamide

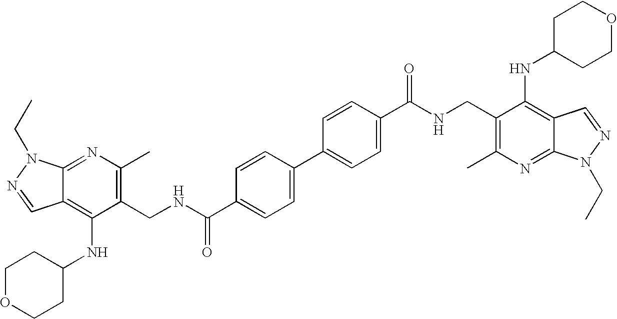

A solution of 4,4′-biphenyldicarboxylic acid (28 mg, 0.12 mmol) in DMF (2 ml) was treated with HATU (88 mg, 0.23 mmol) and DIPEA (92 μL (92 microlitres), 0.53 mmol) and allowed to stir at room temperature for 30 minutes. Intermediate 18 (60 mg, 0.21 mmol) was added and the mixture allowed to stand at room temperature overnight. The solvent was evaporated; the residue dissolved in CHCl₃ and passed through an amino propyl SPE (5 g) cartridge eluting with CHCl₃ and EtOAc:MeOH (4:1). The solvents were evaporated and the residue purified by MDAP. Further purification on an aminopropyl SPE cartridge (2 g) gave the title compound (14 mg) as a white solid. LCMS m/z 393 [(M+2H)²⁺/2]; T_{RET}=2.57 min.

### Example 29

### N,N′-bis{[1-ethyl-6-methyl-4-(tetrahydro-2H-pyran-4-ylamino)-1H-pyrazolo[3,4-b]pyridin-5-yl]methyl}-3,3′-biphenyldicarboxamide

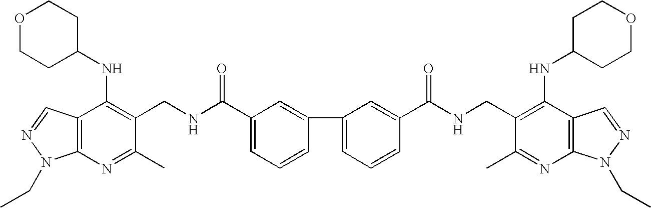

A solution of 3,3′-biphenyldicarboxylic acid (e.g. obtainable by Woods et al.; J. Am. Chem. Soc.; 72; 1950; 3221) (0.625 g, 2.6 mmol) in DMF (20 ml) was treated with HATU (1.45 g, 5.2 mmol) followed by DIPEA (3.5 mL, 20.8 mmol) and the mixture was stirred for 10 minutes at 22° C. Intermediate 18 (1.5 g, 5.2 mmol) was added in one portion and the mixture stirred at 22° C. overnight. The mixture was poured into a mixture of saturated aqueous NaHCO₃ solution/water (1:1, 400 ml) and extracted with DCM. After evaporation to dryness the residue was taken up in EtOAc (150 ml) and washed with saturated LiCl solution (100 ml) and water (100 ml), dried (Na₂SO₄) and evaporated. The residue was purified on a silica SPE cartridge eluting with 0-100% EtOAc in cyclohexane gradient (increasing in EtOAc concentration) followed by a 0-50% MeOH in EtOAc gradient (increasing in MeOH concentration). The product was recrystallised from DCM/cyclohexane to give the title compound (303 mg). LCMS m/z=785 [MH⁺]; T_{RET}=2.52 min.

### Example 30

### N,N′-bis{[1-ethyl-6-methyl-4-(tetrahydro-2H-pyran-4-ylamino)-1H-pyrazolo[3,4-b]pyridin-5-yl]methyl}-2,6-naphthalenedicarboxamide

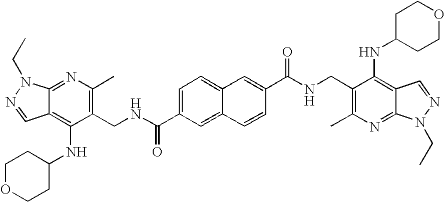

A solution of 2,6-naphthalenedicarboxylic acid (e.g. available from Aldrich, 24 mg, 0.11 mmol) in dry DMF (4 ml) was treated with PyBOP (256 mg, 0.49 mmol) followed by DIPEA (155 mL, 0.89 mmol) and allowed to stir at room temperature for 10 minutes. To the resultant white suspension was added a solution of Intermediate 18 (73.6 mg, 0.245 mmol) in dry DMF (2 ml) to give grey solution which was allowed to stand at room temperature for 4 hours. The solvent was evaporated and the residue was dissolved in CHCl₃ (3 ml), washed with aqueous NaHCO₃ solution (1M, 3 ml), and the organic collected by passing through a hydrophobic frit. The title compound precipitated from the CHCl₃ extract and was collected by filtration as a white solid (80 mg). LCMS m/z 380 [(M+2H)²⁺/2]; T_{RET}=2.42 min.

### Examples 31 to 36

The appropriate di-carboxylic acid (0.13 mmol) in DMF (3 ml) was treated with HATU (0.13 mmol) and DIPEA (0.33 mmol) and allowed to stir or stand at room temperature for 20 minutes. A solution of Intermediate 8 (either 20 mg, 0.066 mmol; or 200 mg, 0.66 mmol) in DMF was added to each of the reaction mixtures. The mixtures were allowed to stir or stand at room temperature for 3 hours. A further portion of HATU (0.13 mmol) and DIPEA (0.33 mmol) was then added to each reaction and the resulting solutions were allowed to stir or stand at room temperature overnight. The solvent was evaporated and the residue dissolved in CHCl₃ and loaded onto a 0.5 g amino-propyl SPE cartridge (pre-conditioned with EtOAc:MeOH 9:1). The cartridge was eluted with 2 column lengths of CHCl₃ and 2 column lengths of EtOAc:MeOH (9:1). The solvent was evaporated and the residues purified by MDAP.

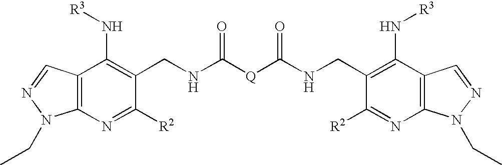

| Example No. | Starting material | R² | R³ | Di-acid starting material 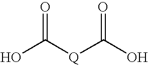 | One possible Source of Di- acid | LCMS m/z [MH]⁺ | LCMS T_{RET} (min) |
|---|---|---|---|---|---|---|---|
| 31 | Inter- mediate 8 | Et | 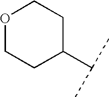 | 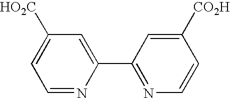 | Aldrich | 815 | 2.68 |
| 32 | Inter- mediate 8 | Et | 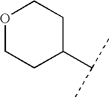 | 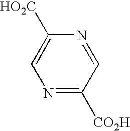 | Tyger | 739 | 2.68 |
| 33 | Inter- mediate 8 | Et | 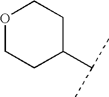 | 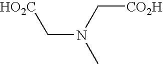 | Aldrich | 718 | 2.68 |
| 34 | Inter- mediate 8 | Et | 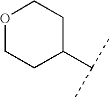 | 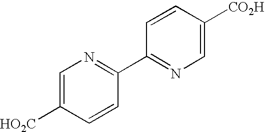 | Aldrich | 408 [(M + 2H)²⁺/ 2] | 2.55 |
| 35 | Inter- mediate 8 | Et | 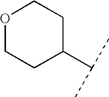 | 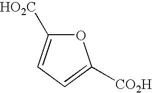 | Apin | 727 | 2.43 |
| 36 | Inter- mediate 8 | Et | 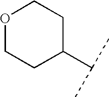 | 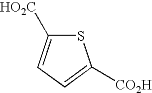 | Aldrich | 743 | 2.51 |

### Example 32

### Alternative Preparation

### N,N′-bis{[1,6-diethyl-4-(tetrahydro-2H-pyran-4-ylamino)-1H-pyrazolo[3,4-b]pyridin-5-yl]methyl}-2,5-pyrazinedicarboxamide

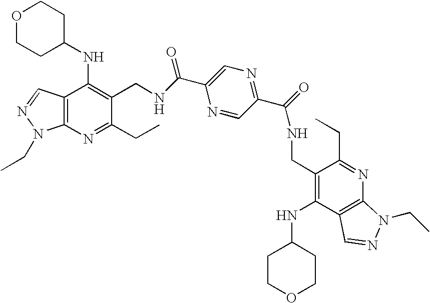

A solution of 2,5-pyrazinedicarboxylic acid (e.g. available from Tyger, 185 mg, 1.1 mmol), HOBT (300 mg, 2.2 mmol), DCC (453 mg, 2.2 mmol) and DIPEA (1.1 ml, 6.6 mmol) in DMF (8 ml) was stirred for 10 minutes. A solution of Intermediate 8 (1.33 g, 4.4 mmol) was added in DMF and the mixture allowed to stir at ambient temperature over the weekend (about 72 hours). The solvent was evaporated and the residue partitioned between water and DCM. The organic fraction was collected through a hydrophobic frit and evaporated to give a brown foam. The foam was purified by pre-absorbing onto silica and passing through a 50 g silica SPE cartridge eluting with a 0-100% EtOAc in cyclohexane gradient (increasing in EtOAc concentration) followed by a 0-20% EtOAc in MeOH gradient (increasing in MeOH concentration). Fractions containing product were pooled and evaporated then recrystallised from IPA/DCM to give the title compound (384 mg) as a yellow solid. LCMS m/z 739 [MH⁺]; T_{RET}=2.28 min.

### Example 35

### Alternative Preparation

### N,N′-bis{[1,6-diethyl-4-(tetrahydro-2H-pyran-4-ylamino)-1H-pyrazolo[3,4-b]pyridin-5-yl]methyl}-2,5furandicarboxamide

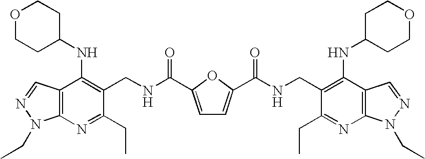

A solution of 2,5furandicarboxylic acid (e.g. available from Apin, 171 mg, 1.1 mmol), HOBT (300 mg, 2.2 mmol), DCC (453 mg, 2.2 mmol) and DIPEA (1.1 ml, 6.6 mmol) in DMF (8 ml) was stirred for 10 minutes. A solution of Intermediate 8 (1.33 g, 4.4 mmol) was added in DMF and the mixture allowed to stir at ambient temperature over the weekend (about 72 hours) followed by heating at 100° C. for 2 hours. The mixture was poured into a rapidly stirred saturated aqueous NaHCO₃ solution and stirring continuing for 10 mins. The resultant solid was collected by filtration. The solid was pre-absorbed onto silica and purified by passing through a 50 g silica SPE cartridge and eluting with 0-50% MeOH in DCM gradient (increasing in MeOH concentration). Fractions containing product were pooled and evaporated. The solid was triturated with IPA to give the title compound as a white solid. LCMS m/z 727 [MH⁺]; T_{RET}=2.41 min.

1-44. (canceled)

45. A compound of formula (I) or a salt thereof: 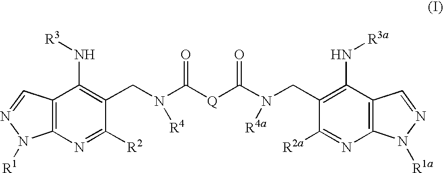
wherein:
Q is —(CH₂)ₘ¹—Ar¹—(CH₂)ₘ²—; —(CMe₂)—Ar²—(CMe₂)-; —(CHMe)—Ar³—(CHMe)-; —(CH₂)ₘ¹—Ar⁴—O—CH₂—; 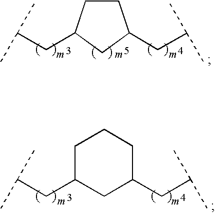
—(CH₂)ₘ⁶—X¹—(CH₂)ₘ⁷—; —(CMe₂)—X²—(CMe₂)-; or —(CHMe)—X³—(CHMe)-;
m¹ is 0, 1 or 2;
m² is 0 or 1;
m³ is 0 or 1;
m⁴ is 0 or 1;
and m⁵ is 1 or 2;
m⁶ is 0, 1, 2, 3, 4 or 5;
m⁷ is 0, 1, 2, 3, 4 or 5;
X¹ is —CH₂—, —CMe₂-, —CHMe-, O, S(O)₂, or NR⁵ wherein R⁵ is H or C₁₋₃alkyl, provided that when X¹ is O or S(O)₂ then m⁶ and m⁷ independently are 1, 2, 3, 4 or 5;
X² and X³ are independently: a bond, —CH₂—, —(CH₂)₂—, or —(CH₂)₃—;
and Ar¹, Ar², Ar³ and Ar⁴ independently have the sub-formula (x1), (x2), (x3), (x4), (x5), (x6), (x7), (x8), (x9), (x10), (x11), (x12), (x13), (x14), (x15), (x16), (×17), (×18), (×19), (x20), (x21), (x22), (x23), (x24), (x25), (x26), (x27), (x28) or (x29): 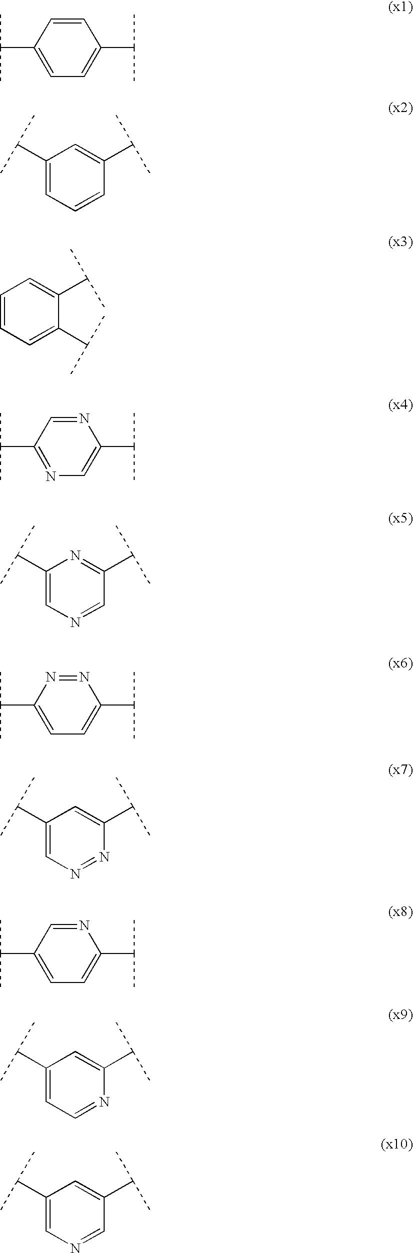 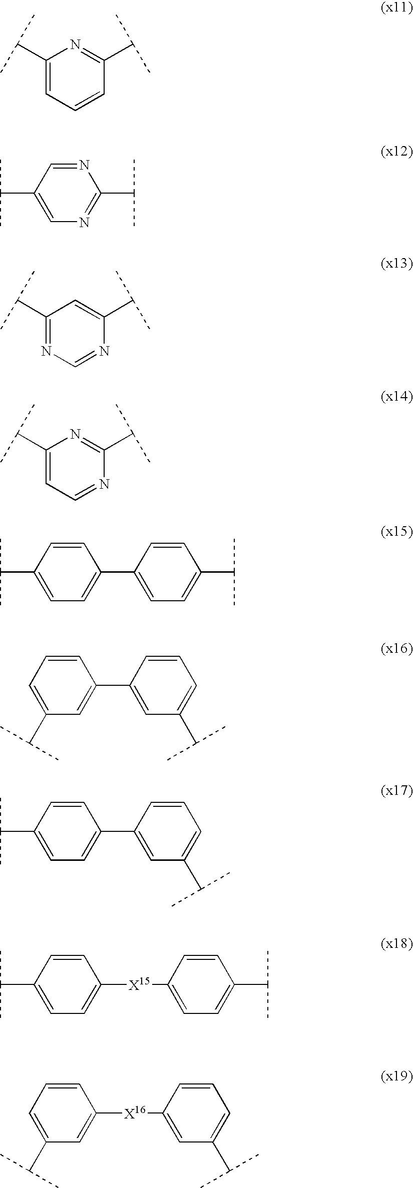 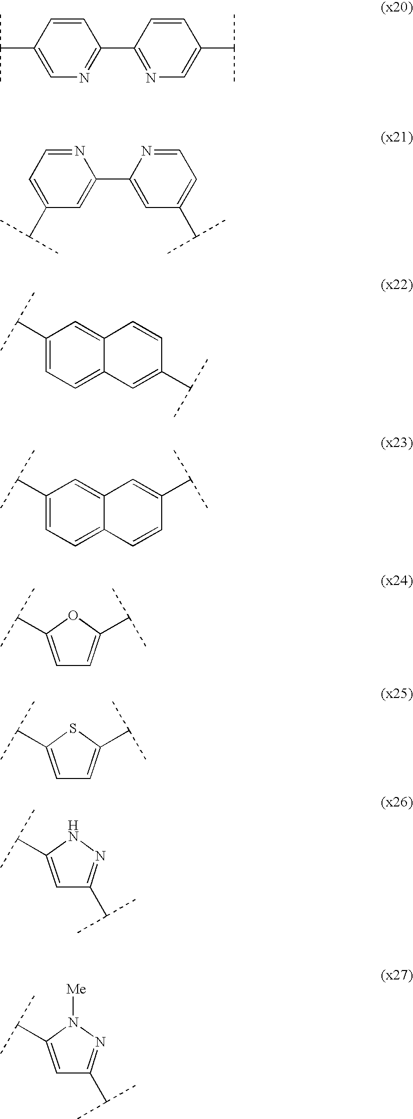 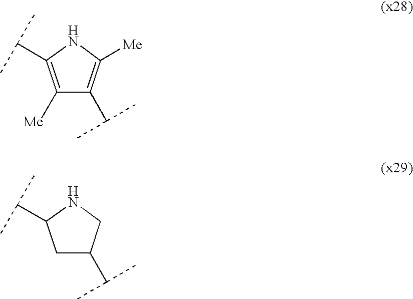
wherein X¹⁵ and X¹⁶ are independently: —CH₂—, —CMe₂-, —CHMe-, —CF₂—, O, C(O), or CHOH;
and wherein:
R¹ and R^{1a} independently are C₁₋₃alkyl, C₁₋₃fluoroalkyl, or —CH₂CH₂OH;
R² and R^{2a} independently are hydrogen, methyl, ethyl, n-propyl, isopropyl, n-butyl, C₁₋₂fluoroalkyl, cyclopropyl, cyclobutyl, or (cyclopropyl)methyl-;
R⁴ and R^{4a} independently are hydrogen, methyl or ethyl;
R³ and R^{3a} independently are optionally substituted C₄₋₇cycloalkyl, or optionally substituted mono-unsaturated-C₅₋₇cycloalkenyl, or an optionally substituted heterocyclic group of sub-formula (aa), (bb) or (cc), or a bicyclic group of sub-formula (ee); 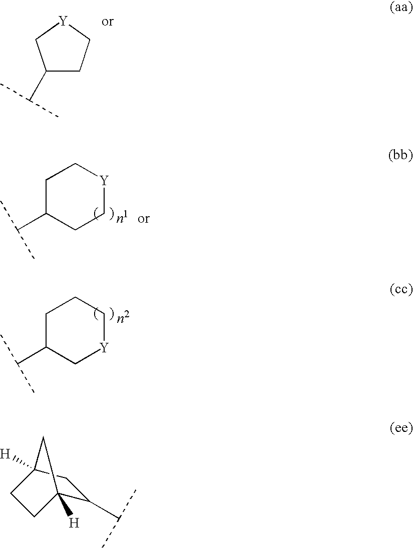
in which n¹ and n² independently are 1 or 2; and in which Y is O, S, SO₂, or NR¹⁰; where R¹⁰ is hydrogen, methyl, C(O)NH₂, C(O)-methyl, or C(O)—C₁fluoroalkyl;
and wherein, when R³ and R^{3a} are independently optionally substituted C₄₋₇cycloalkyl, then R³ and R^{3a} are C₄₋₇cycloalkyl optionally substituted on a ring carbon with one or two substituents independently being: oxo (═O); OH; methoxy; C₁fluoroalkoxy; NH₂; C₁₋₂alkyl; C₁fluoroalkyl; —CH₂OH; —CH(Me)OH; —CH₂CH₂OH; —CH₂NH₂; —C(O)OH; —C(O)NHR²⁴ wherein R²⁴ is H or methyl; —C(O)R²⁵ wherein R²⁵ is methyl; fluoro; hydroxyimino (═N—OH); or (C₁₋₂alkoxy)imino (═N—OR²⁶ where R²⁶ is C₁₋₂alkyl); and wherein any OH, methoxy, fluoroalkoxy or NH₂ substituent is not substituted at the R³ and R^{3a} ring carbon attached (bonded) to the —NH— group of formula (I);
and wherein, when R³ and R^{3a} are independently the optionally substituted heterocyclic group of sub-formula (aa), (bb) or (cc), then R³ and R^{3a} are the heterocyclic group of sub-formula (aa), (bb) or (cc) optionally substituted on a ring carbon with one or two substituents independently being oxo (═O), OH or methyl; and wherein any OH substituent is not substituted at the R³ and/or R^{3a} ring carbon attached (bonded) to the —NH— group of formula (I) and is not substituted at either R³ and R^{3a} ring carbon bonded to the Y group of the heterocyclic group (aa), (bb) or (cc);
and wherein, when R³ and R^{3a} is optionally substituted mono-unsaturated-C₅₋₇cycloalkenyl, then the cycloalkenyl is optionally substituted on a ring carbon with one substituent being fluoro or methyl, and the R³ and/or R^{3a} ring carbon bonded to the —NH— group of formula (I) does not partake in the cycloalkenyl double bond;
provided that:
when R³ and R^{3a} are independently the heterocyclic group of sub-formula (aa) and Y is NR¹⁰, then R¹⁰ is not C(O)-methyl, or C(O)—C₁fluoroalkyl; and
when R³ and R^{3a} are independently the heterocyclic group of sub-formula (bb), and Y is NR¹⁰, then R¹⁰ is not methyl; and
when R³ and/or R^{3a} is or are the heterocyclic group of sub-formula (cc), then Y is O, S, SO₂ or NR¹⁰ wherein R¹⁰ is H or methyl;
and wherein:
when R³ and R^{3a} are independently optionally substituted C₄₋₇cycloalkyl, then any —C(O)NHR²⁴ or —C(O)R²⁵ substituent on a ring carbon is: at the 3-position of a R³ and/or R^{3a} cyclobutyl ring; or at the 3- or 4-position of a R³ and/or R^{3a} cyclopentyl ring; or at the 4-position of a R³ and/or R^{3a} cyclohexyl ring; or at the 3-, 4-, 5- or 6-position of a R³ and/or R^{3a} cycloheptyl ring wherein, in this connection, the 1-position of the R³ and/or R^{3a} cycloalkyl ring is deemed to be the connection point to the —NH— in formula (I), that is the ring atom connecting to the —NH— in formula (I);
and wherein:
when R³ and R^{3a} are independently optionally substituted C₄₋₇cycloalkyl, then any OH, methoxy, fluoroalkoxy, —CH₂OH, —CH(Me)OH, —CH₂CH₂OH, —CH₂NH₂, or —C(O)OH substituent on a ring carbon is: at the 3-position of a R³ and R^{3a} are independently a cyclobutyl ring; or at the 3- or 4-position of a R³ and R^{3a} are independently a cyclopentyl ring; or at the 3-, 4- or 5-position of a R³ and/or R^{3a} cyclohexyl ring; or at the 3-, 4-, 5- or 6-position of a R³ and R^{3a} are independently a cycloheptyl ring; and
and wherein:
when R³ and R^{3a} are independently the heterocyclic group of sub-formula (aa), (bb) or (cc), then any OH substituent on a ring carbon is: at the 5-position of a six-membered R³ and R^{3a} are independently a heterocyclic group of sub-formula (cc) wherein n² is 1; or at the 5- or 6-position of a seven-membered R³ and R^{3a} are independently a heterocyclic group of sub-formula (cc) wherein n² is 2; or at the 6-position of a seven-membered R³ and R^{3a} are independently a heterocyclic group of sub-formula (bb) wherein n¹ is 2 wherein, in this connection, the 1-position of the R³ and/or R^{3a} heterocyclic ring is deemed to be the connection point to the —NH— in formula (I), that is the ring atom connecting to the —NH— in formula (I), and the remaining positions of the ring are then numbered so that the ring heteroatom takes the lowest possible number.

46. A compound or salt as claimed in claim 45, wherein R¹ and R^{1a} are ethyl.

47. A compound or salt as claimed in claim 45, wherein R² and R^{2a} are the same and are methyl or ethyl.

48. A compound or salt as claimed in claim 45, wherein R³ and R^{3a} are independently the optionally substituted C₄₋₇cycloalkyl or the optionally substituted heterocyclic group of sub-formula (aa), (bb) or (cc).

49. A compound or salt as claimed in claim 45, wherein, when R³ and R^{3a} are independently the heterocyclic group of sub-formula (aa), (bb) or (cc), then Y is O or NR¹⁰.

50. A compound or salt as claimed in claim 45, wherein R¹⁰ is C(O)NH₂.

51. A compound or salt as claimed in claim 45, wherein, when R³ and R^{3a} are independently the heterocyclic group of sub-formula (aa), (bb) or (cc), then R³ and R^{3a} are independently the heterocyclic group of sub-formula (bb) and n¹ is 1.

52. A compound or salt as claimed in claim 45, wherein NHR³ and NHR^{3a} are independently sub-formula (c), (h), (k2), (k3), (n), (o), (o2), (p9) or (p13).

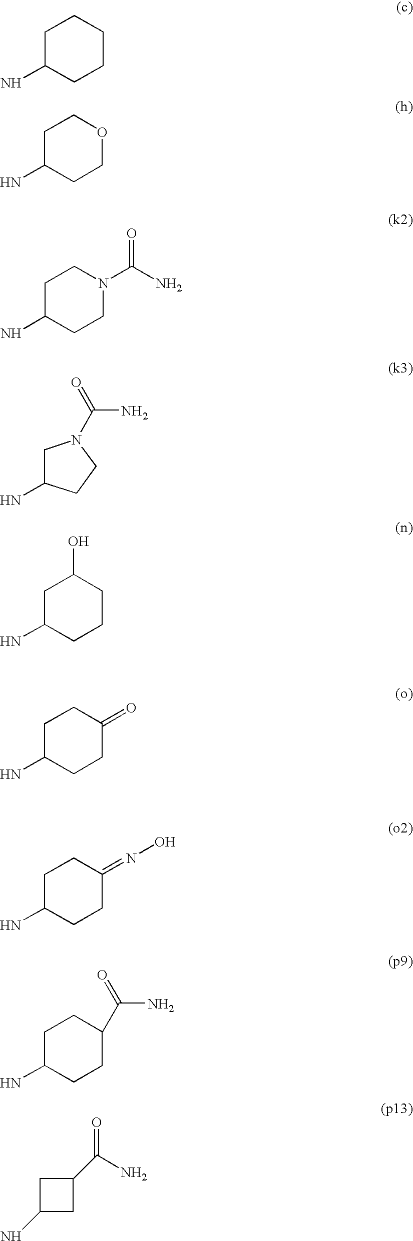

53. A compound or salt as claimed in claim 52, wherein R³ and R^{3a} are independently tetrahydro-2H-pyran-4-yl or 1-(aminocarbonyl)-4-piperidinyl.

54. A compound or a salt thereof as claimed in claim 45, wherein R⁴ and R^{4a} are hydrogen.

55. A compound or salt thereof as claimed in claim 45, wherein Q is —(CH₂)ₘ⁸- wherein m⁸ is 2, 3, 4, 5, 6, 7 or 8, or Q is —CHMe-CHMe-, or Q is —CH₂—X¹—CH₂— wherein X¹ is —CMe₂-, O, S(O)₂ or NMe (e.g. X¹ can be —CMe₂-, O or S(O)₂), or Q is 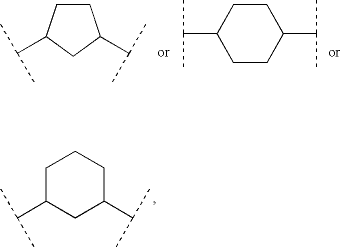
or Q has the sub-formula (q1a), (q1b), (q1), (q1), (q2a), (q2b), (q3a), (q3b), (q4), (q15), (q16), (q18a), (q18b), (q20), (q21), (q22), (q24) or (q25):

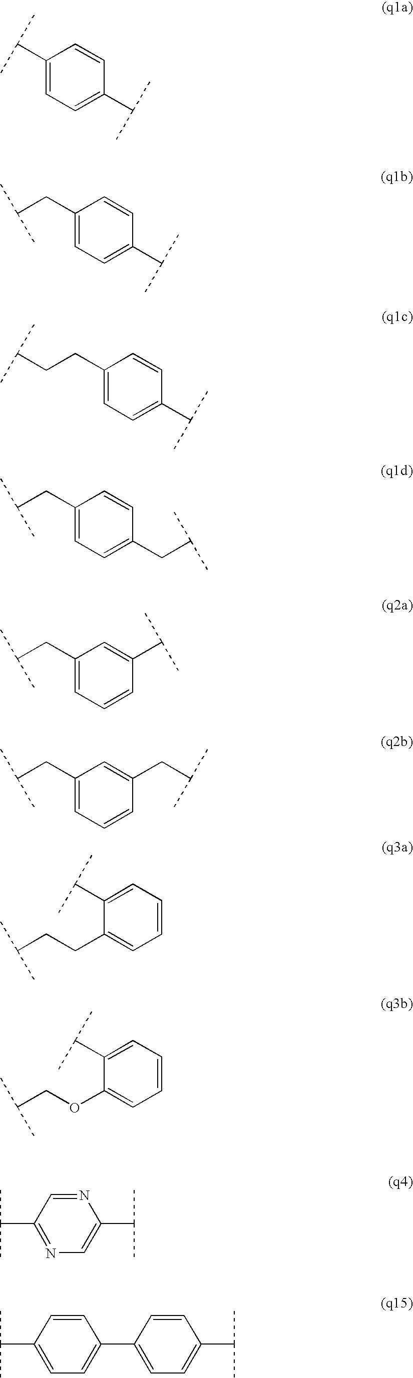

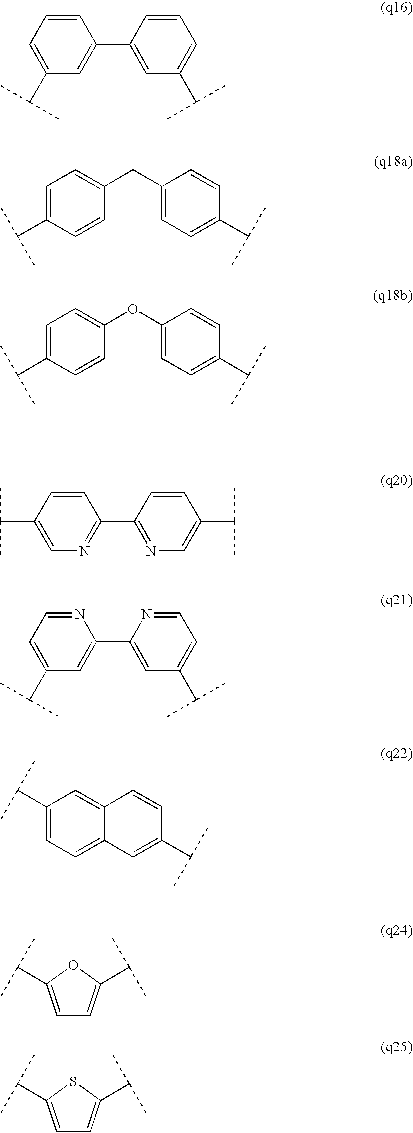

56. A compound or salt as claimed in claim 45, which is:
N-{[1,6-diethyl-4-(tetrahydro-2H-pyran-4-ylamino)-1H-pyrazolo[3,4-b]pyridin-5-yl]methyl}-4-[3-({[1,6-diethyl-4-(tetrahydro-2H-pyran-4-ylamino)-1H-pyrazolo[3,4-b]pyridin-5-yl]methyl}amino)-3-oxopropyl]benzamide,
N,N′-bis{[1,6-diethyl-4-(tetrahydro-2H-pyran-4-ylamino)-1H-pyrazolo[3,4-b]pyridin-5-yl]methyl}pentanediamide,
N,N′-bis{[1,6-diethyl-4-(tetrahydro-2H-pyran-4-ylamino)-1H-pyrazolo[3,4-b]pyridin-5-yl]methyl}pentanediamide,
N,N′-bis{[1,6-diethyl-4-(tetrahydro-2H-pyran-4-ylamino)-1H-pyrazolo[3,4-b]pyridin-5-yl]methyl}-3,3-dimethylpentanediamide,
N,N′-bis{[1,6-diethyl-4-(tetrahydro-2H-pyran-4-ylamino)-1H-pyrazolo[3,4-b]pyridin-5-yl]methyl}-2,3-dimethylbutanediamide,
4,4′-methanediylbis(N-{[1,6-diethyl-4-(tetrahydro-2H-pyran-4-ylamino)-1H-pyrazolo[3,4-b]pyridin-5-yl]methyl}benzamide),
2,2′-benzene-1,4-diylbis(N-{[1,6-diethyl-4-(tetrahydro-2H-pyran-4-ylamino)-1H-pyrazolo[3,4-b]pyridin-5-yl]methyl}acetamide),
N-{[1,6-diethyl-4-(tetrahydro-2H-pyran-4-ylamino)-1H-pyrazolo[3,4-b]pyridin-5-yl]methyl}-2-[3-({[1,6-diethyl-4-(tetrahydro-2H-pyran-4-ylamino)-1H-pyrazolo[3,4-b]pyridin-5-yl]methyl}amino)-3-oxopropyl]benzamide,
2,2′-benzene-1,3-diylbis(N-{[1,6-diethyl-4-(tetrahydro-2H-pyran-4-ylamino)-1H-pyrazolo[3,4-b]pyridin-5-yl]methyl}acetamide),
2,2′-oxybis(N-{[1,6-diethyl-4-(tetrahydro-2H-pyran-4-ylamino)-1H-pyrazolo[3,4-b]pyridin-5-yl]methyl}acetamide),
2,2′-oxybis(N-{[1,6-diethyl-4-(tetrahydro-2H-pyran-4-ylamino)-1H-pyrazolo[3,4-b]pyridin-5-yl]methyl}acetamide),
N,N′-bis{[1,6-diethyl-4-(tetrahydro-2H-pyran-4-ylamino)-1H-pyrazolo[3,4-b]pyridin-5-yl]methyl}-3,3′-biphenyldicarboxamide,
N,N′-bis[(4-{[1-(aminocarbonyl)-4-piperidinyl]amino}-1,6-diethyl-1H-pyrazolo[3,4-b]pyridin-5-yl)methyl]-1,4-cyclohexanedicarboxamide,
4,4′-{sulfonylbis[(1-oxo-2,1-ethanediyl)iminomethanediyl(1,6-diethyl-1H-pyrazolo[3,4-b]pyridine-5,4-diyl)imino]}di(1-piperidinecarboxamide),
4-({5-[({[3-({[(4-{[1-(aminocarbonyl)-4-piperidinyl]amino}-1,6-diethyl-1H-pyrazolo[3,4-b]pyridin-5-yl)methyl]amino}carbonyl)phenyl]acetyl}amino)methyl]-1,6-diethyl-1H-pyrazolo[3,4-b]pyridin-4-yl}amino)-1-piperidinecarboxamide,
4-({5-[({3-[4-({[(4-{[1-(aminocarbonyl)-4-piperidinyl]amino}-1,6-diethyl-1H-pyrazolo[3,4-b]pyridin-5-yl)methyl]amino}carbonyl)phenyl]propanoyl}amino)methyl]-1,6-diethyl-1H-pyrazolo[3,4-b]pyridin-4-yl}amino)-1-piperidinecarboxamide,
4-(5-[({[4-({[(4-{[1-(aminocarbonyl)-4-piperidinyl]amino}-1,6-diethyl-1H-pyrazolo[3,4-b]pyridin-5-yl)methyl]amino}carbonyl)phenyl]acetyl}amino)methyl]-1,6-diethyl-1H-pyrazolo[3,4-b]pyridin-4-yl}amino)-1-piperidinecarboxamide,
4-[(5-{[({[2-({[(4-{[1-(aminocarbonyl)-4-piperidinyl]amino}-1,6-diethyl-1H-pyrazolo[3,4-b]pyridin-5-yl)methyl]amino}carbonyl)phenyl]oxy}acetyl)amino]methyl}-1,6-diethyl-1H-pyrazolo[3,4-b]pyridin-4-yl)amino]-1-piperidinecarboxamide,
N,N′-bis[(4-{[1-(aminocarbonyl)-4-piperidinyl]amino}-1,6-diethyl-1H-pyrazolo[3,4-b]pyridin-5-yl)methyl]butanediamide,
4,4′-{methanediylbis[benzene-4,1-diyl(oxomethanediyl)iminomethanediyl(1,6-diethyl-1H-pyrazolo[3,4-b]pyridine-5,4-diyl)imino]}di(1-piperidinecarboxamide),
N,N′-bis[(4-{[1-(aminocarbonyl)-4-piperidinyl]amino}-1,6-diethyl-1H-pyrazolo[3,4-b]pyridin-5-yl)methyl]-3,3-dimethylpentanediamide,
N,N′-bis[(4-{[1-(aminocarbonyl)-4-piperidinyl]amino}-1,6-diethyl-1H-pyrazolo[3,4-b]pyridin-5-yl)methyl]-1,3-cyclopentanedicarboxamide,
4,4′-{oxybis[(1-oxo-2,1-ethanediyl)iminomethanediyl(1,6-diethyl-1H-pyrazolo[3,4-b]pyridine-5,4-diyl)imino]}di(1-piperidinecarboxamide),
N,N′-bis{[1,6-diethyl-4-(tetrahydro-2H-pyran-4-ylamino)-1H-pyrazolo[3,4-b]pyridin-5-yl]methyl}-1,4-benzenedicarboxamide,
N,N′-bis[(4-{[1-(aminocarbonyl)-4-piperidinyl]amino}-1,6-diethyl-1H-pyrazolo[3,4-b]pyridin-5-yl)methyl]-1,3-cyclohexanedicarboxamide,
N,N′-bis{[1,6-diethyl-4-(tetrahydro-2H-pyran-4-ylamino)-1H-pyrazolo[3,4-b]pyridin-5-yl]methyl}-4,4′-biphenyldicarboxamide,
4,4′-oxybis(N-{[1,6-diethyl-4-(tetrahydro-2H-pyran-4-ylamino)-1H-pyrazolo[3,4-b]pyridin-5-yl]methyl}benzamide),
N,N′-bis[(4-{[1-(aminocarbonyl)-4-piperidinyl]amino}-1,6-diethyl-1H-pyrazolo[3,4-b]pyridin-5-yl)methyl]-1,4-benzenedicarboxamide,
N,N′-bis[(4-{[1-(aminocarbonyl)-4-piperidinyl]amino}-1,6-diethyl-1H-pyrazolo[3,4-b]pyridin-5-yl)methyl]-4,4′-biphenyldicarboxamide,
4,4′-{oxybis[benzene-4,1-diyl(oxomethanediyl)iminomethanediyl(1,6-diethyl-1H-pyrazolo[3,4-b]pyridine-5,4-diyl)imino]}di(1-piperidinecarboxamide),
N,N′-bis{[1-ethyl-6-methyl-4-(tetrahydro-2H-pyran-4-ylamino)-1H-pyrazolo[3,4-b]pyridin-5-yl]methyl}-4,4′-biphenyldicarboxamide,
N,N′-bis{[1-ethyl-6-methyl-4-(tetrahydro-2H-pyran-4-ylamino)-1H-pyrazolo[3,4-b]pyridin-5-yl]methyl}-3,3′-biphenyldicarboxamide,
N,N′-bis{[1-ethyl-6-methyl-4-(tetrahydro-2H-pyran-4-ylamino)-1H-pyrazolo[3,4-b]pyridin-5-yl]methyl}-2,6-naphthalenedicarboxamide,
N,N′-bis{[1,6-diethyl-4-(tetrahydro-2H-pyran-4-ylamino)-1H-pyrazolo[3,4-b]pyridin-5-yl]methyl}-2,2′-bipyridine-4,4′-dicarboxamide,
N,N′-bis{[1,6-diethyl-4-(tetrahydro-2H-pyran-4-ylamino)-1H-pyrazolo[3,4-b]pyridin-5-yl]methyl}-2,5-pyrazinedicarboxamide,
2,2′-(methylimino)bis(N-{[1,6-diethyl-4-(tetrahydro-2H-pyran-4-ylamino)-1H-pyrazolo[3,4-b]pyridin-5-yl]methyl}acetamide),
N,N′-bis{[1,6-diethyl-4-(tetrahydro-2H-pyran-4-ylamino)-1H-pyrazolo[3,4-b]pyridin-5-yl]methyl}-2,2′-bipyridine-5,5′-dicarboxamide,
N,N′-bis{[1,6-diethyl-4-(tetrahydro-2H-pyran-4-ylamino)-1H-pyrazolo[3,4-b]pyridin-5-yl]methyl}-2,5furandicarboxamide, or
N,N′-bis{[1,6-diethyl-4-(tetrahydro-2H-pyran-4-ylamino)-1H-pyrazolo[3,4-b]pyridin-5-yl]methyl}-2,5-thiophenedicarboxamide;
or a pharmaceutically acceptable salt thereof

57. A method of treatment or prophylaxis of an inflammatory or allergic disease in a patient in need thereof, which method comprises administering to the patient a therapeutically effective amount of a compound of formula (I) as defined in claim 45 or a pharmaceutically acceptable salt thereof.

58. A method as claimed in claim 57, wherein the inflammatory and/or allergic disease is chronic obstructive pulmonary disease (COPD), asthma, rhinitis, atopic dermatitis or psoriasis, in the mammal.
